(19) United States
(12) Patent Application Publication
(10) Pub. No.: US 2023/0348871 A1
(43) Pub. Date: Nov. 2, 2023

(54) Targeted RNA cleavage with dCas13-RNase Fusion Proteins

(71) Applicant: University of Rochester, Rochester, NY (US)

(72) Inventor: Douglas Matthew Anderson, Pittsford, NY (US)

(21) Appl. No.: 18/005,311

(22) PCT Filed: Jul. 15, 2021
(86) PCT No.: PCT/US2021/041728

§ 371 Date: Jan. 12, 2023

Related U.S. Application Data
(60) Provisional application No. 63/052,121, filed on Jul. 15, 2020.
(60) Provisional application No. 63/052,238, filed on Jul. 15, 2020.

(51) Int. Cl. C12N 9/22; A61P 31/14; C12N 15/10

(52) U.S. Cl. CPC C12N 9/22; A61P 31/14; C12N 15/102; C12N 2310/20

(57) ABSTRACT

The present invention provides proteins, nucleic acids, systems and methods for modulating RNA. In some embodiments, the proteins, nucleic acids, systems and methods are used for targeted RNA cleavage. In other embodiments, the proteins, nucleic acids, systems and methods are used for trans-splicing of RNA molecules.

### CROSS-REFERENCE TO RELATED APPLICATIONS

This application claims priority to U.S. Provisional Patent Application No. 63/052,121, filed Jul. 15, 2020, and to U.S. Provisional Patent Application No. 63/052,238, filed Jul. 15, 2020, the contents of each of which are incorporated by reference herein in their entirety.

### REFERENCE TO A “SEQUENCE LISTING,” A TABLE, OR A COMPUTER PROGRAM LISTING APPENDIX SUBMITTED AS AN ASCII TEXT FILE

The present application hereby incorporates by reference the entire contents of the text file named “204606-0132-00WO Sequence Listing.txt” in ASCII format. The text file containing the Sequence Listing of the present application was created on Jul. 14, 2021, and is 961,054 bytes in size.

### BACKGROUND

RNA targeting, RNA-activated CRISPR-Cas13 systems are generally composed of a targeting CRISPR guide RNA (crRNA) and CRISPR associated protein, Cas13, which function as a programmable endoribonuclease (O'Connell, 2019, J Mol. Biol 431:66-87; Abudayyeh et al., 2018, Nature 550:280-84; Mohanraju et al., 2016, Science 343:aad5147). Cas13 proteins have two Higher Eukaryotes and Prokaryotes Nucleotide binding (HEPN) domains which allow for cleavage of single stranded RNA. In vitro and in bacteria, binding of a target RNA complementary to the guide-RNA results in constitutive activation of HEPN-RNase activity leading to target RNA degradation and non-specific RNA cleavage (collateral cleavage) (Abudayyeh et al., 2018, Nature 550:280-84; Abudayyeh et al., 2016, Science 343:aaf5573; Konermann et al., 2018, Cell 173:665-76). In mammalian cells, guide-RNA activation is more precise, resulting in cis-cleavage of the target RNA at multiple sites, but no detectable trans/collateral cleavage (FIG. 1A). Remarkably, catalytic inactivating mutations within the HEPN domains prevents Cas13 RNase activity, yet retains RNA targeting.

The rapid programmability, with almost no requirements for flanking crRNA sequence motifs (protospacer-flanking motifs, PFS), impart CRISPR-Cas13 with a remarkable ability for targeted RNA cleavage. However, CRISPR-Cas13 is only capable of cleaving single-stranded RNA (ssRNA) and results in complete target RNA degradation, which may not be optimal in all circumstances. Thus, there is a need in the art for compositions and methods for modulating and/or cleaving RNA.

### BRIEF DESCRIPTION OF THE DRAWINGS

FIG. 1, comprising FIG. 1A through FIG. 1C is a schematic depicting targeted RNA cleavage with dCas13-RNase fusion proteins. FIG. 1A is a schematic depicting CRISPR-Cas13 cleavage mechanisms. FIG. 1B is a schematic depicting CRISPRase cleavage mechanisms. FIG. 1C depicts experimental results demonstrating the relative activity of CRISPR-Cas13 and different CRISPRase fusion proteins targeting Luciferase mRNA in mammalian cells.

FIG. 2, comprising FIG. 2A through FIG. 2E, depicts CRISPRase fusion protein modifications. FIG. 2A through FIG. 2C are schematics depicting fusions of dCas13 to RNases with different RNA substrate specificities. FIG. 2A is a schematic depicting fusion of dCas13 to RNase with substrate specificity to ssRNA. FIG. 2B is a schematic depicting fusion of dCas13 to a RNase tandem dimer with substrate specificity to ssRNA or dsRNA. FIG. 2D and FIG. 2E are schematics depicting structural placement of RNase domains with dCas13 to specify RNA cleavage or allow for multiple cleavage sites. FIG. 2D is a schematic depicting a RNase-dCas13 fusion protein allowing for cleavage 5′ of the crRNA target site. FIG. 2E is a schematic depicting a RNase-dCas13-RNase fusion protein allowing for cleavage sites flanking the crRNA target site.

FIG. 3, comprising FIG. 3A through FIG. 3C, depicts guide-RNA modifications to prevent or enable specific CRISPRase activity. FIG. 3A is a schematic demonstrating that extending guide RNA lengths can be used to inhibit cleavage by CRISPRases specific for ssRNA, or allow for nucleotide-specific cleavage by including an unpaired bulge. FIG. 3B is a schematic demonstrating that extending guide-RNA lengths can allow for cleavage by dsRNA-specific RNases by creating dsRNA substrates, or focus cleavage by creating flanking bulges with unpaired residues. FIG. 3C is a schematic demonstrating that the addition of single or multiple DNA oligos complementary to the target RNA can allow for cleavage by RNAses which are specific for cleaving RNA in RNA:DNA hybrid substrate.

FIG. 4, comprising FIG. 4A through FIG. 4C, is a schematic depicting inducible CRISPRase activity using split RNase complementation. RNase1/RNaseA ribonucleases can be split into components S-peptide and S-protein, each possessing no separate catalytic activity, and reform in trans, regaining catalytic activity. FIG. 4A depicts the fusion of dCas13 to S-protein to allow for ‘inducible’ CRISPRase cleavage when complemented with corresponding S-peptide. Fusion of the S-peptide to small molecule-responsive protein domains, such as ERT2 and tamoxifen (tmx), can be used to create drug-activated CRISPRase cleavage systems. FIG. 4B depicts the fusion of dCas13 to S-peptide to allow for ‘inducible’ CRISPRase cleavage when complemented with corresponding S-protein. Fusion of the S-protein to small molecule-responsive protein domains, such as ERT2 and tamoxifen (tmx), can be used to create drug-activated CRISPRase cleavage systems. FIG. 4C is a schematic demonstrating fusion of dCas13 to multiple S-peptide domains in tandem which can be used to enhance CRISPRase RNA target cleavage.

FIG. 5, comprising FIG. 5A through FIG. 5F, depicts experimental results demonstrating therapeutic application of CRISPRases to both degrade toxic RNA foci and rescue host gene expression. FIG. 5A is a schematic depicting luciferase reporter genes encoding the human DMPK 3′ UTR sequence encoding either 12 or 960 copies of a CUG repeat (pGL3P-DT12a or pGL3P-DT960, respectively. FIG. 5B depicts relative luciferase activities of pGL3P-DT12a and pGL3P-DT960 luciferase reporter genes. (C) Luciferase activity of the pGL3P-DT960 reporter targeted with a CUG targeting guide RNA (CAG crRNA) by eraseR, dCas13, or CRISPRases, relative to non-targeting negative control guide RNA. (D) Number of RNA foci per cell, induced by expression of an RNA encompassing the human DMPK 3′UTR containing 960 CUG repeats, targeted by eraser or CRISPRases, with a CUG targeting guide RNA (CAG crRNA) or non-targeting negative control guide RNA (NC crRNA).

FIG. 6, comprising FIG. 6A through FIG. 6C, depicts a schematic demonstrating the use novel fusion editing proteins for the trans-splicing of RNA via targeted RNAse cleavage. Targeted RNAse cleavage, such as that performed by CRISPRases, generates unique RNA termini that may be subject to trans-RNA splicing in cells or in vitro when catalyzed by RtcB ligase. FIG. 6A depicts the use of CRISPRases targeted with multiple guide RNAs to direct the assembly of independent RNAs. FIG. 6B depicts the use of CRISPRases targeted with multiple guide RNAs to delete a sequence within a single RNA. FIG. 6C depicts the use of CRISPRases with both N and C terminal RNAse fusions for targeting via a single guide RNA to delete a specific internal RNA sequence.

### DETAILED DESCRIPTION

In one aspect, the disclosure is based on the development of novel fusion proteins which provide targeted RNA cleavage. In one embodiment, the fusion protein comprises a catalytically dead CRISPR-associated (dCas) protein and a RNase protein. These fusion proteins combine the catalytic activity of the RNase protein and the programmable DNA targeting capability of catalytically dead Cas. In one embodiment, the RNase protein is txRNase 1, RNase T1, Ribonuclease HI, PIN RNase, or RNase A. In one embodiment, the RNase is a RNase dimer. In one embodiment, the fusion protein further comprises a nuclear localization signal (NLS). In some embodiments, the fusion protein does not comprise an NLS, and is thus suitable for targeting RNA in the cytoplasm.

In one embodiment, the fusion protein comprises a catalytically dead CRISPR-associated (dCas) protein and an s-protein. The dCas-s-Protein fusion protein can be delivered with an s-peptide in trans, to provide RNase catalytic activity. Thus, in one embodiment, the disclosure provides a composition comprising a dCas-s-Protein fusion protein and an s-peptide.

In another aspect, the present invention comprises novel fusions of editing proteins, compositions thereof, and methods of use thereof for trans-splicing RNA molecules. In some embodiments, the fusion editing protein generates 2′,3′ cyclic phosphate and 5′ hydroxyl RNA termini. In one embodiment, the 2′,3′ cyclic phosphate and 5′ hydroxyl RNA termini can be ligated to one another. In one embodiment, ligation is mediated by RtcB ligase.

### Definitions

Unless defined otherwise, all technical and scientific terms used herein have the same meaning as commonly understood by one of ordinary skill in the art to which this invention belongs.

Generally, the nomenclature used herein and the laboratory procedures in cell culture, molecular genetics, organic chemistry, and nucleic acid chemistry and hybridization are those well-known and commonly employed in the art.

Standard techniques are used for nucleic acid and peptide synthesis. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, NY, and Ausubel et al., 2012, Current Protocols in Molecular Biology, John Wiley & Sons, NY), which are provided throughout this document.

The nomenclature used herein and the laboratory procedures used in analytical chemistry and organic syntheses described below are those well-known and commonly employed in the art. Standard techniques or modifications thereof are used for chemical syntheses and chemical analyses.

The term “a,” “an,” “the” and similar terms used in the context of the present invention (especially in the context of the claims) are to be construed to cover both the singular and plural unless otherwise indicated herein or clearly contradicted by the context.

“About” as used herein when referring to a measurable value such as an amount, a temporal duration, and the like, is meant to encompass variations of ±20%, or ±10%, or ±5%, or ±1%, or ±0.1% from the specified value, as such variations are appropriate to perform the disclosed methods.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The terms “patient,” “subject,” “individual,” and the like are used interchangeably herein, and refer to any animal or cell whether in vitro or in vivo, amenable to the methods described herein. In one embodiment, the subjects include vertebrates and invertebrates. Invertebrates include, but are not limited to, Drosophila melanogaster and Caenorhabditis elegans. Vertebrates include, but are not limited to, primates, rodents, domestic animals or game animals. Primates include, but are not limited to, chimpanzees, cynomologous monkeys, spider monkeys, and macaques (e.g., Rhesus). Rodents include, but are not limited to, mice, rats, woodchucks, ferrets, rabbits and hamsters. Domestic and game animals include, but are not limited to, cows, horses, pigs, deer, bison, buffalo, feline species (e.g., domestic cat), canine species (e.g., dog, fox, wolf), avian species (e.g., chicken, emu, ostrich), and fish (e.g., zebrafish, trout, catfish and salmon). In some embodiments, the subject is a mammal, e.g., a primate, e.g., a human. In certain non-limiting embodiments, the patient, subject or individual is a human.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In one embodiment, the first region comprises a first portion and the second region comprises a second portion, whereby, when the first and second portions are arranged in an antiparallel fashion, at least about 50%, at least about 75%, at least about 90%, or at least about 95% of the nucleotide residues of the first portion are capable of base pairing with nucleotide residues in the second portion. In one embodiment, all nucleotide residues of the first portion are capable of base pairing with nucleotide residues in the second portion.

The term “DNA” as used herein is defined as deoxyribonucleic acid.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

As used herein the term “wild type” is a term of the art understood by skilled persons and means the typical form of an organism, strain, gene or characteristic as it occurs in nature as distinguished from mutant or variant forms.

The term “homology” refers to a degree of complementarity. There may be partial homology or complete homology (i.e., identity). Homology is often measured using sequence analysis software (e.g., Sequence Analysis Software Package of the Genetics Computer Group. University of Wisconsin Biotechnology Center. 1710 University Avenue. Madison, Wis. 53705). Such software matches similar sequences by assigning degrees of homology to various substitutions, deletions, insertions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “RNA” as used herein is defined as ribonucleic acid.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Fusion Proteins

In one aspect, the present disclosure is based on the development of novel fusions of editing proteins and RNase proteins which provide targeted RNA cleavage. In some embodiments, the fusion proteins are effectively delivered to a cell. These fusion proteins combine the catalytic activity of the RNase protein and the programmable DNA targeting capability of catalytically dead Cas. In one embodiment, the present invention provides fusion proteins comprising a CRISPR-associated (Cas) protein, and an RNase protein. In one embodiment, the fusion protein comprises a nuclear localization signal, to target RNA in the nucleus. In one embodiments, the fusion protein does not comprise an nuclear export signal (NES), to target RNA in the cytoplasm. In other embodiments, the fusion protein does not comprise an NLS, to target RNA in the cytoplasm. Other localization signals can be used (and which are known in the art) to target RNA in organelles, such as mitochondria. In one embodiment, the fusion protein comprises a linker. In one embodiment, the linker links the Cas protein and RNase protein. In one embodiment, the fusion protein comprises a purification and/or detection tag.

In another aspect, the present invention comprises novel fusions of editing proteins, and compositions thereof, for trans-splicing RNA molecules in cells or in vitro. In one embodiment, the invention relates to a composition comprising one or more novel fusion of an editing protein or a nucleic acid encoding said novel fusion of an editing protein, as described herein, one or more targeting nucleic acid, as described herein, and one or more RNA molecules. In one embodiment, the composition further comprises RtcB ligase or nucleic acid encoding RtcB ligase.

### Editing Protein

In one embodiment, the editing protein includes, but is not limited to, a CRISPR-associated (Cas) protein, a zinc finger nuclease (ZFN) protein, and a protein having a DNA or RNA binding domain.

Non-limiting examples of Cas proteins include Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, SpCas9, StCas9, NmCas9, SaCas9, CjCas9, CjCas9, AsCpf1, LbCpf1, FnCpf1, VRER SpCas9, VQR SpCas9, xCas9 3.7, homologs thereof, orthologs thereof, or modified versions thereof. In some embodiments, the Cas protein has DNA or RNA cleavage activity. In some embodiments, the Cas protein directs cleavage of one or both strands of a nucleic acid molecule at the location of a target sequence, such as within the target sequence and/or within the complement of the target sequence. In some embodiments, the Cas protein directs cleavage of one or both strands within about 1, 2, 3, 4, 5, 6, 7, 8, 9, 10, 15, 20, 25, 50, 100, 200, 500, or more base pairs from the first or last nucleotide of a target sequence. In one embodiment, the Cas protein is Cas9, Cas13, or Cpf1. In one embodiment, Cas protein is catalytically deficient (dCas).

In one embodiment, the Cas protein has RNA binding activity. In one embodiment, Cas protein is Cas13. In one embodiment, the Cas protein is PspCas13b, PspCas13b Truncation, AdmCas13d, AspCas13b, AspCas13c, BmaCas13a, BzoCas13b, CamCas13a, CcaCas13b, Cga2Cas13a, CgaCas13a, EbaCas13a, EreCas13a, EsCas13d, FbrCas13b, FnbCas13c, FndCas13c, FnfCas13c, FnsCas13c, FpeCas13c, FulCas13c, HheCas13a, LbfCas13a, LbmCas13a, LbnCas13a, LbuCas13a, LseCas13a, LshCas13a, LspCas13a, Lwa2cas13a, LwaCas13a, LweCas13a, PauCas13b, PbuCas13b, PgiCas13b, PguCas13b, Pin2Cas13b, Pin3Cas13b, PinCas13b, Pprcas13a, PsaCas13b, PsmCas13b, RaCas13d, RanCas13b, RcdCas13a, RcrCas13a, RcsCas13a, RfxCas13d, UrCas13d, dPspCas13b, PspCas13b_A133H, PspCas13b_A1058H, dPspCas13b truncation, dAdmCas13d, dAspCas13b, dAspCas13c, dBmaCas13a, dBzoCas13b, dCamCas13a, dCcaCas13b, dCga2Cas13a, dCgaCas13a, dEbaCas13a, dEreCas13a, dEsCas13d, dFbrCas13b, dFnbCas13c, dFndCas13c, dFnfCas13c, dFnsCas13c, dFpeCas13c, dFulCas13c, dHheCas13a, dLbfCas13a, dLbmCas13a, dLbnCas13a, dLbuCas13a, dLseCas13a, dLshCas13a, dLspCas13a, dLwa2cas13a, dLwaCas13a, dLweCas13a, dPauCas13b, dPbuCas13b, dPgiCas13b, dPguCas13b, dPin2Cas13b, dPin3Cas13b, dPinCas13b, dPprCas13a, dPsaCas13b, dPsmCas13b, dRaCas13d, dRanCas13b, dRcdCas13a, dRcrCas13a, dRcsCas13a, dRfxCas13d, dUrCas13d, dCas13X.1, or mini-dCas13X.1. Additional Cas proteins are known in the art (e.g., Konermann et al., Cell, 2018, 173:665-676 e14, Yan et al., Mol Cell, 2018, 7:327-339 e5; Cox, D. B. T., et al., Science, 2017, 358: 1019-1027; Abudayyeh et al., Nature, 2017, 550: 280-284, Gootenberg et al., Science, 2017, 356: 438-442; and East-Seletsky et al., Mol Cell, 2017, 66: 373-383 e3, which are herein incorporated by reference).

In one embodiment, the Cas protein comprises a sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs:1-48 and 826. In one embodiment, the Cas protein comprises a sequence of a variant of one of SEQ ID NOs: 1-48 and 826, wherein the variant renders the Cas protein catalytically inactive. In one embodiment, the Cas protein comprises a sequence of one of SEQ ID NOs: 1-46 and 826 having one or more insertions, deletions or substitutions, wherein the one or more insertions, deletions or substitutions renders the Cas protein catalytically inactive. In one embodiment, the Cas protein comprises a sequence of one of SEQ ID NOs:1-48 and 826. In one embodiment, the Cas protein comprises a sequence of one of SEQ ID NOs:47-48.

### RNase

In one embodiment, the fusion protein comprises an RNase protein. In one embodiment, the fusion protein comprises two RNase proteins. In one embodiment, the fusion protein comprises three or more RNase proteins. In one embodiment, the fusion protein comprises two identical RNase proteins. In one embodiment, the fusion protein comprises three or more identical RNase proteins. In one embodiment, the fusion protein comprises two different RNase proteins. In one embodiment, the fusion protein comprises three or more different RNase proteins.

In one embodiment, the RNase protein is heterologous to the Cas protein. In one embodiment, the RNase is capable of cleaving a phosphodiester bond within a polynucleotide chain. In one embodiment, the RNase is capable of cleavage of one or more of single-stranded RNA (ssRNA), double-stranded RNA (dsRNA), or RNA in a hybrid RNA:DNA complex. In one embodiment, the RNase comprises sequence specific cleavage activity. In various embodiments, the RNase is an endonuclease.

In one embodiment, the RNase is RNase 1, RNase 2, RNase 3, RNase 4, RNase 5, RNase 6, RNase 7, RNase 8, RNase A, RNase 1, RNase 1B, txRNase 1 (RNase 1 R39D/N67D/N88A/G89D/R91D), txRNase A (RNase A D38R/R39D/N67R/G88R), RNase T1, RNase T2, Onconase, Erns(C171R), RNase U2, PIN RNase domain, Bovine seminal ribonuclease (SRN), RNase V1, Mini RNase III (MiniR3), RNase III Domain (DICER), Ribonuclease HI (RNase HI*), or Ribonuclease HI(D125N)(RNase HI*D125N).

In one embodiment, the RNase protein comprises a sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs:49-89. In one embodiment, the RNase protein comprises a sequence of one of SEQ ID NOs: 49-89.

In one embodiment, the RNase is a dimer of RNase monomers. In one embodiment, the RNase dimer is capable of cleaving dsRNA or ssRNA. In one embodiment, the RNase dimer is linked together with a linking sequence. In one embodiment, the RNase dimer is a homodimer. In one embodiment, the RNase dimer is a heterodimer. In one embodiment, the RNase dimer is a natural dimer. In one embodiment, the RNase dimer is a synthetic dimer. In one embodiment, the RNase dimer is Synthetic Tandem Dimer RNase 1 (tdRNase 1), Synthetic tandem PIN RNase domain (tdPIN), Synthetic Tandem Dimer Bovine seminal ribonuclease (tdSRN), Synthetic Tandem Dimer Mini RNase III (tdMiniR3), Synthetic Tandem Dimer RNase III Domain (tdDICER), Synthetic tandem RNase III domain (tdRNC), Natural tandem RNase III domain (DROSHA), or Natural tandem RNase III domain Dimer (giDICER). In one embodiment, the RNase dimer comprises a sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 57, 77, 79, 82, and 84-87. In one embodiment, the RNase dimer comprises a sequence of one of SEQ ID NOs: 57, 77, 79, 82, and 84-87.

In one embodiment, the RNase protein is a fragment of a RNase protein. In one embodiment, the fragment of the RNase protein is capable of being complemented with a second fragment of the RNase protein in trans providing inducible catalytic activity. In one embodiment, the fragment of the RNase protein is a fragment of RNase is RNase 1, RNase 2, RNase 3, RNase 4, RNase 5, RNase 6, RNase 7, RNase 8, RNase A, RNase 1, RNase 1B, txRNase 1 (RNase 1 R39D/N67D/N88A/G89D/R91D), txRNase A (RNase A D38R/R39D/N67R/G88R), RNase T1, RNase T2, Onconase, Erns(C171R), RNase U2, PIN RNase domain, Bovine seminal ribonuclease (SRN), RNase V1, Mini RNase III (MiniR3), RNase III Domain (DICER), Ribonuclease HI (RNase HI*), or Ribonuclease HI(D125N)(RNase HI*D125N).

In one embodiment, the fragment of the RNase protein is a fragment of RNase1. In one embodiment, the fragment of the RNase protein is an s-protein. In one embodiment, the s-protein comprises a sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 90, 93, and 96. In one embodiment, the s-protein comprises a sequence of one of SEQ ID NOs: 90, 93, and 96.

In one embodiment, the fragment of the RNase protein is an s-peptide. In one embodiment, the s-peptide comprises a sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 91, 92, 94, 95, and 97-99. In one embodiment, the s-peptide comprises a sequence of one of SEQ ID NOs: 91, 92, 94, 95, and 97-99.

### Localization Signals

In some embodiments, the fusion protein may contain a localization signal, such as an nuclear localization signal (NLS), nuclear export signal (NES) or other localization signals to localize to organelles, such as mitochondria. In one embodiment, the localization signal localizes the fusion protein to the site in which the target RNA is located.

Nuclear Localization Signal

In one embodiment, the fusion protein comprises a NLS. In one embodiment, the NLS is a retrotransposon NLS. In one embodiment, the NLS is derived from Ty1, yeast GAL4, SKI3, L29 or histone H2B proteins, polyoma virus large T protein, VP1 or VP2 capsid protein, SV40 VP1 or VP2 capsid protein, Adenovirus El a or DBP protein, influenza virus NS1 protein, hepatitis vims core antigen or the mammalian lamin, c-myc, max, c-myb, p53, c-erbA, jun, Tax, steroid receptor or Mx proteins, Nucleoplasmin (NPM2), Nucleophosmin (NPM1), or simian vims 40 (“SV40”) T-antigen. In one embodiment, the NLS is a Ty1 or Ty1-derived NLS, a Ty2 or Ty2-derived NLS or a MAK11 or MAK11-derived NLS. In one embodiment, the Ty1 NLS comprises an amino acid sequence of SEQ ID NO:110. In one embodiment, the Ty2 NLS comprises an amino acid sequence of SEQ ID NO:111. In one embodiment, the MAK11 NLS comprises an amino acid sequence of SEQ ID NO:112. In one embodiment, the NLS comprises a sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs:110-730. In one embodiment, the NLS protein comprises a sequence of one of SEQ ID NOs: 110-730.

In one embodiment, the NLS is a Ty1-like NLS. For example, in one embodiment, the Ty1-like NLS comprises KKRX motif. In one embodiment, the Ty1-like NLS comprises KKRX motif at the N-terminal end. In one embodiment, the Ty1-like NLS comprises KKR motif. In one embodiment, the Ty1-like NLS comprises KKR motif at the C-terminal end. In one embodiment, the Ty1-like NLS comprises a KKRX and a KKR motif. In one embodiment, the Ty1-like NLS comprises a KKRX at the N-terminal end and a KKR motif at the C-terminal end. In one embodiment, the Ty1-like NLS comprises at least 20 amino acids. In one embodiment, the Ty1-like NLS comprises between 20 and 40 amino acids. In one embodiment, the Ty1-like NLS comprises a sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs:118-730. In one embodiment, the NLS comprises a sequence of one of SEQ ID NOs:118-730, wherein the sequence comprises one or more, two or more, three or more, four or more, five or more, six or more, seven or more, eight or more, nine or more, or ten or more, insertions, deletions or substitutions. In one embodiment, the Ty1-like NLS protein comprises a sequence of one of SEQ ID NOs:118-730.

In one embodiment, the NLS comprises two copies of the same NLS. For example, in one embodiment, the NLS comprises a multimer of a first Ty1-derived NLS and a second Ty1-derived NLS.

Nuclear Export Signal

In one embodiment, the fusion protein comprises a Nuclear Export Signal (NES). In one embodiment, the NES is attached to the N-terminal end of the Cas protein. In one embodiment, the NES localizes the fusion protein to the cytoplasm for targeting cytoplasmic RNA. In one embodiment, the NES comprises an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO:802 or 803. In one embodiment, the NES comprises an amino acid sequence of SEQ ID NO: 802 or 803.

Organelle Localization Signal

In one embodiment, the fusion protein comprises a localization signal that localizes the fusion protein to an organelle. In one embodiment, the localization signal localizes the protein to the nucleolus, ribosome, vesicle, rough endoplasmic reticulum, Golgi apparatus, cytoskeleton, smooth endoplasmic reticulum, mitochondria, vacuole, cytosol, lysosome, or centriole. A number of localization signals are known in the art.

In one embodiment, the fusion protein comprises a localization signal that localizes the fusion protein to an organelle or extracellularly. In one embodiment, the localization signal localizes the protein to the nucleolus, ribosome, vesicle, rough endoplasmic reticulum, Golgi apparatus, cytoskeleton, smooth endoplasmic reticulum, mitochondria, vacuole, cytosol, lysosome, or centriole.

A number of localization signals are known in the art. Exemplary localization signals include, but are not limited to 1x mitochondrial targeting sequence, 4x mitochondrial targeting sequence, secretory signal sequence (IL-2), myristylation, Calsequestrin leader, KDEL retention and peroxisome targeting sequence.

In one embodiment, the fusion protein comprises sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO:806-812. In one embodiment, the fusion protein comprises sequence of SEQ ID NO: 806-812.

### Linker

In one embodiment, the fusion protein comprises a linker. In one embodiment, the linker links the Cas protein and RNase protein. In one embodiment, the linker is connected to the C-terminal end of the Cas protein and to the N-terminal end of the RNase protein. In one embodiment, the linker is connected to the N-terminal end of the Cas protein and to the C-terminal end of the RNase protein. Linkers can be flexible linkers, such as linkers composed predominately of Gly and Ser amino acid residues, or more rigid linkers, which may include amino acids such as Ala and Pro (among others). In one embodiment, the linker comprises a sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs:100-108. In one embodiment, the linker comprises a sequence at of one of SEQ ID NOs: 100-108.

Purification and/or Detection Tag

In one embodiment, the fusion protein comprises a purification and/or detection tag. In one embodiment, the tag is on the N-terminal end of the fusion protein. In one embodiment, the tag is a 3×FLAG tag. In one embodiment, the tag comprises an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO:109. In one embodiment, the tag comprises an amino acid sequence of SEQ ID NO:109.

### Proteins, Peptides and Fusion Proteins

The proteins of the present disclosure may be made using chemical methods. For example, protein can be synthesized by solid phase techniques (Roberge J Y et al (1995) Science 269: 202-204), cleaved from the resin, and purified by preparative high-performance liquid chromatography. Automated synthesis may be achieved, for example, using the ABI 431 A Peptide Synthesizer (Perkin Elmer) in accordance with the instructions provided by the manufacturer.

The proteins of the present disclosure may be made using recombinant protein expression. The recombinant expression vectors of the disclosure comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequences in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for production of variant proteins in prokaryotic or eukaryotic cells. For example, proteins of the invention can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, to the amino or C terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin, PreScission, TEV and enterokinase. Typical fusion expression vectors include pGEX (Pharmacia Biotech Inc; Smith and Johnson, 1988. Gene 67: 31-40), pMAL (New England Biolabs, Beverly, Mass.) and pRITS (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., Gene Expression Technology: Methods in Enzymology 185, Academic Press, San Diego, Calif. (1990) 60-89)—not accurate, pET11a-d have N terminal T7 tag.

One strategy to maximize recombinant protein expression in E. coli is to express the protein in a host bacterium with an impaired capacity to proteolytically cleave the recombinant protein. See, e.g., Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques. Another strategy to solve codon bias is by using BL21-codon plus bacterial strains (Invitrogen) or Rosetta bacterial strain (Novagen), these strains contain extra copies of rare E. coli tRNA genes.

In another embodiment, the expression vector encoding for the protein of the disclosure is a yeast expression vector. Examples of vectors for expression in yeast Saccharomyces cerevisiae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, polypeptides of the present invention can be produced in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the disclosure is expressed in mammalian cells using a mammalian expression vector. Mammalian cell lines available in the art for expression of a heterologous polypeptide include Chinese hamster ovary (CHO) cells, HeLa cells, baby hamster kidney cells, NSO mouse melanoma cells, YB2/0 rat myeloma cells, human embryonic kidney cells, human embryonic retina cells and many others. Examples of mammalian expression vectors include pCDM8 (Seed, 1987. Nature 329: 840) and pMT2PC (Kaufman, et al., 1987. EMBO J. 6: 187-195), pIRESpuro (Clontech), pUB6 (Invitrogen), pCEP4 (Invitrogen) pREP4 (Invitrogen), pcDNA3 (Invitrogen). When used in mammalian cells, the expression vector's control functions are often provided by viral regulatory elements. For example, commonly used promoters are derived from polyoma, adenovirus 2, cytomegalovirus, Rous Sarcoma Virus, and simian virus 40. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murinehox promoters (Kessel and Gruss, 1990. Science 249: 374-379) and the alpha-fetoprotein promoter (Campes and Tilghman, 1989. Genes Dev. 3: 537-546).

The invention should also be construed to include any form of a protein having substantial homology to a protein disclosed herein. In one embodiment, a protein which is “substantially homologous” is about 50% homologous, about 70% homologous, about 80% homologous, about 90% homologous, about 91% homologous, about 92% homologous, about 93% homologous, about 94% homologous, about 95% homologous, about 96% homologous, about 97% homologous, about 98% homologous, or about 99% homologous to amino acid sequence of a fusion-protein disclosed herein.

The protein may alternatively be made by recombinant means or by cleavage from a longer polypeptide. The composition of a protein may be confirmed by amino acid analysis or sequencing.

The variants of the protein according to the present invention may be (i) one in which one or more of the amino acid residues are substituted with a conserved or non-conserved amino acid residue and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the protein of the present invention, (iv) fragments of the peptides and/or (v) one in which the protein is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two fusion proteins is determined by comparing the amino acid sequence and its conserved amino acid substitutes of one polypeptide to a sequence of a second polypeptide. Variants are defined to include peptide sequences different from the original sequence. In one embodiment, variants are different from the original sequence in less than 40% of residues per segment of interest different from the original sequence in less than 25% of residues per segment of interest, different by less than 10% of residues per segment of interest, or different from the original protein sequence in just a few residues per segment of interest and at the same time sufficiently homologous to the original sequence to preserve the functionality of the original sequence and/or the ability to stimulate the differentiation of a stem cell into the osteoblast lineage. The present invention includes amino acid sequences that are at least 60%, 65%, 70%, 72%, 74%, 76%, 78%, 80%, 90%, 91%, 92%, 93%, 94%, 95%, 96%, 97%, 98%, or 99% similar or identical to the original amino acid sequence. The degree of identity between two peptides is determined using computer algorithms and methods that are widely known for the persons skilled in the art. The identity between two amino acid sequences may be determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The protein of the disclosure can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The protein of the disclosure may include unnatural amino acids formed by post-translational modification or by introducing unnatural amino acids during translation. A variety of approaches are available for introducing unnatural amino acids during protein translation.

A protein of the disclosure may be phosphorylated using conventional methods such as the method described in Reedijk et al. (The EMBO Journal 11(4):1365, 1992).

Cyclic derivatives of the fusion proteins of the invention are also part of the present invention. Cyclization may allow the protein to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic protein which is more flexible than the cyclic peptides containing peptide bond linkages as described above. A more flexible peptide may be prepared by introducing cysteines at the right and left position of the peptide and forming a disulfide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The invention also relates to peptides comprising a fusion protein comprising Cas13 and a RNase protein, wherein the fusion protein is itself fused to, or integrated into, a target protein, and/or a targeting domain capable of directing the chimeric protein to a desired cellular component or cell type or tissue. The chimeric proteins may also contain additional amino acid sequences or domains. The chimeric proteins are recombinant in the sense that the various components are from different sources, and as such are not found together in nature (i.e., are heterologous).

In one embodiment, the targeting domain can be a membrane spanning domain, a membrane binding domain, or a sequence directing the protein to associate with for example vesicles or with the nucleus. In one embodiment, the targeting domain can target a peptide to a particular cell type or tissue. For example, the targeting domain can be a cell surface ligand or an antibody against cell surface antigens of a target tissue. A targeting domain may target the peptide of the invention to a cellular component.

A peptide of the invention may be synthesized by conventional techniques. For example, the peptides or chimeric proteins may be synthesized by chemical synthesis using solid phase peptide synthesis. These methods employ either solid or solution phase synthesis methods (see for example, J. M. Stewart, and J. D. Young, Solid Phase Peptide Synthesis, 2nd Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis). By way of example, a peptide of the invention may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide or chimeric protein of the invention conjugated with other molecules may be prepared by fusing, through recombinant techniques, the N-terminal or C-terminal of the peptide or chimeric protein, and the sequence of a selected protein or selectable marker with a desired biological function. The resultant fusion proteins contain the protein fused to the selected protein or marker protein as described herein. Examples of proteins which may be used to prepare fusion proteins include immunoglobulins, glutathione-S-transferase (GST), hemagglutinin (HA), and truncated myc.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptides and chimeric proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

In one embodiment, the fusion protein comprises an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 827, C-terminally linked to an RNase as described above. In one embodiment, the fusion protein comprises the amino acid sequence of SEQ ID NO: 827, C-terminally linked to an RNase.

In one embodiment, the fusion protein comprises an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 828, N-terminally linked to a first RNAase, and C-terminally linked to a second RNase. In one embodiment, the fusion protein comprises the amino acid sequence of SEQ ID NO: 828, N-terminally linked to a first RNAase, and C-terminally linked to a second RNase.

### Proteins for Trans-Splicing of RNA Molecules

In some embodiments, the present invention relates to novel fusions of editing proteins for trans-splicing RNA molecules in cells or in vitro. In one embodiment, the present invention comprises a composition comprising one or more novel fusion of an editing protein, as described herein, one or more targeting nucleic acid, as described herein, and one or more RNA molecule. In some embodiments, said one or more RNA molecule comprises a single RNA molecule. In some embodiments, said one or more RNA molecule comprises at least two RNA molecules. In one embodiment, the composition further comprises RtcB ligase or a nucleic acid encoding RtcB ligase. Exemplary RtcB ligase proteins and their corresponding amino acid sequences can be found in International Application No. PCT/US2021/016885 (incorporated by reference herein in its entirety).

### Nucleic Acids

In one aspect, the present disclosure provides nucleic acids encoding fusion proteins of the disclosure. In one embodiment, the nucleic acids encoding a fusion protein comprising an editing protein and a RNase protein. In one embodiment, the nucleic acids encoding a fusion protein comprising a Cas and a RNase protein. In one embodiment, the fusion proteins combine the catalytic activity of the RNase protein and the programmable nucleic acid targeting capability of catalytically dead Cas.

The present disclosure also provides targeting nucleic acids, including CRISPR RNAs (crRNAs), for targeting the fusion protein of the disclosure to a target RNA. In one embodiment, the crRNA is selected based on the RNase activity of the fusion protein. For example, in one the RNase of the fusion protein may be capable of cleaving one or more of ssRNA, dsRNA, or RNA:DNA complexes. Thus, the present disclosure provides crRNA allowing for targeted cleavage of ssRNA, dsRNA, or RNA:DNA complexes to be used with the fusion proteins of the disclosure.

### Nucleic Acids Encoding Fusion Proteins

In one aspect, the present disclosure is based on the development of novel nucleic acid molecules encoding fusions of editing proteins and RNase proteins, which are effectively delivered to the cell and provide targeted RNA cleavage. These fusion proteins combine the catalytic activity of the RNase protein and the programmable nucleic acid targeting capability of catalytically dead Cas. In one embodiment, nucleic acid molecule comprises a nucleic acid sequence encoding an editing protein; and a nucleic acid sequence encoding a RNase protein. In one embodiment, the nucleic acid molecule comprises a nucleic acid sequence encoding a localization signal (e.g., an NLS). In one embodiment, the nucleic acid molecule comprises a nucleic acid sequence encoding a linker. In one embodiment, the nucleic acid molecule comprises a nucleic acid sequence encoding a purification and/or detection tag.

Editing Protein

In one embodiment, the nucleic acid molecule comprises a sequence nucleic acid encoding an editing protein. In one embodiment, the editing protein includes, but is not limited to, a CRISPR-associated (Cas) protein, a zinc finger nuclease (ZFN) protein, and a protein having a DNA or RNA binding domain.

Non-limiting examples of Cas proteins include Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, SpCas9, StCas9, NmCas9, SaCas9, CjCas9, CjCas9, AsCpf1, LbCpf1, FnCpf1, VRER SpCas9, VQR SpCas9, xCas9 3.7, homologs thereof, orthologs thereof, or modified versions thereof. In some embodiments, the Cas protein has DNA or RNA cleavage activity. In some embodiments, the Cas protein directs cleavage of one or both strands of a nucleic acid molecule at the location of a target sequence, such as within the target sequence and/or within the complement of the target sequence. In some embodiments, the Cas protein directs cleavage of one or both strands within about 1, 2, 3, 4, 5, 6, 7, 8, 9, 10, 15, 20, 25, 50, 100, 200, 500, or more base pairs from the first or last nucleotide of a target sequence. In one embodiment, the Cas protein is Cas9, Cas13, or Cpf1. In one embodiment, Cas protein is catalytically deficient (dCas).

In one embodiment, the Cas protein has RNA binding activity. In one embodiment, Cas protein is Cas13. In one embodiment, the Cas protein is PspCas13b, PspCas13b Truncation, AdmCas13d, AspCas13b, AspCas13c, BmaCas13a, BzoCas13b, CamCas13a, CcaCas13b, Cga2Cas13a, CgaCas13a, EbaCas13a, EreCas13a, EsCas13d, FbrCas13b, FnbCas13c, FndCas13c, FnfCas13c, FnsCas13c, FpeCas13c, FulCas13c, HheCas13a, LbfCas13a, LbmCas13a, LbnCas13a, LbuCas13a, LseCas13a, LshCas13a, LspCas13a, Lwa2cas13a, LwaCas13a, LweCas13a, PauCas13b, PbuCas13b, PgiCas13b, PguCas13b, Pin2Cas13b, Pin3Cas13b, PinCas13b, Pprcas13a, PsaCas13b, PsmCas13b, RaCas13d, RanCas13b, RcdCas13a, RcrCas13a, RcsCas13a, RfxCas13d, UrCas13d, dPspCas13b, PspCas13b_A133H, PspCas13b_A1058H, dPspCas13b truncation, dAdmCas13d, dAspCas13b, dAspCas13c, dBmaCas13a, dBzoCas13b, dCamCas13a, dCcaCas13b, dCga2Cas13a, dCgaCas13a, dEbaCas13a, dEreCas13a, dEsCas13d, dFbrCas13b, dFnbCas13c, dFndCas13c, dFnfCas13c, dFnsCas13c, dFpeCas13c, dFulCas13c, dHheCas13a, dLbfCas13a, dLbmCas13a, dLbnCas13a, dLbuCas13a, dLseCas13a, dLshCas13a, dLspCas13a, dLwa2cas13a, dLwaCas13a, dLweCas13a, dPauCas13b, dPbuCas13b, dPgiCas13b, dPguCas13b, dPin2Cas13b, dPin3Cas13b, dPinCas13b, dPprCas13a, dPsaCas13b, dPsmCas13b, dRaCas13d, dRanCas13b, dRcdCas13a, dRcrCas13a, dRcsCas13a, dRfxCas13d, dUrCas13d, dCas13X.1, or mini-dCas13X.1. Additional Cas proteins are known in the art (e.g., Konermann et al., Cell, 2018, 173:665-676 e14, Yan et al., Mol Cell, 2018, 7:327-339 e5; Cox, D. B. T., et al., Science, 2017, 358: 1019-1027; Abudayyeh et al., Nature, 2017, 550: 280-284, Gootenberg et al., Science, 2017, 356: 438-442; and East-Seletsky et al., Mol Cell, 2017, 66: 373-383 e3, which are herein incorporated by reference).

In one embodiment, the nucleic acid sequence encoding a Cas protein comprises a nucleic acid sequence encoding an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 1-48 and 826. In one embodiment, the nucleic acid sequence encoding a Cas protein comprises a nucleic acid sequence encoding an amino acid sequence of a variant of one of SEQ ID NOs:1-48 and 826, wherein the variant renders the Cas protein catalytically inactive. In one embodiment, the nucleic acid sequence encoding a Cas protein comprises a nucleic acid sequence encoding an amino acid sequence of one of SEQ ID NOs:1-46 and 826 having one or more insertions, deletions or substitutions, wherein the one or more insertions, deletions or substitutions renders the Cas protein catalytically inactive. In one embodiment, the nucleic acid sequence encoding a Cas protein comprises a nucleic acid sequence encoding an amino acid sequence of one of SEQ ID NOs:1-48 and 826. In one embodiment, the nucleic acid sequence encoding a Cas protein comprises a nucleic acid sequence encoding an amino acid sequence of one of SEQ ID NOs:47-48.

In one embodiment, the nucleic acid sequence encoding a Cas protein comprises a nucleic acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 734-737 and 823. In one embodiment, the nucleic acid sequence encoding a Cas protein comprises a of a variant of one of SEQ ID NOs: 734-735 and 823, wherein the variant renders the encoded Cas protein catalytically inactive. In one embodiment, the nucleic acid sequence encoding a Cas protein comprises a nucleic acid sequence of one of SEQ ID NOs: 734-735 and 823. In one embodiment, the nucleic acid sequence encoding a Cas protein comprises a nucleic acid sequence of one of SEQ ID NOs:736-737.

RNase

In one embodiment, the nucleic acid molecule comprises a nucleic acid sequence encoding an RNase protein. In one embodiment, the nucleic acid molecule encodes two RNase proteins. In one embodiment, the nucleic acid molecule encodes three or more RNase proteins. In one embodiment, the nucleic acid molecule encodes two identical RNase proteins. In one embodiment, the nucleic acid molecule encodes three or more identical RNase proteins. In one embodiment, the nucleic acid molecule encodes two different RNase proteins. In one embodiment, the nucleic acid molecule encodes three or more different RNase proteins.

In one embodiment, the RNase protein is heterologous to the Cas protein. In one embodiment, the RNase is capable of cleaving a phosphodiester bond within a polynucleotide chain. In one embodiment, the RNase is capable of cleavage of one or more of single-stranded RNA (ssRNA), double-stranded RNA (dsRNA), or RNA in a hybrid RNA:DNA complex. In one embodiment, the RNase comprises sequence specific cleavage activity.

In one embodiment, the RNase is RNase 1, RNase 2, RNase 3, RNase 4, RNase 5, RNase 6, RNase 7, RNase 8, RNase A, RNase 1, RNase 1B, txRNase 1 (RNase 1 R39D/N67D/N88A/G89D/R91D), txRNase A (RNase A D38R/R39D/N67R/G88R), RNase T1, RNase T2, Onconase, Erns(C171R), RNase U2, PIN RNase domain, Bovine seminal ribonuclease (SRN), RNase V1, Mini RNase III (MiniR3), RNase III Domain (DICER), Ribonuclease HI (RNase HI*), or Ribonuclease HI(D125N)(RNase HI*D125N).

In one embodiment, the nucleic acid molecule comprises a nucleic acid sequence encoding an RNase having an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs:49-89. In one embodiment, the nucleic acid molecule comprises a nucleic acid sequence encoding an RNase having an amino acid sequence of one of SEQ ID NOs: 49-89.

In one embodiment, the nucleic acid sequence encoding a RNase protein comprises a nucleic acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 738-779. In one embodiment, the nucleic acid sequence encoding a RNase protein comprises a nucleic acid sequence of one of SEQ ID NOs: 738-779.

In one embodiment, the nucleic acid molecule encodes a dimer of RNase monomers. In one embodiment, the RNase dimer is linked together with a linking sequence. In one embodiment, the RNase dimer is a homodimer. In one embodiment, the RNase dimer is a heterodimer. In one embodiment, the RNase dimer is a natural dimer. In one embodiment, the RNase dimer is a synthetic dimer. In one embodiment, the RNase dimer is Synthetic Tandem Dimer RNase 1 (tdRNase 1), Synthetic tandem PIN RNase domain (tdPIN), Synthetic Tandem Dimer Bovine seminal ribonuclease (tdSRN), Synthetic Tandem Dimer Mini RNase III (tdMiniR3), Synthetic Tandem Dimer RNase III Domain (tdDICER), Synthetic tandem RNase III domain (tdRNC), Natural tandem RNase III domain (DROSHA), or Natural tandem RNase III domain Dimer (giDICER).

In one embodiment, the nucleic acid molecule comprises a nucleic acid sequence encoding an RNase dimer comprising an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 57, 77, 79, 82, and 84-87. In one embodiment, the nucleic acid molecule comprises a nucleic acid sequence encoding an RNase dimer comprising an amino acid of one of SEQ ID NOs: 57, 77, 79, 82, and 84-87.

In one embodiment, the nucleic acid sequence encoding an RNase dimer comprises a nucleic acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 746, 767, 769, 772, and 774-777. In one embodiment, the nucleic acid sequence encoding an RNase dimer comprises a nucleic acid sequence of one of SEQ ID NOs: 746, 767, 769, 772, and 774-777.

In one embodiment, the nucleic acid molecule encodes a fragment of a RNase protein. In one embodiment, the fragment of the RNase protein is capable of being complemented with a second fragment of the RNase protein in trans providing inducible catalytic activity. In one embodiment, the fragment of the RNase protein is a fragment of RNase is RNase 1, RNase 2, RNase 3, RNase 4, RNase 5, RNase 6, RNase 7, RNase 8, RNase A, RNase 1, RNase 1B, txRNase 1 (RNase 1 R39DN67D/N88A/G89D/R91D), txRNase A (RNase A D38R/R39D/N67R/G88R), RNase T1, RNase T2, Onconase, Erns(C171R), RNase U2, PIN RNase domain, Bovine seminal ribonuclease (SRN), RNase V1, Mini RNase III (MiniR3), RNase III Domain (DICER), Ribonuclease HI (RNase HI*), or Ribonuclease HI(D125N)(RNase HI*D125N).

In one embodiment, the nucleic acid molecule encodes a fragment of RNase1. In one embodiment, the nucleic acid molecule encodes an s-protein. In one embodiment, the nucleic acid molecule comprises a nucleic acid sequence encoding s-protein comprising an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 90, 93, and 96. In one embodiment, the nucleic acid molecule comprises a nucleic acid sequence encoding s-protein comprising an amino acid sequence of one of SEQ ID NOs: 90, 93, and 96.

In one embodiment, the nucleic acid sequence encoding an s-protein comprises a nucleic acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 780, 783, and 786. In one embodiment, the nucleic acid sequence encoding an s-protein comprises a nucleic acid sequence of one of SEQ ID NOs: 780, 783, and 786.

In one embodiment, the fragment of the RNase protein is an s-peptide. In one embodiment, the nucleic acid molecule comprises a nucleic acid sequence encoding s-peptide comprising an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 91, 92, 94, 95, and 97-99. In one embodiment, the nucleic acid molecule comprises a nucleic acid sequence encoding s-peptide comprising an amino acid sequence of one of SEQ ID NOs: 91, 92, 94, 95, and 97-99.

In one embodiment, the nucleic acid sequence encoding an s-peptide comprises a nucleic acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 781, 782, 784, 785, and 787-789. In one embodiment, the nucleic acid sequence encoding an s-peptide comprises a nucleic acid sequence of one of SEQ ID NOs: 781, 782, 784, 785, and 787-789.

Localization Signal

In some embodiments, the nucleic acid molecule comprises a nucleic acid sequence encoding a localization signal, such as an nuclear localization signal (NLS), nuclear export signal (NES) or other localization signals to localize to organelles, such as mitochondria. In one embodiment, the localization signal localizes the fusion protein to the site in which the target RNA is located.

Nuclear Localization Signals

In one embodiment, the nucleic acid molecule comprises a nucleic acid sequence encoding a nuclear localization signal (NLS). In one embodiment, the NLS is a retrotransposon NLS. In one embodiment, the NLS is derived from Ty1, yeast GAL4, SKI3, L29 or histone H2B proteins, polyoma virus large T protein, VP1 or VP2 capsid protein, SV40 VP1 or VP2 capsid protein, Adenovirus El a or DBP protein, influenza virus NS1 protein, hepatitis vims core antigen or the mammalian lamin, c-myc, max, c-myb, p53, c-erbA, jun, Tax, steroid receptor or Mx proteins, Nucleoplasmin (NPM2), Nucleophosmin (NPM1), or simian vims 40 (“SV40”) T-antigen.

In one embodiment, the NLS is a Ty1 or Ty1-derived NLS, a Ty2 or Ty2-derived NLS or a MAK11 or MAK11-derived NLS. In one embodiment, the Ty1 NLS comprises an amino acid sequence of SEQ ID NO:110. In one embodiment, the Ty2 NLS comprises an amino acid sequence of SEQ ID NO:111. In one embodiment, the MAK11 NLS comprises an amino acid sequence of SEQ ID NO:112. In one embodiment, the nucleic acid sequence encoding a NLS comprises a nucleic acid sequence encoding an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 110-730. In one embodiment, the nucleic acid sequence encoding a NLS comprises a nucleic acid sequence encoding an amino acid sequence of one of SEQ ID NOs: 110-730.

In one embodiment, the NLS is a Ty1-like NLS. For example, in one embodiment, the Ty1-like NLS comprises KKRX motif. In one embodiment, the Ty1-like NLS comprises KKRX motif at the N-terminal end. In one embodiment, the Ty1-like NLS comprises KKR motif. In one embodiment, the Ty1-like NLS comprises KKR motif at the C-terminal end. In one embodiment, the Ty1-like NLS comprises a KKRX and a KKR motif. In one embodiment, the Ty1-like NLS comprises a KKRX at the N-terminal end and a KKR motif at the C-terminal end. In one embodiment, the Ty1-like NLS comprises at least 20 amino acids. In one embodiment, the Ty1-like NLS comprises between 20 and 40 amino acids. In one embodiment, the nucleic acid sequence encoding a Ty1-like NLS comprises a nucleic acid sequence encoding an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 118-730. In one embodiment, the nucleic acid sequence encoding a Ty1-like NLS comprises a nucleic acid sequence encoding an amino acid sequence of one of SEQ ID NOs: 118-730, wherein the sequence comprises one or more, two or more, three or more, four or more, five or more, six or more, seven or more, eight or more, nine or more, or ten or more, insertions, deletions or substitutions. In one embodiment, the nucleic acid sequence encoding a Ty1-like NLS comprises a nucleic acid sequence encoding an amino acid sequence of one of SEQ ID NOs: 118-730.

In one embodiment, the nucleic acid sequence encoding an NLS encodes two copies of the same NLS. For example, in one embodiment, the nucleic acid sequence encodes a multimer of a first Ty1-derived NLS and a second Ty1-derived NLS.

In one embodiment, the nucleic acid sequence encoding a NLS comprises a nucleic acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO:794. In one embodiment, the nucleic acid sequence encoding a NLS comprises a nucleic acid sequence of SEQ ID NO: 794.

Nuclear Export Signal

In one embodiment, the nucleic acid molecule comprises a nucleic acid sequence encoding a Nuclear Export Signal (NES). In one embodiment, the NES localizes the fusion protein to the cytoplasm for targeting cytoplasmic RNA. In one embodiment, In one embodiment, the nucleic acid sequence encoding the NES comprises a sequence encoding an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO:802 or 803. In one embodiment, the nucleic acid sequence encoding the NES comprises a sequence encoding an amino acid sequence of SEQ ID NO: 802 or 803.

In one embodiment, the nucleic acid sequence encoding the NES comprises a sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO:804 or 805. In one embodiment, the nucleic acid sequence encoding the NES comprises a sequence of SEQ ID NO: 804 or 805.

Organelle and Extracellular Localization Signal

In one embodiment, the nucleic acid molecule comprises a nucleic acid sequence encoding a localization signal that localizes the fusion protein to an organelle or extracellularly. In one embodiment, the localization signal localizes the protein to the nucleolus, ribosome, vesicle, rough endoplasmic reticulum, Golgi apparatus, cytoskeleton, smooth endoplasmic reticulum, mitochondria, vacuole, cytosol, lysosome, or centriole. A number of localization signals are known in the art.

Exemplary localization signals include, but are not limited to 1x mitochondrial targeting sequence, 4x mitochondrial targeting sequence, secretory signal sequence (IL-2), myristylation, Calsequestrin leader, KDEL retention and peroxisome targeting sequence.

In one embodiment, the nucleic acid molecule comprises a nucleic acid sequence encoding a localization signal. In one embodiment, the localization signal localizes the fusion protein to an organelle or extracellularly. In one embodiment, In one embodiment, the nucleic acid sequence encoding the localization signal comprises a sequence encoding an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO:806-812. In one embodiment, the nucleic acid sequence encoding the localization signal comprises a sequence encoding an amino acid sequence of SEQ ID NO: 806-812.

In one embodiment, the nucleic acid sequence encoding the localization signal comprises a sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO:813-819. In one embodiment, the nucleic acid sequence encoding the localization signal comprises a sequence of SEQ ID NO:813-819.

Linker

In one embodiment, the nucleic acid molecule comprises a nucleic acid sequence encoding a linker peptide. In one embodiment, the linker links the Cas protein and RNase protein. In one embodiment, the linker is connected to the C-terminal end of the Cas protein and to the N-terminal end of the RNase protein. In one embodiment, the linker is connected to the N-terminal end of the Cas protein and to the C-terminal end of the RNase protein.

In one embodiment, the nucleic acid sequence encoding a linker peptide encodes an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs:100-108. In one embodiment, the nucleic acid sequence encoding a linker peptide encodes an amino acid sequence of one of SEQ ID NOs: 100-108.

In one embodiment, the nucleic acid sequence encoding a linker peptide comprises a sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs:790-792. In one embodiment, the nucleic acid sequence encoding a linker peptide comprises sequence of one of SEQ ID NOs: 790-792.

Purification and/or Detection Tag

In one embodiment, the nucleic acid molecule comprises a nucleic acid sequence encoding a purification and/or detection tag. In one embodiment, the tag is on the N-terminal end of the fusion protein. In one embodiment, the tag is a 3×FLAG tag. In one embodiment, nucleic acid sequence encoding a purification and/or detection tag encodes an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO:109. In one embodiment, nucleic acid sequence encoding a purification and/or detection tag encodes an amino acid sequence of SEQ ID NO:109.

In one embodiment, nucleic acid sequence encoding a purification and/or detection tag comprises sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO:793. In one embodiment, nucleic acid sequence encoding a purification and/or detection tag comprises a sequence of SEQ ID NO: 793.

Targeting Nucleic Acids and CRISPR RNAs (crRNAs)

In one aspect, the invention provides targeting nucleic acids, including CRISPR RNAs (crRNAs) for targeting Cas to a target RNA. In one embodiment, targeting nucleic acids is a crRNA. In one embodiment, the crRNA comprises guide sequence. In one embodiment, the crRNA comprises a direct repeat (DR) sequence. In one embodiment the crRNA comprises a direct repeat sequence and a guide sequence fused or linked to a guide sequence or spacer sequence. In one embodiment the direct repeat sequence may be located upstream (i.e., 5′) from the guide sequence or spacer sequence. In other embodiments, the direct repeat sequence may be located downstream (i.e., 3′) from the guide sequence or spacer sequence.

In some embodiments, the crRNA comprises a stem loop. In one embodiment, the crRNA comprises a single stem loop. In one embodiment, the direct repeat sequence forms a stem loop. In one embodiment, the direct repeat sequence forms a single stem loop.

In one embodiment, the crRNA is complementary to the RNase of the fusion protein. For example, in one embodiment, the RNase is capable of cleaving ssRNA and the crRNA guide sequence comprises a sequence having sufficient complementarity to a sequence adjacent to the target sequence. In one embodiment, the RNase is capable of cleaving ssRNA and the crRNA guide sequence comprises a sequence having sufficient complementarity to the target sequence and creating a bulge ssRNA at the target site.

In one embodiment, the RNase is capable of cleaving dsRNA and the crRNA guide sequence comprises a sequence having sufficient complementarity to the target sequence, thereby creating dsRNA capable of being cleaved by the RNase. In one embodiment, the RNase is capable of cleaving dsRNA and the crRNA guide sequence comprises a sequence having sufficient complementarity to the target sequence and mismatches 5′ and 3′ of the target site creating ssRNA bulge 5′ and 3′ of the target site, thereby creating dsRNA at the target site capable of being cleaved by the RNase.

In one embodiment, the targeting nucleic acid is a targeting DNA oligo. In one embodiment, the DNA oligo comprises a guide sequence comprises a sequence having sufficient complementarity to the target sequence. In one embodiment, the targeting DNA oligo can be delivered in combination with a crRNA. In one embodiment, the crRNA directs the Cas13-RNase fusion protein to the target site. In one embodiment, the crRNA directs the Cas13-RNase fusion protein to the target site, wherein the RNase is capable of cleaving an RNA:DNA complex. In one embodiment, the DNA oligo binds to the target site, thereby allowing the RNase of the Cas13-RNase fusion protein to cleave the target site.

In one embodiment, the spacer length of the guide RNA is from 15 to 35 nt. In one embodiment, the spacer length of the guide RNA is at least 15 nucleotides. In one embodiment the spacer length is from 15 to 17 nt, e.g., 15, 16, or 17 nt, from 17 to 20 nt, e.g., 17, 18, 19, or nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments of CRISPR-Cas systems, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and advantageously tracr RNA is 30 or 50 nucleotides in length. However, an aspect of the invention is to reduce off-target interactions, e.g., reduce the guide interacting with a target sequence having low complementarity. Indeed, in the examples, it is shown that the invention involves mutations that result in the CRISPR-Cas system being able to distinguish between target and off-target sequences that have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches). Accordingly, in the context of the present invention the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

### Ef1a2 Promotors

In one aspect, the nucleic acid molecules of the disclosure comprise a Ef1a2 promotor to drive the expression of a protein or gene described herein. In one embodiment, the promotor is Ef1a2 promotor is capable of driving expression in heart, skeletal muscle and neural tissues, such as brain and motor neurons. In one embodiment, the Ef1a2 promotor comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs: 820-822. In one embodiment, the Ef1a2 promotor comprises a sequence of one of SEQ ID NOs: 820-822.

### Nucleic Acids

The isolated nucleic acid sequences of the disclosure can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The isolated nucleic acid may comprise any type of nucleic acid, including, but not limited to DNA and RNA. For example, in one embodiment, the composition comprises an isolated DNA molecule, including for example, an isolated cDNA molecule, encoding a protein of the disclosure. In one embodiment, the composition comprises an isolated RNA molecule encoding a fusion of the disclosure, or a functional fragment thereof.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

In one embodiment of the present invention the nucleic acid molecule may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In some instances, the nucleic acid molecule comprises at least one of the following chemical modifications: 2′-H, 2′-O-methyl, or 2′-OH modification of one or more nucleotides. In certain embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

In certain embodiments, the nucleic acid molecule of the invention has one or more of the following properties:

Nucleic acid agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, or as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, or different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone.

Modifications of the nucleic acid of the invention may be present at one or more of, a phosphate group, a sugar group, backbone, N-terminus, C-terminus, or nucleobase.

The present invention also includes a vector in which the isolated nucleic acid of the present invention is inserted. The art is replete with suitable vectors that are useful in the present invention.

In brief summary, the expression of natural or synthetic nucleic acids encoding a protein of the disclosure is typically achieved by operably linking a nucleic acid encoding the protein of the disclosure or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In one embodiment, the nucleic acid encoding one or more fusion protein of the present invention comprises a nucleic acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 824, linked at its 3′ end to a nucleic acid sequence encoding an RNase, as described above. In one embodiment, the nucleic acid encoding one or more fusion protein comprises a nucleic acid sequence of SEQ ID NO: 824, linked at its 3′ end to a nucleic acid sequence encoding an RNase.

In one embodiment, the nucleic acid encoding one or more fusion protein of the present invention comprises a nucleic acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 825, linked at its 5′ end to a nucleic acid sequence encoding a first RNase, and linked at its 3′ end to a nucleic acid sequence encoding a second RNase. In one embodiment, the nucleic acid encoding one or more fusion protein comprises a nucleic acid sequence of SEQ ID NO: 825, linked at its 5′ end to a nucleic acid sequence encoding a first RNase, and linked at its 3′ end to a nucleic acid sequence encoding a second RNase.

### Nucleic Acids Encoding Proteins for Trans-Splicing of RNA Molecules

In some embodiments, the present invention relates to nucleic acids encoding novel fusions of editing proteins for trans-splicing RNA molecules in cells or in vitro. In one embodiment, the present invention comprises a composition comprising one or more nucleic acid encoding one or more novel fusion of an editing protein, as described herein, one or more targeting nucleic acid, as described herein, and one or more RNA molecule. In some embodiments, said one or more RNA molecule comprises a single RNA molecule. In some embodiments, said one or more RNA molecule comprises at least two RNA molecules. In one embodiment, the composition further comprises a RtcB ligase or a nucleic acid encoding RtcB ligase. Exemplary RtcB ligases and corresponding nucleic acid sequences encoding said RtcB ligases can be found in International Application No. PCT/US2021/016885 (incorporated by reference herein in its entirety).

### Delivery Systems and Methods

In one aspect, the invention relates to the development of novel lentiviral packaging and delivery systems. The lentiviral particle delivers the viral enzymes as proteins. In this fashion, lentiviral enzymes are short lived, thus limiting the potential for off-target editing due to long term expression though the entire life of the cell. The incorporation of editing components, or traditional CRISPR-Cas editing components as proteins in lentiviral particles is advantageous, given that their required activity is only required for a short period of time. Thus, in one embodiment, the invention provides a lentiviral delivery system and methods of delivering the compositions of the invention, editing genetic material, and nucleic acid delivery using lentiviral delivery systems.

In one embodiment, the delivery system comprises (1) a packaging plasmid (2) a transfer plasmid, and (3) an envelope plasmid. In one embodiment, the delivery system comprises (1) a packaging plasmid (2) an envelope plasmid, and (3) a VPR plasmid. In one embodiment, the packaging plasmid comprises a nucleic acid sequence encoding a gag-pol polyprotein. In one embodiment, the gag-pol polyprotein comprises catalytically dead integrase. In one embodiment, the gag-pol polyprotein comprises a mutation selected from D116N and D64V. In one embodiment, the transfer plasmid comprises a nucleic acid sequence encoding a crRNA sequence and Cas protein of the invention

In one embodiment, the envelope plasmid comprises a nucleic acid sequence encoding an envelope protein. In one embodiment, the envelope plasmid comprises a nucleic acid sequence encoding an HIV envelope protein. In one embodiment, the envelope plasmid comprises a nucleic acid sequence encoding a vesicular stomatitis virus g-protein (VSV-g) envelope protein. In one embodiment, the envelope protein can be selected based on the desired cell type.

In one embodiment, the VPR plasmid comprises a nucleic acid sequence encoding a fusion protein comprising VPR, a Cas protein, and RNase protein. In one embodiment, the VPR plasmid comprises a nucleic acid sequence encoding a fusion protein comprising VPR, a Cas protein, a RNase protein and an NLS. In one embodiment, the VPR plasmid comprises a nucleic acid sequence encoding a fusion protein comprising VPR, a Cas protein, a RNase protein and an NES. In one embodiment, the fusion protein comprises a protease cleavage site between VPR and the Cas protein, and RNase protein. In one embodiment, the VPR plasmid packaging plasmid further comprises a sequence encoding a targeting nucleic acid sequence.

In one embodiment, the packaging plasmid, transfer plasmid, envelope plasmid, and VPR plasmid are introduced into a cell. In one embodiment, the cell transcribes and translates the nucleic acid sequence encoding the gag-pol protein to produce the gag-pol polyprotein. In one embodiment, the cell transcribes and translates the nucleic acid sequence encoding the envelope protein to produce the envelope protein. In one embodiment, the cell transcribes and translates the fusion protein to produce the VPR-fusion protein. In one embodiment, the cell transcribes the nucleic acid sequence encoding the guide RNA. In one embodiment, the transcribed transfer plasmid and gag-pol proteins are packaged into a lentiviral vector. In one embodiment, the lentiviral vectors are collected from the cell media. In one embodiment, the viral particles transduce a target cell, wherein the transcribed the crRNA and Cas protein are cleaved and the translated thereby generating the Cas protein and crRNA, wherein the crRNA binds to the Cas protein and directs it to an RNA having a sequence substantially complementary to the crRNA sequence.

In one embodiment, the gag-pol protein, envelope polyprotein, and VPR-fusion protein, which is bound to the guide RNA, are packaged into a viral particle. In one embodiment, the viral particles are collected from the cell media. In one embodiment, VPR is cleaved from the fusion protein in the viral particle via the protease site to provide a Cas-fusion protein. In one embodiment, the viral particles transduce a target cell, wherein the guide RNA binds a target region of an RNA thereby targeting the Cas fusion protein.

Further, a number of additional viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In one embodiment, the composition includes a vector derived from an adeno-associated virus (AAV). The term “AAV vector” means a vector derived from an adeno-associated virus serotype, including without limitation, AAV-1, AAV-2, AAV-3, AAV-4, AAV-5, AAV-6, AAV-7, AAV-8, and AAV-9. AAV vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

AAV vectors can have one or more of the AAV wild-type genes deleted in whole or part, preferably the rep and/or cap genes, but retain functional flanking ITR sequences. Despite the high degree of homology, the different serotypes have tropisms for different tissues. The receptor for AAV1 is unknown; however, AAV1 is known to transduce skeletal and cardiac muscle more efficiently than AAV2. Since most of the studies have been done with pseudotyped vectors in which the vector DNA flanked with AAV2 ITR is packaged into capsids of alternate serotypes, it is clear that the biological differences are related to the capsid rather than to the genomes. Recent evidence indicates that DNA expression cassettes packaged in AAV 1 capsids are at least 1 log more efficient at transducing cardiomyocytes than those packaged in AAV2 capsids. In one embodiment, the viral delivery system is an adeno-associated viral delivery system. The adeno-associated virus can be of serotype 1 (AAV 1), serotype 2 (AAV2), serotype 3 (AAV3), serotype 4 (AAV4), serotype 5 (AAV5), serotype 6 (AAV6), serotype 7 (AAV7), serotype 8 (AAV8), or serotype 9 (AAV9).

Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid. Thus exemplary AAVs, or artificial AAVs, suitable for expression of one or more proteins, include AAV2/8 (see U.S. Pat. No. 7,282,199), AAV2/5 (available from the National Institutes of Health), AAV2/9 (International Patent Publication No. WO2005/033321), AAV2/6 (U.S. Pat. No. 6,156,303), and AAVrh8 (International Patent Publication No. WO2003/042397), among others.

In certain embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

In order to assess the expression of a fusion protein of the invention, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). An exemplary method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Nanoparticles

In some embodiments, the present disclosure provides fusion proteins, nucleic acids, or a combination thereof of any of the preceding paragraphs formulated in a nanoparticle (e.g., a lipid nanoparticle).

In some embodiments, the fusion proteins, nucleic acids, or a combination thereof is formulated in a lipid nanoparticle. In some embodiments, the fusion proteins, nucleic acids, or a combination thereof is formulated in a lipid-polycation complex, referred to as a cationic lipid nanoparticle. As a non-limiting example, the polycation may include a cationic peptide or a polypeptide such as, but not limited to, polylysine, polyornithine and/or polyarginine. In some embodiments, the fusion proteins, nucleic acids, or a combination thereof is formulated in a lipid nanoparticle that includes a non-cationic lipid such as, but not limited to, cholesterol or dioleoyl phosphatidyl-ethanolamine (DOPE). In some embodiments, the lipid nanoparticle comprises at least one ionizable cationic lipid, at least one non-cationic lipid, at least one sterol, and/or at least one polyethylene glycol (PEG)-modified lipid.

A lipid nanoparticle formulation may be influenced by, but not limited to, the selection of the cationic lipid component, the degree of cationic lipid saturation, the nature of the PEGylation, ratio of all components and biophysical parameters such as size. In one example by Semple et al. (Nature Biotech. 2010 28:172-176), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid can more effectively deliver siRNA to various antigen presenting cells (Basha et al. Mol Ther. 2011 19:2186-2200).

In some embodiments, lipid nanoparticle formulations may comprise 35 to 45% cationic lipid, 40% to 50% cationic lipid, 50% to 60% cationic lipid and/or 55% to 65% cationic lipid. In some embodiments, the ratio of lipid to RNA (e.g., mRNA) in lipid nanoparticles may be 5:1 to 20:1, 10:1 to 25:1, 15:1 to 30:1 and/or at least 30:1.

In some embodiments, the ratio of PEG in the lipid nanoparticle formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain 0.5% to 3.0%, 1.0% to 3.5%, 1.5% to 4.0%, 2.0% to 4.5%, 2.5% to 5.0% and/or 3.0% to 6.0% of the lipid molar ratio of PEG-c-DOMG (R-3-[(ω-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In some embodiments, the fusion proteins, nucleic acids, or a combination thereof is formulated as a nanoparticle that comprises at least one lipid selected from, but not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some embodiments, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in U.S. Patent Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-lol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In some embodiments, a lipid nanoparticle formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of 20-60% cationic lipid:5-25% neutral lipid: 25-55% sterol; 0.5-15% PEG-lipid.

In some embodiments, a lipid nanoparticle formulation includes 25% to 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., 35 to 65%, to 65%, 60%, 57.5%, 50% or 40% on a molar basis.

In some embodiments, a lipid nanoparticle formulation includes 0.5% to 15% on a molar basis of the neutral lipid, e.g., 3 to 12%, 5 to 10% or 15%, 10%, or 7.5% on a molar basis. Examples of neutral lipids include, without limitation, DSPC, POPC, DPPC, DOPE and SM. In some embodiments, the formulation includes 5% to 50% on a molar basis of the sterol (e.g., 15 to 45%, 20 to 40%, 40%, 38.5%, 35%, or 31% on a molar basis. A non-limiting example of a sterol is cholesterol. In some embodiments, a lipid nanoparticle formulation includes 0.5% to 20% on a molar basis of the PEG or PEG-modified lipid (e.g., 0.5 to 10%, 0.5 to 5%, 1.5%, 0.5%, 1.5%, 3.5%, or 5% on a molar basis. In some embodiments, a PEG or PEG modified lipid comprises a PEG molecule of an average molecular weight of 2,000 Da. In some embodiments, a PEG or PEG modified lipid comprises a PEG molecule of an average molecular weight of less than 2,000, for example around 1,500 Da, around 1,000 Da, or around 500 Da. Non-limiting examples of PEG-modified lipids include PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in their entirety).

In some embodiments, the molar lipid ratio is 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-CDMA) or 52/13/30/5 (mol % cationic lipid/neutral lipid, e.g., DSPC/Chol/PEG-modified lipid, e.g., PEG-DMG or PEG-cDMA).

Non-limiting examples of lipid nanoparticle compositions and methods of making them are described, for example, in Semple et al. (2010) Nat. Biotechnol. 28:172-176; Jayarama et al. (2012), Angew. Chem. Int. Ed., Si: 8529-8533; and Maier et al. (2013) Molecular Therapy 21, 1570-1578 (the contents of each of which are incorporated herein by reference in their entirety).

In some embodiments, lipid nanoparticle formulations may comprise a cationic lipid, a PEG lipid and a structural lipid and optionally comprise a non-cationic lipid. As a non-limiting example, a lipid nanoparticle may comprise 40-60% of cationic lipid, 5-15% of a non-cationic lipid, 1-2% of a PEG lipid and 30-50% of a structural lipid. As another non-limiting example, the lipid nanoparticle may comprise 50% cationic lipid, 10% non-cationic lipid, 1.5% PEG lipid and 38.5% structural lipid. As yet another non-limiting example, a lipid nanoparticle may comprise 55% cationic lipid, 10% non-cationic lipid, 2.5% PEG lipid and 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may be 4 component lipid nanoparticles. The lipid nanoparticle may comprise a cationic lipid, a non-cationic lipid, a PEG lipid and a structural lipid. As a non-limiting example, the lipid nanoparticle may comprise 40-60% of cationic lipid, 5-15% of a non-cationic lipid, 1-2% of a PEG lipid and 30-50% of a structural lipid. As another non-limiting example, the lipid nanoparticle may comprise 50% cationic lipid, 10% non-cationic lipid, 1.5% PEG lipid and 38.5% structural lipid. As yet another non-limiting example, the lipid nanoparticle may comprise 55% cationic lipid, 10% non-cationic lipid, 2.5% PEG lipid and 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may comprise a cationic lipid, a non-cationic lipid, a PEG lipid and a structural lipid. As a non-limiting example, the lipid nanoparticle comprise 50% of the cationic lipid DLin-KC2-DMA, 10% of the non-cationic lipid DSPC, 1.5% of the PEG lipid PEG-DOMG and 38.5% of the structural lipid cholesterol. As a non-limiting example, the lipid nanoparticle comprise 50% of the cationic lipid DLin-MC3-DMA, 10% of the non-cationic lipid DSPC, 1.5% of the PEG lipid PEG-DOMG and 38.5% of the structural lipid cholesterol. As a non-limiting example, the lipid nanoparticle comprise 50% of the cationic lipid DLin-MC3-DMA, 10% of the non-cationic lipid DSPC, 1.5% of the PEG lipid PEG-DMG and 38.5% of the structural lipid cholesterol. As yet another non-limiting example, the lipid nanoparticle comprise 55% of the cationic lipid L319, 10% of the non-cationic lipid DSPC, 2.5% of the PEG lipid PEG-DMG and 32.5% of the structural lipid cholesterol.

In some embodiments, a nanoparticle (e.g., a lipid nanoparticle) has a mean diameter of 10-500 nm, 20-400 nm, 30-300 nm, 40-200 nm. In some embodiments, a nanoparticle (e.g., a lipid nanoparticle) has a mean diameter of 50-150 nm, 50-200 nm, 80-100 nm or 80-200 nm.

### Systems

In one aspect, the present invention provides a system for decreasing the number of an RNA transcript in a subject. In one embodiment the system comprises, in one or more vectors, a nucleic acid sequence encoding a fusion protein, wherein the fusion protein comprises a CRISPR-associated (Cas) protein, a RNase protein, and optionally a localization sequence, such as an NLS or NES; and a nucleic acid sequence encoding a CRISPR-Cas system crRNA. In one embodiment, the system further comprises, on the same or different vector, a nucleic acid sequence encoding a second targeting nucleic acid. In one embodiment, the CRISPR-Cas system crRNA substantially hybridizes to a target RNA sequence in the RNA transcript. In one embodiment, the nucleic acid sequence encoding the fusion protein and the nucleic acid sequence encoding a CRISPR-Cas system crRNA are in the same vector. In one embodiment, the nucleic acid sequence encoding the fusion protein and the nucleic acid sequence encoding a CRISPR-Cas system crRNA are in different vectors.

In one embodiment, the nucleic acid sequence encoding a fusion protein comprises (1) a nucleic acid sequence encoding an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 47-48; (2) a nucleic acid sequence encoding an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 49-89; and (3) a nucleic acid sequence encoding an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 110-730. In one embodiment, the nucleic acid sequence encoding a fusion protein comprises (1) a nucleic acid sequence encoding an amino acid of one of SEQ ID NOs: 47-48; (2) a nucleic acid sequence encoding an amino acid of one of SEQ ID NOs: 49-89; and (3) a nucleic acid sequence encoding an amino acid of one of SEQ ID NOs: 110-730.

In one embodiment the system comprises, in one or more vectors, a nucleic acid sequence encoding a fusion protein, wherein the fusion protein comprises a CRISPR-associated (Cas) protein, a s-protein, and optionally a localization signal such as an NLS or NES; and a nucleic acid sequence encoding a CRISPR-Cas system crRNA; and a nucleic acid sequence encoding a s-peptide. In one embodiment, the s-peptide further comprises ERT2. In one embodiment, the nucleic acid sequence encoding a s-peptide is on a different vector. In one embodiment, the s-peptide binds to the s-protein of the fusion protein thereby forming a catalytically active RNase. In one embodiment, the CRISPR-Cas system crRNA substantially hybridizes to a target RNA sequence in the RNA transcript. In one embodiment, the nucleic acid sequence encoding the fusion protein and the nucleic acid sequence encoding a CRISPR-Cas system crRNA are in the same vector. In one embodiment, the nucleic acid sequence encoding the fusion protein and the nucleic acid sequence encoding a CRISPR-Cas system crRNA are in different vectors.

In one embodiment the system comprises, in one or more vectors, a nucleic acid sequence encoding a fusion protein, wherein the fusion protein comprises a CRISPR-associated (Cas) protein, a s-peptide, and optionally a localization sequence, such as an NLS or NES; and a nucleic acid sequence encoding a CRISPR-Cas system crRNA; and a nucleic acid sequence encoding a s-protein. In one embodiment, the s-peptide further comprises ERT2. In one embodiment, the nucleic acid sequence encoding a s-protein is on a different vector. In one embodiment, the s-protein binds to the s-peptide of the fusion protein thereby forming a catalytically active RNase. In one embodiment, the CRISPR-Cas system crRNA substantially hybridizes to a target RNA sequence in the RNA transcript. In one embodiment, the nucleic acid sequence encoding the fusion protein and the nucleic acid sequence encoding a CRISPR-Cas system crRNA are in the same vector. In one embodiment, the nucleic acid sequence encoding the fusion protein and the nucleic acid sequence encoding a CRISPR-Cas system crRNA are in different vectors.

In one embodiment, the nucleic acid sequence encoding a fusion protein comprises (1) a nucleic acid sequence encoding an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 47-48; (2) a nucleic acid sequence encoding an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 90, 93, and 96; (3) a nucleic acid sequence encoding an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 83, 84, 86, 87, 89 and 90; and (4) a nucleic acid sequence encoding an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 110-730. In one embodiment, the nucleic acid sequence encoding a fusion protein comprises (1) a nucleic acid sequence encoding an amino acid of one of SEQ ID NOs: 47-48; (2) a nucleic acid sequence encoding an amino acid of one of SEQ ID NOs: 90, 93, and 96; (3) a nucleic acid sequence encoding an amino acid of one of SEQ ID NOs: 83, 84, 86, 87, 89 and 90; and (4) a nucleic acid sequence encoding an amino acid of one of SEQ ID NOs: 110-730.

### Compositions and Formulations

In one aspect, the present invention provides compositions for decreasing the number of an RNA transcript in a subject. In one embodiment, the composition comprises a fusion protein, wherein the fusion protein comprises a CRISPR-associated (Cas) protein, a RNase protein, and optionally a localization sequence, such as an NLS or NES. In one embodiment, the composition comprises a CRISPR-Cas system crRNA. In one embodiment, the composition a second targeting nucleic acid. In one embodiment, the CRISPR-Cas system crRNA substantially hybridizes to a target RNA sequence in the RNA transcript.

In one embodiment, the composition comprises a fusion protein comprising (1) an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 47-48; (2) an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 49-89; and (3) an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 110-730. In one embodiment, composition comprises a fusion protein comprising (1) an amino acid of one of SEQ ID NOs: 47-48; (2) amino acid of one of SEQ ID NOs: 49-89; and (3) an amino acid of one of SEQ ID NOs: 110-730.

In one embodiment, composition comprises a fusion protein, wherein the fusion protein comprises a CRISPR-associated (Cas) protein, a s-protein, and optionally a localization sequence, such as an NLS or NES; a CRISPR-Cas system crRNA; and an s-peptide. In one embodiment, the s-peptide further comprises ERT2. In one embodiment, the s-peptide binds to the s-protein of the fusion protein thereby forming a catalytically active RNase. In one embodiment, the CRISPR-Cas system crRNA substantially hybridizes to a target RNA sequence in the RNA transcript. In one embodiment, the fusion protein and CRISPR-Cas system crRNA are separate from the s-peptide. In one embodiment, the s-peptide is subsequently added to the composition comprising the fusion protein and CRISPR-Cas system crRNA thereby providing inducible catalytic activity.

In one embodiment, composition comprises a fusion protein, wherein the fusion protein comprises a CRISPR-associated (Cas) protein, a s-peptide, and optionally a localization sequence, such as an NLS or NES; a CRISPR-Cas system crRNA; and an s-protein. In one embodiment, the s-peptide further comprises ERT2. In one embodiment, the s-protein binds to the s-peptide of the fusion protein thereby forming a catalytically active RNase. In one embodiment, the CRISPR-Cas system crRNA substantially hybridizes to a target RNA sequence in the RNA transcript. In one embodiment, the fusion protein and CRISPR-Cas system crRNA are separate from the s-protein. In one embodiment, the s-protein is subsequently added to the composition comprising the fusion protein and CRISPR-Cas system crRNA thereby providing inducible catalytic activity.

In one embodiment, composition comprises a fusion protein comprising (1) an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 47-48; (2) an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 90, 93, and 96; (3) an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 83, 84, 86, 87, 89 and 90; and (4) an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to one of SEQ ID NOs: 110-730. In one embodiment, the composition comprises a fusion protein comprising (1) amino acid of one of SEQ ID NOs: 47-48; (2) an amino acid of one of SEQ ID NOs: 90, 93, and 96; (3) an amino acid of one of SEQ ID NOs: 83, 84, 86, 87, 89 and 90; and (4) an amino acid of one of SEQ ID NOs: 110-730.

The disclosure also encompasses the use of pharmaceutical compositions of the disclosure to practice the methods of the disclosure. Such a pharmaceutical composition may consist of at least one modulator (e.g., inhibitor or activator) composition of the invention or a salt thereof in a form suitable for administration to a subject, or the pharmaceutical composition may comprise at least one modulator (e.g., inhibitor or activator) composition of the invention or a salt thereof, and one or more pharmaceutically acceptable carriers, one or more additional ingredients, or some combination of these. The compound of the invention may be present in the pharmaceutical composition in the form of a physiologically acceptable salt, such as in combination with a physiologically acceptable cation or anion, as is well known in the art.

In an embodiment, the pharmaceutical compositions useful for practicing the methods of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for oral, rectal, vaginal, parenteral, topical, pulmonary, intranasal, buccal, ophthalmic, or another route of administration. A composition useful within the methods of the invention may be directly administered to the skin, or any other tissue of a mammal. Other contemplated formulations include liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human subject being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol are included in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. An exemplary preservative is a combination of about 0.5% to 2.0% benzyl alcohol and 0.05% to 0.5% sorbic acid.

In one embodiment, the composition includes an anti-oxidant and a chelating agent that inhibits the degradation of the compound. Exemplary antioxidants for some compounds are BHT, BHA, alpha-tocopherol and ascorbic acid in the range of about 0.01% to 0.3% and BHT in the range of 0.03% to 0.1% by weight by total weight of the composition. In one embodiment, the chelating agent is present in an amount of from 0.01% to 0.5% by weight by total weight of the composition. Exemplary chelating agents include edetate salts (e.g. disodium edetate) and citric acid in the weight range of about 0.01% to 0.20%. In some embodiments, the chelating agent is in the range of 0.02% to 0.10% by weight by total weight of the composition. The chelating agent is useful for chelating metal ions in the composition that may be detrimental to the shelf life of the formulation. While BHT and disodium edetate are exemplary antioxidants and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, include a mammal, for example a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments there between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Routes of administration of any of the compositions of the invention include oral, nasal, parenteral, sublingual, transdermal, transmucosal (e.g., sublingual, lingual, (trans)buccal, and (intra)nasal,), intravesical, intraduodenal, intragastrical, rectal, intra-peritoneal, subcutaneous, intramuscular, intradermal, intra-arterial, intravenous, or administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Methods of Decreasing RNA & Methods of Treatment

In one aspect, the invention provides methods of decreasing the number of a nuclear RNA in a subject. In one embodiment, nuclear RNA is abnormal nuclear RNA. In one embodiment, the method comprises administering to the subject (1) a nucleic acid molecule encoding a fusion protein of the disclosure comprising a Cas protein, an RNase protein, and optionally a localization sequence, such as an NLS or NES, or a fusion protein of the disclosure comprising a Cas protein, an RNase protein, and optionally a localization sequence, such as an NLS or NES; and (2) a nucleic acid molecule encoding a guide nucleic acid molecule comprising a targeting nucleotide sequence complimentary to a target RNA sequence in the RNA or a guide nucleic acid molecule comprising a targeting nucleotide sequence complimentary to a target RNA sequence in the RNA.

In some embodiments, the RNA comprises cytoplasmic RNA. In such embodiments, the method comprises administering to the subject (1) a nucleic acid molecule encoding a fusion protein of the disclosure comprising a Cas protein, and an RNase protein, or a fusion protein of the disclosure comprising a Cas protein, and an RNase protein; and (2) a nucleic acid molecule encoding a guide nucleic acid molecule comprising a targeting nucleotide sequence complimentary to a target RNA sequence, or a guide nucleic acid molecule comprising a targeting nucleotide sequence complimentary to a target RNA sequence.

In some embodiments, the method comprises administering to the subject (1) a nucleic acid molecule encoding a fusion protein of the disclosure comprising a Cas protein, an RNase protein and an NES or a fusion protein of the disclosure comprising a Cas protein, an RNase protein, and an NES; and (2) a nucleic acid molecule encoding a guide nucleic acid molecule comprising a targeting nucleotide sequence complimentary to a target RNA sequence, or a guide nucleic acid molecule comprising a targeting nucleotide sequence complimentary to a target RNA sequence.

In some embodiments, the RNA comprises nuclear RNA. In such embodiments, the method comprises administering to the subject (1) a nucleic acid molecule encoding a fusion protein of the disclosure comprising a Cas protein, an RNase protein and an NLS or a fusion protein of the disclosure comprising a Cas protein, an RNase protein, and an NLS; and (2) a nucleic acid molecule encoding a guide nucleic acid molecule comprising a targeting nucleotide sequence complimentary to a target RNA sequence, or a guide nucleic acid molecule comprising a targeting nucleotide sequence complimentary to a target RNA sequence.

In one embodiment, the subject is a cell. In one embodiment, the cell is a prokaryotic cell or eukaryotic cell. In one embodiment, the cell is a eukaryotic cell. In one embodiment, the cell is a plants, animals, or fungi cell. In one embodiment, the cell is a plant cell. In one embodiment, the cell is an animal cell. In one embodiment, the cell is a yeast cell.

In one embodiment, the subject is a mammal. For example, in one embodiment, the subject is a human, non-human primate, dog, cat, horse, cow, goat, sheep, rabbit, pig, rat, or mouse. In one embodiment, the subject is a non-mammalian subject. For example, in one embodiment, the subject is a zebrafish, fruit fly, or roundworm.

In one embodiment, the amount of nuclear RNA is reduced in vitro. In one embodiment, the amount of nuclear RNA is reduced in vivo.

In one embodiment, the nuclear RNA is nuclear RNA foci. In one embodiment, the nuclear RNA foci include a CUG repeat. In one embodiment, the guide nucleic acid comprises a sequence complementary to a CUG repeat expansion. In one embodiment, the guide nucleic acid comprises a sequence complementary to a CTG repeat expansion. In one embodiment, the guide nucleic acid comprises a sequence complementary to a CTG repeat expansion in the 3′UTR of the human dystrophia myotonica-protein kinase (DMPK) gene. In one embodiment, the guide nucleic acid comprises a sequence of one of SEQ ID NOs:798-800.

In one aspect, the present invention provides methods of treating a subject with a disease or disorder associated with abnormal nuclear RNA. In one embodiment, the method comprises administering to the subject (1) a nucleic acid molecule encoding a fusion protein of the disclosure comprising a Cas protein, an RNase protein, and optionally a localization sequence, such as an NLS or NES, or a fusion protein of the disclosure comprising a Cas protein, an RNase protein, and optionally a localization sequence, such as an NLS or NES; and (2) a nucleic acid molecule encoding a guide nucleic acid molecule comprising a targeting nucleotide sequence complimentary to a target RNA sequence in the nuclear RNA or a guide nucleic acid molecule comprising a targeting nucleotide sequence complimentary to a target RNA sequence in the nuclear RNA.

In one embodiment, the disease or disorder associated with abnormal nuclear RNA is selected from the group consisting of Myotonic Dystrophy type 2 (DM2), Amyotrophic lateral sclerosis (ALS), Huntington's disease-like 2 (HDL2), Spinocerebellar ataxias 8, 31 and 10 (SCAB, -31, -10) and fragile X-associated tremor ataxia syndrome (FXTAS).

In one embodiment, the abnormal nuclear RNA is toxic nuclear RNA foci. In one embodiment, the disease or disorder associated with toxic nuclear RNA foci Myotonic Dystrophy type 1. In one embodiment, the targeting nucleotide sequence comprises a sequence complementary to a CTG repeat expansion in the 3′UTR of the human dystrophia myotonica-protein kinase (DMPK) gene. In one embodiment, the targeting nucleotide sequence comprises a sequence selected from the group consisting of SEQ ID NOs: 798-800.

In one aspect, the present invention provides methods cleaving of nuclear RNA in a subject. In one embodiment, the method comprises administering to the subject (1) a nucleic acid molecule encoding a fusion protein of the disclosure comprising a Cas protein, an RNase protein, and optionally a localization sequence, such as an NLS or NES, or a fusion protein of the disclosure comprising a Cas protein, an RNase protein, and optionally a localization sequence, such as an NLS or NES; and (2) a nucleic acid molecule encoding a guide nucleic acid molecule comprising a targeting nucleotide sequence complimentary to a target RNA sequence in the nuclear RNA or a guide nucleic acid molecule comprising a targeting nucleotide sequence complimentary to a target RNA sequence in the nuclear RNA.

In one aspect, the present invention provides methods of treating a disease or disorder associated with increased gene expression. In one embodiment, the method comprises administering to the subject (1) a nucleic acid molecule encoding a fusion protein of the disclosure comprising a Cas protein, an RNase protein, and optionally a localization sequence, such as an NLS or NES, or a fusion protein of the disclosure comprising a Cas protein, an RNase protein, and optionally a localization sequence, such as an NLS or NES; and (2) a nucleic acid molecule encoding a guide nucleic acid molecule comprising a targeting nucleotide sequence complimentary to a target RNA sequence in the RNA transcript of the gene or a guide nucleic acid molecule comprising a targeting nucleotide sequence complimentary to a target RNA sequence in the RNA transcript of the gene. In one embodiment, the Cas protein cleaves the RNA transcript thereby preventing translation and protein expression.

In one aspect, the present invention provides methods of treating a disease or disorder associated with RNA. For example, in one embodiment, the invention provides a method of treating an RNA virus infection. In one embodiment, the method comprises administering to the subject (1) a nucleic acid molecule encoding a fusion protein of the disclosure comprising a Cas protein, an RNase protein, and optionally a localization sequence, such as an NLS or NES, or a fusion protein of the disclosure comprising a Cas protein, an RNase protein, and optionally a localization sequence, such as an NLS or NES; and (2) a nucleic acid molecule encoding a guide nucleic acid molecule comprising a targeting nucleotide sequence complimentary to a target RNA sequence in the viral RNA or a guide nucleic acid molecule comprising a targeting nucleotide sequence complimentary to a target RNA sequence in the viral RNA. In one embodiment, the Cas protein binds the crRNA, the crRNA binds a target RNA sequence, and the RNase cleaves the RNA sequence thereby preventing translation and expression of viral protein.

### Methods of Treatment and Use

The present invention provides methods of treating, reducing the symptoms of, and/or reducing the risk of developing a disease or disorder in a subject. For example, in one embodiment, methods of the invention can be used to treat, reduce the symptoms of, and/or reduce the risk of developing a disease or disorder in a mammal. In one embodiment, the methods of the invention can be used to treat, reduce the symptoms of, and/or reduce the risk of developing a disease or disorder in a plant. In one embodiment, the methods of the invention can be used treat, reduce the symptoms of, and/or reduce the risk of developing a disease or disorder in a yeast organism.

In one embodiment, the subject is a cell. In one embodiment, the cell is a prokaryotic cell or eukaryotic cell. In one embodiment, the cell is a eukaryotic cell. In one embodiment, the cell is a plants, animals, or fungi cell. In one embodiment, the cell is a plant cell. In one embodiment, the cell is an animal cell. In one embodiment, the cell is a yeast cell.

In one embodiment, the subject is a mammal. For example, in one embodiment, the subject is a human, non-human primate, dog, cat, horse, cow, goat, sheep, rabbit, pig, rat, or mouse. In one embodiment, the subject is a non-mammalian subject. For example, in one embodiment, the subject is a zebrafish, fruit fly, or roundworm.

In one embodiment, the disease or disorder is caused by one or more mutations in a genomic locus. Thus, in one embodiment, the disease or disorder is may be treated, reduced, or the risk can be reduced via an element that prevents or reduces mRNA transcript, or prevents or reduces translation of the protein. Thus, in one embodiment, the method comprises manipulation of an RNA transcript.

In one embodiment, the disease or disorder is caused by abnormal RNA. Thus, in one embodiment, the disease or disorder is may be treated, reduced, or the risk can be reduced via an element that prevents or reduces RNA transcript. Thus, in one embodiment, the method comprises manipulation of an RNA transcript.

In one embodiment, the method comprises administering to the subject (1) a fusion protein of the disclosure or a nucleic acid molecule encoding a fusion protein of the disclosure, and (2) one or more targeting nucleic acid molecules comprising a targeting nucleotide sequence complimentary to a target region in a gene, wherein the gene encodes the RNA transcript. In one embodiment, the RNase cleaves the RNA transcript.

In one embodiment, the method comprises administering to the subject (1) a fusion protein of the disclosure or a nucleic acid molecule encoding a fusion protein of the disclosure, and (2) one or more targeting nucleic acid molecules comprising a targeting nucleotide sequence complimentary to a target region in an RNA transcript. In one embodiment, the RNase cleaves the RNA transcript.

In one embodiment, the disease or disorder is associated with abnormal RNA or increased RNA transcription. For example, in one embodiment, the disease or disorder is an endocrine disease. For example, in one embodiment, endocrine diseases include but are not limited to, β-thalassemias, neonatal diabetes, IPEX syndrome, Mayer-Rokitanski-Kiister-Hausersyndrome, Hypothalamic-pituitary-adrenal axis dysregulation, Adrenal dysfunction, Gonadal dysfunction, Ectopic Cushing syndrome, Pre-eclampsia, Diabetic nephropathy, Type I diabetes, Type II diabetes, and IGF-1 deficiency.

In one embodiment, the disease or disorder is a tumorigenic disease. For example, in one embodiment, tumorigenic diseases include but are not limited to, mantle cell lymphoma, hereditary & sporadic parathyroid tumors, Medullary thyroid carcinoma, poliverative conditions, colorectal cancer, gliblastoma, Chronic lymphocytic leukemia, and Breast cancer.

In one embodiment, the disease or disorder is a neurological disease or disorder. For example, in one embodiment, neurological diseases include but are not limited to, Parkinsons diseases, Oculopharyngeal muscular dystrophy, Huntington's disease, Fabry disease, Fragile X syndrome, spinal muscular atrophy, Amyotrophic Lateral Sclerosis, Spinocerebellar ataxia Spinocerebellar ataxia 1, Spinocerebellar ataxia 2, Spinocerebellar ataxia 3, Spinocerebellar ataxia 6, Spinocerebellar ataxia 7, Spinocerebellar ataxia 8, Spinocerebellar ataxia 10, Spinocerebellar ataxia 17, Spinocerebellar ataxia 31, and Alzheimer's disease,

In one embodiment, the disease or disorder is a hematological disease or disorder. For example, in one embodiment, hematological diseases include but are not limited to, (3-Thalassemia, and a-Thalassemia.

In one embodiment, the disease or disorder is an infection or immunological disease or disorder. For example, in one embodiment, infection or immunological diseases include but are not limited to, B-cell differentiation, T-cell activation, systemic lupus erythematosus, Wiskott-Aldrich syndrome, Osteoarthritis, scleroderma, and IPEX syndrome.

In one embodiment, the disease or disorder is a musculoskeletal disease or disorder. For example, in one embodiment, infection or immunological diseases include Myotonic dystrophy type 1, Spinal and bulbar muscular atrophy, and Dentatorubral-pallidoluysian atrophy.

Exemplary diseases or disorders and corresponding targets include, but are not limited to those listed in Table 1. Additional diseases and disorders and corresponding genes are known in the art, for example in Rehfeld et al., Alternations in Polyadenylation and its Implications for Endocrine Disease, Front. Endocrinol. 4:53 (2013), Chang et al., Alternative Polyadenylation in Human Diseases, Endocrinol Metab. 32:413-421 (2017), and Curinha et al., Implications of polyadenylation in health and disease, Nucleus 5:508-519 (2014), which are herein incorporated by reference in their entireties.

TABLE 2
Diseases or disorders and target gene

| Condition | Target |
|---|---|
| Adrenal dysfunction | STAR |
| Alzheimer's disease | ABCA3, EIF2B3, MSTO2P, OGDHL, PARP6, |
| | SLC33A1, SUPV3L1, TAF3, WASH7P, JAK1, |
| | ABCA3, UBR1, ALDOC, C10ord10, |
| | GABARAPL2, KAT2A, POLR3A, |
| | SH3BGRL2, TIMM23, TMC6, UNC80, WTAP |
| Amyotrophic Lateral Sclerosis | ALS (GGGGCC repeat), CCDC92, CRYAB, |
| | MAP7D2, PRPH, RTN4, SEC22B, SNAP25, |
| | UCHL1, YWHAB, C14ord2, C6orf203, |
| | ALDOC, ARL6IP1, CSDE1, CMC2, GAP43, |
| | LDHB, MLLT11, NCL, PFN2, TMOD1, |
| | VAMP1 |
| B-cell differentiation | IGHM |
| Breast cancer | |
| Cancer, Colorectal | DMKN, PDXK, PPIE |
| Cancer, Various | TP53 |
| Chronic lymphocytic leukaemia | Intronic loci |
| Colorectal cancer | DMKN, PDXK, PPIE |
| Dentatorubral-pallidoluysian atrophy | DRPLA (CAG repeat) |
| Diabetic nephropathy | HGRG-14 |
| Ectopic Cushing syndrome | ACTH |
| Fabry disease | α-GalA |
| Fragile X Syndrome | FMR1, FXTAS (CGG repeat) |
| Friedreich's Ataxia | YSH1 |
| Glioblastoma | CCND1, MECP2 |
| Gonadal dysfunction | STAR |
| Huntington's disease | HTT, HD (CAG repeat) |
| Huntington's disease-like 2 | HDL2 (CTG repeat) |
| Hypothalamic-pituitary-adrenal axis dysregulation | SERT |
| IGF-1 deficiency | IGF-1 |
| IPEX syndrome | FOP3 |
| Mayer-Rokitanski-Küster-Hausersyndrome | AMH |
| Myotonic Dystrophy Type I | DMPK, DM1 (CTG Repeat), DM2 (CCTG |
| | repeat) |
| Myotonic Dystrophy Type II | ZFN9 |
| Neonatal diabetes | INS |
| Oculopharyngeal muscular dystrophy | CCND1, PABPN1 |
| Parkinson disease | SNCA |
| Pre-eclampsia | SFLT-1 |
| Proliferative conditions | RBX1 |
| Spinal Muscular Atrophy | SMN |
| Spinocerebellar Ataxia 1 | ATXN1, SCA1 (CAG repeat) |
| Spinocerebellar Ataxia 10 | ATXN10, SCA10 (ATTCT repeat) |
| Spinocerebellar Ataxia 17 | TATA-box binding protein, SCA17 (CAG |
| | repeat) |
| Spinocerebellar Ataxia 2 | ATXN2, SCA2 (CAG repeat) |
| Spinocerebellar Ataxia 3 | ATXN3, SCA3 (CAG repeat) |
| Spinocerebellar Ataxia 31 | BEAN1, SCA31 (TGGA repeat) |
| Spinocerebellar Ataxia 6 | CACNA1A, SCA6 (CAG repeat) |
| Spinocerebellar Ataxia 7 | TPP1, SCA7 (CAG repeat) |
| Spinocerebellar Ataxia 8 | ATXN8OS, SCA8 (CTG/CAG repeat) |
| Spinal and bulbar muscular atrophy | SBMA (CAG repeat) |
| Steroidogenesis | STAR |
| Systemic lupus erythematosus | GIMAP5 |
| Systemic lupus erythematosus | IRF5 |
| T-cell activation | NF-ATC1 |
| Thrombophilia | F2 |
| Type I diabetes | GIMAP5 |
| Type II diabetes | TCF7L2 |
| Wiskott-Aldrich Syndrome | WAS |
| α-Thalassemia | HBA1, HBA2 |
| β-Thalassemia | HBB |

In one embodiment, the disease or disorder is a viral infection. Thus, in one embodiment, the disease or disorder is may be treated, reduced, or the risk can be reduced via an element that prevents or reduces viral mRNA transcript, or prevents or reduces translation of viral protein. Thus, in one embodiment, the method comprises manipulation of a viral RNA transcript.

In one embodiment, the method comprises administering to the subject (1) a fusion protein of the disclosure or a nucleic acid molecule encoding a fusion protein of the disclosure, and (2) one or more targeting nucleic acid molecules comprising a targeting nucleotide sequence complimentary to a viral RNA transcript. In one embodiment, the RNase cleaves the viral RNA transcript.

In one embodiment, the virus is an RNA virus. In one embodiment, the virus produces RNA during its lifecycle. In one embodiment, the virus is a human virus, a plant virus or an animal virus. Exemplary viruses include, but are not limited to, viruses of families Adenoviridae, Adenoviridae, Alphaflexiviridae, Anelloviridae, Arenavirus, Arteriviridae, Asfarviridae, Astroviridae, Benyviridae, Betaflexiviridae, Birnaviridae, Bornaviridae, Bromoviridae, Caliciviridae, Caulimoviridae, Circoviridae, Closteroviridae, Coronaviridae, Filoviridae, Flaviviridae, Geminiviridae, Hantaviridae, Hepadnaviridae, Hepeviridae, Herpesviridae, Kitaviridae, Luteoviridae, Nairoviridae, Nanoviridae, Nimaviridae, Orthomyxoviridae, Paramyxoviridae, Phenuiviridae, Picornaviridae, Polyomaviridae, Pospiviridae, Potyviridae, Poxviridae, Reoviridae, Retroviridae, Retrovirus, Rhabdoviridae, Secoviridae, Togaviridae, Tombusviridae, Tospoviridae, Tymoviridae, and Virgaviridae. For example, exemplary viruses include, but are not limited to, African swine fever, Avian hepatitis E, Avian infectious laryngotracheitis, Avian nephritis virus, Bamboo mosaic virus, Banana bunchy top virus, Barley stripe mosaic virus, Barley yellow dwarf virus, Potato leafroll virus, Borna disease, Brome mosaic virus, wheat, Cauliflower mosaic virus, Chikungunya, Eastern equine encephalitis virus, Citrus leprosis, Citrus sudden death associated virus, Citrus tristeza virus, Coconut cadang-cadang viroid, Curly top virus, African cassava mosaic virus, Cytomegalovirus, Epstein-Barr virus, Dengue, Yellow fever, West Nile, Zika, Ebola virus, Marburg virus, Equine arteritis virus, Porcine reproductive and respiratory syndrome virus, Equine infectious anemia, Foot and mouth disease, Foot and mouth disease, Enteroviruses, Rhinoviruses, Hepatitis B virus, Hepatitis E virus, HIV, HIV-1, HIV-2, Infectious bursal disease virus (poultry), Infectious pancreatic necrosis (salmon), Infectious canine hepatitis, aviadenoviruses of fowl, Influenza viruses, Lassa virus, Lymphocytic choriomeningitis virus, Monkeypox, Nairobi sheep disease, Newcastle disease virus (poultry), Norwalk virus, Numerous examples of crop damaging viruses, including Potato virus Y, Porcine circovirus 2, Beak and feather disease virus (poultry), Potato virus M, Rabies virus, Respiratory and enteric adenoviruses, Respiratory syncytial virus, Rice stripe necrosis virus, Rift Valley fever, rotaviruses, SARS-CoV-2, MERS, Sheeppox virus, Lumpy skin disease virus, Sin Nombre virus, Andes virus, SV40, Tobacco ringspot virus, Tomato bushy stunt virus, Tomato spotted wilt virus, Torque teno virus, Venezuelan equine encephalitis virus, Vesicular stomatitis Indiana virus, Viral hemorrhagic septicemia (trout), and White spot syndrome virus (shrimp).

In one embodiment, exemplary viruses include, but are not limited to, Primate T-lymphotropic virus 1, Primate T-lymphotropic virus 2, Primate T-lymphotropic virus 3, Human immunodeficiency virus 1, Human immunodeficiency virus 2, Simian foamy virus, Human picobirnavirus, Colorado tick fever virus, Changuinola virus, Great Island virus, Lebombo virus, Orungo virus, Rotavirus A, Rotavirus B, Rotavirus C, Banna virus, Borna disease virus, Lake Victoria Marburgvirus, Reston ebolavirus, Sudan ebolavirus, Tai forest ebolavirus, Zaire virus, Human parainfluenza virus 2, Human parainfluenza virus 4, Mumps virus, Newcastle disease virus, Human parainfluenza virus 1, Human parainfluenza virus 3, Hendra virus, Nipah virus, Measles virus, Human respiratory syncytial virus, Human metapneumovirus, Chandipura virus, Isfahan virus, Piry virus, Vesicular stomatitis Alagoas virus, Vesicular stomatitis Indiana virus, Vesicular stomatitis New Jersey virus, Australian bat lyssavirus, Duvenhage virus, European bat lyssavirus 1, European bat lyssavirus 2, Mokola virus, Rabies virus, Guanarito virus, Junin virus, Lassa virus, Lymphocytic choriomeningitis virus, Machupo virus, Pichinde virus, Sabia virus, Whitewater Arroyo virus, Bunyamwera virus, Bwamba virus, California encephalitis virus, Caraparu virus, Catu virus, Guama virus, Guaroa virus, Kairi virus, Marituba virus, Oriboca virus, Oropouche virus, Shuni virus, Tacaiuma virus, Wyeomyia virus, Andes virus, Bayou virus, Black creek canal virus, Dobrava-Belgrade virus, Hantaan virus, Laguna Negra virus, New York virus, Puumala virus, Seoul virus, Sin Nombre virus, Crimean-Congo haemorrhagic fever virus, Dugbe virus, Candiru virus, Punta Toro virus, Rift Valley fever virus, Sandfly fever Naples virus, Influenza A virus, Influenza B virus, Influenza C virus, Dhori virus, Thogoto virus, Hepatitis delta virus, Human coronavirus 229E, Human coronavirus NL63, Human coronavirus HKU1, Human coronavirus OC43, SARS coronavirus, Human torovirus, Human enterovirus A, Human enterovirus B, Human enterovirus C, Human enterovirus D, Human rhinovirus A, Human rhinovirus B, Human rhinovirus C, Encephalomyocarditis virus, Theilovirus, Equine rhinitis A virus, Foot and mouth disease virus, Hepatitis A virus, Human parechovirus, Ljungan virus, Aichi virus, Human astrovirus, Human astrovirus 2, Human astrovirus 3, Human astrovirus 4, Human astrovirus 5, Human astrovirus 6, Human astrovirus 7, Human astrovirus 8, Norwalk virus, Sapporo virus, Aroa virus, Banzi virus, Dengue virus, Ilheus virus, Japanese encephalitis virus, Kokobera virus, Kyasanur forest disease virus, Louping ill virus, Murray Valley encephalitis virus, Ntaya virus, Omsk haemorrhagic fever virus, Powassan virus, Rio Bravo virus, St Louis encephalitis virus, Tick-borne encephalitis virus, Usutu virus, Wesselsbron virus, West Nile virus, Yellow fever virus, Zika virus, Hepatitis C virus, Hepatitis E virus, Barmah Forest virus, Chikungunya virus, Eastern equine encephalitis virus, Everglades virus, Getah virus, Mayaro virus, Mucambo virus, O'nyong-nyong virus, Pixuna virus, Ross River virus, Semliki Forest virus, Sindbis virus, Venezuelan equine encephalitis virus, Western equine encephalitis virus, Whataroa virus, Rubella virus.

In one embodiment, exemplary viruses include, but are not limited to, Ranid herpesvirus 1, Ranid herpesvirus 2, Ranid herpesvirus 3, Anguillid herpesvirus 1, Cyprinid herpesvirus 1, Cyprinid herpesvirus 2, Cyprinid herpesvirus 3, Acipenserid herpesvirus 2, Ictalurid herpesvirus 1, Ictalurid herpesvirus 2, Salmonid herpesvirus 1, Salmonid herpesvirus 2, Salmonid herpesvirus 3, Gallid alphaherpesvirus 1, Psittacid alphaherpesvirus 1, Anatid alphaherpesvirus 1, Columbid alphaherpesvirus 1, Gallid alphaherpesvirus 2, Gallid alphaherpesvirus 3, Meleagrid alphaherpesvirus 1, Spheniscid alphaherpesvirus 1, Chelonid alphaherpesvirus 5, Testudinid alphaherpesvirus 3, Ateline alphaherpesvirus 1, Bovine alphaherpesvirus 2, Cercopithecine alphaherpesvirus 2, Human alphaherpesvirus 1, Human alphaherpesvirus 2, Leporid alphaherpesvirus 4, Macacine alphaherpesvirus 1, Macropodid alphaherpesvirus 1, Macropodid alphaherpesvirus 2, Panine alphaherpesvirus 3, Papiine alphaherpesvirus 2, Pteropodid alphaherpesvirus 1, Saimiriine alphaherpesvirus 1, Bovine alphaherpesvirus 1, Bovine alphaherpesvirus 5, Bubaline alphaherpesvirus 1, Canid alphaherpesvirus 1, Caprine alphaherpesvirus 1, Cercopithecine alphaherpesvirus 9, Cervid alphaherpesvirus 1, Cervid alphaherpesvirus 2, Equid alphaherpesvirus 1, Equid alphaherpesvirus 3, Equid alphaherpesvirus 4, Equid alphaherpesvirus 8, Equid alphaherpesvirus 9, Felid alphaherpesvirus 1, Human alphaherpesvirus 3, Monodontid alphaherpesvirus 1, Phocid alphaherpesvirus 1, Suid alphaherpesvirus 1, Chelonid alphaherpesvirus 6, Aotine betaherpesvirus 1, Cebine betaherpesvirus 1, Cercopithecine betaherpesvirus 5, Human betaherpesvirus 5, Macacine betaherpesvirus 3, Macacine betaherpesvirus 8, Mandrilline betaherpesvirus 1, Panine betaherpesvirus 2, Papiine betaherpesvirus 3, Papiine betaherpesvirus 4, Saimiriine betaherpesvirus 4, Murid betaherpesvirus 1, Murid betaherpesvirus 2, Murid betaherpesvirus 8, Elephantid betaherpesvirus 1, Elephantid betaherpesvirus 4, Elephantid betaherpesvirus 5, Human betaherpesvirus 7, Human betaherpesvirus 6A, Human betaherpesvirus 6B, Macacine betaherpesvirus 9, Murid betaherpesvirus 3, Suid betaherpesvirus 2, Caviid betaherpesvirus 2, Tupaiid betaherpesvirus 1, Callitrichine gammaherpesvirus 3, Cercopithecine gammaherpesvirus 14, Gorilline gammaherpesvirus 1, Human gammaherpesvirus 4, Macacine gammaherpesvirus 4, Macacine gammaherpesvirus 10, Panine gammaherpesvirus 1, Papiine gammaherpesvirus 1, Pongine gammaherpesvirus 2, Alcelaphine gammaherpesvirus 1, Alcelaphine gammaherpesvirus 2, Bovine gammaherpesvirus 6, Caprine gammaherpesvirus 2, Hippotragine gammaherpesvirus 1, Ovine gammaherpesvirus 2, Suid gammaherpesvirus 3, Suid gammaherpesvirus 4, Suid gammaherpesvirus 5, Equid gammaherpesvirus 2, Equid gammaherpesvirus 5, Felid gammaherpesvirus 1, Mustelid gammaherpesvirus 1, Phocid gammaherpesvirus 3, Vespertilionid gammaherpesvirus 1, Ateline gammaherpesvirus 2, Ateline gammaherpesvirus 3, Bovine gammaherpesvirus 4, Cricetid gammaherpesvirus 2, Human gammaherpesvirus 8, Macacine gammaherpesvirus 5, Macacine gammaherpesvirus 8, Macacine gammaherpesvirus 11, Macacine gammaherpesvirus 12, Murid gammaherpesvirus 4, Murid gammaherpesvirus 7, Saimiriine gammaherpesvirus 2, Equid gammaherpesvirus 7, Phocid gammaherpesvirus 2, Saguinine gammaherpesvirus 1, Iguanid herpesvirus 2, Haliotid herpesvirus 1, Ostreid herpesvirus 1, Salmonella virus SKML39, Shigella virus AG3, Dickeya virus Limestone, Dickeya virus RC2014, Escherichia virus CBA120, Escherichia virus PhaxI, Salmonella virus 38, Salmonella virus Det7, Salmonella virus GG32, Salmonella virus PM10, Salmonella virus SFP10, Salmonella virus SH19, Salmonella virus SJ3, Escherichia virus KWBSE43-6, Klebsiella virus 0507KN21, Klebsiella virus KpS110, Klebsiella virus May, Klebsiella virus Menlow, Serratia virus IME250, Erwinia virus Ea2809, Serratia virus MAM1, Acinetobacter virus Acibel007, Acinetobacter virus AB3, Acinetobacter virus AbKT21III, Acinetobacter virus Abp1, Acinetobacter virus Aci07, Acinetobacter virus Aci08, Acinetobacter virus AS11, Acinetobacter virus AS12, Acinetobacter virus Fri1, Acinetobacter virus IME200, Acinetobacter virus PD6A3, Acinetobacter virus PDAB9, Acinetobacter virus phiAB1, Acinetobacter virus SH-Ab 15519, Acinetobacter virus SWHAb1, Acinetobacter virus SWHAb3, Acinetobacter virus WCHABP5, Acintetobacter virus B1, Acintetobacter virus B2, Acintetobacter virus B5, Acintetobacter virus D2, Acintetobacter virus P1, Acintetobacter virus P2, Acintetobacter virus phiAB6, Acinetobacter virus Petty, Vibrio virus Vc1, Vibrio virus A318, Vibrio virus AS51, Vibrio virus Vp670, Marinomonas virus CBSA, Marinomonas virus CPP1m, Vibrio virus VEN, Pseudomonas virus Achelous, Pseudomonas virus Alpheus, Pseudomonas virus Nerthus, Pseudomonas virus Njord, Pseudomonas virus uligo, Pseudomonas virus C171, Pectobacterium virus PP16, Pectobacterium virus PPWS1, Pectobacterium virus PPWS2, Pectobacterium virus CBS, Pectobacterium virus Clickz, Pectobacterium virus fM1, Pectobacterium virus Gaspode, Pectobacterium virus Khlen, Pectobacterium virus Koot, Pectobacterium virus Lelidair, Pectobacterium virus Nobby, Pectobacterium virus Peatl, Pectobacterium virus Phoria, Pectobacterium virus PP90, Pectobacterium virus Zenivior, Dickeya virus BF25-12, Pseudomonas virus NV3, Pseudomonas virus 130-113, Pseudomonas virus 15pyo, Pseudomonas virus Ab05, Pseudomonas virus ABTNL, Pseudomonas virus DL62, Pseudomonas virus kF77, Pseudomonas virus LKD16, Pseudomonas virus LUZ19, Pseudomonas virus MPK6, Pseudomonas virus MPK7, Pseudomonas virus NFS, Pseudomonas virus PAXYB1, Pseudomonas virus phiKMV, Pseudomonas virus PT2, Pseudomonas virus PTS, Pseudomonas virus RLP, Pseudomonas virus LKA1, Pseudomonas virus f2, Aeromonas virus 25AhydR2PP, Aeromonas virus AS7, Aeromonas virus ZPAH7, Yersinia virus ISAO8, Aeromonas virus Ahp1, Aeromonas virus CF7, Cronobacter virus DevCD23823, Cronobacter virus GAP227, Salmonella virus Spp16, Yersinia virus R8-01, Yersinia virus fHeYen301, Yersinia virus Phi80-18, Pectobacterium virus Arno160, Pectobacterium virus PP2, Proteus virus PM85, Proteus virus PM93, Proteus virus PM116, Proteus virus Pm5460, Pectobacterium virus PP1, Erwinia virus Era103, Erwinia virus S2, Lelliottia virus phD2B, Citrobacter CrRp3, Escherchia virus LL11, Escherichia virus AAPEc6, Escherichia virus ACGC91, Escherichia virus B, Escherichia virus C, Escherichia virus K, Escherichia virus K1-5, Escherichia virus K1E, Escherichia virus mutPK1A2, Escherichia virus VEc3, Escherichia virus UAB78, Salmonella virus BP12B, Salmonella virus SP6, Burkholderia virus BpAMP1, Ralstonia virus RSPI1, Ralstonia virus RSB1, Ralstonia virus RsoP1IDN, Burkholderia virus JG068, Ralstonia virus RSJ2, Ralstonia virus RSJ5, Ralstonia virus RSPII1, Shigella virus Buco, Escherichia virus Minorna, Klebsiella virus AltoGao, Klebsiella virus BO1E, Klebsiella virus F19, Klebsiella virus K244, Klebsiella virus Kp2, Klebsiella virus KP34, Klebsiella virus KPRio2015, Klebsiella virus KpS2, Klebsiella virus KpV41, Klebsiella virus KpV48, Klebsiella virus KpV71, Klebsiella virus KpV74, Klebsiella virus KpV475, Klebsiella virus KPV811, Klebsiella virus myPSH1235, Klebsiella virus SU503, Klebsiella virus SU552A, Shigella virus SFN6B, Enterobacter virus KDA1, Proteus virus PM16, Proteus virus PM75, Dickeya virus Dagda, Dickeya virus Katbat, Dickeya virus Luksen, Dickeya virus Mysterion, Yersinia virus AP10, Erwinia virus FE44, Escherichia virus 285P, Escherichia virus BA14, Escherichia virus P483, Escherichia virus P694, Escherichia virus S523, Kluyvera virus Kvp1, Pectobacterium virus PP74, Salmonella virus BP12A, Salmonella virus BSP161, Shigella virus A7, Yersinia virus Berlin, Yersinia virus PYPS50, Yersinia virus Yepe2, Yersinia virus Yepf, Citrobacter virus CR8, Vibrio virus ICP3, Vibrio virus N4, Vibrio virus VP4, Enterobacter virus Eapa, Erwinia virus L1, Escherichia virus SRT7, Pseudomonas virus 17A, Pseudomonas virus ghl, Pseudomonas virus Henninger, Pseudomonas virus KNP, Pseudomonas virus Pf1ERZ2017, Pseudomonas virus PhiPSA2, Pseudomonas virus PhiPsa17, Pseudomonas virus PPPL1, Pseudomonas virus sh12, Pseudomonas virus WRT, Yersinia virus fPS9, Yersinia virus fPS53, Yersinia virus fPS59, Yersinia virus fPS54ocr, Pectobacterium virus Jarilo, Citrobacter virus CR44b, Citrobacter virus SH3, Citrobacter virus SH4, Cronobacter virus Dev2, Cronobacter virus GW1, Enterobacter virus EcpYZU01, Escherichia virus EcoDS1, Escherichia virus F, Escherichia virus GA2A, Escherichia virus IMM002, Escherichia virus K1F, Escherichia virus LM33P1, Escherichia virus PE3-1, Escherichia virus Ro451w, Escherichia virus ST31, Escherichia virus Vec13, Escherichia virus YZ1, Escherichia virus ZG49, Shigella virus SFPH2, Morganella virus MmP1, Morganella virus MP2, Dickeya virus JA10, Dickeya virus Ninurta, Pectobacterium virus PP47, Pectobacterium virus PP81, Pectobacterium virus PPWS4, Pseudomonas virus PPpW4, Pseudomonas virus 22PfluR64PP, Pseudomonas virus IBBPF7A, Pseudomonas virus Pf10, Pseudomonas virus PFP1, Pseudomonas virus PhiS1, Pseudomonas virus UNOSLW1, Pseudomonas virus PspYZU08, Escherichia virus K30, Klebsiella virus 2044-307w, Klebsiella virus BIS33, Klebsiella virus Henu1, Klebsiella virus IL33, Klebsiella virus IME205, Klebsiella virus IME321, Klebsiella virus K5, Klebsiella virus K11, Klebsiella virus K5-2, Klebsiella virus K5-4, Klebsiella virus KN1-1, Klebsiella virus KN3-1, Klebsiella virus KN4-1, Klebsiella virus Kp1, Klebsiella virus KP32, Klebsiella virus KP32i192, Klebsiella virus KP32i194, Klebsiella virus KP32i195, Klebsiella virus KP32i196, Klebsiella virus kpssk3, Klebsiella virus KpV289, Klebsiella virus KpV763, Klebsiella virus KpV766, Klebsiella virus KpV767, Klebsiella virus Pharr, Klebsiella virus PRA33, Klebsiella virus SHKp152234, Klebsiella virus SHKp152410, Citrobacter virus CFP1, Citrobacter virus SH1, Citrobacter virus SH2, Enterobacter virus E2, Enterobacter virus E3, Enterobacter virus KPN3, Enterobacteria virus T7M, Escherichia virus ECA2, Escherichia virus LL2, Escherichia virus T3, Escherichia virus T3Luria, Leclercia virus 10164-302, Salmonella virus SG-JL2, Serratia virus 2050H2, Serratia virus SM9-3Y, Yersinia virus APS, Yersinia virus YeF10, Yersinia virus YeO3-12, Enterobacteria virus IME390, Escherichia virus 13a, Escherichia virus 64795ec1, Escherichia virus C5, Escherichia virus CICC80001, Escherichia virus Ebrios, Escherichia virus EG1, Escherichia virus HZ2R8, Escherichia virus HZP2, Escherichia virus N30, Escherichia virus NCA, Escherichia virus T7, Salmonella virus 3A8767, Salmonella virus Vi06, Stenotrophomonas virus IME15, Yersinia virus YpPY, Yersinia virus YpsPG, Pseudomonas virus Phi15, Pectobacterium virus DUPPII, Synechococcus virus SCBP42, Aquamicrobium virus P14, Ashivirus S45C4, Agrobacterium virus Atuph02, Agrobacterium virus Atuph03, Ralstonia virus Ap1, Ayaqvirus S45C18, Prochlorococcus virus SS120-1, Pseudomonas virus Andromeda, Pseudomonas virus Bf7, Escherichia virus J8-65, Escherichia virus Lidtsur, Prochlorococcus virus NATL1A7, Chosvirus KM23C739, Rhizobium virus RHEph02, Rhizobium virus RHEph08, Rhizobium virus RHEph09, Vibrio virus Cyclit, Escherichia virus PGT2, Escherichia virus PhiKT, Alteromonas virus H4-4, Foussvirus S46C10, Fussvirus S30C28, Escherichia virus ECBP5, Pectobacterium virus PP99, Ralstonia virus DURPI, Ralstonia virus RsoP1EGY, Synechococcus STIP37, Jalkavirus S08C159, Ralstonia virus RSB3, Kawavirus SWce1C56, Synechococcus virus SRIP1, Providencia virus PS3, Curvibacter virus P26059B, Ralstonia virus RSB2, Synechococcus virus SCBP2, Krakvirus S39C11, Podovirus Lau218, Pantoea virus LIMElight, Prochlorococcus virus PGSP1, Synechococcus virus SCBP3, Caulobacter virus Lullwater, Vibrio virus KF1, Vibrio virus KF2, Vibrio virus OWB, Vibrio virus VP93, Pseudomonas virus VSW3, Nohivirus S31C1, Oinezvirus S37C6, Rhizobium virus RHEph01, Pagavirus S05C849, Mesorhizobium virus Lo5R7ANS, Pedosvirus S28C3, Pekhitvirus S04C24, Pelagibacter virus HTVC019P, Pelagivirus S35C6, Caulobacter virus Percy, Delftia virus IMEDE1, Podivirus S05C243, Pseudomonas virus PollyC, Synechococcus virus SCBP4, Powvirus S08C41, Xanthomonas virus f20, Xanthomonas virus f30, Xanthomonas virus XAJ24, Xanthomonas virus Xc10, Xylella virus Prado, Synechococcus virus SB28, Sphingomonas virus Scott, Synechococcus virus SRIP2, Ralstonia virus ITL1, Sieqvirus S42C7, Ralstonia virus RPSC1, Stopalavirus S38C3, Pelagibacter virus HTVC011P, Stupnyavirus KM16C193, Prochlorococcus virus 951510a, Prochlorococcus virus NATL2A133, Prochlorococcus virus PSSP10, Vibrio virus JSF7, Prochlorococcus virus PSSP7, Synechococcus virus P60, Prochlorococcus virus PSSP3, Synechococcus virus PSSP2, Synechococcus virus Syn5, Votkovvirus S28C10, Pantoea virus LIMEzero, Pasteurella virus PHB01, Pasteurella virus PHB02, Escherichia virus GJ1, Escherichia virus ST32, Erwinia virus Faunus, Erwinia virus Y2, Aeromonas virus pAh6C, Pectobacterium virus PM1, Pectobacterium virus PP101, Shewanella virus Spp001, Shewanella virus SppYZU05, Vibrio virus Ceto, Vibrio virus Thalassa, Vibrio virus JSF10, Vibrio virus JSF12, Vibrio virus phi3, Vibrio virus pVp1, Escherichia virus EPS7, Escherichia virus mar003J3, Escherichia virus saus132, Salmonella virus 123, Salmonella virus 329, Salmonella virus 118970sa12, Salmonella virus LVR16A, Salmonella virus S113, Salmonella virus S114, Salmonella virus S116, Salmonella virus S124, Salmonella virus S126, Salmonella virus S132, Salmonella virus S133, Salmonella virus S147, Salmonella virus Seafire, Salmonella virus SH9, Salmonella virus STG2, Salmonella virus Stitch, Salmonella virus Sw2, Yersinia virus phiR201, Escherichia virus AKFV33, Escherichia virus BF23, Escherichia virus chee24, Escherichia virus DT5712, Escherichia virus DT57C, Escherichia virus FFH1, Escherichia virus Gostya9, Escherichia virus H8, Escherichia virus mar004NP2, Escherichia virus OSYSP, Escherichia virus phiAPCEc03, Escherichia virus phiLLS, Escherichia virus s1ur09, Escherichia virus T5, Salmonella virus NR01, Salmonella virus S131, Salmonella virus Shivani, Salmonella virus SP01, Salmonella virus SP3, Salmonella virus SPC35, Shigella virus SHSML45, Shigella virus SSP1, Pectobacterium virus DUPPV, Pectobacterium virus My1, Proteus virus PM135, Proteus virus Stubb, Vibrio virus PG07, Vibrio virus VspSw1, Aeromonas virus AhSzq1, Aeromonas virus AhSzw1, Klebsiella virus IME260, Klebsiella virus Sugarland, Escherichia virus IME542, Escherichia virus ACGM12, Escherichia virus EC3a, Escherichia virus DTL, Escherichia virus IME253, Escherichia virus Rtp, Shigella virus Sf12, Escherichia virus phiEB49, Escherichia virus AHP42, Escherichia virus AHS24, Escherichia virus AKS96, Escherichia virus C119, Escherichia virus E41c, Escherichia virus Eb49, Escherichia virus Jk06, Escherichia virus KP26, Escherichia virus phiJLA23, Escherichia virus Rogue1, Shigella virus Sd1, Shigella virus pSf1, Citrobacter virus DK2017, Citrobacter virus Sazh, Citrobacter virus Stevie, Escherichia virus LL5, Escherichia virus TLS, Salmonella virus 36, Salmonella virus PHB07, Salmonella virus phSE2, Salmonella virus SP126, Salmonella virus YSP2, Escherichia virus 95, Escherichia virus mar001J1, Escherichia virus mar002J2, Escherichia virus SECphi27, Escherichia virus swan01, Escherichia virus IME347, Escherichia virus SRT8, Escherichia virus ADB2, Escherichia virus BIFF, Escherichia virus IME18, Escherichia virus JMPW1, Escherichia virus JMPW2, Escherichia virus SH2, Escherichia virus T1, Shigella virus 008, Shigella virus ISF001, Shigella virus PSf2, Shigella virus Sfin1, Shigella virus SH6, Shigella virus Shfl1, Shigella virus ISF002, Cronobacter virus Esp2949-1, Enterobacter virus EcL1, Cronobacter virus PhiCS01, Escherichia virus ESCO41, Pantoea virus AAS23, Escherichia virus NBD2, Enterobacter virus F20, Klebsiella virus 1513, Klebsiella virus GHK3, Klebsiella virus KLPN1, Klebsiella virus KOX1, Klebsiella virus KP36, Klebsiella virus KpCol1, Klebsiella virus KpKT2lphi1, Klebsiella virus KPN N141, Klebsiella virus KpV522, Klebsiella virus MezzoGao, Klebsiella virus NJR15, Klebsiella virus NJS1, Klebsiella virus NJS2, Klebsiella virus PKP126, Klebsiella virus Sushi, Klebsiella virus TAH8, Klebsiella virus TSK1, Bacillus virus Agate, Bacillus virus Bobb, Bacillus virus Bp8pC, Bacillus virus Bastille, Bacillus virus CAM003, Bacillus virus Evoli, Bacillus virus HoodyT, Bacillus virus AvesoBmore, Bacillus virus B4, Bacillus virus Bigbertha, Bacillus virus Riley, Bacillus virus Spock, Bacillus virus Troll, Bacillus virus Bc431, Bacillus virus Bcp1, Bacillus virus BCP82, Bacillus virus BM15, Bacillus virus Deepblue, Bacillus virus JBP901, Bacillus virus Grass, Bacillus virus NIT1, Bacillus virus SPG24, Bacillus virus BCP78, Bacillus virus TsarBomba, Bacillus virus BPS13, Bacillus virus BPS10C, Bacillus virus Hakuna, Bacillus virus Megatron, Bacillus virus WPh, Bacillus virus Mater, Bacillus virus Moonbeam, Bacillus virus SIOphi, Enterococcus virus ECP3, Enterococcus virus EF24C, Enterococcus virus EFLK1, Enterococcus virus EFDG1, Enterococcus virus EFP01, Enterococcus virus EfV12, Listeria virus A511, Listeria virus AG20, Listeria virus List36, Listeria virus LMSP25, Listeria virus LMTA34, Listeria virus LMTA148, Listeria virus LP048, Listeria virus LP064, Listeria virus LP083-2, Listeria virus P100, Listeria virus WILL Bacillus virus Camphawk, Bacillus virus SPO1, Bacillus virus CP51, Bacillus virus JL, Bacillus virus Shanette, Staphylococcus virus BS1, Staphylococcus virus BS2, Lactobacillus virus Bacchae, Lactobacillus virus Bromius, Lactobacillus virus Iacchus, Lactobacillus virus Lpa804, Lactobacillus virus Semele, Staphylococcus virus G1, Staphylococcus virus G15, Staphylococcus virus JD7, Staphylococcus virus K, Staphylococcus virus MCE2014, Staphylococcus virus P108, Staphylococcus virus Rodi, Staphylococcus virus S253, Staphylococcus virus S25-4, Staphylococcus virus SA12, Staphylococcus virus Sb1, Staphylococcus virus SscM1, Staphylococcus virus IPLAC1C, Staphylococcus virus SEP1, Staphylococcus virus Remus, Staphylococcus virus SA11, Staphylococcus virus Stau2, Staphylococcus virus Twort, Brochothrix virus A9, Lactobacillus virus Lb338-1, Lactobacillus virus LP65, Campylobacter virus CP21, Campylobacter virus CP220, Campylobacter virus CPt10, Campylobacter virus IBB35, Campylobacter virus CP81, Campylobacter virus CP30A, Campylobacter virus CPX, Campylobacter virus Los1, Campylobacter virus NCTC12673, Escherichia virus Alf5, Escherichia virus AYO145A, Escherichia virus EC6, Escherichia virus HY02, Escherichia virus JH2, Escherichia virus TP1, Escherichia virus VpaE1, Escherichia virus wV8, Salmonella virus BPS15Q2, Salmonella virus BPS17L1, Salmonella virus BPS17W1, Salmonella virus FelixO1, Salmonella virus Mushroom, Salmonella virus Si3, Salmonella virus SP116, Salmonella virus UAB87, Erwinia virus Ea214, Erwinia virus M7, Citrobacter virus Moogle, Citrobacter virus Mordin, Shigella virus Sf13, Shigella virus Sf14, Shigella virus Sf17, Escherichia virus SUSP1, Escherichia virus SUSP2, Ralstonia virus RSA1, Ralstonia virus RSY1, Mannheimia virus 1127AP1, Mannheimia virus PHL101, Aeromonas virus phiO18P, Vibrio virus Canoe, Pseudoalteromonas virus C5a, Pseudomonas virus Dobby, Pseudomonas virus phiCTX, Erwinia virus EtG, Escherichia virus 186, Salmonella virus PsP3, Salmonella virus SEN1, Erwinia virus ENT90, Klebsiella virus 4LV2017, Salmonella virus Fels2, Salmonella virus RE2010, Salmonella virus SEN8, Salmonella virus SopEphi, Haemophilus virus HP1, Haemophilus virus HP2, Vibrio virus Kappa, Pasteurella virus F108, Burkholderia virus KS14, Burkholderia virus AP3, Burkholderia virus KS5, Vibrio virus K139, Burkholderia virus ST79, Escherichia virus fiAA91ss, Escherichia virus P2, Escherichia virus pro147, Escherichia virus pro483, Escherichia virus Wphi, Yersinia virus L413C, Pseudomonas virus phi3, Salinivibrio virus SMHB1, Klebsiella virus 3LV2017, Salmonella virus SEN4, Cronobacter virus ESSI2, Stenotrophomonas virus Smp131, Salmonella virus FSLSP004, Burkholderia virus KL3, Burkholderia virus phi52237, Burkholderia virus phiE122, Burkholderia virus phiE202, Vibrio virus PV94, Escherichia virus P88, Escherichia virus Bp7, Escherichia virus IME08, Escherichia virus JS10, Escherichia virus J598, Escherichia virus MX01, Escherichia virus QL01, Escherichia virus VR5, Escherichia virus WG01, Escherichia virus VR7, Escherichia virus VR20, Escherichia virus VR25, Escherichia virus VR26, Shigella virus SP18, Salmonella virus Melville, Salmonella virus S16, Salmonella virus STML198, Salmonella virus STP4a, Klebsiella virus JD18, Klebsiella virus PKO111, Enterobacter virus PG7, Escherichia virus CC31, Escherichia virus ECD7, Escherichia virus GEC3S, Escherichia virus JSE, Escherichia virus phi1, Escherichia virus RB49, Citrobacter virus CF1, Citrobacter virus Merlin, Citrobacter virus Moon, Escherichia virus APCEc01, Escherichia virus HP3, Escherichia virus HX01, Escherichia virus JS09, Escherichia virus 0157tp3, Escherichia virus 0157tp6, Escherichia virus PhAPEC2, Escherichia virus RB69, Escherichia virus ST0, Shigella virus SHSML521, Shigella virus UTAM, Vibrio virus KVP40, Vibrio virus nt1, Vibrio virus ValKK3, Enterobacter virus Eap3, Klebsiella virus KP15, Klebsiella virus KP27, Klebsiella virus Matisse, Klebsiella virus Miro, Klebsiella virus PMBT1, Escherichia virus AR1, Escherichia virus C40, Escherichia virus CF2, Escherichia virus E112, Escherichia virus ECML134, Escherichia virus HY01, Escherichia virus HY03, Escherichia virus Ime09, Escherichia virus RB3, Escherichia virus RB14, Escherichia virus s1ur03, Escherichia virus slur04, Escherichia virus T4, Shigella virus Pss1, Shigella virus Sf21, Shigella virus Sf22, Shigella virus Sf24, Shigella virus SHBML501, Shigella virus Shf12, Yersinia virus D1, Yersinia virus PST, Acinetobacter virus 133, Aeromonas virus 65, Aeromonas virus Aeh1, Escherichia virus RB16, Escherichia virus RB32, Escherichia virus RB43, Pseudomonas virus 42, Escherichia virus Av05, Cronobacter virus CR3, Cronobacter virus CR8, Cronobacter virus CR9, Cronobacter virus PBES02, Pectobacterium virus phiTE, Cronobacter virus GAP31, Escherichia virus 4MG, Salmonella virus PVPSE1, Salmonella virus SSE121, Escherichia virus APECc02, Escherichia virus FFH2, Escherichia virus FV3, Escherichia virus JES2013, Escherichia virus Murica, Escherichia virus s1ur16, Escherichia virus V5, Escherichia virus V18, Brevibacillus virus Abouo, Brevibacillus virus Davies, Synechococcus virus SMbCM100, Erwinia virus Deimos, Erwinia virus Desertfox, Erwinia virus Ea35-70, Erwinia virus RAY, Erwinia virus Simmy50, Erwinia virus SpecialG, Synechococcus virus SShM2, Klebsiella virus K64-1, Klebsiella virus RaK2, Dickeya virus AD1, Erwinia virus Alexandra, Lactobacillus virus LBR48, Synechococcus virus SCAM1, Synechococcus virus SCBWM1, Vibrio virus Aphrodite1, Escherichia virus 121Q, Eschierichia virus PBECO4, Synechococcus virus AC2014fSyn7803C8, Synechococcus virus ACG2014f, Synechococcus virus ACG2014fSyn7803US26, Synechococcus virus STIMS, Pseudomonas virus PaBG, Rheinheimera virus Barba18A, Rheinheimera virus Barba19A, Rheinheimera virus Barba21A, Rheinheimera virus Barba5S, Rheinheimera virus Barba8S, Burkholderia virus BcepMu, Burkholderia virus phiE255, Synechococcus virus Bellamy, Gordonia virus GMA6, Aeromonas virus 44RR2, Mycobacterium virus Alice, Mycobacterium virus Bxz1, Mycobacterium virus Dandelion, Mycobacterium virus HyRo, Mycobacterium virus 13, Mycobacterium virus Lukilu, Mycobacterium virus Nappy, Mycobacterium virus Sebata, Faecalibacterium virus Brigit, Prochlorococcus virus Syn33, Synechococcus virus SRIM12-01, Synechococcus virus SRIM12-06, Synechococcus virus SRIM12-08, Salmonella virus SEN34, Acidovorax virus ACP17, Xanthomonas virus Carpasina, Xanthomonas virus XcP1, Pseudomonas virus pf16, Synechococcus virus SCAM3, Ralstonia virus RSF1, Ralstonia virus RSL2, Synechococcus virus SWAM2, Erwinia virus Derbicus, Pseudomonas virus EL, Sinorhizobium virus M7, Sinorhizobium virus M12, Sinorhizobium virus N3, Serratia virus BF, Yersinia virus Yen9-04, Faecalibacterium virus Epona, Erwinia virus Asesino, Erwinia virus EaH2, Prochlorococcus virus MED4-213, Prochlorococcus virus PHM1, Prochlorococcus virus PHM2, Flavobacterium virus FCL2, Flavobacterium virus FCV1, Pseudomonas virus KIL2, Pseudomonas virus KIL4, Edwardsiella virus GF2, Escherichia virus Goslar, Halomonas virus HAP1, Vibrio virus VP882, Lactobacillus virus Lb, Erwinia virus EaH1, Iodobacter virus PLPE, Delftia virus PhiW14, Klebsiella virus JD001, Klebsiella virus KpV52, Klebsiella virus KpV80, Escherichia virus CVM10, Escherichia virus EC0078, Escherichia virus ep3, Brevibacillus virus Jimmer, Brevibacillus virus Osiris, Synechococcus virus SCAMS, Rhizobium virus RHEph4, Faecalibacterium virus Lagaffe, Synechoccus virus SP4, Synechococcus virus Syn30, Prochlorococcus virus PTIM40, Synechococcus virus SSKS1, Salmonella virus ZCSE2, Clostridium virus phiC2, Clostridium virus phiCD27, Clostridium virus phiCD119, Erwinia virus Machina, Arthrobacter virus BarretLemon, Arthrobacter virus Beans, Arthrobacter virus Brent, Arthrobacter virus Jawnski, Arthrobacter virus Martha, Arthrobacter virus Piccoletto, Arthrobacter virus Shade, Arthrobacter virus Sonny, Synechococcus virus SCAM7, Acinetobacter virus ME3, Ralstonia virus RSL1, Cronobacter virus GAP32, Pectinobacterium virus CBB, Faecalibacterium virus Mushu, Escherichia virus Mu, Shigella virus SfMu, Halobacterium virus phiH, Burkholderia virus Bcepl, Burkholderia virus Bcep43, Burkholderia virus Bcep781, Burkholderia virus BcepNY3, Xanthomonas virus OP2, Synechococcus virus SMbCM6, Pseudomonas virus Ab03, Pseudomonas virus G1, Pseudomonas virus KPP10, Pseudomonas virus PAKP3, Pseudomonas virus PS24, Synechococcus virus SRIM8, Synechococcus virus SRIM50, Synechococcus virus ACG2014bSyn7803C61, Synechococcus virus ACG2014bSyn9311C4, Synechococcus virus SRIM2, Synechococcus virus SPM2, Pseudomonas virus Noxifer, Acinetobacter virus AB1, Acinetobacter virus AB2, Acinetobacter virus AbC62, Acinetobacter virus AbP2, Acinetobacter virus AP22, Acinetobacter virus LZ35, Acinetobacter virus WCHABP1, Acinetobacter virus WCHABP12, Pseudomonas virus Psa374, Pseudomonas virus VCM, Pseudomonas virus CAb 1, Pseudomonas virus CAb02, Pseudomonas virus JG004, Pseudomonas virus MAGI, Pseudomonas virus PA10, Pseudomonas virus PAKP1, Pseudomonas virus PAKP2, Pseudomonas virus PAKP4, Pseudomonas virus PaP1, Pseudomonas virus phiMK, Pseudomonas virus Zigelbrucke, Prochlorococcus virus PSSM7, Burkholderia virus BcepF1, Pseudomonas virus 141, Pseudomonas virus Ab28, Pseudomonas virus CEBDP1, Pseudomonas virus DL60, Pseudomonas virus DL68, Pseudomonas virus E215, Pseudomonas virus E217, Pseudomonas virus F8, Pseudomonas virus JG024, Pseudomonas virus KPP12, Pseudomonas virus KTN6, Pseudomonas virus LBL3, Pseudomonas virus LMA2, Pseudomonas virus NH4, Pseudomonas virus PA5, Pseudomonas virus PB1, Pseudomonas virus PS44, Pseudomonas virus SN, Pectinobacterium virus PEAT2, Edwardsiella virus pEtSU, Bordetella virus PHB04, Escherichia phage ESCO13, Escherichia virus ESCO5, Escherichia virus phAPEC8, Escherichia virus Schickermooser, Klebsiella virus ZCKP1, Pseudomonas virus PA7, Pseudomonas virus phiKZ, Pseudomonas virus SL2, Pseudomonas virus PMW, Agrobacterium virus Atuph07, Synechococcus virus Syn19, Aeromonas virus 56, Aeromonas virus 43, Escherichia virus P1, Escherichia virus RCS47, Salmonella virus SJ46, Pseudoalteromonas virus J2-1, Arthrobacter virus ArV1, Arthrobacter virus Colucci, Arthrobacter virus Trina, Ralstonia virus RP12, Erwinia virus Risingsun, Salmonella virus BP63, Acinetobacter virus Aci05, Acinetobacter virus Aci01-1, Acinetobacter virus Aci02-2, Prochlorococcus virus PSSM2, Dickeya virus JA11, Dickeya virus JA29, Erwinia virus Y3, Agrobacterium virus 7-7-1, Salmonella virus SPN3US, Bacillus virus Shbh1, Bacillus virus 1, Geobacillus virus GBSV1, Pseudomonas virus tabernarius, Synechococcus virus ST4, Faecalibacterium virus Taranis, Synechococcus virus SIOM18, Yersinia virus R1RT, Yersinia virus TG1, Synechococcus virus STIM4, Synechococcus virus SSM1, Bacillus virus SP15, Vibrio virus pTD1, Vibrio virus VP4B, Tetrasphaera virus TJEL Faecalibacterium virus Toutatis, Aeromonas virus 25, Aeromonas virus Aes12, Aeromonas virus Aes508, Aeromonas virus AS4, Aeromonas virus Asgz, Stenotrophomonas virus IME13, Prochlorococcus virus Syn1, Synechococcus virus SRIM44, Vibrio virus MAR, Vibrio virus VHML, Vibrio virus VP585, Escherichia virus ECML4, Salmonella virus Marshall, Salmonella virus Maynard, Salmonella virus SJ2, Salmonella virus STML131, Salmonella virus ViI, Erwinia virus Wellington, Escherichia virus ECML-117, Escherichia virus FEC19, Escherichia virus WFC, Escherichia virus WFH, Serratia virus CHI14, Edwardsiella virus MSW3, Edwardsiella virus PEi21, Erwinia virus Yoloswag, Bacillus virus G, Bacillus virus PBS1, Microcystis virus Ma-LMM01, Streptococcus virus Cp1, Streptococcus virus Cp1, Lactococcus virus WP2, Bacillus virus B103, Bacillus virus GA1, Bacillus virus phi29, Kurthia virus 6, Actinomyces virus Av1, Mycoplasma virus P1, Staphylococcus virus Andhra, Staphylococcus virus St134, Staphylococcus virus 66, Staphylococcus virus 44AHJD, Staphylococcus virus BP39, Staphylococcus virus CSA13, Staphylococcus virus GRCS, Staphylococcus virus Pabna, Staphylococcus virus phiAGO13, Staphylococcus virus PSa3, Staphylococcus virus S24-1, Staphylococcus virus SAP2, Staphylococcus virus SCH1, Staphylococcus virus SLPW, Shigella virus 7502Stx, Shigella virus POCJ13, Escherichia virus 191, Escherichia virus PA2, Escherichia virus TL2011, Shigella virus VASD, Escherichia virus 24B, Escherichia virus 933W, Escherichia virus Min27, Escherichia virus PA28, Escherichia virus Stx2 II, Dinoroseobacter virus DFL12, Pseudomonas virus Bjorn, Pseudomonas virus Ab22, Pseudomonas virus CHU, Pseudomonas virus LUZ24, Pseudomonas virus PAA2, Pseudomonas virus PaP3, Pseudomonas virus PaP4, Pseudomonas virus TL, Vibrio virus VC8, Vibrio virus VP2, Vibrio virus VP5, Escherichia virus N4, Flavobacterium virus Fpv1, Flavobacterium virus Fpv4, Streptococcus virus C1, Escherichia virus APEC5, Escherichia virus APEC7, Escherichia virus Bp4, Escherichia virus EC1UPM, Escherichia virus ECBP1, Escherichia virus G7C, Escherichia virus IME11, Shigella virus Sb1, Escherichia virus C1302, Pseudomonas virus F116, Pseudomonas virus H66, Escherichia virus Pollock, Salmonella virus FSL SP-058, Salmonella virus FSL SP-076, Arthrobacter virus Adat, Arthrobacter virus Jasmine, Erwinia virus Ea9-2, Erwinia virus Frozen, Achromobacter virus Axp3, Achromobacter virus JWAlpha, Edwardsiella virus KF1, Burkholderia virus KL4, Pseudomonas virus KPP25, Pseudomonas virus R18, Pseudomonas virus tf, Escherichia virus 172-1, Escherichia virus ECB2, Escherichia virus NJ01, Escherichia virus phiEco32, Escherichia virus Septima11, Escherichia virus SU10, Escherichia virus HK620, Salmonella virus BTP1, Salmonella virus P22, Salmonella virus SE1Spa, Salmonella virus ST64T, Shigella virus Sf6, Burkholderia virus Bcep22, Burkholderia virus Bcepi102, Burkholderia virus Bcepmigl, Burkholderia virus DC1, Cellulophaga virus Cba41, Cellulophaga virus Cba172, Pseudomonas virus Ab09, Pseudomonas virus LIT1, Pseudomonas virus PA26, Pseudomonas virus KPP21, Pseudomonas virus LUZ7, Vibrio virus 48B1, Vibrio virus 51A6, Vibrio virus 51A7, Vibrio virus 52B1, Myxococcus virus Mx8, Bacillus virus Page, Bacillus virus Palmer, Bacillus virus Pascal, Bacillus virus Pony, Bacillus virus Pookie, Brucella virus Pr, Brucella virus Tb, Bordetella virus BPP1, Burkholderia virus BcepC6B, Helicobacter virus 1961P, Helicobacter virus KHP30, Helicobacter virus KHP40, Pseudomonas virus phCDa, Escherichia virus Skarpretter, Escherichia virus Sortsne, Klebsiella virus IME279, Escherichia virus phiV10, Salmonella virus Epsilon15, Salmonella virus SPN1S, Pseudomonas virus NV1, Pseudomonas virus UFVP2, Escherichia virus PTXU04, Hamiltonella virus APSE1, Lactococcus virus KSY1, Phormidium virus WMP3, Phormidium virus WMP4, Pseudomonas virus 119X, Roseobacter virus SIO1, Vibrio virus VpV262, Streptomyces virus ELB20, Streptomyces virus R4, Streptomyces virus Amela, Streptomyces virus phiCAM, Streptomyces virus Aaronocolus, Streptomyces virus Caliburn, Streptomyces virus Danzina, Streptomyces virus Hydra, Streptomyces virus Izzy, Streptomyces virus Lannister, Streptomyces virus Lika, Streptomyces virus Sujidade, Streptomyces virus Zemlya, Streptomyces virus phiHau3, Mycobacterium virus Acadian, Mycobacterium virus Baee, Mycobacterium virus Reprobate, Mycobacterium virus Adawi, Mycobacterium virus Banel, Mycobacterium virus BrownCNA, Mycobacterium virus Chrisnmich, Mycobacterium virus Cooper, Mycobacterium virus JAMaL, Mycobacterium virus Nigel, Mycobacterium virus Stinger, Mycobacterium virus Vincenzo, Mycobacterium virus Zemanar, Mycobacterium virus Apizium, Mycobacterium virus Manad, Mycobacterium virus Oline, Mycobacterium virus Osmaximus, Mycobacterium virus Pg1, Mycobacterium virus Soto, Mycobacterium virus Suffolk, Mycobacterium virus Athena, Mycobacterium virus Bernardo, Mycobacterium virus Gadjet, Mycobacterium virus Pipefish, Mycobacterium virus Godines, Mycobacterium virus Rosebush, Mycobacterium virus TA17a, Mycobacterium virus Babsiella, Mycobacterium virus Brujita, Mycobacterium virus Hawkeye, Mycobacterium virus Plot, Caulobacter virus CcrBL9, Caulobacter virus CcrSC, Caulobacter virus CcrColossus, Caulobacter virus CcrPW, Caulobacter virus CcrBL10, Caulobacter virus CcrRogue, Caulobacter virus phiCbK, Caulobacter virus Swift, Salmonella virus SP31, Salmonella virus AG11, Salmonella virus Ent1, Salmonella virus f18SE, Salmonella virus Jersey, Salmonella virus L13, Salmonella virus LSPA1, Salmonella virus SE2, Salmonella virus SETP3, Salmonella virus SETP7, Salmonella virus SETP13, Salmonella virus SP101, Salmonella virus SS3e, Salmonella virus wks13, Escherichia virus K1G, Escherichia virus K1H, Escherichia virus Klind1, Escherichia virus Klind2, Esherichia virus Golestan, Raoultella virus RP180, Gordonia virus Asapag, Gordonia virus BENtherdunthat, Gordonia virus Getalong, Gordonia virus Kenna, Gordonia virus Horus, Gordonia virus Phistory, Leuconostoc virus Lmd1, Leuconostoc virus LN03, Leuconostoc virus LN04, Leuconostoc virus LN12, Leuconostoc virus LN6B, Leuconostoc virus P793, Leuconostoc virus 1A4, Leuconostoc virus Ln8, Leuconostoc virus Ln9, Leuconostoc virus LN25, Leuconostoc virus LN34, Leuconostoc virus LNTR3, Mycobacterium virus Bongo, Mycobacterium virus Rey, Mycobacterium virus Butters, Mycobacterium virus Michelle, Mycobacterium virus Charlie, Mycobacterium virus Pipsqueaks, Mycobacterium virus Xeno, Mycobacterium virus Panchino, Mycobacterium virus Phrann, Mycobacterium virus Redi, Mycobacterium virus Skinnyp, Gordonia virus BaxterFox, Gordonia virus Yeezy, Gordonia virus Kita, Gordonia virus Nymphadora, Gordonia virus Zirinka, Mycobacterium virus Bignuz, Mycobacterium virus Brusacoram, Mycobacterium virus Donovan, Mycobacterium virus Fishburne, Mycobacterium virus Jebeks, Mycobacterium virus Malithi, Mycobacterium virus Phayonce, Lactobacillus virus B2, Lactobacillus virus Lenus, Lactobacillus virus Nyseid, Lactobacillus virus SAC12, Lactobacillus virus Ldl1, Lactobacillus virus ViSo2018a, Lactobacillus virus Maenad, Lactobacillus virus P1, Lactobacillus virus Satyr, Streptomyces virus AbbeyMikolon, Pseudomonas virus Ab18, Pseudomonas virus Ab19, Pseudomonas virus PaMx11, Burkholderia virus AH2, Arthrobacter virus Amigo, Arthrobacteria virus Molivia, Propionibacterium virus Anatole, Propionibacterium virus B3, Arthrobacter virus Andrew, Bacillus virus Andromeda, Bacillus virus Blastoid, Bacillus virus Curly, Bacillus virus Eoghan, Bacillus virus Finn, Bacillus virus Glittering, Bacillus virus Riggi, Bacillus virus Taylor, Microbacterium virus Appa, Gordonia virus Apricot, Microbacterium virus Armstrong, Gordonia virus Attis, Streptomyces virus Attoomi, Streptomyces virus Austintatious, Streptomyces virus Ididsumtinwong, Streptomyces virus PapayaSalad, Gordonia virus Bantam, Mycobacterium virus Barnyard, Mycobacterium virus Konstantine, Mycobacterium virus Predator, Pseudomonas virus B3, Pseudomonas virus JBD67, Pseudomonas virus JD18, Pseudomonas virus PM105, Mycobacterium virus Bernal13, Gordonia virus BetterKatz, Streptomyces virus Bing, Staphylococcus virus 13, Staphylococcus virus 77, Staphylococcus virus 108PVL, Gordonia virus Bowser, Arthrobacter virus Bridgette, Arthrobacter virus Constance, Arthrobacter virus Eileen, Arthrobacter virus Judy, Arthrobacter virus Peas, Gordonia virus Britbrat, Mycobacterium virus Bron, Mycobacterium virus Faith1, Mycobacterium virus JoeDirt, Mycobacterium virus Rumpelstiltskin, Streptococcus virus 858, Streptococcus virus 2972, Streptococcus virus ALQ132, Streptococcus virus 01205, Streptococcus virus Sfi11, Pseudomonas virus D3112, Pseudomonas virus DMS3, Pseudomonas virus FHA0480, Pseudomonas virus LPB1, Pseudomonas virus MP22, Pseudomonas virus MP29, Pseudomonas virus MP38, Pseudomonas virus PA1KOR, Cellulophaga virus ST, Bacillus virus 250, Bacillus virus IEBH, Lactococcus virus bIL67, Lactococcus virus c2, Corynebacterium virus C3PO, Corynebacterium virus Darwin, Corynebacterium virus Zion, Lactobacillus virus c5, Lactobacillus virus Ld3, Lactobacillus virus Ld17, Lactobacillus virus Ld25A, Lactobacillus virus LLKu, Lactobacillus virus phiLdb, Mycobacterium virus Che9c, Mycobacterium virus Sbash, Mycobacterium virus Ardmore, Mycobacterium virus Avani, Mycobacterium virus Boomer, Mycobacterium virus Che8, Mycobacterium virus Che9d, Mycobacterium virus DeadP, Mycobacterium virus Dlane, Mycobacterium virus Dorothy, Mycobacterium virus DotProduct, Mycobacterium virus Drago, Mycobacterium virus Fruitloop, Mycobacterium virus GUmbie, Mycobacterium virus Ibhubesi, Mycobacterium virus Llij, Mycobacterium virus Mozy, Mycobacterium virus Mutaforma13, Mycobacterium virus Pacc40, Mycobacterium virus PMC, Mycobacterium virus Ramsey, Mycobacterium virus RockyHorror, Mycobacterium virus SG4, Mycobacterium virus Shauna1, Mycobacterium virus Shilan, Mycobacterium virus Spartacus, Mycobacterium virus Taj, Mycobacterium virus Tweety, Mycobacterium virus Wee, Mycobacterium virus Yoshi, Salmonella virus Chi, Salmonella virus FSLSP030, Salmonella virus FSLSP088, Salmonella virus iEPS5, Salmonella virus SPN19, Corynebacterium virus P1201, Clavibacter virus CMP1, Clavibacter virus CN1A, Lactobacillus virus ATCC8014, Lactobacillus virus phiJL1, Pediococcus virus cIP1, Arthrobacter virus Coral, Arthrobacter virus Kepler, Mycobacterium virus Corndog, Mycobacterium virus Firecracker, Rhodobacter virus RcCronus, Gordonia virus DareDevil, Arthrobacter virus Decurro, Stenotrophomonas virus DLPS, Gordonia virus Demosthenes, Gordonia virus Katyusha, Gordonia virus Kvothe, Pseudomonas virus D3, Pseudomonas virus PMG1, Escherichia virus EK99P1, Escherichia virus HK578, Escherichia virus JL1, Escherichia virus SSL2009a, Escherichia virus YD2008s, Shigella virus EP23, Sodalis virus S01, Microbacterium virus Dismas, Propionibacterium virus B22, Propionibacterium virus Doucette, Propionibacterium virus E6, Propionibacterium virus G4, Microbacterium virus Eden, Enterococcus virus AL2, Enterococcus virus AL3, Enterococcus virus AUEF3, Enterococcus virus EcZZ2, Enterococcus virus EF3, Enterococcus virus EF4, Enterococcus virus EfaCPT1, Enterococcus virus IME196, Enterococcus virus LY0322, Enterococcus virus phiSHEF2, Enterococcus virus phiSHEF4, Enterococcus virus phiSHEF5, Enterococcus virus PMBT2, Enterococcus virus SANTOR1, Edwardsiella virus eiAU, Xanthomonas virus PhiL7, Microbacterium virus Eleri, Gordonia virus Cozz, Gordonia virus Emalyn, Gordonia virus GTE2, Gordonia virus Troje, Gordonia virus Eyre, Gordonia virus Fairfaxidumvirus, Microbacterium virus ISF9, Erwinia virus Eho49, Erwinia virus Eho59, Staphylococcus virus 2638A, Staphylococcus virus QT1, Colwellia virus 9A, Mycobacterium virus Alma, Mycobacterium virus Arturo, Mycobacterium virus Astro, Mycobacterium virus Backyardigan, Mycobacterium virus Benedict, Mycobacterium virus Bethlehem, Mycobacterium virus Billknuckles, Mycobacterium virus BPBiebs31, Mycobacterium virus Bruns, Mycobacterium virus Bxb 1, Mycobacterium virus Bxz2, Mycobacterium virus Che12, Mycobacterium virus Cuco, Mycobacterium virus D29, Mycobacterium virus Doom, Mycobacterium virus Ericb, Mycobacterium virus Euphoria, Mycobacterium virus George, Mycobacterium virus Gladiator, Mycobacterium virus Goose, Mycobacterium virus Hammer, Mycobacterium virus Heldan, Mycobacterium virus Jasper, Mycobacterium virus JC27, Mycobacterium virus Jeffabunny, Mycobacterium virus JHC117, Mycobacterium virus KBG, Mycobacterium virus Kssjeb, Mycobacterium virus Kugel, Mycobacterium virus L5, Mycobacterium virus Lesedi, Mycobacterium virus LHTSCC, Mycobacterium virus lockley, Mycobacterium virus Marcell, Mycobacterium virus Microwolf, Mycobacterium virus Mrgordo, Mycobacterium virus Museum, Mycobacterium virus Nepal, Mycobacterium virus Packman, Mycobacterium virus Peaches, Mycobacterium virus Perseus, Mycobacterium virus Pukovnik, Mycobacterium virus Rebeuca, Mycobacterium virus Redrock, Mycobacterium virus Ridgecb, Mycobacterium virus Rockstar, Mycobacterium virus Saintus, Mycobacterium virus Skipole, Mycobacterium virus Solon, Mycobacterium virus Switzer, Mycobacterium virus SWU1, Mycobacterium virus Tiger, Mycobacterium virus Timshel, Mycobacterium virus Trixie, Mycobacterium virus Turbido, Mycobacterium virus Twister, Mycobacterium virus U2, Mycobacterium virus Violet, Mycobacterium virus Wonder, Mycobacterium virus Gaia, Arthrobacter virus Abidatro, Arthrobacter virus Galaxy, Gordonia virus GAL1, Gordonia virus GMA3, Gordonia virus Gsput1, Gordonia virus GMAT, Gordonia virus GTE7, Gordonia virus Ghobes, Mycobacterium virus Giles, Microbacterium virus OneinaGillian, Gordonia virus GodonK, Microbacterium virus Goodman, Arthrobacter virus Captnmurica, Arthrobacter virus Gordon, Gordonia virus GordTnk2, Proteus virus Isfahan, Gordonia virus Jumbo, Gordonia virus Gustav, Gordonia virus Mandia, Paenibacillus virus Harrison, Gordonia virus Hedwig, Cellulophaga virus Cba121, Cellulophaga virus Cba171, Cellulophaga virus Cba181, Escherichia virus HK022, Escherichia virus HK75, Escherichia virus HK97, Escherichia virus HK106, Escherichia virus HK446, Escherichia virus HK542, Escherichia virus HK544, Escherichia virus HK633, Escherichia virus mEp234, Escherichia virus mEpX1, Escherichia virus mEpX2, Streptomyces virus Hiyaa, Salinibacter virus M1EM1, Salinibacter virus M8CR30-2, Listeria virus LP26, Listeria virus LP37, Listeria virus LP110, Listeria virus LP114, Listeria virus P70, Corynebacterium virus phi673, Corynebacterium virus phi674, Microbacterium virus Hamlet, Microbacterium virus Ilzat, Polaribacter virus P12002L, Polaribacter virus P12002S, Nonlabens virus P12024L, Nonlabens virus P12024S, Gordonia virus Jace, Brevibacillus virus Jenst, Corynebacterium virus Juicebox, Salinibacter virus M31CR41-2, Salinibacter virus SRUTV1, Arthrobacter virus Kellezzio, Arthrobacter virus Kitkat, Burkholderia virus KL1, Xanthomonas virus CP1, Microbacterium virus Golden, Microbacterium virus Koji, Arthrobacter virus Bennie, Arthrobacter virus DrRobert, Arthrobacter virus Glenn, Arthrobacter virus HunterDalle, Arthrobacter virus Joann, Arthrobacter virus Korra, Arthrobacter virus Preamble, Arthrobacter virus Pumancara, Arthrobacter virus Wayne, Mycobacterium virus 244, Mycobacterium virus Bask21, Mycobacterium virus CJW1, Mycobacterium virus Eureka, Mycobacterium virus Kostya, Mycobacterium virus Porky, Mycobacterium virus Pumpkin, Mycobacterium virus Sirduracell, Mycobacterium virus Toto, Microbacterium virus Krampus, Salinibacter virus M8CC19, Salinibacter virus M8CRM1, Sphingobium virus Lacusarx, Escherichia virus DE3, Escherichia virus HK629, Escherichia virus HK630, Escherichia virus Lambda, Pseudomonas virus Lana, Arthrobacter virus Laroye, Eggerthella virus PMBT5, Arthobacter virus Liebe, Mycobacterium virus Halo, Mycobacterium virus Liefie, Acinetobacter virus IMEAB3, Acinetobacter virus Loki, Streptomyces virus phiBT1, Streptomyces virus phiC31, Brevibacterium virus LuckyBarnes, Gordonia virus Lucky10, Faecalibacterium virus Lugh, Bacillus virus BMBtp2, Bacillus virus TP21, Bacillus virus Mgbh1, Arthrobacter virus Maj a, Arthrobacter virus DrManhattan, Mycobacterium virus Ff47, Mycobacterium virus Muddy, Vibrio virus MAR10, Vibrio virus SSP002, Mycobacterium virus Marvin, Mycobacterium virus Mosmoris, Pseudomonas virus PMBT3, Microbacterium virus MementoMori, Microbacterium virus Fireman, Microbacterium virus Metamorphoo, Microbacterium virus RobsFeet, Microbacterium virus Min1, Streptococcus virus 7201, Streptococcus virus DT1, Streptococcus virus phiAbc2, Streptococcus virus Sfi19, Streptococcus virus Sfi21, Gordinia virus Birksandsocks, Gordonia virus Flakey, Gordonia virus Monty, Gordonia virus Stevefrench, Arthrobacter virus Circum, Arthrobacter virus Mudcat, Escherichia virus EC2, Salmonella virus Lumpael, Dinoroseobacter virus D5C, Burkholderia virus BcepNazgul, Microbacterium virus Neferthena, Pseudomonas virus nickie, Pseudomonas virus NP1, Pseudomonas virus PaMx25, Escherichia virus 9g, Escherichia virus JenK1, Escherichia virus JenP1, Escherichia virus JenP2, Salmonella virus SE1Kor, Salmonella virus 9NA, Salmonella virus SP069, Gordonia virus Nyceirae, Faecalibacterium virus Oengus, Mycobacterium virus Baka, Mycobacterium virus Courthouse, Mycobacterium virus Littlee, Mycobacterium virus Omega, Mycobacterium virus Optimus, Mycobacterium virus Thibault, Gordonia virus BrutonGaster, Gordonia virus OneUp, Gordonia virus Orchid, Thermus virus P23-45, Thermus virus P74-26, Propionibacterium virus ATCC29399BC, Propionibacterium virus ATCC29399BT, Propionibacterium virus Attacne, Propionibacterium virus Keiki, Propionibacterium virus Kubed, Propionibacterium virus Lauchelly, Propionibacterium virus MrAK, Propionibacterium virus Ouroboros, Propionibacterium virus P91, Propionibacterium virus P105, Propionibacterium virus P144, Propionibacterium virus P1001, Propionibacterium virus P1.1, Propionibacterium virus P100A, Propionibacterium virus P100D, Propionibacterium virus P101A, Propionibacterium virus P104A, Propionibacterium virus PA6, Propionibacterium virus Pacnes201215, Propionibacterium virus PAD20, Propionibacterium virus PAS50, Propionibacterium virus PHL009M11, Propionibacterium virus PHL025M00, Propionibacterium virus PHL037M02, Propionibacterium virus PHL041M10, Propionibacterium virus PHL060L00, Propionibacterium virus PHL067M01, Propionibacterium virus PHL070N00, Propionibacterium virus PHL071N05, Propionibacterium virus PHL082M03, Propionibacterium virus PHL092M00, Propionibacterium virus PHL095N00, Propionibacterium virus PHL111M01, Propionibacterium virus PHL112N00, Propionibacterium virus PHL113M01, Propionibacterium virus PHL114L00, Propionibacterium virus PHL116M00, Propionibacterium virus PHL117M00, Propionibacterium virus PHL117M01, Propionibacterium virus PHL132N00, Propionibacterium virus PHL141N00, Propionibacterium virus PHL151M00, Propionibacterium virus PHL151N00, Propionibacterium virus PHL152M00, Propionibacterium virus PHL163M00, Propionibacterium virus PHL171M01, Propionibacterium virus PHL179M00, Propionibacterium virus PHL194M00, Propionibacterium virus PHL199M00, Propionibacterium virus PHL301M00, Propionibacterium virus PHL308M00, Propionibacterium virus Pirate, Propionibacterium virus Procrass1, Propionibacterium virus SKKY, Propionibacterium virus Solid, Propionibacterium virus Stormborn, Propionibacterium virus Wizzo, Pseudomonas virus PaMx28, Pseudomonas virus PaMx74, Mycobacterium virus Papyrus, Mycobacterium virus Send513, Mycobacterium virus Patience, Mycobacterium virus PBI1, Rhodococcus virus Pepy6, Rhodococcus virus Poco6, Staphylococcus virus 11, Staphylococcus virus 29, Staphylococcus virus 37, Staphylococcus virus 53, Staphylococcus virus 55, Staphylococcus virus 69, Staphylococcus virus 71, Staphylococcus virus 80, Staphylococcus virus 85, Staphylococcus virus 88, Staphylococcus virus 92, Staphylococcus virus 96, Staphylococcus virus 187, Staphylococcus virus 52a, Staphylococcus virus 80alpha, Staphylococcus virus CNPH82, Staphylococcus virus EW, Staphylococcus virus IPLA5, Staphylococcus virus IPLA7, Staphylococcus virus IPLA88, Staphylococcus virus PH15, Staphylococcus virus phiETA, Staphylococcus virus phiETA2, Staphylococcus virus phiETA3, Staphylococcus virus phiMR11, Staphylococcus virus phiMR25, Staphylococcus virus phiNM1, Staphylococcus virus phiNM2, Staphylococcus virus phiNM4, Staphylococcus virus SAP26, Staphylococcus virus X2, Enterococcus virus FL1, Enterococcus virus FL2, Enterococcus virus FL3, Streptomyces virus Picard, Microbacterium virus Pikmin, Corynebacterium virus Poushou, Providencia virus PR1, Listeria virus LP302, Listeria virus PSA, Psimunavirus psiM2, Propionibacterium virus PFR1, Microbacterium phage KaiHaiDragon, Microbacterium phage Paschalis, Microbacterium phage Quhwah, Streptomyces virus Darolandstone, Streptomyces virus Raleigh, Escherichia virus N15, Rhodococcus virus RER2, Rhizobium virus P106B, Strepomyces virus Drgrey, Strepomyces virus Rima, Microbacterium virus Hendrix, Gordonia virus Fryberger, Gordonia virus Ronaldo, Aeromonas virus pIS4A, Streptomyces virus Rowa, Gordonia virus Ruthy, Streptomyces virus Jay2Jay, Streptomyces virus Mildred21, Streptomyces virus NootNoot, Streptomyces virus Paradiddles, Streptomyces virus Peebs, Streptomyces virus Samisti12, Pseudomonas virus SM1, Corynebacterium virus SamW, Xylella virus Salvo, Xylella virus Sano, Caulobacter virus Sansa, Enterococcus virus BC611, Enterococcus virus IMEEF1, Enterococcus virus SAP6, Enterococcus virus VD13, Streptococcus virus SPQS1, Salmonella virus Sasha, Corynebacterium virus BFK20, Geobacillus virus Tp84, Streptomyces virus Scap1, Gordonia virus Schnabeltier, Microbacterium virus Schubert, Pseudomonas virus 73, Pseudomonas virus Ab26, Pseudomonas virus Kakheti25, Escherichia virus Cajan, Escherichia virus Seurat, Caulobacter virus Seuss, Staphylococcus virus SEP9, Staphylococcus virus Sextaec, Paenibacillus virus Diva, Paenibacillus virus Hb10c2, Paenibacillus virus Rani, Paenibacillus virus Shelly, Paenibacillus virus Sitara, Paenibacillus virus Willow, Lactococcus virus 712, Lactococcus virus ASCC191, Lactococcus virus ASCC273, Lactococcus virus ASCC281, Lactococcus virus ASCC465, Lactococcus virus ASCC532, Lactococcus virus Bibb29, Lactococcus virus bIL170, Lactococcus virus CB13, Lactococcus virus CB14, Lactococcus virus CB19, Lactococcus virus CB20, Lactococcus virus jj50, Lactococcus virus P2, Lactococcus virus P008, Lactococcus virus sk1, Lactococcus virus S14, Bacillus virus Slash, Bacillus virus Stahl, Bacillus virus Staley, Bacillus virus Stills, Gordonia virus Bachita, Gordonia virus ClubL, Gordonia virus Smoothie, Arthobacter virus Sonali, Gordonia virus Soups, Gordonia virus Strosahl, Gordonia virus Wait, Gordonia virus Sour, Bacillus virus SPbeta, Microbacterium virus Hyperion, Microbacterium virus Squash, Burkholderia virus phi6442, Burkholderia virus phi1026b, Burkholderia virus phiE125, Achromobacter virus 83-24, Achromobacter virus JWX, Arthrobacter virus Tank, Gordonia virus Suzy, Gordonia virus Terapin, Streptomyces virus TG1, Mycobacterium virus Anaya, Mycobacterium virus Angelica, Mycobacterium virus CrimD, Mycobacterium virus Fionnbharth, Mycobacterium virus JAWS, Mycobacterium virus Larva, Mycobacterium virus MacnCheese, Mycobacterium virus Pixie, Mycobacterium virus TM4, Tsukamurella virus TIN2, Tsukamurella virus TIN3, Tsukamurella virus TIN4, Rhodobacter virus RcSpartan, Rhodobacter virus RcTitan, Mycobacterium virus Tortellini, Staphylococcus virus 47, Staphylococcus virus 3a, Staphylococcus virus 42e, Staphylococcus virus IPLA35, Staphylococcus virus phi12, Staphylococcus virus phiSLT, Mycobacterium virus 32HC, Rhodococcus virus Trina, Gordonia virus Trine, Paenibacillus virus Tripp, Flavobacterium virus 1H, Flavobacterium virus 23T, Flavobacterium virus 2A, Flavobacterium virus 6H, Streptomyces virus Lilbooboo, Streptomyces virus Vash, Paenibacillus virus Vegas, Gordonia virus Vendetta, Paracoccus virus Shpa, Pantoea virus Vid5, Acinetobacter virus B1251, Acinetobacter virus R3177, Gordonia virus Brandonk123, Gordonia virus Lennon, Gordonia virus Vivi2, Bordetella virus CN1, Bordetella virus CN2, Bordetella virus FP1, Bordetella virus MW2, Bacillus virus Wbeta, Rhodococcus virus Weasel, Mycobacterium virus Wildcat, Gordonia virus Billnye, Gordonia virus Twister6, Gordonia virus Wizard, Gordonia virus Hotorobo, Gordonia virus Woes, Streptomyces virus TP1604, Streptomyces virus YDN12, Roseobacter virus RDJL1, Roseobacter virus RDJL2, Xanthomonas virus OP1, Xanthomonas virus Xop411, Xanthomonas virus Xp10, Arthobacter virus Yang, Alphaproteobacteria virus phiJ1001, Pseudomonas virus LKO4, Pseudomonas virus M6, Pseudomonas virus MP1412, Pseudomonas virus PAE1, Pseudomonas virus Yua, Gordonia virus Yvonnetastic, Microbacterium virus Zeta1847, Rhodococcus virus RGL3, Paenibacillus virus Lily, Vibrio virus CTXphi, Propionibacterium virus B5, Vibrio virus KSF1, Xanthomonas virus Cf1c, Vibrio virus fs1, Vibrio virus VGJ, Ralstonia virus RS551, Ralstonia virus RS603, Ralstonia virus RSM1, Ralstonia virus RSM3, Escherichia virus If1, Escherichia virus M13, Escherichia virus 122, Salmonella virus IKe, Ralstonia virus PE226, Pseudomonas virus Pf1, Stenotrophomonas virus PSH1, Ralstonia virus RSS1, Vibrio virus fs2, Vibrio virus VFJ, Stenotrophomonas virus SMA6, Stenotrophomonas virus SMA5, Stenotrophomonas virus SMA7, Pseudomonas virus Pf3, Thermus virus OH3, Vibrio virus VfO3K6, Vibrio virus VCY, Vibrio virus Vf33, Xanthomonas virus Xf109, Acholeplasma virus L51, Spiroplasma virus SVTS2, Spiroplasma virus C74, Spiroplasma virus R8A2B, Spiroplasma virus SkV1CR23x, Escherichia virus alpha3, Escherichia virus ID21, Escherichia virus ID32, Escherichia virus ID62, Escherichia virus NC28, Escherichia virus NC29, Escherichia virus NC35, Escherichia virus phiK, Escherichia virus St1, Escherichia virus WA45, Escherichia virus G4, Escherichia virus ID52, Escherichia virus Talmos, Escherichia virus phiX174, Bdellovibrio virus MAC1, Bdellovibrio virus MI-12K, Chlamydia virus Chp1, Chlamydia virus Chp2, Chlamydia virus CPAR39, Chlamydia virus CPG1, Spiroplasma virus SpV4, Bombyx mori bidensovirus, Acerodon celebensis polyomavirus 1, Artibeus planirostris polyomavirus 2, Artibeus planirostris polyomavirus 3, Ateles paniscus polyomavirus 1, Cardioderma cor polyomavirus 1, Carollia perspicillata polyomavirus 1, Chlorocebus pygerythrus polyomavirus 1, Chlorocebus pygerythrus polyomavirus 3, Dobsonia moluccensis polyomavirus 1, Eidolon helvum polyomavirus 1, Gorilla gorilla polyomavirus 1, Human polyomavirus 5, Human polyomavirus 8, Human polyomavirus 9, Human polyomavirus 13, Human polyomavirus 14, Macaca fascicularis polyomavirus 1, Mesocricetus auratus polyomavirus 1, Miniopterus schreibersii polyomavirus 1, Miniopterus schreibersii polyomavirus 2, Molossus molossus polyomavirus 1, Mus musculus polyomavirus 1, Otomops martiensseni polyomavirus 1, Otomops martiensseni polyomavirus 2, Pan troglodytes polyomavirus 1, Pan troglodytes polyomavirus 2, Pan troglodytes polyomavirus 3, Pan troglodytes polyomavirus 4, Pan troglodytes polyomavirus 5, Pan troglodytes polyomavirus 6, Pan troglodytes polyomavirus 7, Papio cynocephalus polyomavirus 1, Piliocolobus badius polyomavirus 1, Piliocolobus rufomitratus polyomavirus 1, Pongo abelii polyomavirus 1, Pongo pygmaeus polyomavirus 1, Procyon lotor polyomavirus 1, Pteropus vampyrus polyomavirus 1, Rattus norvegicus polyomavirus 1, Sorex araneus polyomavirus 1, Sorex coronatus polyomavirus 1, Sorex minutus polyomavirus 1, Sturnira lilium polyomavirus 1, Tupaia belangeri polyomavirus 1, Acerodon celebensis polyomavirus 2, Artibeus planirostris polyomavirus 1, Canis familiaris polyomavirus 1, Cebus albifrons polyomavirus 1, Cercopithecus erythrotis polyomavirus 1, Chlorocebus pygerythrus polyomavirus 2, Desmodus rotundus polyomavirus 1, Dobsonia moluccensis polyomavirus 2, Dobsonia moluccensis polyomavirus 3, Enhydra lutris polyomavirus 1, Equus caballus polyomavirus 1, Human polyomavirus 1, Human polyomavirus 2, Human polyomavirus 3, Human polyomavirus 4, Leptonychotes weddellii polyomavirus 1, Loxodonta africana polyomavirus 1, Macaca mulatta polyomavirus 1, Mastomys natalensis polyomavirus 1, Meles meles polyomavirus 1, Microtus arvalis polyomavirus 1, Miniopterus africanus polyomavirus 1, Mus musculus polyomavirus 2, Mus musculus polyomavirus 3, Myodes glareolus polyomavirus 1, Myotis lucifugus polyomavirus 1, Pan troglodytes polyomavirus 8, Papio cynocephalus polyomavirus 2, Pteronotus davyi polyomavirus 1, Pteronotus parnellii polyomavirus 1, Rattus norvegicus polyomavirus 2, Rousettus aegyptiacus polyomavirus 1, Saimiri boliviensis polyomavirus 1, Saimiri sciureus polyomavirus 1, Vicugna pacos polyomavirus 1, Zalophus californianus polyomavirus 1, Human polyomavirus 6, Human polyomavirus 7, Human polyomavirus 10, Human polyomavirus 11, Anser anser polyomavirus 1, Ayes polyomavirus 1, Corvus monedula polyomavirus 1, Cracticus torquatus polyomavirus 1, Erythrura gouldiae polyomavirus 1, Lonchura maja polyomavirus 1, Pygoscelis adeliae polyomavirus 1, Pyrrhula pyrrhula polyomavirus 1, Serinus canaria polyomavirus 1, Ailuropoda melanoleuca polyomavirus 1, Bos taurus polyomavirus 1, Centropristis striata polyomavirus 1, Delphinus delphis polyomavirus 1, Procyon lotor polyomavirus 2, Rhynchobatus djiddensis polyomavirus 1, Sparus aurata polyomavirus 1, Trematomus bernacchii polyomavirus 1, Trematomus pennellii polyomavirus 1, Alphapapillomavirus 1, Alphapapillomavirus 2, Alphapapillomavirus 3, Alphapapillomavirus 4, Alphapapillomavirus 5, Alphapapillomavirus 6, Alphapapillomavirus 7, Alphapapillomavirus 8, Alphapapillomavirus 9, Alphapapillomavirus 10, Alphapapillomavirus 11, Alphapapillomavirus 12, Alphapapillomavirus 13, Alphapapillomavirus 14, Betapapillomavirus 1, Betapapillomavirus 2, Betapapillomavirus 3, Betapapillomavirus 4, Betapapillomavirus 5, Betapapillomavirus 6, Chipapillomavirus 1, Chipapillomavirus 2, Chipapillomavirus 3, Deltapapillomavirus 1, Deltapapillomavirus 2, Deltapapillomavirus 3, Deltapapillomavirus 4, Deltapapillomavirus 5, Deltapapillomavirus 6, Deltapapillomavirus 7, Dyochipapillomavirus 1, Dyodeltapapillomavirus 1, Dyoepsilonpapillomavirus 1, Dyoetapapillomavirus 1, Dyoiotapapillomavirus 1, Dyoiotapapillomavirus 2, Dyokappapapillomavirus 1, Dyokappapapillomavirus 2, Dyokappapapillomavirus 3, Dyokappapapillomavirus 4, Dyokappapapillomavirus 5, Dyolambdapapillomavirus 1, Dyomupapillomavirus 1, Dyonupapillomavirus 1, Dyoomegapapillomavirus 1, Dyoomikronpapillomavirus 1, Dyophipapillomavirus 1, Dyopipapillomavirus 1, Dyopsipapillomavirus 1, Dyorhopapillomavirus 1, Dyosigmapapillomavirus 1, Dyotaupapillomavirus 1, Dyothetapapillomavirus 1, Dyoupsilonpapillomavirus 1, Dyoxipapillomavirus 1, Dyoxipapillomavirus 2, Dyozetapapillomavirus 1, Epsilonpapillomavirus 1, Epsilonpapillomavirus 2, Etapapillomavirus 1, Gammapapillomavirus 1, Gammapapillomavirus 2, Gammapapillomavirus 3, Gammapapillomavirus 4, Gammapapillomavirus 5, Gammapapillomavirus 6, Gammapapillomavirus 7, Gammapapillomavirus 8, Gammapapillomavirus 9, Gammapapillomavirus 10, Gammapapillomavirus 11, Gammapapillomavirus 12, Gammapapillomavirus 13, Gammapapillomavirus 14, Gammapapillomavirus 15, Gammapapillomavirus 16, Gammapapillomavirus 17, Gammapapillomavirus 18, Gammapapillomavirus 19, Gammapapillomavirus 20, Gammapapillomavirus 21, Gammapapillomavirus 22, Gammapapillomavirus 23, Gammapapillomavirus 24, Gammapapillomavirus 25, Gammapapillomavirus 26, Gammapapillomavirus 27, Iotapapillomavirus 1, Iotapapillomavirus 2, Kappapapillomavirus 1, Kappapapillomavirus 2, Lambdapapillomavirus 1, Lambdapapillomavirus 2, Lambdapapillomavirus 3, Lambdapapillomavirus 4, Lambdapapillomavirus 5, Mupapillomavirus 1, Mupapillomavirus 2, Mupapillomavirus 3, Nupapillomavirus 1, Omegapapillomavirus 1, Omikronpapillomavirus 1, Phipapillomavirus 1, Pipapillomavirus 1, Pipapillomavirus 2, Psipapillomavirus 1, Psipapillomavirus 2, Psipapillomavirus 3, Rhopapillomavirus 1, Rhopapillomavirus 2, Sigmapapillomavirus 1, Taupapillomavirus 1, Taupapillomavirus 2, Taupapillomavirus 3, Taupapillomavirus 4, Thetapapillomavirus 1, Treisdeltapapillomavirus 1, Treisepsilonpapillomavirus 1, Treisetapapillomavirus 1, Treisiotapapillomavirus 1, Treiskappapapillomavirus 1, Treisthetapapillomavirus 1, Treiszetapapillomavirus 1, Upsilonpapillomavirus 1, Upsilonpapillomavirus 2, Upsilonpapillomavirus 3, Xipapillomavirus 1, Xipapillomavirus 2, Xipapillomavirus 3, Xipapillomavirus 4, Xipapillomavirus 5, Zetapapillomavirus 1, Alefpapillomavirus 1, Asteroid aquambidensovirus 1, Decapod aquambidensovirus 1, Blattodean blattambidensovirus 1, Hemipteran hemiambidensovirus 1, Hemipteran hemiambidensovirus 2, Lepidopteran iteradensovirus 1, Lepidopteran iteradensovirus 2, Lepidopteran iteradensovirus 3, Lepidopteran iteradensovirus 4, Lepidopteran iteradensovirus 5, Orthopteran miniambidensovirus 1, Blattodean pefuambidensovirus 1, Dipteran protoambidensovirus 1, Lepidopteran protoambidensovirus 1, Hemipteran scindoambidensovirus 1, Hymenopteran scindoambidensovirus 1, Orthopteran scindoambidensovirus 1, Dipteran brevihamaparvovirus 1, Dipteran brevihamaparvovirus 2, Carnivore chaphamaparvovirus 1, Chiropteran chaphamaparvovirus 1, Galliform chaphamaparvovirus 1, Galliform chaphamaparvovirus 2, Galliform chaphamaparvovirus 3, Rodent chaphamaparvovirus 1, Rodent chaphamaparvovirus 2, Ungulate chaphamaparvovirus 1, Decapod hepanhamaparvovirus 1, Syngnathid ichthamaparvovirus 1, Decapod penstylhamaparvovirus 1, Carnivore amdoparvovirus 1, Carnivore amdoparvovirus 2, Carnivore amdoparvovirus 3, Carnivore amdoparvovirus 4, Carnivore amdoparvovirus 5, Chiropteran artiparvovirus 1, Galliform aveparvovirus 1, Gruiform aveparvovirus 1, Carnivore bocaparvovirus 1, Carnivore bocaparvovirus 2, Carnivore bocaparvovirus 3, Carnivore bocaparvovirus 4, Carnivore bocaparvovirus 5, Carnivore bocaparvovirus 6, Chiropteran bocaparvovirus 1, Chiropteran bocaparvovirus 2, Chiropteran bocaparvovirus 3, Chiropteran bocaparvovirus 4, Lagomorph bocaparvovirus 1, Pinniped bocaparvovirus 1, Pinniped bocaparvovirus 2, Primate bocaparvovirus 1, Primate bocaparvovirus 2, Rodent bocaparvovirus 1, Rodent bocaparvovirus 2, Ungulate bocaparvovirus 1, Ungulate bocaparvovirus 2, Ungulate bocaparvovirus 3, Ungulate bocaparvovirus 4, Ungulate bocaparvovirus 5, Ungulate bocaparvovirus 6, Ungulate bocaparvovirus 7, Ungulate bocaparvovirus 8, Pinniped copiparvovirus 1, Ungulate copiparvovirus 1, Ungulate copiparvovirus 2, Ungulate copiparvovirus 3, Ungulate copiparvovirus 4, Ungulate copiparvovirus 5, Ungulate copiparvovirus 6, Adeno-associated dependoparvovirus A, Adeno-associated dependoparvovirus B, Anseriform dependoparvovirus 1, Avian dependoparvovirus 1, Chiropteran dependoparvovirus 1, Pinniped dependoparvovirus 1, Rodent dependoparvovirus 1, Rodent dependoparvovirus 2, Squamate dependoparvovirus 1, Squamate dependoparvovirus 2, Pinniped erythroparvovirus 1, Primate erythroparvovirus 1, Primate erythroparvovirus 2, Primate erythroparvovirus 3, Primate erythroparvovirus 4, Rodent erythroparvovirus 1, Ungulate erythroparvovirus 1, Primate loriparvovirus 1, Carnivore protoparvovirus, Carnivore protoparvovirus 1, Chiropteran protoparvovirus 1, Eulipotyphla protoparvovirus 1, Primate protoparvovirus 1, Primate protoparvovirus 2, Primate protoparvovirus 3, Primate protoparvovirus 4, Rodent protoparvovirus 1, Rodent protoparvovirus 2, Rodent protoparvovirus 3, Ungulate protoparvovirus 1, Ungulate protoparvovirus 2, Chiropteran tetraparvovirus 1, Primate tetraparvovirus 1, Ungulate tetraparvovirus 1, Ungulate tetraparvovirus 2, Ungulate tetraparvovirus 3, Ungulate tetraparvovirus 4, Chaetoceros diatodnavirus 1, Avon-Heathcote Estuary associated kieseladnavirus, Chaetoceros protobacilladnavirus 1, Chaetoceros protobacilladnavirus 2, Chaetoceros protobacilladnavirus 3, Chaetoceros protobacilladnavirus 4, Marine protobacilladnavirus 1, Snail associated protobacilladnavirus 1, Snail associated protobacilladnavirus 2, Barbel circovirus, Bat associated circovirus 1, Bat associated circovirus 2, Bat associated circovirus 3, Bat associated circovirus 4, Bat associated circovirus 5, Bat associated circovirus 6, Bat associated circovirus 7, Bat associated circovirus 8, Bat associated circovirus 9, Bat associated circovirus 10, Bat associated circovirus 11, Bat associated circovirus 12, Beak and feather disease virus, Canary circovirus, Canine circovirus, Chimpanzee associated circovirus 1, Civet circovirus, Duck circovirus, European catfish circovirus, Finch circovirus, Goose circovirus, Gull circovirus, Human associated circovirus 1, Mink circovirus, Mosquito associated circovirus 1, Pigeon circovirus, Porcine circovirus 1, Porcine circovirus 2, Porcine circovirus 3, Raven circovirus, Rodent associated circovirus 1, Rodent associated circovirus 2, Rodent associated circovirus 3, Rodent associated circovirus 4, Rodent associated circovirus 5, Rodent associated circovirus 6, Rodent associated circovirus 7, Starling circovirus, Swan circovirus, Tick associated circovirus 1, Tick associated circovirus 2, Zebra finch circovirus, Ant associated cyclovirus 1, Bat associated cyclovirus 1, Bat associated cyclovirus 2, Bat associated cyclovirus 3, Bat associated cyclovirus 4, Bat associated cyclovirus 5, Bat associated cyclovirus 6, Bat associated cyclovirus 7, Bat associated cyclovirus 8, Bat associated cyclovirus 9, Bat associated cyclovirus 10, Bat associated cyclovirus 11, Bat associated cyclovirus 12, Bat associated cyclovirus 13, Bat associated cyclovirus 14, Bat associated cyclovirus 15, Bat associated cyclovirus 16, Bovine associated cyclovirus 1, Chicken associated cyclovirus 1, Chicken associated cyclovirus 2, Chimpanzee associated cyclovirus 1, Cockroach associated cyclovirus 1, Dragonfly associated cyclovirus 1, Dragonfly associated cyclovirus 2, Dragonfly associated cyclovirus 3, Dragonfly associated cyclovirus 4, Dragonfly associated cyclovirus 5, Dragonfly associated cyclovirus 6, Dragonfly associated cyclovirus 7, Dragonfly associated cyclovirus 8, Duck associated cyclovirus 1, Feline associated cyclovirus 1, Goat associated cyclovirus 1, Horse associated cyclovirus 1, Human associated cyclovirus 1, Human associated cyclovirus 2, Human associated cyclovirus 3, Human associated cyclovirus 4, Human associated cyclovirus 5, Human associated cyclovirus 6, Human associated cyclovirus 7, Human associated cyclovirus 8, Human associated cyclovirus 9, Human associated cyclovirus 10, Human associated cyclovirus 11, Human associated cyclovirus 12, Mouse associated cyclovirus 1, Rodent associated cyclovirus 1, Rodent associated cyclovirus 2, Spider associated cyclovirus 1, Squirrel associated cyclovirus 1, Bovine associated bovismacovirus 1, Bovine associated bovismacovirus 2, Dragonfly associated bovismacovirus 1, Bovine associated cosmacovirus 1, Dragonfly associated dragsmacovirus 1, Bovine associated drosmacovirus 1, Camel associated drosmacovirus 1, Camel associated drosmacovirus2, Bovine associated huchismacovirus 1, Bovine associated huchismacovirus 2, Chicken associated huchismacovirus 1, Chicken associated huchismacovirus 2, Human associated huchismacovirus 1, Human associated huchismacovirus 2, Human associated huchismacovirus 3, Bovine associated porprismacovirus 1, Camel associated porprismacovirus 1, Camel associated porprismacovirus 2, Camel associated porprismacovirus 3, Camel associated porprismacovirus 4, Chimpanzee associated porprismacovirus 1, Chimpanzee associated porprismacovirus 2, Gorilla associated porprismacovirus 1, Howler monkey associated porprismacovirus 1, Human associated porprismacovirus 1, Human associated porprismacovirus 2, Lemur associated porprismacovirus 1, Porcine associated porprismacovirus 1, Porcine associated porprismacovirus 2, Porcine associated porprismacovirus 3, Porcine associated porprismacovirus 4, Porcine associated porprismacovirus 5, Porcine associated porprismacovirus 6, Porcine associated porprismacovirus 7, Porcine associated porprismacovirus 8, Porcine associated porprismacovirus 9, Porcine associated porprismacovirus 10, Rat associated porprismacovirus 1, Sheep associated porprismacovirus 1, Sheep associated porprismacovirus 2, Sheep associated porprismacovirus 3, Turkey associated porprismacovirus 1, Abaca bunchy top virus, Banana bunchy top virus, Cardamom bushy dwarf virus, Black medic leaf roll virus, Faba bean necrotic stunt virus, Faba bean necrotic yellows virus, Faba bean yellow leaf virus, Milk vetch dwarf virus, Pea necrotic yellow dwarf virus, Pea yellow stunt virus, Subterranean clover stunt virus, Coconut foliar decay virus, Brisavirus, Vientovirus, Beet curly top Iran virus, Exomis microphylla latent virus, Spinach curly top Arizona virus, Abutilon golden mosaic virus, Abutilon mosaic Bolivia virus, Abutilon mosaic Brazil virus, Abutilon mosaic virus, African cassava mosaic Burkina Faso virus, African cassava mosaic virus, Ageratum enation virus, Ageratum leaf curl Sichuan virus, Ageratum leaf curl virus, Ageratum yellow vein Hualian virus, Ageratum yellow vein Sri Lanka virus, Ageratum yellow vein virus, Allamanda leaf curl virus, Allamanda leaf mottle distortion virus, Alternanthera yellow vein virus, Andrographis yellow vein leaf curl virus, Asystasia mosaic Madagascar virus, Bean calico mosaic virus, Bean chlorosis virus, Bean dwarf mosaic virus, Bean golden mosaic virus, Bean golden yellow mosaic virus, Bean leaf crumple virus, Bean white chlorosis mosaic virus, Bean yellow mosaic Mexico virus, Bhendi yellow vein Bhubhaneswar virus, Bhendi yellow vein Haryana virus, Bhendi yellow vein mosaic Delhi virus, Bhendi yellow vein mosaic virus, Bitter gourd yellow mosaic virus, Blainvillea yellow spot virus, Blechum interveinal chlorosis virus, Blechum yellow vein virus, Boerhavia yellow spot virus, Cabbage leaf curl Jamaica virus, Cabbage leaf curl virus, Capraria yellow spot virus, Cassava mosaic Madagascar virus, Catharanthus yellow mosaic virus, Centrosema yellow spot virus, Chayote yellow mosaic virus, Chenopodium leaf curl virus, Chilli leaf curl Ahmedabad virus, Chilli leaf curl Bhavanisagar virus, Chilli leaf curl Gonda virus, Chilli leaf curl India virus, Chilli leaf curl Kanpur virus, Chilli leaf curl Sri Lanka virus, Chilli leaf curl Vellanad virus, Chilli leaf curl virus, Chino del tomate Amazonas virus, Chino del tomate virus, Cleome golden mosaic virus, Cleome leaf crumple virus, Clerodendron golden mosaic virus, Clerodendron yellow mosaic virus, Clerodendrum golden mosaic China virus, Clerodendrum golden mosaic Jiangsu virus, Cnidoscolus mosaic leaf deformation virus, Coccinia mosaic Tamil Nadu virus, Common bean mottle virus, Common bean severe mosaic virus, Corchorus golden mosaic virus, Corchorus yellow spot virus, Corchorus yellow vein mosaic virus, Corchorus yellow vein virus, Cotton chlorotic spot virus, Cotton leaf crumple virus, Cotton leaf curl Alabad virus, Cotton leaf curl Bangalore virus, Cotton leaf curl Barasat virus, Cotton leaf curl Gezira virus, Cotton leaf curl Kokhran virus, Cotton leaf curl Multan virus, Cotton yellow mosaic virus, Cowpea bright yellow mosaic virus, Cowpea golden mosaic virus, Crassocephalum yellow vein virus, Croton golden mosaic virus, Croton yellow vein mosaic virus, Cucurbit leaf crumple virus, Dalechampia chlorotic mosaic virus, Datura leaf curl virus, Datura leaf distortion virus, Deinbollia mosaic virus, Desmodium leaf distortion virus, Desmodium mottle virus, Dicliptera yellow mottle Cuba virus, Dicliptera yellow mottle virus, Dolichos yellow mosaic virus, Duranta leaf curl virus, East African cassava mosaic Cameroon virus, East African cassava mosaic Kenya virus, East African cassava mosaic Malawi virus, East African cassava mosaic virus, East African cassava mosaic Zanzibar virus, Eclipta yellow vein virus, Emilia yellow vein Fujian virus, Emilia yellow vein Thailand virus, Emilia yellow vein virus, Erectites yellow mosaic virus, Eupatorium yellow vein mosaic virus, Eupatorium yellow vein virus, Euphorbia leaf curl Guangxi virus, Euphorbia leaf curl virus, Euphorbia mosaic Peru virus, Euphorbia mosaic virus, Euphorbia yellow leaf curl virus, Euphorbia yellow mosaic virus, French bean leaf curl virus, Hedyotis uncinella yellow mosaic virus, Hemidesmus yellow mosaic virus, Hibiscus golden mosaic virus, Hollyhock leaf curl virus, Hollyhock yellow vein mosaic virus, Hollyhock yellow vein virus, Honeysuckle yellow vein virus, Horsegram yellow mosaic virus, Indian cassava mosaic virus, Jacquemontia mosaic Yucatan virus, Jacquemontia yellow mosaic virus, Jacquemontia yellow vein virus, Jatropha leaf curl Gujarat virus, Jatropha leaf curl virus, Jatropha leaf yellow mosaic virus, Jatropha mosaic India virus, Jatropha mosaic Nigeria virus, Jatropha mosaic virus, Jatropha yellow mosaic virus, Kudzu mosaic virus, Leonurus mosaic virus, Lindernia anagallis yellow vein virus, Lisianthus enation leaf curl virus, Ludwigia yellow vein Vietnam virus, Ludwigia yellow vein virus, Luffa yellow mosaic virus, Lycianthes yellow mosaic virus, Macroptilium bright mosaic virus, Macroptilium common mosaic virus, Macroptilium golden mosaic virus, Macroptilium mosaic Puerto Rico virus, Macroptilium yellow mosaic Florida virus, Macroptilium yellow mosaic virus, Macroptilium yellow spot virus, Macroptilium yellow vein virus, Malvastrum bright yellow mosaic virus, Malvastrum leaf curl Philippines virus, Malvastrum leaf curl virus, Malvastrum yellow mosaic Helshire virus, Malvastrum yellow mosaic Jamaica virus, Malvastrum yellow mosaic virus, Malvastrum yellow vein Cambodia virus, Malvastrum yellow vein Honghe virus, Malvastrum yellow vein Lahore virus, Malvastrum yellow vein virus, Malvastrum yellow vein Yunnan virus, Melochia mosaic virus, Melochia yellow mosaic virus, Melon chlorotic leaf curl virus, Melon chlorotic mosaic virus, Melon yellow mosaic virus, Merremia mosaic Puerto Rico virus, Merremia mosaic virus, Mesta yellow vein mosaic Bahraich virus, Mimosa yellow leaf curl virus, Mirabilis leaf curl virus, Mungbean yellow mosaic India virus, Mungbean yellow mosaic virus, Okra enation leaf curl virus, Okra leaf curl Oman virus, Okra mottle virus, Okra yellow crinkle virus, Okra yellow mosaic Mexico virus, Oxalis yellow vein virus, Papaya leaf crumple virus, Papaya leaf curl China virus, Papaya leaf curl Guandong virus, Papaya leaf curl virus, Passionfruit leaf curl virus, Passionfruit leaf distortion virus, Passionfruit severe leaf distortion virus, Pavonia mosaic virus, Pavonia yellow mosaic virus, Pea leaf distortion virus, Pedilanthus leaf curl virus, Pepper golden mosaic virus, Pepper huasteco yellow vein virus, Pepper leaf curl Bangladesh virus, Pepper leaf curl Lahore virus, Pepper leaf curl virus, Pepper leaf curl Yunnan virus, Pepper leafroll virus, Pepper yellow leaf curl Aceh virus, Pepper yellow leaf curl Indonesia virus, Pepper yellow leaf curl Indonesia virus 2, Pepper yellow leaf curl Thailand virus, Pepper yellow leaf curl virus, Pepper yellow vein Mali virus, Potato yellow mosaic Panama virus, Potato yellow mosaic virus, Pouzolzia golden mosaic virus, Pouzolzia mosaic Guangdong virus, Pouzolzia yellow mosaic virus, Premna leaf curl virus, Pumpkin yellow mosaic virus, Radish leaf curl virus, Ramie mosaic Yunnan virus, Rhynchosia golden mosaic Havana virus, Rhynchosia golden mosaic Sinaloa virus, Rhynchosia golden mosaic virus, Rhynchosia mild mosaic virus, Rhynchosia rugose golden mosaic virus, Rhynchosia yellow mosaic India virus, Rhynchosia yellow mosaic virus, Rose leaf curl virus, Sauropus leaf curl virus, Senecio yellow mosaic virus, Senna leaf curl virus, Sida angular mosaic virus, Sida bright yellow mosaic virus, Sida chlorotic mottle virus, Sida chlorotic vein virus, Sida ciliaris golden mosaic virus, Sida common mosaic virus, Sida golden mosaic Braco virus, Sida golden mosaic Brazil virus, Sida golden mosaic Buckup virus, Sida golden mosaic Costa Rica virus, Sida golden mosaic Florida virus, Sida golden mosaic Lara virus, Sida golden mosaic virus, Sida golden mottle virus, Sida golden yellow spot virus, Sida golden yellow vein virus, Sida leaf curl virus, Sida micrantha mosaic virus, Sida mosaic Alagoas virus, Sida mosaic Bolivia virus 1, Sida mosaic Bolivia virus 2, Sida mosaic Sinaloa virus, Sida mottle Alagoas virus, Sida mottle virus, Sida yellow blotch virus, Sida yellow leaf curl virus, Sida yellow mosaic Alagoas virus, Sida yellow mosaic China virus, Sida yellow mosaic virus, Sida yellow mosaic Yucatan virus, Sida yellow mottle virus, Sida yellow net virus, Sida yellow vein Vietnam virus, Sida yellow vein virus, Sidastrum golden leaf spot virus, Siegesbeckia yellow vein Guangxi virus, Siegesbeckia yellow vein virus, Solanum mosaic Bolivia virus, South African cassava mosaic virus, Soybean blistering mosaic virus, Soybean chlorotic blotch virus, Soybean mild mottle virus, Spilanthes yellow vein virus, Spinach yellow vein virus, Squash leaf curl China virus, Squash leaf curl Philippines virus, Squash leaf curl virus, Squash leaf curl Yunnan virus, Squash mild leaf curl virus, Sri Lankan cassava mosaic virus, Stachytarpheta leaf curl virus, Sunn hemp leaf distortion virus, Sweet potato golden vein Korea virus, Sweet potato leaf curl Canary virus, Sweet potato leaf curl China virus, Sweet potato leaf curl Georgia virus, Sweet potato leaf curl Guangxi virus, Sweet potato leaf curl Henan virus, Sweet potato leaf curl Hubei virus, Sweet potato leaf curl Sao Paulo virus, Sweet potato leaf curl Shandong virus, Sweet potato leaf curl Sichuan virus 1, Sweet potato leaf curl Sichuan virus 2, Sweet potato leaf curl South Carolina virus, Sweet potato leaf curl virus, Sweet potato mosaic virus, Synedrella yellow vein clearing virus, Telfairia golden mosaic virus, Tobacco curly shoot virus, Tobacco leaf curl Comoros virus, Tobacco leaf curl Cuba virus, Tobacco leaf curl Dominican Republic virus, Tobacco leaf curl Pusa virus, Tobacco leaf curl Thailand virus, Tobacco leaf curl Yunnan virus, Tobacco leaf curl Zimbabwe virus, Tobacco leaf rugose virus, Tobacco mottle leaf curl virus, Tobacco yellow crinkle virus, Tomato bright yellow mosaic virus, Tomato bright yellow mottle virus, Tomato chino La Paz virus, Tomato chlorotic leaf curl virus, Tomato chlorotic leaf distortion virus, Tomato chlorotic mottle Guyane virus, Tomato chlorotic mottle virus, Tomato common mosaic virus, Tomato curly stunt virus, Tomato dwarf leaf virus, Tomato enation leaf curl virus, Tomato golden leaf distortion virus, Tomato golden leaf spot virus, Tomato golden mosaic virus, Tomato golden mottle virus, Tomato golden vein virus, Tomato interveinal chlorosis virus, Tomato latent virus, Tomato leaf curl Anjouan virus, Tomato leaf curl Arusha virus, Tomato leaf curl Bangalore virus, Tomato leaf curl Bangladesh virus, Tomato leaf curl Burkina Faso virus, Tomato leaf curl Cebu virus, Tomato leaf curl China virus, Tomato leaf curl Comoros virus, Tomato leaf curl Diana virus, Tomato leaf curl Ghana virus, Tomato leaf curl Guangdong virus, Tomato leaf curl Guangxi virus, Tomato leaf curl Gujarat virus, Tomato leaf curl Hainan virus, Tomato leaf curl Hanoi virus, Tomato leaf curl Hsinchu virus, Tomato leaf curl Iran virus, Tomato leaf curl Japan virus, Tomato leaf curl Java virus, Tomato leaf curl Joydebpur virus, Tomato leaf curl Karnataka virus, Tomato leaf curl Karnataka virus 2, Tomato leaf curl Karnataka virus 3, Tomato leaf curl Kerala virus, Tomato leaf curl Laos virus, Tomato leaf curl Liwa virus, Tomato leaf curl Madagascar virus, Tomato leaf curl Mahe virus, Tomato leaf curl Malaysia virus, Tomato leaf curl Mali virus, Tomato leaf curl Mindanao virus, Tomato leaf curl Moheli virus, Tomato leaf curl Namakely virus, Tomato leaf curl New Delhi virus, Tomato leaf curl New Delhi virus 2, Tomato leaf curl New Delhi virus 4, Tomato leaf curl New Delhi virus 5, Tomato leaf curl Nigeria virus, Tomato leaf curl Palampur virus, Tomato leaf curl Patna virus, Tomato leaf curl Philippines virus, Tomato leaf curl Pune virus, Tomato leaf curl purple vein virus, Tomato leaf curl Rajasthan virus, Tomato leaf curl Seychelles virus, Tomato leaf curl Sinaloa virus, Tomato leaf curl Sri Lanka virus, Tomato leaf curl Sudan virus, Tomato leaf curl Sulawesi virus, Tomato leaf curl Taiwan virus, Tomato leaf curl Tanzania virus, Tomato leaf curl Toliara virus, Tomato leaf curl Uganda virus, Tomato leaf curl Vietnam virus, Tomato leaf curl virus, Tomato leaf deformation virus, Tomato leaf distortion virus, Tomato mild mosaic virus, Tomato mild yellow leaf curl Aragua virus, Tomato mosaic Havana virus, Tomato mottle leaf curl virus, Tomato mottle Taino virus, Tomato mottle virus, Tomato mottle wrinkle virus, Tomato rugose mosaic virus, Tomato rugose yellow leaf curl virus, Tomato severe leaf curl Kalakada virus, Tomato severe leaf curl virus, Tomato severe rugose virus, Tomato twisted leaf virus, Tomato wrinkled mosaic virus, Tomato yellow leaf curl Axarquia virus, Tomato yellow leaf curl China virus, Tomato yellow leaf curl Guangdong virus, Tomato yellow leaf curl Indonesia virus, Tomato yellow leaf curl Kanchanaburi virus, Tomato yellow leaf curl Malaga virus, Tomato yellow leaf curl Mali virus, Tomato yellow leaf curl Sardinia virus, Tomato yellow leaf curl Shuangbai virus, Tomato yellow leaf curl Thailand virus, Tomato yellow leaf curl Vietnam virus, Tomato yellow leaf curl virus, Tomato yellow leaf curl Yunnan virus, Tomato yellow leaf distortion virus, Tomato yellow margin leaf curl virus, Tomato yellow mottle virus, Tomato yellow spot virus, Tomato yellow vein streak virus, Triumfetta yellow mosaic virus, Velvet bean golden mosaic virus, Velvet bean severe mosaic virus, Vernonia crinkle virus, Vernonia yellow vein Fujian virus, Vernonia yellow vein virus, Vigna yellow mosaic virus, Vinca leaf curl virus, Watermelon chlorotic stunt virus, West African Asystasia virus 1, West African Asystasia virus 2, West African Asystasia virus 3, Whitefly-associated begomovirus 1, Whitefly-associated begomovirus 2, Whitefly-associated begomovirus 3, Whitefly-associated begomovirus 4, Whitefly-associated begomovirus 6, Whitefly-associated begomovirus 7, Wissadula golden mosaic virus, Wissadula yellow mosaic virus, Alfalfa leaf curl virus, Euphorbia caput-medusae latent virus, French bean severe leaf curl virus, Plantago lanceolata latent virus, Beet curly top virus, Horseradish curly top virus, Spinach severe curly top virus, Eragrostis curvula streak virus, Grapevine red blotch virus, Prunus latent virus, Wild Vitis latent virus, Axonopus compressus streak virus, Bromus catharticus striate mosaic virus, Chickpea chlorosis Australia virus, Chickpea chlorosis virus, Chickpea chlorotic dwarf virus, Chickpea redleaf virus, Chickpea yellow dwarf virus, Chickpea yellows virus, Chloris striate mosaic virus, Digitaria ciliaris striate mosaic virus, Digitaria didactyla striate mosaic virus, Digitaria streak virus, Dragonfly-associated mastrevirus, Eragrostis minor streak virus, Eragrostis streak virus, Maize streak dwarfing virus, Maize streak Reunion virus, Maize streak virus, Maize striate mosaic virus, Miscanthus streak virus, Oat dwarf virus, Panicum streak virus, Paspalum dilatatum striate mosaic virus, Paspalum striate mosaic virus, Rice latent virus 1, Rice latent virus 2, Saccharum streak virus, Sporobolus striate mosaic virus 1, Sporobolus striate mosaic virus 2, Sugarcane chlorotic streak virus, Sugarcane streak Egypt virus, Sugarcane streak Reunion virus, Sugarcane streak virus, Sugarcane striate virus, Sugarcane white streak virus, Sweet potato symptomless virus 1, Switchgrass mosaic-associated virus, Tobacco yellow dwarf virus, Urochloa streak virus, Wheat dwarf India virus, Wheat dwarf virus, Tomato pseudo-curly top virus, Sesame curly top virus, Turnip curly top virus, Turnip leaf roll virus, Citrus chlorotic dwarf associated virus, Mulberry mosaic dwarf associated virus, Blackbird associated gemycircularvirus 1, Bovine associated gemycircularvirus 1, Bromus associated gemycircularvirus 1, Cassava associated gemycircularvirus 1, Chickadee associated gemycircularvirus 1, Chicken associated gemycircularvirus 1, Chicken associated gemycircularvirus 2, Dragonfly associated gemycircularvirus 1, Equine associated gemycircularvirus 1, Fur seal associated gemycircularvirus 1, Gerygone associated gemycircularvirus 1, Gerygone associated gemycircularvirus 2, Gerygone associated gemycircularvirus 3, Hypericum associated gemycircularvirus 1, Lama associated gemycircularvirus 1, Mallard associated gemycircularvirus 1, Miniopterus associated gemycircularvirus 1, Mongoose associated gemycircularvirus 1, Mosquito associated gemycircularvirus 1, Odonata associated gemycircularvirus 1, Odonata associated gemycircularvirus 2, Poaceae associated gemycircularvirus 1, Porcine associated gemycircularvirus 1, Porcine associated gemycircularvirus 2, Pteropus associated gemycircularvirus 1, Pteropus associated gemycircularvirus 2, Pteropus associated gemycircularvirus 3, Pteropus associated gemycircularvirus 4, Pteropus associated gemycircularvirus 5, Pteropus associated gemycircularvirus 6, Pteropus associated gemycircularvirus 7, Pteropus associated gemycircularvirus 8, Pteropus associated gemycircularvirus 9, Pteropus associated gemycircularvirus 10, Rat associated gemycircularvirus 1, Sclerotinia gemycircularvirus 1, Sewage derived gemycircularvirus 1, Sewage derived gemycircularvirus 2, Sewage derived gemycircularvirus 3, Sewage derived gemycircularvirus 4, Sewage derived gemycircularvirus 5, Sheep associated gemycircularvirus 1, Soybean associated gemycircularvirus 1, Dragonfly associated gemyduguivirus 1, Canine associated gemygorvirus 1, Mallard associated gemygorvirus 1, Pteropus associated gemygorvirus 1, Sewage derived gemygorvirus 1, Starling associated gemygorvirus 1, Badger associated gemykibivirus 1, Black robin associated gemykibivirus 1, Blackbird associated gemykibivirus 1, Bovine associated gemykibivirus 1, Dragonfly associated gemykibivirus 1, Human associated gemykibivirus 1, Human associated gemykibivirus 2, Human associated gemykibivirus 3, Human associated gemykibivirus 4, Human associated gemykibivirus 5, Mongoose associated gemykibivirus 1, Pteropus associated gemykibivirus 1, Rhinolophus associated gemykibivirus 1, Rhinolophus associated gemykibivirus 2, Sewage derived gemykibivirus 1, Sewage derived gemykibivirus 2, Pteropus associated gemykolovirus 1, Pteropus associated gemykolovirus 2, Bovine associated gemykrogvirus 1, Caribou associated gemykrogvirus 1, Sewage derived gemykrogvirus 1, Rabbit associated gemykroznavirus 1, Ostrich associated gemytondvirus 1, Human associated gemyvongvirus 1, Alphapleolipovirus HHPV1, Alphapleolipovirus HHPV2, Alphapleolipovirus HRPV1, Alphapleolipovirus HRPV2, Alphapleolipovirus HRPV6, Betapleolipovirus HGPV1, Betapleolipovirus HHPV3, Betapleolipovirus HHPV4, Betapleolipovirus HRPV3, Betapleolipovirus HRPV9, Betapleolipovirus HRPV10, Betapleolipovirus HRPV11, Betapleolipovirus HRPV12, Betapleolipovirus SNJ2, Gammapleolipovirus His2, Amasya cherry disease associated chrysovirus, Anthurium mosaic-associated chrysovirus, Aspergillus fumigatus chrysovirus, Brassica campestris chrysovirus, Colletotrichum gloeosporioides chrysovirus, Cryphonectria nitschkei chrysovirus 1, Fusarium oxysporum chrysovirus 1, Helminthosporium victoriae virus 145S, Isaria javanica chrysovirus, Macrophomina phaseolina chrysovirus, Penicillium brevicompactum virus, Penicillium chrysogenum virus, Penicillium cyaneofulvum virus, Persea americana chrysovirus, Raphanus sativus chrysovirus, Shuangao insect-associated chrysovirus, Verticillium dahliae chrysovirus 1, Alternaria alternata chrysovirus, Botryosphaeria dothidea chrysovirus, Colletotrichum fructicola chrysovirus 1, Fusarium graminearum chrysovirus, Fusarium oxysporum chrysovirus 2, Magnaporthe oryzae chrysovirus, Penicillium janczewskii chrysovirus 1, Penicillium janczewskii chrysovirus 2, Rosellinia necatrix megabirnavirus 1, Rosellinia necatrix quadrivirus 1, Giardia lamblia virus, Leishmania RNA virus 1, Leishmania RNA virus 2, Saccharomyces cerevisiae virus L-A, Saccharomyces cerevisiae virus LBCLa, Scheffersomyces segobiensis virus L, Tuber aestivum virus 1, Ustilago maydis virus H1, Xanthophyllomyces dendrorhous virus L₁A, Xanthophyllomyces dendrorhous virus L1B, Trichomonas vaginalis virus 1, Trichomonas vaginalis virus 2, Trichomonas vaginalis virus 3, Trichomonas vaginalis virus 4, Aspergillus foetidus slow virus 1, Beauveria bassiana victorivirus 1, Chalara elegans RNA Virus 1, Coniothyrium minitans RNA virus, Epichloe festucae virus 1, Gremmeniella abietina RNA virus L1, Helicobasidium mompa totivirus 1-17, Helminthosporium victoriae virus 190S, Magnaporthe oryzae virus 1, Magnaporthe oryzae virus 2, Rosellinia necatrix victorivirus 1, Sphaeropsis sapinea RNA virus 1, Sphaeropsis sapinea RNA virus 2, Tolypocladium cylindrosporum virus 1, Eriocheir sinensis reovirus, Micromonas pusilla reovirus, African horse sickness virus, Bluetongue virus, Changuinola virus, Chenuda virus, Chobar Gorge virus, Corriparta virus, Epizootic hemorrhagic disease virus, Equine encephalosis virus, Eubenangee virus, Great Island virus, Teri virus, Lebombo virus, Orungo virus, Palyam virus, Peruvian horse sickness virus, St Croix River virus, Umatilla virus, Wad Medani virus, Wallal virus, Warrego virus, Wongorr virus, Yunnan orbivirus, Rice dwarf virus, Rice gall dwarf virus, Wound tumor virus, Rotavirus A, Rotavirus B, Rotavirus C, Rotavirus D, Rotavirus F, Rotavirus G, Rotavirus H, Rotavirus I, Rotavirus J, Banna virus, Kadipiro virus, Liao ning virus, Aquareovirus A, Aquareovirus B, Aquareovirus C, Aquareovirus D, Aquareovirus E, Aquareovirus F, Aquareovirus G, Colorado tick fever coltivirus, Eyach coltivirus, Kundal coltivirus, Tai Forest coltivirus, Tarumizu coltivirus, Cypovirus 1, Cypovirus 2, Cypovirus 3, Cypovirus 4, Cypovirus 5, Cypovirus 6, Cypovirus 7, Cypovirus 8, Cypovirus 9, Cypovirus 10, Cypovirus 11, Cypovirus 12, Cypovirus 13, Cypovirus 14, Cypovirus 15, Cypovirus 16, Aedes pseudoscutellaris reovirus, Fiji disease virus, Garlic dwarf virus, Maize rough dwarf virus, Mal de Rio Cuarto virus, Nilaparvata lugens reovirus, Oat sterile dwarf virus, Pangola stunt virus, Rice black streaked dwarf virus, Southern rice black-streaked dwarf virus, Idnoreovirus 1, Idnoreovirus 2, Idnoreovirus 3, Idnoreovirus 4, Idnoreovirus 5, Mycoreovirus 1, Mycoreovirus 2, Mycoreovirus 3, Avian orthoreovirus, Baboon orthoreovirus, Broome orthoreovirus, Mahlapitsi orthoreovirus, Mammalian orthoreovirus, Nelson Bay orthoreovirus, Neoavian orthoreovirus, Piscine orthoreovirus, Reptilian orthoreovirus, Testudine orthoreovirus, Echinochloa ragged stunt virus, Rice ragged stunt virus, Pseudomonas virus phi6, Pseudomonas virus phi8, Pseudomonas virus phi12, Pseudomonas virus phi13, Pseudomonas virus phi2954, Pseudomonas virus phiNN, Pseudomonas virus phiYY, Antheraea eucalypti virus, Darna trima virus, Dasychira pudibunda virus, Nudaurelia capensis beta virus, Philosamia cynthia x ricini virus, Pseudoplusia includens virus, Trichoplusia ni virus, Dendrolimus punctatus virus, Helicoverpa armigera stunt virus, Nudaurelia capensis omega virus, Beet necrotic yellow vein virus, Beet soil-borne mosaic virus, Burdock mottle virus, Rice stripe necrosis virus, Orthohepevirus A, Orthohepevirus B, Orthohepevirus C, Orthohepevirus D, Piscihepevirus A, Rubella virus, Alfalfa mosaic virus, Amazon lily mild mottle virus, Pelargonium zonate spot virus, Broad bean mottle virus, Brome mosaic virus, Cassia yellow blotch virus, Cowpea chlorotic mottle virus, Melandrium yellow fleck virus, Spring beauty latent virus, Cucumber mosaic virus, Gayfeather mild mottle virus, Peanut stunt virus, Tomato aspermy virus, Ageratum latent virus, American plum line pattern virus, Apple mosaic virus, Asparagus virus 2, Blackberry chlorotic ringspot virus, Blueberry shock virus, Citrus leaf rugose virus, Citrus variegation virus, Elm mottle virus, Fragaria chiloensis latent virus, Humulus japonicus latent virus, Lilac leaf chlorosis virus, Lilac ring mottle virus, Parietaria mottle virus, Privet ringspot virus, Prune dwarf virus, Prunus necrotic ringspot virus, Spinach latent virus, Strawberry necrotic shock virus, Tobacco streak virus, Tomato necrotic streak virus, Tulare apple mosaic virus, Olive latent virus 2, Air potato ampelovirus 1, Blackberry vein banding-associated virus, Grapevine leafroll-associated virus 1, Grapevine leafroll-associated virus 3, Grapevine leafroll-associated virus 4, Grapevine leafroll-associated virus 13, Little cherry virus 2, Pineapple mealybug wilt-associated virus 1, Pineapple mealybug wilt-associated virus 2, Pineapple mealybug wilt-associated virus 3, Pistachio ampelovirus A, Plum bark necrosis stem pitting-associated virus, Arracacha virus 1, Beet yellow stunt virus, Beet yellows virus, Blackcurrant closterovirus 1, Burdock yellows virus, Carnation necrotic fleck virus, Carrot yellow leaf virus, Citrus tristeza virus, Grapevine leafroll-associated virus 2, Mint virus 1, Raspberry leaf mottle virus, Rehmannia virus 1, Rose leaf rosette-associated virus, Strawberry chlorotic fleck-associated virus, Tobacco virus 1, Wheat yellow leaf virus, Abutilon yellows virus, Bean yellow disorder virus, Beet pseudoyellows virus, Blackberry yellow vein-associated virus, Cucurbit yellow stunting disorder virus, Diodia vein chlorosis virus, Lettuce chlorosis virus, Lettuce infectious yellows virus, Potato yellow vein virus, Strawberry pallidosis-associated virus, Sweet potato chlorotic stunt virus, Tetterwort vein chlorosis virus, Tomato chlorosis virus, Tomato infectious chlorosis virus, Areca palm velarivirus 1, Cordyline virus 1, Cordyline virus 2, Cordyline virus 3, Cordyline virus 4, Grapevine leafroll-associated virus 7, Little cherry virus 1, Actinidia virus 1, Alligatorweed stunting virus, Blueberry virus A, Megakepasma mosaic virus, Mint vein banding-associated virus, Olive leaf yellowing-associated virus, Persimmon virus B, Agaricus bisporus alphaendornavirus 1, Basella alba alphaendornavirus 1, Bell pepper alphaendornavirus, Cluster bean alphaendornavirus 1, Cucumis melo alphaendornavirus, Erysiphe cichoracearum alphaendornavirus, Grapevine endophyte alphaendornavirus, Helianthus annuus alphaendornavirus, Helicobasidium mompa alphaendornavirus 1, Hordeum vulgare alphaendornavirus, Hot pepper alphaendornavirus, Lagenaria siceraria alphaendornavirus, Oryza rufipogon alphaendornavirus, Oryza sativa alphaendornavirus, Persea americana alphaendornavirus 1, Phaseolus vulgaris alphaendornavirus 1, Phaseolus vulgaris alphaendornavirus 2, Phaseolus vulgaris alphaendornavirus 3, Phytophthora alphaendornavirus 1, Rhizoctonia cerealis alphaendornavirus 1, Rhizoctonia solani alphaendornavirus 2, Vicia faba alphaendornavirus, Winged bean alphaendornavirus 1, Yerba mate alphaendornavirus, Alternaria brassicicola betaendornavirus 1, Botrytis cinerea betaendornavirus 1, Gremmeniella abietina betaendornavirus 1, Rosellinia necatrix betaendornavirus 1, Sclerotinia minor betaendornavirus 1, Sclerotinia sclerotiorum betaendornavirus 1, Tuber aestivum betaendornavirus, Blueberry necrotic ring blotch virus, Tea plant necrotic ring blotch virus, Citrus leprosis virus C, Citrus leprosis virus C2, Hibiscus green spot virus 2, Privet idaeovirus, Raspberry bushy dwarf virus, Japanese holly fern mottle pteridovirus, Maize associated pteridovirus, Aura virus, Barmah Forest virus, Bebaru virus, Cabassou virus, Chikungunya virus, Eastern equine encephalitis virus, Eilat virus, Everglades virus, Fort Morgan virus, Getah virus, Highlands J virus, Madariaga virus, Mayaro virus, Middelburg virus, Mosso das Pedras virus, Mucambo virus, Ndumu virus, Onyong-nyong virus, Pixuna virus, Rio Negro virus, Ross River virus, Salmon pancreas disease virus, Semliki Forest virus, Sindbis virus, Southern elephant seal virus, Tonate virus, Trocara virus, Una virus, Venezuelan equine encephalitis virus, Western equine encephalitis virus, Whataroa virus, Chinese wheat mosaic virus, Japanese soil-borne wheat mosaic virus, Oat golden stripe virus, Soil-borne cereal mosaic virus, Soil-borne wheat mosaic virus, Sorghum chlorotic spot virus, Drakaea virus A, Gentian ovary ringspot virus, Anthoxanthum latent blanching virus, Barley stripe mosaic virus, Lychnis ringspot virus, Poa semilatent virus, Indian peanut clump virus, Peanut clump virus, Beet soil-borne virus, Beet virus Q, Broad bean necrosis virus, Colombian potato soil-borne virus, Potato mop-top virus, Bell pepper mottle virus, Brugmansia mild mottle virus, Cactus mild mottle virus, Clitoria yellow mottle virus, Cucumber fruit mottle mosaic virus, Cucumber green mottle mosaic virus, Cucumber mottle virus, Frangipani mosaic virus, Hibiscus latent Fort Pierce virus, Hibiscus latent Singapore virus, Kyuri green mottle mosaic virus, Maracuj a mosaic virus, Obuda pepper virus, Odontoglossum ringspot virus, Opuntia chlorotic ringspot virus, Paprika mild mottle virus, Passion fruit mosaic virus, Pepper mild mottle virus, Plumeria mosaic virus, Rattail cactus necrosis-associated virus, Rehmannia mosaic virus, Ribgrass mosaic virus, Streptocarpus flower break virus, Sunn-hemp mosaic virus, Tobacco latent virus, Tobacco mild green mosaic virus, Tobacco mosaic virus, Tomato brown rugose fruit virus, Tomato mosaic virus, Tomato mottle mosaic virus, Tropical soda apple mosaic virus, Turnip vein-clearing virus, Ullucus mild mottle virus, Wasabi mottle virus, Yellow tailflower mild mottle virus, Youcai mosaic virus, Zucchini green mottle mosaic virus, Pea early-browning virus, Pepper ringspot virus, Tobacco rattle virus, Alfalfa virus S, Arachis pintoi virus, Blackberry virus E, Garlic mite-borne filamentous virus, Garlic virus A, Garlic virus B, Garlic virus C, Garlic virus D, Garlic virus E, Garlic virus X, Shallot virus X, Vanilla latent virus, Botrytis virus X, Lolium latent virus, Citrus yellow vein clearing virus, Indian citrus ringspot virus, Donkey orchid symptomless virus, Actinidia virus X, Allium virus X, Alstroemeria virus X, Alternanthera mosaic virus, Asparagus virus 3, Bamboo mosaic virus, Cactus virus X, Cassava common mosaic virus, Cassava virus X, Clover yellow mosaic virus, Cymbidium mosaic virus, Foxtail mosaic virus, Hosta virus X, Hydrangea ringspot virus, Lagenaria mild mosaic virus, Lettuce virus X, Lily virus X, Malva mosaic virus, Mint virus X, Narcissus mosaic virus, Nerine virus X, Opuntia virus X, Papaya mosaic virus, Pepino mosaic virus, Phaius virus X, Pitaya virus X, Plantago asiatica mosaic virus, Plantain virus X, Potato aucuba mosaic virus, Potato virus X, Schlumbergera virus X, Strawberry mild yellow edge virus, Tamus red mosaic virus, Tulip virus X, Vanilla virus X, White clover mosaic virus, Yam virus X, Zygocactus virus X, Sclerotinia sclerotiorum debilitation-associated RNA virus, Aconitum latent virus, American hop latent virus, Atractylodes mottle virus, Blueberry scorch virus, Butterbur mosaic virus, Cactus virus 2, Caper latent virus, Carnation latent virus, Chrysanthemum virus B, Cole latent virus, Coleus vein necrosis virus, Cowpea mild mottle virus, Cucumber vein-clearing virus, Daphne virus S, Gaillardia latent virus, Garlic common latent virus, Helenium virus S, Helleborus mosaic virus, Helleborus net necrosis virus, Hippeastrum latent virus, Hop latent virus, Hop mosaic virus, Hydrangea chlorotic mottle virus, Kalanchoe latent virus, Ligustrum necrotic ringspot virus, Ligustrum virus A, Lily symptomless virus, Melon yellowing-associated virus, Mirabilis jalapa mottle virus, Narcissus common latent virus, Nerine latent virus, Passiflora latent virus, Pea streak virus, Phlox virus B, Phlox virus M, Phlox virus S, Poplar mosaic virus, Potato latent virus, Potato virus H, Potato virus M, Potato virus P, Potato virus S, Red clover vein mosaic virus, Sambucus virus C, Sambucus virus D, Sambucus virus E, Shallot latent virus, Sint-Jan onion latent virus, Strawberry pseudo mild yellow edge virus, Sweet potato C6 virus, Sweet potato chlorotic fleck virus, Verbena latent virus, Yam latent virus, Apple stem pitting virus, Apricot latent virus, Asian prunus virus 1, Asian prunus virus 2, Grapevine rupestris stem pitting-associated virus, Grapevine virus T, Peach chlorotic mottle virus, Rubus canadensis virus 1, African oil palm ringspot virus, Cherry green ring mottle virus, Cherry necrotic rusty mottle virus, Cherry rusty mottle associated virus, Cherry twisted leaf associated virus, Banana mild mosaic virus, Banana virus X, Sugarcane striate mosaic-associated virus, Apple stem grooving virus, Cherry virus A, Currant virus A, Mume virus A, Carrot Ch virus 1, Carrot Ch virus 2, Citrus leaf blotch virus, Diuris virus A, Diuris virus B, Hardenbergia virus A, Actinidia seed borne latent virus, Apricot vein clearing associated virus, Caucasus prunus virus, Ribes americanum virus A, Potato virus T, Prunus virus T, Apple chlorotic leaf spot virus, Apricot pseudo-chlorotic leaf spot virus, Cherry mottle leaf virus, Grapevine berry inner necrosis virus, Grapevine Pinot gris virus, Peach mosaic virus, Phlomis mottle virus, Actinidia virus A, Actinidia virus B, Arracacha virus V, Blackberry virus A, Grapevine virus A, Grapevine virus B, Grapevine virus D, Grapevine virus E, Grapevine virus F, Grapevine virus G, Grapevine virus H, Grapevine virus I, Grapevine virus J, Heracleum latent virus, Mint virus 2, Watermelon virus A, Fusarium deltaflexivirus 1, Sclerotinia deltaflexivirus 1, Soybean-associated deltaflexivirus 1, Botrytis virus F, Grapevine fleck virus, Bermuda grass etched-line virus, Blackberry virus S, Citrus sudden death-associated virus, Grapevine asteroid mosaic associated virus, Grapevine Syrah virus 1, Maize rayado fino virus, Nectarine marafivirus M, Oat blue dwarf virus, Olive latent virus 3, Peach marafivirus D, Anagyris vein yellowing virus, Andean potato latent virus, Andean potato mild mosaic virus, Belladonna mottle virus, Cacao yellow mosaic virus, Calopogonium yellow vein virus, Chayote mosaic virus, Chiltepin yellow mosaic virus, Clitoria yellow vein virus, Desmodium yellow mottle virus, Dulcamara mottle virus, Eggplant mosaic virus, Erysimum latent virus, Kennedya yellow mosaic virus, Melon rugose mosaic virus, Nemesia ring necrosis virus, Okra mosaic virus, Ononis yellow mosaic virus, Passion fruit yellow mosaic virus, Peanut yellow mosaic virus, Petunia vein banding virus, Physalis mottle virus, Plantago mottle virus, Scrophularia mottle virus, Tomato blistering mosaic tymovirus, Turnip yellow mosaic virus, Voandzeia necrotic mosaic virus, Wild cucumber mosaic virus, Bombyx mori latent virus, Poinsettia mosaic virus, Apoi virus, Aroa virus, Bagaza virus, Banzi virus, Bouboui virus, Bukalasa bat virus, Cacipacore virus, Carey Island virus, Cowbone Ridge virus, Dakar bat virus, Dengue virus, Edge Hill virus, Entebbe bat virus, Gadgets Gully virus, Ilheus virus, Israel turkey meningoencephalomyelitis virus, Japanese encephalitis virus, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Montana myotis leukoencephalitis virus, Murray Valley encephalitis virus, Ntaya virus, Omsk hemorrhagic fever virus, Phnom Penh bat virus, Powassan virus, Rio Bravo virus, Royal Farm virus, Saboya virus, Saint Louis encephalitis virus, Sal Vieja virus, San Perlita virus, Saumarez Reef virus, Sepik virus, Tembusu virus, Tick-borne encephalitis virus, Tyuleniy virus, Uganda S virus, Usutu virus, Wesselsbron virus, West Nile virus, Yaounde virus, Yellow fever virus, Yokose virus, Zika virus, Hepacivirus A, Hepacivirus B, Hepacivirus C, Hepacivirus D, Hepacivirus E, Hepacivirus F, Hepacivirus G, Hepacivirus H, Hepacivirus I, Hepacivirus J, Hepacivirus K, Hepacivirus L, Hepacivirus M, Hepacivirus N, Pegivirus A, Pegivirus B, Pegivirus C, Pegivirus D, Pegivirus E, Pegivirus F, Pegivirus G, Pegivirus H, Pegivirus I, Pegivirus J, Pegivirus K, Pestivirus A, Pestivirus B, Pestivirus C, Pestivirus D, Pestivirus E, Pestivirus F, Pestivirus G, Pestivirus H, Pestivirus I, Pestivirus J, Pestivirus K, Black beetle virus, Boolarra virus, Flock House virus, Nodamura virus, Pariacoto virus, Barfin flounder nervous necrosis virus, Redspotted grouper nervous necrosis virus, Striped jack nervous necrosis virus, Tiger puffer nervous necrosis virus, Lake Sinai virus 1, Lake Sinai virus 2, Providence virus, Alfalfa enamovirus 1, Birdsfoot trefoil enamovirus 1, Citrus vein enation virus, Grapevine enamovirus 1, Pea enation mosaic virus 1, Apple associated luteovirus, Apple luteovirus 1, Barley yellow dwarf virus kerII, Barley yellow dwarf virus kerIII, Barley yellow dwarf virus MAV, Barley yellow dwarf virus PAS, Barley yellow dwarf virus PAV, Bean leafroll virus, Cherry associated luteovirus, Nectarine stem pitting associated virus, Red clover associated luteovirus, Rose spring dwarf-associated virus, Soybean dwarf virus, Beet chlorosis virus, Beet mild yellowing virus, Beet western yellows virus, Carrot red leaf virus, Cereal yellow dwarf virus RPS, Cereal yellow dwarf virus RPV, Chickpea chlorotic stunt virus, Cotton leafroll dwarf virus, Cucurbit aphid-borne yellows virus, Faba bean polerovirus 1, Maize yellow dwarf virus RMV, Maize yellow mosaic virus, Melon aphid-borne yellows virus, Pepo aphid-borne yellows virus, Pepper vein yellows virus 1, Pepper vein yellows virus 2, Pepper vein yellows virus 3, Pepper vein yellows virus 4, Pepper vein yellows virus 5, Pepper vein yellows virus 6, Potato leafroll virus, Pumpkin polerovirus, Suakwa aphid-borne yellows virus, Sugarcane yellow leaf virus, Tobacco vein distorting virus, Turnip yellows virus, Barley yellow dwarf virus GPV, Barley yellow dwarf virus SGV, Chickpea stunt disease associated virus, Groundnut rosette assistor virus, Indonesian soybean dwarf virus, Sweet potato leaf speckling virus, Tobacco necrotic dwarf virus, Carrot mottle mimic virus, Carrot mottle virus, Ethiopian tobacco bushy top virus, Groundnut rosette virus, Lettuce speckles mottle virus, Opium poppy mosaic virus, Pea enation mosaic virus 2, Tobacco bushy top virus, Tobacco mottle virus, Angelonia flower break virus, Calibrachoa mottle virus, Carnation mottle virus, Honeysuckle ringspot virus, Nootka lupine vein clearing virus, Pelargonium flower break virus, Saguaro cactus virus, Olive latent virus 1, Olive mild mosaic virus, Potato necrosis virus, Tobacco necrosis virus A, Cucumber leaf spot virus, Johnsongrass chlorotic stripe mosaic virus, Maize white line mosaic virus, Pothos latent virus, Yam spherical virus, Oat chlorotic stunt virus, Cardamine chlorotic fleck virus, Hibiscus chlorotic ringspot virus, Japanese iris necrotic ring virus, Turnip crinkle virus, Beet black scorch virus, Leek white stripe virus, Tobacco necrosis virus D, Galinsoga mosaic virus, Cowpea mottle virus, Melon necrotic spot virus, Pea stem necrosis virus, Soybean yellow mottle mosaic virus, Furcraea necrotic streak virus, Maize chlorotic mottle virus, Cocksfoot mild mosaic virus, Panicum mosaic virus, Thin paspalum asymptomatic virus, Clematis chlorotic mottle virus, Elderberry latent virus, Pelargonium chlorotic ring pattern virus, Pelargonium line pattern virus, Pelargonium ringspot virus, Rosa rugosa leaf distortion virus, Artichoke mottled crinkle virus, Carnation Italian ringspot virus, Cucumber Bulgarian latent virus, Cucumber necrosis virus, Cymbidium ringspot virus, Eggplant mottled crinkle virus, Grapevine Algerian latent virus, Havel River virus, Lato River virus, Limonium flower distortion virus, Moroccan pepper virus, Neckar River virus, Pelargonium leaf curl virus, Pelargonium necrotic spot virus, Petunia asteroid mosaic virus, Sitke waterborne virus, Tomato bushy stunt virus, Maize necrotic streak virus, Ahlum waterborne virus, Bean mild mosaic virus, Chenopodium necrosis virus, Cucumber soil-borne virus, Trailing lespedeza virus 1, Weddel waterborne virus, Carnation ringspot virus, Red clover necrotic mosaic virus, Sweet clover necrotic mosaic virus, Escherichia virus FI, Escherichia virus Qbeta, Escherichia virus BZ13, Escherichia virus MS2, Saccharomyces 20S RNA narnavirus, Saccharomyces 23S RNA narnavirus, Cryphonectria mitovirus 1, Ophiostoma mitovirus 4, Ophiostoma mitovirus 5, Ophiostoma mitovirus 6, Ophiostoma mitovirus 3a, Botrytis botoulivirus, Sclerotinia botoulivirus 2, Magnaporthe magoulivirus 1, Rhizoctonia magoulivirus 1, Cassava virus C, Epirus cherry virus, Ourmia melon virus, Sclerotinia scleroulivirus 1, Soybean scleroulivirus 1, Soybean scleroulivirus 2, Beihai yingvirus, Charybdis yingvirus, Hubei yingvirus, Sanxia yingvirus, Shahe yingvirus, Wenzhou yingvirus, Wuhan yingvirus, Xinzhou yingvirus, Blueberry mosaic associated ophiovirus, Citrus psorosis ophiovirus, Freesia sneak ophiovirus, Lettuce ring necrosis ophiovirus, Mirafiori lettuce big-vein ophiovirus, Ranunculus white mottle ophiovirus, Tulip mild mottle mosaic ophiovirus, Argas mivirus, Barnacle mivirus, Beetle mivirus, Bole mivirus, Brunnich mivirus, Changping mivirus, Charybdis mivirus, Cockroach mivirus, Crab mivirus, Crustacean mivirus, Dermacentor mivirus, Hermit mivirus, Hippoboscid mivirus, Hubei mivirus, Hubei odonate mivirus, Imjin mivirus, Lacewing mivirus, Lishi mivirus, Lonestar mivirus, Louse fly mivirus, Mosquito mivirus, Myriapod mivirus, Odonate mivirus, Sanxia mivirus, Shayang mivirus, Suffolk mivirus, Taiyuan mivirus, Wenling mivirus, Wuhan mivirus, Xinzhou mivirus, Barnacle hexartovirus, Caligid hexartovirus, Beihai peropuvirus, Hubei peropuvirus, Odonate peropuvirus, Pillworm peropuvirus, Pteromalus puparum peropuvirus, Woodlouse peropuvirus, Queensland carbovirus, Southwest carbovirus, Sharpbelly cultervirus, Elapid 1 orthobornavirus, Mammalian 1 orthobornavirus, Mammalian 2 orthobornavirus, Passeriform 1 orthobornavirus, Passeriform 2 orthobornavirus, Psittaciform 1 orthobornavirus, Psittaciform 2 orthobornavirus, Waterbird 1 orthobornavirus, Lloviu cuevavirus, Mengla dianlovirus, Bombali ebolavirus, Bundibugyo ebolavirus, Reston ebolavirus, Sudan ebolavirus, Tai Forest ebolavirus, Zaire ebolavirus, Marburg marburgvirus, Xilang striavirus, Huangjiao thamnovirus, Gerrid arlivirus, Hubei arlivirus, Lishi arlivirus, Odonate arlivirus, Tacheng arlivirus, Wuchang arlivirus, Hubei hubramonavirus, Lentinula hubramonavirus, Dadou sclerotimonavirus, Drop sclerotimonavirus, Glycine sclerotimonavirus, Hubei sclerotimonavirus, Illinois sclerotimonavirus, Phyllosphere sclerotimonavirus, Sclerotinia sclerotimonavirus, Beihai berhavirus, Echinoderm berhavirus, Sipunculid berhavirus, Beihai crustavirus, Wenling crustavirus, Wenzhou crustavirus, Midway nyavirus, Nyamanini nyavirus, Sierra Nevada nyavirus, Orinoco orinovirus, Soybean cyst nematode socyvirus, Tapeworm tapwovirus, Avian metaavulavirus 2, Avian metaavulavirus 5, Avian metaavulavirus 6, Avian metaavulavirus 7, Avian metaavulavirus 8, Avian metaavulavirus 10, Avian metaavulavirus 11, Avian metaavulavirus 14, Avian metaavulavirus 15, Avian metaavulavirus 20, Avian orthoavulavirus 1, Avian orthoavulavirus 9, Avian orthoavulavirus 12, Avian orthoavulavirus 13, Avian orthoavulavirus 16, Avian orthoavulavirus 17, Avian orthoavulavirus 18, Avian orthoavulavirus 19, Avian orthoavulavirus 21, Avian orthovulavirus 21, Avian paraavulavirus 3, Avian paraavulavirus 4, Synodus synodonvirus, Oncorhynchus aquaparamyxovirus, Salmo aquaparamyxovirus, Reptilian ferlavirus, Cedar henipavirus, Ghanaian bat henipavirus, Hendra henipavirus, Mojiang henipavirus, Nipah henipavirus, Beilong jeilongvirus, Jun jeilongvirus, Lophuromys jeilongvirus 1, Lophuromys jeilongvirus 2, Miniopteran jeilongvirus, Myodes jeilongvirus, Tailam jeilongvirus, Canine morbillivirus, Cetacean morbillivirus, Feline morbillivirus, Measles morbillivirus, Phocine morbillivirus, Rinderpest morbillivirus, Small ruminant morbillivirus, Mossman narmovirus, Myodes narmovirus, Nariva narmovirus, Tupaia narmovirus, Bovine respirovirus 3, Caprine respirovirus 3, Human respirovirus 1, Human respirovirus 3, Murine respirovirus, Porcine respirovirus 1, Squirrel respirovirus, Salem salemvirus, Human orthorubulavirus 2, Human orthorubulavirus 4, Mammalian orthorubulavirus 5, Mammalian orthorubulavirus 6, Mapuera orthorubulavirus, Mumps orthorubulavirus, Porcine orthorubulavirus, Simian orthorubulavirus, Achimota pararubulavirus 1, Achimota pararubulavirus 2, Hervey pararubulavirus, Menangle pararubulavirus, Sosuga pararubulavirus, Teviot pararubulavirus, Tioman pararubulavirus, Tuhoko pararubulavirus 1, Tuhoko pararubulavirus 2, Tuhoko pararubulavirus 3, Cynoglossus cynoglossusvirus, Hoplichthys hoplichthysvirus, Scoliodon scoliodonvirus, Avian metapneumovirus, Human metapneumovirus, Bovine orthopneumovirus, Human orthopneumovirus, Murine orthopneumovirus, Arboretum almendravirus, Balsa almendravirus, Coot Bay almendravirus, Menghai almendravirus, Puerto Almendras almendravirus, Rio Chico almendravirus, Xingshan alphanemrhavirus, Xinzhou alphanemrhavirus, Eggplant mottled dwarf alphanucleorhabdovirus, Maize Iranian mosaic alphanucleorhabdovirus, Maize mosaic alphanucleorhabdovirus, Morogoro maize-associated alphanucleorhabdovirus, Physostegia chlorotic mottle alphanucleorhabdovirus, Potato yellow dwarf alphanucleorhabdovirus, Rice yellow stunt alphanucleorhabdovirus, Taro vein chlorosis alphanucleorhabdovirus, Wheat yellow striate alphanucleorhabdovirus, Aruac arurhavirus, Inhangapi arurhavirus, Santabarbara arurhavirus, Xiburema arurhavirus, Bahia barhavirus, Muir barhavirus, Alfalfa betanucleorhabdovirus, Blackcurrant betanucleorhabdovirus, Datura yellow vein betanucleorhabdovirus, Sonchus yellow net betanucleorhabdovirus, Sowthistle yellow vein betanucleorhabdovirus, Trefoil betanucleorhabdovirus, Caligus caligrhavirus, Lepeophtheirus caligrhavirus, Salmonlouse caligrhavirus, Curionopolis curiovirus, Iriri curiovirus, Itacaiunas curiovirus, Rochambeau curiovirus, Alfalfa dwarf cytorhabdovirus, Barley yellow striate mosaic cytorhabdovirus, Broccoli necrotic yellows cytorhabdovirus, Cabbage cytorhabdovirus, Colocasia bobone disease-associated cytorhabdovirus, Festuca leaf streak cytorhabdovirus, Lettuce necrotic yellows cytorhabdovirus, Lettuce yellow mottle cytorhabdovirus, Maize yellow striate cytorhabdovirus, Maize-associated cytorhabdovirus, Northern cereal mosaic cytorhabdovirus, Papaya cytorhabdovirus, Persimmon cytorhabdovirus, Raspberry vein chlorosis cytorhabdovirus, Rice stripe mosaic cytorhabdovirus, Sonchus cytorhabdovirus 1, Strawberry crinkle cytorhabdovirus, Tomato yellow mottle-associated cytorhabdovirus, Wheat American striate mosaic cytorhabdovirus, Wuhan 4 insect cytorhabdovirus, Wuhan 5 insect cytorhabdovirus, Wuhan 6 insect cytorhabdovirus, Yerba mate chlorosis-associated cytorhabdovirus, Citrus chlorotic spot dichorhavirus, Citrus leprosis N dichorhavirus, Clerodendrum chlorotic spot dichorhavirus, Coffee ringspot dichorhavirus, Orchid fleck dichorhavirus, Adelaide River ephemerovirus, Berrimah ephemerovirus, Bovine fever ephemerovirus, Kimberley ephemerovirus, Koolpinyah ephemerovirus, Kotonkan ephemerovirus, Obodhiang ephemerovirus, Yata ephemerovirus, Maize fine streak gammanucleorhabdovirus, Flanders hapavirus, Gray Lodge hapavirus, Hart Park hapavirus, Holmes hapavirus, Joinjakaka hapavirus, Kamese hapavirus, La Joya hapavirus, Landjia hapavirus, Manitoba hapavirus, Marco hapavirus, Mosqueiro hapavirus, Mossuril hapavirus, Ngaingan hapavirus, Ord River hapavirus, Parry Creek hapavirus, Wongabel hapavirus, Barur ledantevirus, Fikirini ledantevirus, Fukuoka ledantevirus, Kanyawara ledantevirus, Kern Canyon ledantevirus, Keuraliba ledantevirus, Kolente ledantevirus, Kumasi ledantevirus, Le Dantec ledantevirus, Mount Elgon bat ledantevirus, Nishimuro ledantevirus, Nkolbisson ledantevirus, Oita ledantevirus, Vaprio ledantevirus, Wuhan ledantevirus, Yongjia ledantevirus, Lonestar zarhavirus, Aravan lyssavirus, Australian bat lyssavirus, Bokeloh bat lyssavirus, Duvenhage lyssavirus, European bat 1 lyssavirus, European bat 2 lyssavirus, Gannoruwa bat lyssavirus, Ikoma lyssavirus, Irkut lyssavirus, Khuj and lyssavirus, Lagos bat lyssavirus, Lleida bat lyssavirus, Mokola lyssavirus, Rabies lyssavirus, Shimoni bat lyssavirus, Taiwan bat lyssavirus, West Caucasian bat lyssavirus, Moussa mousrhavirus, Hirame novirhabdovirus, Piscine novirhabdovirus, Salmonid novirhabdovirus, Snakehead novirhabdovirus, Culex ohlsrhavirus, Northcreek ohlsrhavirus, Ohlsdorf ohlsrhavirus, Riverside ohlsrhavirus, Tongilchon ohlsrhavirus, Anguillid perhabdovirus, Perch perhabdovirus, Sea trout perhabdovirus, Connecticut sawgrhavirus, Island sawgrhavirus, Minto sawgrhavirus, Sawgrass sawgrhavirus, Drosophila affinis sigmavirus, Drosophila ananassae sigmavirus, Drosophila immigrans sigmavirus, Drosophila melanogaster sigmavirus, Drosophila obscura sigmavirus, Drosophila tristis sigmavirus, Muscina stabulans sigmavirus, Carp sprivivirus, Pike fry sprivivirus, Almpiwar sripuvirus, Chaco sripuvirus, Charleville sripuvirus, Cuiaba sripuvirus, Hainan sripuvirus, Niakha sripuvirus, Sena Madureira sripuvirus, Sripur sripuvirus, Garba sunrhavirus, Harrison sunrhavirus, Kwatta sunrhavirus, Oakvale sunrhavirus, Sunguru sunrhavirus, Walkabout sunrhavirus, Bas-Congo tibrovirus, Beatrice Hill tibrovirus, Coastal Plains tibrovirus, Ekpoma 1 tibrovirus, Ekpoma 2 tibrovirus, Sweetwater Branch tibrovirus, Tibrogargan tibrovirus, Durham tupavirus, Klamath tupavirus, Tupaia tupavirus, Lettuce big-vein associated varicosavirus, Alagoas vesiculovirus, American bat vesiculovirus, Carajas vesiculovirus, Chandipura vesiculovirus, Cocal vesiculovirus, Indiana vesiculovirus, Isfahan vesiculovirus, Jurona vesiculovirus, Malpais Spring vesiculovirus, Maraba vesiculovirus, Morreton vesiculovirus, New Jersey vesiculovirus, Perinet vesiculovirus, Piry vesiculovirus, Radi vesiculovirus, Yug Bogdanovac vesiculovirus, Zahedan zarhavirus, Reptile sunshinevirus 1, Bolahun anphevirus, Dipteran anphevirus, Drosophilid anphevirus, Odonate anphevirus, Orthopteran anphevirus, Shuangao anphevirus, Xincheng anphevirus, Beihai yuyuevirus, Shahe yuyuevirus, Hairy antennavirus, Striated antennavirus, Haartman hartmanivirus, Muikkunen hartmanivirus, Schoolhouse hartmanivirus, Zurich hartmanivirus, Allpahuayo mammarenavirus, Alxa mammarenavirus, Argentinian mammarenavirus, Bear Canyon mammarenavirus, Brazilian mammarenavirus, Cali mammarenavirus, Chapare mammarenavirus, Chevrier mammarenavirus, Cupixi mammarenavirus, Flexal mammarenavirus, Gairo mammarenavirus, Guanarito mammarenavirus, Ippy mammarenavirus, Lassa mammarenavirus, Latino mammarenavirus, Loei River mammarenavirus, Lujo mammarenavirus, Luna mammarenavirus, Lunk mammarenavirus, Lymphocytic choriomeningitis mammarenavirus, Machupo mammarenavirus, Mariental mammarenavirus, Merino Walk mammarenavirus, Mobala mammarenavirus, Mopeia mammarenavirus, Okahandj a mammarenavirus, Oliveros mammarenavirus, Paraguayan mammarenavirus, Pirital mammarenavirus, Planalto mammarenavirus, Ryukyu mammarenavirus, Serra do Navio mammarenavirus, Solwezi mammarenavirus, Souris mammarenavirus, Tacaribe mammarenavirus, Tamiami mammarenavirus, Wenzhou mammarenavirus, Whitewater Arroyo mammarenavirus, Xapuri mammarenavirus, California reptarenavirus, Giessen reptarenavirus, Golden reptarenavirus, Ordinary reptarenavirus, Rotterdam reptarenavirus, Crustacean lincruvirus, Actinidia chlorotic ringspot-associated emaravirus, Blackberry leaf mottle associated emaravirus, European mountain ash ringspot-associated emaravirus, Fig mosaic emaravirus, High Plains wheat mosaic emaravirus, Pigeonpea sterility mosaic emaravirus 1, Pigeonpea sterility mosaic emaravirus 2, Pistacia emaravirus B, Raspberry leaf blotch emaravirus, Redbud yellow ringspot-associated emaravirus, Rose rosette emaravirus, Batfish actinovirus, Goosefish actinovirus, Spikefish actinovirus, Hagfish agnathovirus, Brno loanvirus, Longquan loanvirus, Laibin mobatvirus, Nova mobatvirus, Quezon mobatvirus, Andes orthohantavirus, Asama orthohantavirus, Asikkala orthohantavirus, Bayou orthohantavirus, Black Creek Canal orthohantavirus, Bowe orthohantavirus, Bruges orthohantavirus, Cano Delgadito orthohantavirus, Cao Bang orthohantavirus, Choclo orthohantavirus, Dabieshan orthohantavirus, Dobrava-Belgrade orthohantavirus, El Moro Canyon orthohantavirus, Fugong orthohantavirus, Fusong orthohantavirus, Hantaan orthohantavirus, Jeju orthohantavirus, Kenkeme orthohantavirus, Khabarovsk orthohantavirus, Laguna Negra orthohantavirus, Luxi orthohantavirus, Maporal orthohantavirus, Montano orthohantavirus, Necocli orthohantavirus, Oxbow orthohantavirus, Prospect Hill orthohantavirus, Puumala orthohantavirus, Rockport orthohantavirus, Sangassou orthohantavirus, Seewis orhtohantavirus, Seoul orthohantavirus, Sin Nombre orthohantavirus, Thailand orthohantavirus, Tigray orthohantavirus, Tula orthohantavirus, Yakeshi orthohantavirus, Imjin thottimvirus, Thottopalayam thottimvirus, Gecko reptillovirus, Leptomonas shilevirus, Myriapod hubavirus, Artashat orthonairovirus, Chim orthonairovirus, Crimean-Congo hemorrhagic fever orthonairovirus, Dera Ghazi Khan orthonairovirus, Dugbe orthonairovirus, Estero Real orthonairovirus, Hazara orthonairovirus, Hughes orthonairovirus, Kasokero orthonairovirus, Keterah orthonairovirus, Nairobi sheep disease orthonairovirus, Qalyub orthonairovirus, Sakhalin orthonairovirus, Tamdy orthonairovirus, Thiafora orthonairovirus, Spider shaspivirus, Strider striwavirus, Herbert herbevirus, Kibale herbevirus, Tai herbevirus, Acara orthobunyavirus, Aino orthobunyavirus, Akabane orthobunyavirus, Alajuela orthobunyavirus, Anadyr orthobunyavirus, Anhembi orthobunyavirus, Anopheles A orthobunyavirus, Anopheles B orthobunyavirus, Bakau orthobunyavirus, Batai orthobunyavirus, Batama orthobunyavirus, Bellavista orthobunyavirus, Benevides orthobunyavirus, Bertioga orthobunyavirus, Bimiti orthobunyavirus, Birao orthobunyavirus, Botambi orthobunyavirus, Bozo orthobunyavirus, Bunyamwera orthobunyavirus, Bushbush orthobunyavirus, Buttonwillow orthobunyavirus, Bwamba orthobunyavirus, Cache Valley orthobunyavirus, Cachoeira Porteira orthobunyavirus, California encephalitis orthobunyavirus, Capim orthobunyavirus, Caraparu orthobunyavirus, Cat Que orthobunyavirus, Catu orthobunyavirus, Enseada orthobunyavirus, Faceys paddock orthobunyavirus, Fort Sherman orthobunyavirus, Gamboa orthobunyavirus, Guajara orthobunyavirus, Guama orthobunyavirus, Guaroa orthobunyavirus, Iaco orthobunyavirus, Ilesha orthobunyavirus, Ingwavuma orthobunyavirus, Jamestown Canyon orthobunyavirus, Jatobal orthobunyavirus, Kaeng Khoi orthobunyavirus, Kairi orthobunyavirus, Keystone orthobunyavirus, Koongol orthobunyavirus, La Crosse orthobunyavirus, Leanyer orthobunyavirus, Lumbo orthbunyavirus, Macaua orthobunyavirus, Madrid orthobunyavirus, Maguari orthobunyavirus, Main Drain orthobunyavirus, Manzanilla orthobunyavirus, Marituba orthobunyavirus, Melao orthobunyavirus, Mermet orthobunyavirus, Minatitlan orthobunyavirus, MPoko orthobunyavirus, Nyando orthobunyavirus, Olifantsvlei orthobunyavirus, Oriboca orthobunyavirus, Oropouche orthobunyavirus, Patois orthobunyavirus, Peaton orthobunyavirus, Potosi orthobunyavirus, Sabo orthobunyavirus, San Angelo orthobunyavirus, Sango orthobunyavirus, Schmallenberg orthobunyavirus, Serra do Navio orthobunyavirus, Shuni orthobunyavirus, Simbu orthobunyavirus, Snowshoe hare orthobunyavirus, Sororoca orthobunyavirus, Tacaiuma orthobunyavirus, Tahyna orthobunyavirus, Tataguine orthobunyavirus, Tensaw orthobunyavirus, Tete orthobunyavirus, Thimiri orthobunyavirus, Timboteua orthobunyavirus, Trivittatus orthobunyavirus, Turlock orthobunyavirus, Utinga orthobunyavirus, Witwatersrand orthobunyavirus, Wolkberg orthobunyavirus, Wyeomyia orthobunyavirus, Zegla orthobunyavirus, Caimito pacuvirus, Chilibre pacuvirus, Pacui pacuvirus, Rio Preto da Eva pacuvirus, Tapirape pacuvirus, Insect shangavirus, Ferak feravirus, Jonchet jonvirus, Anopheles orthophasmavirus, Culex orthophasmavirus, Ganda orthophasmavirus, Kigluaik phantom orthophasmavirus, Odonate orthophasmavirus, Qingling orthophasmavirus, Wuchang cockroach orthophasmavirus 1, Wuhan mosquito orthophasmavirus 1, Wuhan mosquito orthophasmavirus 2, Sanxia sawastrivirus, Insect wuhivirus, Bhanja bandavirus, Dabie bandavirus, Guertu bandavirus, Heartland bandavirus, Hunter Island bandavirus, Kismaayo bandavirus, Lone Star bandavirus, Dipteran beidivirus, Citrus coguvirus, Coguvirus eburi, Entoleuca entovirus, Cumuto goukovirus, Gouleako goukovirus, Yichang insect goukovirus, Horsefly horwuvirus, Dipteran hudivirus, Lepidopteran hudovirus, Blackleg ixovirus, Norway ixovirus, Scapularis ixovirus, Laurel Lake laulavirus, Lentinula lentinuvirus, Mothra mobuvirus, Badu phasivirus, Dipteran phasivirus, Fly phasivirus, Phasi Charoen-like phasivirus, Wutai mosquito phasivirus, Adana phlebovirus, Aguacate phlebovirus, Alcube phlebovirus, Alenquer phlebovirus, Ambe phlebovirus, Anhanga phlebovirus, Arumowot phlebovirus, Buenaventura phlebovirus, Buj am phlebovirus, Cacao phlebovirus, Campana phlebovirus, Candiru phlebovirus, Chagres phlebovirus, Code phlebovirus, Dashli phlebovirus, Durania phlebovirus, Echarate phlebovirus, Frijoles phlebovirus, Gabek phlebovirus, Gordil phlebovirus, Icoaraci phlebovirus, Itaituba phlebovirus, Itaporanga phlebovirus, Ixcanal phlebovirus, Karimabad phlebovirus, La Gloria phlebovirus, Lara phlebovirus, Leticia phlebovirus, Maldonado phlebovirus, Massilia phlebovirus, Medjerda phlebovirus, Mona Grita phlebovirus, Mukawa phlebovirus, Munguba phlebovirus, Naples phlebovirus, Nique phlebovirus, Ntepes phlebovirus, Odrenisrou phlebovirus, Oriximina phlebovirus, Pena Blanca phlebovirus, Punique phlebovirus, Punta Toro phlebovirus, Rift Valley fever phlebovirus, Rio Grande phlebovirus, Saint Floris phlebovirus, Salanga phlebovirus, Salehabad phlebovirus, Salobo phlabovirus, Sicilian phlebovirus, Tapara phlebovirus, Tehran phlebovirus, Tico phebovirus, Toros phlebovirus, Toscana phlebovirus, Tres Almendras phlebovirus, Turuna phlebovirus, Uriurana phlebovirus, Urucuri phlebovirus, Viola phlebovirus, Zerdali phlebovirus, Pidgey pidchovirus, Apple rubodvirus 1, Apple rubodvirus 2, Echinochloa hoja blanca tenuivirus, Iranian wheat stripe tenuivirus, Maize stripe tenuivirus, Melon tenuivirus, Rice grassy stunt tenuivirus, Rice hoja blanca tenuivirus, Rice stripe tenuivirus, Urochloa hoja blanca tenuivirus, American dog uukuvirus, Dabieshan uukuvirus, Grand Arbaud uukuvirus, Huangpi uukuvirus, Kabuto mountain uukuvirus, Kaisodi uukuvirus, Lihan uukuvirus, Murre uukuvirus, Pacific coast uukuvirus, Precarious Point uukuvirus, Rukutama uukuvirus, Schmidt uukuvirus, Silverwater uukuvirus, Tacheng uukuvirus, Uukuniemi uukuvirus, Yongjia uukuvirus, Zaliv Terpeniya uukuvirus, Shrimp wenrivirus, Alstroemeria necrotic streak orthotospovirus, Alstroemeria yellow spot orthotospovirus, Bean necrotic mosaic orthotospovirus, Calla lily chlorotic spot orthotospovirus, Capsicum chlorosis orthotospovirus, Chrysanthemum stem necrosis orthotospovirus, Groundnut bud necrosis orthotospovirus, Groundnut chlorotic fan spot orthotospovirus, Groundnut ringspot orthotospovirus, Groundnut yellow spot orthotospovirus, Hippeastrum chlorotic ringspot orthotospovirus, Impatiens necrotic spot orthotospovirus, Iris yellow spot orthotospovirus, Melon severe mosaic orthotospovirus, Melon yellow spot orthotospovirus, Mulberry vein banding associated orthotospovirus, Pepper chlorotic spot orthotospovirus, Polygonum ringspot orthotospovirus, Soybean vein necrosis orthotospovirus, Tomato chlorotic spot orthotospovirus, Tomato spotted wilt orthotospovirus, Tomato yellow ring orthotospovirus, Tomato zonate spot orthotospovirus, Watermelon bud necrosis orthotospovirus, Watermelon silver mottle orthotospovirus, Zucchini lethal chlorosis orthotospovirus, Millipede wumivirus, Tilapia tilapinevirus, Influenza A virus, Influenza B virus, Influenza D virus, Influenza C virus, Salmon isavirus, Johnston Atoll quaranjavirus, Quaranfil quaranjavirus, Dhori thogotovirus, Thogoto thogotovirus, Allium cepa amalgavirus 1, Allium cepa amalgavirus 2, Blueberry latent virus, Rhododendron virus A, Southern tomato virus, Spinach amalgavirus 1, Vicia cryptic virus M, Zoostera marina amalgavirus 1, Zoostera marina amalgavirus 2, Zygosaccharomyces bailii virus Z, Cryphonectria hypovirus 1, Cryphonectria hypovirus 2, Cryphonectria hypovirus 3, Cryphonectria hypovirus 4, Beet cryptic virus 1, Carrot cryptic virus, Cherry chlorotic rusty spot associated partitivirus, Chondrostereum purpureum cryptic virus 1, Flammulina velutipes browning virus, Helicobasidium mompa partitivirus V70, Heterobasidion partitivirus 1, Heterobasidion partitivirus 3, Heterobasidion partitivirus 12, Heterobasidion partitivirus 13, Heterobasidion partitivirus 15, Rosellinia necatrix partitivirus 2, Vicia cryptic virus, White clover cryptic virus 1, Atkinsonella hypoxylon virus, Cannabis cryptic virus, Ceratocystis resinifera virus 1, Crimson clover cryptic virus 2, Dill cryptic virus 2, Fusarium poae virus 1, Heterobasidion partitivirus 2, Heterobasidion partitivirus 7, Heterobasidion partitivirus 8, Heterobasidion partitivirus P, Hop trefoil cryptic virus 2, Pleurotus ostreatus virus 1, Primula malacoides virus 1, Red clover cryptic virus 2, Rhizoctonia solani virus 717, Rosellinia necatrix virus 1, White clover cryptic virus 2, Cryptosporidium parvum virus 1, Beet cryptic virus 2, Beet cryptic virus 3, Fig cryptic virus, Pepper cryptic virus 1, Pepper cryptic virus 2, Aspergillus ochraceous virus, Discula destructiva virus 1, Discula destructiva virus 2, Fusarium solani virus 1, Gremmeniella abietina RNA virus MS1, Ophiostoma partitivirus 1, Penicillium stoloniferum virus F, Penicillium stoloniferum virus S, Agaricus bisporus virus 4, Alfalfa cryptic virus 1, Carnation cryptic virus 1, Carrot temperate virus 1, Carrot temperate virus 2, Carrot temperate virus 3, Carrot temperate virus 4, Gaeumannomyces graminis virus 0196A, Gaeumannomyces graminis virus T1A, Hop trefoil cryptic virus 1, Hop trefoil cryptic virus 3, Radish yellow edge virus, Ryegrass cryptic virus, Spinach temperate virus, White clover cryptic virus 3, Beihai picobirnavirus, Equine picobirnavirus, Human picobirnavirus, Aplysia abyssovirus 1, Muarterivirus afrigant, Alphaarterivirus equid, Lambdaarterivirus afriporav, Deltaarterivirus hemfev, Epsilonarterivirus hemcep, Epsilonarterivirus safriver, Epsilonarterivirus zamalb, Etaarterivirus ugarco 1, Iotaarterivirus debrazmo, Iotaarterivirus kibreg 1, Iotaarterivirus pejah, Thetaarterivirus kafuba, Thetaarterivirus mikelba 1, Zetaarterivirus ugarco 1, Betaarterivirus suid 2, Betaarterivirus chinrav 1, Betaarterivirus ninrav, Betaarterivirus sheoin, Betaarterivirus suid 1, Betaarterivirus timiclar, Gammaarterivirus lacdeh, Nuarterivirus guemel, Kappaarterivirus wobum, Chinturpovirus 1, Ptyasnivirus 1, Oligodon snake nidovirus 1, Microhyla letovirus 1, Bat coronavirus CDPHE15, Bat coronavirus HKU10, Rhinolophus ferrumequinum alphacoronavirus HuB-2013, Human coronavirus 229E, Lucheng Rn rat coronavirus, Mink coronavirus 1, Miniopterus bat coronavirus 1, Miniopterus bat coronavirus HKU8, Myotis ricketti alphacoronavirus Sax-2011, Nyctalus velutinus alphacoronavirus SC-2013, Pipistrellus kuhlii coronavirus 3398, Porcine epidemic diarrhea virus, Scotophilus bat coronavirus 512, Rhinolophus bat coronavirus HKU2, Human coronavirus NL63, NL63-related bat coronavirus strain BtKYNL63-9b, Sorex araneus coronavirus T14, Suncus murinus coronavirus X74, Alphacoronavirus 1, Betacoronavirus 1, China Rattus coronavirus HKU24, Human coronavirus HKU1, Murine coronavirus, Myodes coronavirus 2JL14, Bat Hp-betacoronavirus Zhejiang2013, Hedgehog coronavirus 1, Middle East respiratory syndrome-related coronavirus, Pipistrellus bat coronavirus HKU5, Tylonycteris bat coronavirus HKU4, Eidolon bat coronavirus C704, Rousettus bat coronavirus GCCDC1, Rousettus bat coronavirus HKU9, Severe acute respiratory syndrome-related coronavirus, Wigeon coronavirus HKU20, Bulbul coronavirus HKU11, Common moorhen coronavirus HKU21, Coronavirus HKU15, Munia coronavirus HKU13, White-eye coronavirus HKU16, Night heron coronavirus HKU19, Goose coronavirus CB17, Beluga whale coronavirus SW1, Avian coronavirus, Avian coronavirus 9203, Duck coronavirus 2714, Turrinivirus 1, Botrylloides leachii nidovirus, Alphamesonivirus 4, Alphamesonivirus 8, Alphamesonivirus 5, Alphamesonivirus 7, Alphamesonivirus 2, Alphamesonivirus 3, Alphamesonivirus 9, Alphamesonivirus 1, Alphamesonivirus 10, Alphamesonivirus 6, Planidovirus 1, Nangarvirus 1, Halfbeak nidovirus 1, Charybnivirus 1, Decronivirus 1, Paguronivirus 1, Gill-associated virus, Okavirus 1, Yellow head virus, White bream virus, Fathead minnow nidovirus 1, Chinook salmon nidovirus 1, Bovine nidovirus 1, Hebius tobanivirus 1, Infratovirus 1, Lycodon tobanivirus 1, Ball python nidovirus 1, Morelia tobanivirus 1, Berisnavirus 1, Shingleback nidovirus 1, Sectovirus 1, Bovine torovirus, Equine torovirus, Porcine torovirus, Bavaria virus, European brown hare syndrome virus, Rabbit hemorrhagic disease virus, Minovirus A, Nacovirus A, Newbury 1 virus, Norwalk virus, Recovirus A, Nordland virus, Sapporo virus, Saint Valerien virus, Feline calicivirus, Vesicular exanthema of swine virus, Acute bee paralysis virus, Israeli acute paralysis virus, Kashmir bee virus, Mud crab virus, Solenopsis invicta virus 1, Taura syndrome virus, Aphid lethal paralysis virus, Cricket paralysis virus, Drosophila C virus, Rhopalosiphum padi virus, Black queen cell virus, Himetobi P virus, Homalodisca coagulata virus 1, Plautia stali intestine virus, Triatoma virus, Antheraea pernyi iflavirus, Brevicoryne brassicae virus, Deformed wing virus, Dinocampus coccinellae paralysis virus, Ectropis obliqua virus, Infectious flacherie virus, Lygus lineolaris virus 1, Lymantria dispar iflavirus 1, Nilaparvata lugens honeydew virus 1, Perina nuda virus, Sacbrood virus, Slow bee paralysis virus, Spodoptera exigua iflavirus 1, Spodoptera exigua iflavirus 2, Varroa destructor virus 1, Chaetoceros socialis forma radians RNA virus 1, Chaetoceros tenuissimus RNA virus 01, Rhizosolenia setigera RNA virus 01, Astarnavirus, Aurantiochytrium single-stranded RNA virus 01, Jericarnavirus B, Sanfarnavirus 1, Sanfarnavirus 2, Sanfarnavirus 3, Heterosigma akashiwo RNA virus, Britarnavirus 1, Britarnavirus 4, Palmarnavirus 128, Palmarnavirus 473, Britarnavirus 2, Britarnavirus 3, Chaetarnavirus 2, Chaetenuissarnavirus II, Jericarnavirus A, Palmarnavirus 156, Aalivirus A, Ailurivirus A, Ampivirus A, Anativirus A, Anativirus B, Bovine rhinitis A virus, Bovine rhinitis B virus, Equine rhinitis A virus, Foot-and-mouth disease virus, Aquamavirus A, Avihepatovirus A, Avisivirus A, Avisivirus B, Avisivirus C, Boosepivirus A, Boosepivirus B, Boosepivirus C, Bopivirus A, Cardiovirus A, Cardiovirus B, Cardiovirus C, Cardiovirus D, Cardiovirus E, Cardiovirus F, Cosavirus A, Cosavirus B, Cosavirus D, Cosavirus E, Cosavirus F, Crahelivirus A, Crohivirus A, Crohivirus B, Cadicivirus A, Cadicivirus B, Diresapivirus A, Diresapivirus B, Enterovirus A, Enterovirus B, Enterovirus C, Enterovirus D, Enterovirus E, Enterovirus F, Enterovirus G, Enterovirus H, Enterovirus I, Enterovirus J, Enterovirus K, Enterovirus L, Rhinovirus A, Rhinovirus B, Rhinovirus C, Erbovirus A, Felipivirus A, Fipivirus A, Fipivirus B, Fipivirus C, Fipivirus D, Fipivirus E, Gallivirus A, Gruhelivirus A, Grusopivirus A, Grusopivirus B, Harkavirus A, Hemipivirus A, Hepatovirus A, Hepatovirus B, Hepatovirus C, Hepatovirus D, Hepatovirus E, Hepatovirus F, Hepatovirus G, Hepatovirus H, Hepatovirus I, Hunnivirus A, Aichivirus A, Aichivirus B, Aichivirus C, Aichivirus D, Aichivirus E, Aichivirus F, Kunsagivirus A, Kunsagivirus B, Kunsagivirus C, Limnipivirus A, Limnipivirus B, Limnipivirus C, Livupivirus A, Ludopivirus A, Malagasivirus A, Malagasivirus B, Megrivirus A, Megrivirus B, Megrivirus C, Megrivirus D, Megrivirus E, Mischivirus A, Mischivirus B, Mischivirus C, Mischivirus D, Mosavirus A, Mosavirus B, Mupivirus A, Myrropivirus A, Orivirus A, Oscivirus A, Parabovirus A, Parabovirus B, Parabovirus C, Parechovirus A, Parechovirus B, Parechovirus C, Parechovirus D, Parechovirus E, Parechovirus F, Pasivirus A, Passerivirus A, Passerivirus B, Pemapivirus A, Poecivirus A, Potamipivirus A, Potamipivirus B, Rabovirus A, Rabovirus B, Rabovirus C, Rabovirus D, Rafivirus A, Rafivirus B, Rafivirus C, Rohelivirus A, Rosavirus A, Rosavirus B, Rosavirus C, Sakobuvirus A, Salivirus A, Sapelovirus A, Sapelovirus B, Senecavirus A, Shanbavirus A, Sicinivirus A, Symapivirus A, Teschovirus A, Teschovirus B, Torchivirus A, Tottorivirus A, Tremovirus A, Tremovirus B, Tropivirus A, Chironomus riparius virus 1, Hubei chipolycivirus, Hubei hupolycivirus, Formica exsecta virus 3, Lasius neglectus virus 1, Lasius neglectus virus 2, Lasius niger virus 1, Linepithema humile virus 2, Monomorium pharaonis virus 1, Monomorium pharaonis virus 2, Myrmica scabrinodis virus 1, Shuangao insect virus 8, Solenopsis invicta virus 2, Solenopsis invicta virus 4, Andean potato mottle virus, Bean pod mottle virus, Bean rugose mosaic virus, Broad bean stain virus, Broad bean true mosaic virus, Cowpea mosaic virus, Cowpea severe mosaic virus, Glycine mosaic virus, Pea green mottle virus, Pea mild mosaic virus, Quail pea mosaic virus, Radish mosaic virus, Red clover mottle virus, Squash mosaic virus, Ullucus virus C, Broad bean wilt virus 1, Broad bean wilt virus 2, Cucurbit mild mosaic virus, Gentian mosaic virus, Grapevine fabavirus, Lamium mild mosaic virus, Prunus virus F, Aeonium ringspot virus, Apricot latent ringspot virus, Arabis mosaic virus, Arracacha virus A, Artichoke Aegean ringspot virus, Artichoke Italian latent virus, Artichoke yellow ringspot virus, Beet ringspot virus, Blackcurrant reversion virus, Blueberry latent spherical virus, Blueberry leaf mottle virus, Cassava American latent virus, Cassava green mottle virus, Cherry leaf roll virus, Chicory yellow mottle virus, Cocoa necrosis virus, Crimson clover latent virus, Cycas necrotic stunt virus, Grapevine Anatolian ringspot virus, Grapevine Bulgarian latent virus, Grapevine chrome mosaic virus, Grapevine deformation virus, Grapevine fanleaf virus, Grapevine Tunisian ringspot virus, Hibiscus latent ringspot virus, Lucerne Australian latent virus, Melon mild mottle virus, Mulberry mosaic leaf roll associated virus, Mulberry ringspot virus, Myrobalan latent ringspot virus, Olive latent ringspot virus, Peach rosette mosaic virus, Potato black ringspot virus, Potato virus B, Potato virus U, Raspberry ringspot virus, Soybean latent spherical virus, Tobacco ringspot virus, Tomato black ring virus, Tomato ringspot virus, Apple latent spherical virus, Arracacha virus B, Cherry rasp leaf virus, Currant latent virus, Stocky prune virus, Chocolate lily virus A, Dioscorea mosaic associated virus, Satsuma dwarf virus, Black raspberry necrosis virus, Strawberry mottle virus, Carrot necrotic dieback virus, Dandelion yellow mosaic virus, Parsnip yellow fleck virus, Carrot torradovirus 1, Lettuce necrotic leaf curl virus, Motherwort yellow mottle virus, Squash chlorotic leaf spot virus, Tomato marchitez virus, Tomato torrado virus, Anthriscus yellows virus, Bellflower vein chlorosis virus, Maize chlorotic dwarf virus, Rice tungro spherical virus, Strawberry latent ringspot virus, Solenopsis invicta virus 3, Nylanderia fulva virus 1, Heterocapsa circularisquama RNA virus 01, Mushroom bacilliform virus, Poinsettia latent virus, Artemisia virus A, Blueberry shoestring virus, Cocksfoot mottle virus, Cymbidium chlorotic mosaic virus, Imperata yellow mottle virus, Lucerne transient streak virus, Papaya lethal yellowing virus, Rice yellow mottle virus, Rottboellia yellow mottle virus, Ryegrass mottle virus, Sesbania mosaic virus, Solanum nodiflorum mottle virus, Southern bean mosaic virus, Southern cowpea mosaic virus, Sowbane mosaic virus, Soybean yellow common mosaic virus, Subterranean clover mottle virus, Turnip rosette virus, Velvet tobacco mottle virus, Areca palm necrotic ringspot virus, Areca palm necrotic spindle-spot virus, Bellflower veinal mottle virus, Blackberry virus Y, Barley mild mosaic virus, Barley yellow mosaic virus, Oat mosaic virus, Rice necrosis mosaic virus, Wheat spindle streak mosaic virus, Wheat yellow mosaic virus, Celery latent virus, Cassava brown streak virus, Coccinia mottle virus, Cucumber vein yellowing virus, Squash vein yellowing virus, Sweet potato mild mottle virus, Tomato mild mottle virus, Ugandan cassava brown streak virus, Alpinia mosaic virus, Alpinia oxyphylla mosaic virus, Artichoke latent virus, Broad-leafed dock virus A, Cardamom mosaic virus, Chinese yam necrotic mosaic virus, Maclura mosaic virus, Narcissus latent virus, Yam chlorotic mosaic virus, Yam chlorotic necrosis virus, Caladenia virus A, Sugarcane streak mosaic virus, Triticum mosaic virus, African eggplant mosaic virus, Algerian watermelon mosaic virus, Alstroemeria mosaic virus, Alternanthera mild mosaic virus, Amaranthus leaf mottle virus, Amazon lily mosaic virus, Angelica virus Y, Apium virus Y, Araujia mosaic virus, Arracacha mottle virus, Asparagus virus 1, Banana bract mosaic virus, Barbacena virus Y, Basella rugose mosaic virus, Bean common mosaic necrosis virus, Bean common mosaic virus, Bean yellow mosaic virus, Beet mosaic virus, Bidens mosaic virus, Bidens mottle virus, Blue squill virus A, Brugmansia mosaic virus, Brugmansia suaveolens mottle virus, Butterfly flower mosaic virus, Calanthe mild mosaic virus, Calla lily latent virus, Callistephus mottle virus, Canna yellow streak virus, Carnation vein mottle virus, Carrot thin leaf virus, Carrot virus Y, Catharanthus mosaic virus, Celery mosaic virus, Ceratobium mosaic virus, Chilli ringspot virus, Chilli veinal mottle virus, Chinese artichoke mosaic virus, Clitoria virus Y, Clover yellow vein virus, Cocksfoot streak virus, Colombian datura virus, Commelina mosaic virus, Cowpea aphid-borne mosaic virus, Cucurbit vein banding virus, Cypripedium virus Y, Cyrtanthus elatus virus A, Daphne mosaic virus, Daphne virus Y, Dasheen mosaic virus, Datura shoestring virus, Dendrobium chlorotic mosaic virus, Dioscorea mosaic virus, Diuris virus Y, Donkey orchid virus A, East Asian Passiflora distortion virus, East Asian Passiflora virus, Endive necrotic mosaic virus, Euphorbia ringspot virus, Freesia mosaic virus, Fritillary virus Y, Gloriosa stripe mosaic virus, Gomphocarpus mosaic virus, Habenaria mosaic virus, Hardenbergia mosaic virus, Henbane mosaic virus, Hibbertia virus Y, Hippeastrum mosaic virus, Hyacinth mosaic virus, Impatiens flower break virus, Iris fulva mosaic virus, Iris mild mosaic virus, Iris severe mosaic virus, Japanese yam mosaic virus, Jasmine virus T, Johnsongrass mosaic virus, Kalanchoe mosaic virus, Keunjorong mosaic virus, Konjac mosaic virus, Leek yellow stripe virus, Lettuce Italian necrotic virus, Lettuce mosaic virus, Lily mottle virus, Lily virus Y, Lupinus mosaic virus, Lycoris mild mottle virus, Maize dwarf mosaic virus, Malva vein clearing virus, Mashua virus Y, Meadow saffron breaking virus, Mediterranean ruda virus, Moroccan watermelon mosaic virus, Narcissus degeneration virus, Narcissus late season yellows virus, Narcissus yellow stripe virus, Nerine yellow stripe virus, Nothoscordum mosaic virus, Onion yellow dwarf virus, Ornithogalum mosaic virus, Ornithogalum virus 2, Ornithogalum virus 3, Panax virus Y, Papaya leaf distortion mosaic virus, Papaya ringspot virus, Paris mosaic necrosis virus, Parsnip mosaic virus, Passiflora chlorosis virus, Passion fruit woodiness virus, Pea seed-borne mosaic virus, Peanut mottle virus, Pecan mosaic-associated virus, Pennisetum mosaic virus, Pepper mottle virus, Pepper severe mosaic virus, Pepper veinal mottle virus, Pepper yellow mosaic virus, Peru tomato mosaic virus, Pfaffia mosaic virus, Platycodon mild mottle virus, Pleione virus Y, Plum pox virus, Pokeweed mosaic virus, Potato virus A, Potato virus V, Potato virus Y, Potato yellow blotch virus, Ranunculus leaf distortion virus, Ranunculus mild mosaic virus, Ranunculus mosaic virus, Rhopalanthe virus Y, Saffron latent virus, Sarcochilus virus Y, Scallion mosaic virus, Shallot yellow stripe virus, Sorghum mosaic virus, Soybean mosaic virus, Spiranthes mosaic virus 3, Sudan watermelon mosaic virus, Sugarcane mosaic virus, Sunflower chlorotic mottle virus, Sunflower mild mosaic virus, Sunflower mosaic virus, Sunflower ring blotch virus, Sweet potato feathery mottle virus, Sweet potato latent virus, Sweet potato mild speckling virus, Sweet potato virus 2, Sweet potato virus C, Sweet potato virus G, Tamarillo leaf malformation virus, Telfairia mosaic virus, Telosma mosaic virus, Thunberg fritillary mosaic virus, Tobacco etch virus, Tobacco mosqueado virus, Tobacco vein banding mosaic virus, Tobacco vein mottling virus, Tomato necrotic stunt virus, Tradescantia mild mosaic virus, Tuberose mild mosaic virus, Tuberose mild mottle virus, Tulip breaking virus, Tulip mosaic virus, Turnip mosaic virus, Twisted-stalk chlorotic streak virus, Vallota mosaic virus, Vanilla distortion mosaic virus, Verbena virus Y, Watermelon leaf mottle virus, Watermelon mosaic virus, Wild melon banding virus, Wild onion symptomless virus, Wild potato mosaic virus, Wild tomato mosaic virus, Wisteria vein mosaic virus, Yam mild mosaic virus, Yam mosaic virus, Yambean mosaic virus, Zantedeschia mild mosaic virus, Zea mosaic virus, Zucchini shoestring virus, Zucchini tigre mosaic virus, Zucchini yellow fleck virus, Zucchini yellow mosaic virus, Passiflora edulis symptomless virus, Rose yellow mosaic virus, Agropyron mosaic virus, Hordeum mosaic virus, Ryegrass mosaic virus, Brome streak mosaic virus, Oat necrotic mottle virus, Tall oatgrass mosaic virus, Wheat eqlid mosaic virus, Wheat streak mosaic virus, Yellow oat grass mosaic virus, Common reed chlorotic stripe virus, Longan witches broom-associated virus, Spartina mottle virus, Avastrovirus 1, Avastrovirus 2, Avastrovirus 3, Mamastrovirus 1, Mamastrovirus 2, Mamastrovirus 3, Mamastrovirus 4, Mamastrovirus 5, Mamastrovirus 6, Mamastrovirus 7, Mamastrovirus 8, Mamastrovirus 9, Mamastrovirus 10, Mamastrovirus 11, Mamastrovirus 12, Mamastrovirus 13, Mamastrovirus 14, Mamastrovirus 15, Mamastrovirus 16, Mamastrovirus 17, Mamastrovirus 18, Mamastrovirus 19, Infectious pancreatic necrosis virus, Tellina virus, Yellowtail ascites virus, Infectious bursal disease virus, Blotched snakehead virus, Lates calcarifer birnavirus, Drosophina B birnavirus, Drosophila X virus, Mosquito X virus, Rotifer birnavirus, Tellina virus 1, Euprosterna elaeasa virus, Thosea asigna virus, Botrytis porri botybirnavirus 1, Duck hepatitis B virus, Heron hepatitis B virus, Parrot hepatitis B virus, Tibetan frog hepatitis B virus, Blue gill hepatitis B virus, Capuchin monkey hepatitis B virus, Chinese shrew hepatitis B virus, Domestic cat hepatitis B virus, Ground squirrel hepatitis virus, Hepatitis B virus, Long-fingered bat hepatitis B virus, Pomona bat hepatitis B virus, Roundleaf bat hepatitis B virus, Tai Forest hepatitis B virus, Tent-making bat hepatitis B virus, Woodchuck hepatitis virus, Woolly monkey hepatitis B virus, White sucker hepatitis B virus, Anopheles gambiae Moose virus, Antheraea semotivirus Tamy, Ascaris lumbricoides Tas virus, Bombyx mori Pao virus, Caenorhabditis elegans Cer13 virus, Drosophila melanogaster Bel virus, Drosophila melanogaster Roo virus, Drosophila semotivirus Max, Drosophila simulans Ninja virus, Schistosoma semotivirus Sinbad, Takifugu rubripes Suzu virus, Aglaonema bacilliform virus, Banana streak GF virus, Banana streak IM virus, Banana streak MY virus, Banana streak OL virus, Banana streak UA virus, Banana streak UT virus, Banana streak UL virus, Banana streak UM virus, Banana streak VN virus, Birch leaf roll-associated virus, Blackberry virus F, Bougainvillea chlorotic vein banding virus, Cacao bacilliform Sri Lanka virus, Cacao mild mosaic virus, Cacao swollen shoot CD virus, Cacao swollen shoot CE virus, Cacao swollen shoot Ghana M virus, Cacao swollen shoot Ghana N virus, Cacao swollen shoot Ghana Q virus, Cacao swollen shoot Togo A virus, Cacao swollen shoot Togo B virus, Cacao yellow vein banding virus, Canna yellow mottle associated virus, Canna yellow mottle virus, Citrus yellow mosaic virus, Codonopsis vein clearing virus, Commelina yellow mottle virus, Dioscorea bacilliform AL virus, Dioscorea bacilliform AL virus 2, Dioscorea bacilliform ES virus, Dioscorea bacilliform RT virus 1, Dioscorea bacilliform RT virus 2, Dioscorea bacilliform SN virus, Dioscorea bacilliform TR virus, Fig badnavirus 1, Gooseberry vein banding associated virus, Grapevine badnavirus 1, Grapevine Roditis leaf discoloration-associated virus, Grapevine vein clearing virus, Jujube mosaic-associated virus, Kalanchoe top-spotting virus, Mulberry badnavirus 1, Pagoda yellow mosaic associated virus, Pineapple bacilliform CO virus, Pineapple bacilliform ER virus, Piper yellow mottle virus, Rubus yellow net virus, Schefflera ringspot virus, Spiraea yellow leafspot virus, Sugarcane bacilliform Guadeloupe A virus, Sugarcane bacilliform Guadeloupe D virus, Sugarcane bacilliform IM virus, Sugarcane bacilliform MO virus, Sweet potato pakakuy virus, Taro bacilliform CH virus, Taro bacilliform virus, Wisteria badnavirus 1, Yacon necrotic mottle virus, Angelica bushy stunt virus, Atractylodes mild mottle virus, Carnation etched ring virus, Cauliflower mosaic virus, Dahlia mosaic virus, Figwort mosaic virus, Horseradish latent virus, Lamium leaf distortion virus, Mirabilis mosaic virus, Rudbeckia flower distortion virus, Soybean Putnam virus, Strawberry vein banding virus, Thistle mottle virus, Cassava vein mosaic virus, Sweet potato collusive virus, Dioscorea nummularia associated virus, Petunia vein clearing virus, Rose yellow vein virus, Sweet potato vein clearing virus, Tobacco vein clearing virus, Blueberry red ringspot virus, Cestrum yellow leaf curling virus, Peanut chlorotic streak virus, Soybean chlorotic mottle virus, Rice tungro bacilliform virus, Blueberry fruit drop associated virus, Ceratitis capitata Yoyo virus, Drosophila ananassae Tom virus, Drosophila melanogaster 17-6 virus, Drosophila melanogaster 297 virus, Drosophila melanogaster Gypsy virus, Drosophila melanogaster Idefix virus, Drosophila melanogaster Tirant virus, Drosophila melanogaster Zam virus, Drosophila virilis Tv1 virus, Trichoplusia ni TED virus, Arabidopsis thaliana Athila virus, Arabidopsis thaliana Tat4 virus, Bombyx mori Mag virus, Caenorhabditis elegans Cer1 virus, Cladosporium fulvum T-1 virus, Dictyostelium discoideum Skipper virus, Drosophila buzzatii Osvaldo virus, Drosophila melanogaster 412 virus, Drosophila melanogaster Blastopia virus, Drosophila melanogaster Mdg1 virus, Drosophila melanogaster Mdg3 virus, Drosophila melanogaster Micropia virus, Drosophila virilis Ulysses virus, Fusarium oxysporum Skippy virus, Lilium henryi Del1 virus, Saccharomyces cerevisiae Ty3 virus, Schizosaccharomyces pombe Tf1 virus, Schizosaccharomyces pombe Tf2 virus, Takifugu rubripes Sushi virus, Tribolium castaneum Woot virus, Tripneustis gratilla SURL virus, Aedes aegypti Mosqcopia virus, Candida albicans Tca2 virus, Candida albicans Tca5 virus, Drosophila melanogaster 1731 virus, Drosophila melanogaster copia virus, Saccharomyces cerevisiae Ty5 virus, Volvox carteri Lueckenbuesser virus, Volvox carteri Osser virus, Arabidopsis thaliana Art1 virus, Arabidopsis thaliana AtRE1 virus, Arabidopsis thaliana evelknievel virus, Arabidopsis thaliana Tal virus, Brassica oleracea Melmoth virus, Cajanus cajan Panzee virus, Glycine max Tgmr virus, Hordeum vulgare BARE-1 virus, Nicotiana tabacum Tnt1 virus, Nicotiana tabacum Tto1 virus, Oryza australiensis RIRE1 virus, Oryza longistaminata Retrofit virus, Physarum polycephalum Tp1 virus, Saccharomyces cerevisiae Ty1 virus, Saccharomyces cerevisiae Ty2 virus, Saccharomyces cerevisiae Ty4 virus, Solanum tuberosum Tst1 virus, Triticum aestivum WIS2 virus, Zea mays Hopscotch virus, Zea mays Sto4 virus, Arabidopsis thaliana Endovir virus, Glycine max SIRE1 virus, Lycopersicon esculentum ToRTL1 virus, Zea mays Opie2 virus, Zea mays Prem2 virus, Phaseolus vulgaris Tpv2-6 virus, Avian carcinoma Mill Hill virus 2, Avian leukosis virus, Avian myeloblastosis virus, Avian myelocytomatosis virus 29, Avian sarcoma virus CT10, Fujinami sarcoma virus, Rous sarcoma virus, UR2 sarcoma virus, Y73 sarcoma virus, Jaagsiekte sheep retrovirus, Langur virus, Mason-Pfizer monkey virus, Mouse mammary tumor virus, Squirrel monkey retrovirus, Bovine leukemia virus, Primate T-lymphotropic virus 1, Primate T-lymphotropic virus 2, Primate T-lymphotropic virus 3, Walleye dermal sarcoma virus, Walleye epidermal hyperplasia virus 1, Walleye epidermal hyperplasia virus 2, Chick syncytial virus, Feline leukemia virus, Finkel-Biskis-Jinkins murine sarcoma virus, Gardner-Arnstein feline sarcoma virus, Gibbon ape leukemia virus, Guinea pig type-C oncovirus, Hardy-Zuckerman feline sarcoma virus, Harvey murine sarcoma virus, Kirsten murine sarcoma virus, Koala retrovirus, Moloney murine sarcoma virus, Murine leukemia virus, Porcine type-C oncovirus, Reticuloendotheliosis virus, Snyder-Theilen feline sarcoma virus, Trager duck spleen necrosis virus, Viper retrovirus, Woolly monkey sarcoma virus, Bovine immunodeficiency virus, Caprine arthritis encephalitis virus, Equine infectious anemia virus, Feline immunodeficiency virus, Human immunodeficiency virus 1, Human immunodeficiency virus 2, Jembrana disease virus, Puma lentivirus, Simian immunodeficiency virus, Visna-maedi virus, Bovine foamy virus, Equine foamy virus, Feline foamy virus, Brown greater galago prosimian foamy virus, Bornean orangutan simian foamy virus, Central chimpanzee simian foamy virus, Cynomolgus macaque simian foamy virus, Eastern chimpanzee simian foamy virus, Grivet simian foamy virus, Guenon simian foamy virus, Japanese macaque simian foamy virus, Rhesus macaque simian foamy virus, Spider monkey simian foamy virus, Squirrel monkey simian foamy virus, Taiwanese macaque simian foamy virus, Western chimpanzee simian foamy virus, Western lowland gorilla simian foamy virus, White-tufted-ear marmoset simian foamy virus, Yellow-breasted capuchin simian foamy virus, Aspergillus fumigatus polymycovirus 1, Aspergillus spelaeus polymycovirus 1, Beauveria bassiana polymycovirus 1, Botryoshaeria dothidea polymycovirus 1, Cladosporium cladosporioides polymycovirus 1, Colletotrichum camelliae polymycovirus 1, Fusarium redolens polymycovirus 1, Magnaporthe oryzae polymycovirus 1, Penicillium digitatum polymycovirus 1, Penicillum brevicompactum polymycovirus 1, Macrobrachium satellite virus 1, Tobacco albetovirus 1, Tobacco albetovirus 2, Tobacco albetovirus 3, Maize aumaivirus 1, Panicum papanivirus 1, Tobacco virtovirus 1, Acanthocystis turfacea chlorella virus 1, Hydra viridis Chlorella virus 1, Paramecium bursaria Chlorella virus 1, Paramecium bursaria Chlorella virus A1, Paramecium bursaria Chlorella virus AL1A, Paramecium bursaria Chlorella virus AL2A, Paramecium bursaria Chlorella virus BJ2C, Paramecium bursaria Chlorella virus CA4A, Paramecium bursaria Chlorella virus CA4B, Paramecium bursaria Chlorella virus IL3A, Paramecium bursaria Chlorella virus NC1A, Paramecium bursaria Chlorella virus NE8A, Paramecium bursaria Chlorella virus NY2A, Paramecium bursaria Chlorella virus NYs1, Paramecium bursaria Chlorella virus SC1A, Paramecium bursaria Chlorella virus XY6E, Paramecium bursaria Chlorella virus XZ3A, Paramecium bursaria Chlorella virus XZ4A, Paramecium bursaria Chlorella virus XZ4C, Emiliania huxleyi virus 86, Ectocarpus fasciculatus virus a, Ectocarpus siliculosus virus 1, Ectocarpus siliculosus virus a, Feldmannia irregularis virus a, Feldmannia species virus, Feldmannia species virus a, Hincksia hinckiae virus a, Myriotrichia clavaeformis virus a, Pilayella littoralis virus 1, Micromonas pusilla virus SP1, Ostreococcus tauri virus OtV5, Chrysochromulina brevifilum virus PW1, Heterosigma akashiwo virus 01, Cafeteria roenbergensis virus, Acanthamoeba polyphaga mimivirus, Heliothis virescens ascovirus 3a, Spodoptera frugiperda ascovirus 1a, Trichoplusia ni ascovirus 2a, Diadromus pulchellus toursvirus, Lymphocystis disease virus 1, Lymphocystis disease virus 2, Lymphocystis disease virus 3, Infectious spleen and kidney necrosis virus, Scale drop disease virus, Ambystoma tigrinum virus, Common midwife toad virus, Epizootic haematopoietic necrosis virus, Frog virus 3, Santee-Cooper ranavirus, Singapore grouper iridovirus, Anopheles minimus iridovirus, Invertebrate iridescent virus 3, Invertebrate iridescent virus 9, Invertebrate iridescent virus 22, Invertebrate iridescent virus 25, Decapod iridescent virus 1, Invertebrate iridescent virus 6, Invertebrate iridescent virus 31, Marseillevirus marseillevirus, Senegalvirus marseillevirus, Lausannevirus, Tunisvirus, African swine fever virus, Canarypox virus, Flamingopox virus, Fowlpox virus, Juncopox virus, Mynahpox virus, Penguinpox virus, Pigeonpox virus, Psittacinepox virus, Quailpox virus, Sparrowpox virus, Starlingpox virus, Turkeypox virus, Goatpox virus, Lumpy skin disease virus, Sheeppox virus, Murmansk microtuspox virus, Yokapox virus, Mule deerpox virus, Nile crocodilepox virus, Hare fibroma virus, Myxoma virus, Rabbit fibroma virus, Squirrel fibroma virus, Eastern kangaroopox virus, Western kangaroopox virus, Molluscum contagiosum virus, Sea otterpox virus, Abatino macacapox virus, Akhmeta virus, Camelpox virus, Cowpox virus, Ectromelia virus, Monkeypox virus, Raccoonpox virus, Skunkpox virus, Taterapox virus, Vaccinia virus, Variola virus, Volepox virus, Cotia virus, Bovine papular stomatitis virus, Grey sealpox virus, Orf virus, Pseudocowpox virus, Red deerpox virus, Pteropox virus, Salmon gillpox virus, Squirrelpox virus, Swinepox virus, Eptesipox virus, Tanapox virus, Yaba monkey tumor virus, Anomala cuprea entomopoxvirus, Aphodius tasmaniae entomopoxvirus, Demodema bonariensis entomopoxvirus, Dermolepida albohirtum entomopoxvirus, Figulus sublaevis entomopoxvirus, Geotrupes sylvaticus entomopoxvirus, Melolontha melolontha entomopoxvirus, Acrobasis zelleri entomopoxvirus, Adoxophyes honmai entomopoxvirus, Amsacta moorei entomopoxvirus, Arphia conspersa entomopoxvirus, Choristoneura biennis entomopoxvirus, Choristoneura conflicta entomopoxvirus, Choristoneura diversuma entomopoxvirus, Choristoneura fumiferana entomopoxvirus, Choristoneura rosaceana entomopoxvirus, Chorizagrotis auxiliaris entomopoxvirus, Heliothis armigera entomopoxvirus, Locusta migratoria entomopoxvirus, Mythimna separata entomopoxvirus, Oedaleus senegalensis entomopoxvirus, Operophtera brumata entomopoxvirus, Schistocerca gregaria entomopoxvirus, Melanoplus sanguinipes entomopoxvirus, Aedes aegypti entomopoxvirus, Camptochironomus tentans entomopoxvirus, Chironomus attenuatus entomopoxvirus, Chironomus luridus entomopoxvirus, Chironomus plumosus entomopoxvirus, Goeldichironomus holoprasinus entomopoxvirus, Diachasmimorpha entomopoxvirus, Cafeteriavirus-dependent mavirus, Mimivirus-dependent virus Sputnik, Mimivirus-dependent virus Zamilon, Sulfolobus turreted icosahedral virus 1, Sulfolobus turreted icosahedral virus 2, Pseudomonas virus PR4, Pseudomonas virus PRD1, Bacillus virus AP50, Bacillus virus Bam35, Bacillus virus GIL16, Bacillus virus Wipl, Gluconobacter virus GC1, Bovine atadenovirus D, Deer atadenovirus A, Duck atadenovirus A, Lizard atadenovirus A, Ovine atadenovirus D, Possum atadenovirus A, Psittacine atadenovirus A, Snake atadenovirus A, Duck aviadenovirus B, Falcon aviadenovirus A, Fowl aviadenovirus A, Fowl aviadenovirus B, Fowl aviadenovirus C, Fowl aviadenovirus D, Fowl aviadenovirus E, Goose aviadenovirus A, Pigeon aviadenovirus A, Pigeon aviadenovirus B, Psittacine aviadenovirus B, Psittacine aviadenovirus C, Turkey aviadenovirus B, Turkey aviadenovirus C, Turkey aviadenovirus D, Sturgeon ichtadenovirus A, Bat mastadenovirus A, Bat mastadenovirus B, Bat mastadenovirus C, Bat mastadenovirus D, Bat mastadenovirus E, Bat mastadenovirus F, Bat mastadenovirus G, Bat mastadenovirus H, Bat mastadenovirus I, Bat mastadenovirus J, Bovine mastadenovirus A, Bovine mastadenovirus B, Bovine mastadenovirus C, Canine mastadenovirus A, Deer mastadenovirus B, Dolphin mastadenovirus A, Dolphin mastadenovirus B, Equine mastadenovirus A, Equine mastadenovirus B, Human mastadenovirus A, Human mastadenovirus B, Human mastadenovirus C, Human mastadenovirus D, Human mastadenovirus E, Human mastadenovirus F, Human mastadenovirus G, Murine mastadenovirus A, Murine mastadenovirus B, Murine mastadenovirus C, Ovine mastadenovirus A, Ovine mastadenovirus B, Ovine mastadenovirus C, Platyrrhini mastadenovirus A, Polar bear mastadenovirus A, Porcine mastadenovirus A, Porcine mastadenovirus B, Porcine mastadenovirus C, Sea lion mastadenovirus A, Simian mastadenovirus A, Simian mastadenovirus B, Simian mastadenovirus C, Simian mastadenovirus D, Simian mastadenovirus E, Simian mastadenovirus F, Simian mastadenovirus G, Simian mastadenovirus H, Simian mastadenovirus I, Skunk mastadenovirus A, Squirrel mastadenovirus A, Tree shrew mastadenovirus A, Frog siadenovirus A, Great tit siadenovirus A, Penguin siadenovirus A, Raptor siadenovirus A, Skua siadenovirus A, Turkey siadenovirus A, Pseudoalteromonas virus Cr39582, Pseudoalteromonas virus PM2, Haloarcula hispanica icosahedral virus 2, Haloarcula hispanica virus PH1, Haloarcula hispanica virus SH1, Haloarcula virus HCIV1, Natrinema virus SNJ1, Thermus virus IN93, Thermus virus P23-77, Alphalipothrixvirus SBFV2, Alphalipothrixvirus SFV1, Acidianus filamentous virus 3, Acidianus filamentous virus 6, Acidianus filamentous virus 7, Acidianus filamentous virus 8, Acidianus filamentous virus 9, Sulfolobus islandicus filamentous virus, Acidianus filamentous virus 2, Deltalipothrixvirus SBFV3, Acidianus filamentous virus 1, Acidianus rod-shaped virus 1, Sulfolobus islandicus rod-shaped virus 1, Sulfolobus islandicus rod-shaped virus 2, Ageratum yellow vein Singapore alphasatellite, Cotton leaf curl Saudi Arabia alphasatellite, Ash gourd yellow vein mosaic alphasatellite, Capsicum India alphasatellite, Cleome leaf crumple alphasatellite, Croton yellow vein mosaic alphasatellite, Euphorbia yellow mosaic alphasatellite, Melon chlorotic mosaic alphasatellite, Sida Cuba alphasatellite, Tomato leaf curl New Delhi alphasatellite, Tomato leaf curl Virudhunagar alphasatellite, Tomato yellow spot alphasatellite, Whitefly associated Guatemala alphasatellite 2, Whitefly associated Puerto Rico alphasatellite 1, Ageratum enation alphasatellite, Ageratum yellow vein alphasatellite, Ageratum yellow vein China alphasatellite, Ageratum yellow vein India alphasatellite, Bhendi yellow vein alphasatellite, Cassava mosaic Madagascar alphasatellite, Chilli leaf curl alphasatellite, Cotton leaf curl Egypt alphasatellite, Cotton leaf curl Gezira alphasatellite, Cotton leaf curl Lucknow alphasatellite, Cotton leaf curl Multan alphasatellite, Gossypium darwinii symptomless alphasatellite, Malvastrum yellow mosaic alphasatellite, Malvastrum yellow mosaic Cameroon alphasatellite, Pedilanthus leaf curl alphasatellite, Sida leaf curl alphasatellite, Sida yellow vein Vietnam alphasatellite, Sunflower leaf curl Karnataka alphasatellite, Synedrella leaf curl alphasatellite, Tobacco curly shoot alphasatellite, Tomato leaf curl Buea alphasatellite, Tomato leaf curl Cameroon alphasatellite, Tomato leaf curl Pakistan alphasatellite, Tomato yellow leaf curl China alphasatellite, Tomato yellow leaf curl Thailand alphasatellite, Tomato yellow leaf curl Yunnan alphasatellite, Eclipta yellow vein alphasatellite, Gossypium mustelinum symptomless alphasatellite, Hollyhock yellow vein alphasatellite, Mesta yellow vein mosaic alphasatellite, Okra enation leaf curl alphasatellite, Okra yellow crinkle Cameroon alphasatellite, Vernonia yellow vein Fujian alphasatellite, Dragonfly associated alphasatellite, Whitefly associated Guatemala alphasatellite 1, Banana bunchy top alphasatellite 1, Banana bunchy top alphasatellite 2, Banana bunchy top alphasatellite 3, Cardamom bushy dwarf alphasatellite, Milk vetch dwarf alphasatellite 2, Pea necrotic yellow dwarf alphasatellite 2, Sophora yellow stunt alphasatellite 4, Sophora yellow stunt alphasatellite 5, Subterranean clover stunt alphasatellite 2, Faba bean necrotic yellows alphasatellite 2, Milk vetch dwarf alphasatellite 3, Faba bean necrotic stunt alphasatellite, Milk vetch dwarf alphasatellite 1, Pea necrotic yellow dwarf alphasatellite 1, Sophora yellow stunt alphasatellite 2, Cow vetch latent alphasatellite, Sophora yellow stunt alphasatellite 3, Faba bean necrotic yellows alphasatellite 1, Faba bean necrotic yellows alphasatellite 3, Sophora yellow stunt alphasatellite 1, Subterranean clover stunt alphasatellite 1, Coconut foliar decay alphasatellite, Acidianus bottle-shaped virus, Torque teno virus 1, Torque teno virus 2, Torque teno virus 3, Torque teno virus 4, Torque teno virus 5, Torque teno virus 6, Torque teno virus 7, Torque teno virus 8, Torque teno virus 9, Torque teno virus 10, Torque teno virus 11, Torque teno virus 12, Torque teno virus 13, Torque teno virus 14, Torque teno virus 15, Torque teno virus 16, Torque teno virus 17, Torque teno virus 18, Torque teno virus 19, Torque teno virus 20, Torque teno virus 21, Torque teno virus 22, Torque teno virus 23, Torque teno virus 24, Torque teno virus 25, Torque teno virus 26, Torque teno virus 27, Torque teno virus 28, Torque teno virus 29, Torque teno mini virus 1, Torque teno mini virus 2, Torque teno mini virus 3, Torque teno mini virus 4, Torque teno mini virus 5, Torque teno mini virus 6, Torque teno mini virus 7, Torque teno mini virus 8, Torque teno mini virus 9, Torque teno mini virus 10, Torque teno mini virus 11, Torque teno mini virus 12, Torque teno tupaia virus, Torque teno tamarin virus, Torque teno felis virus, Torque teno felis virus 2, Torque teno midi virus 1, Torque teno midi virus 2, Torque teno midi virus 3, Torque teno midi virus 4, Torque teno midi virus 5, Torque teno midi virus 6, Torque teno midi virus 7, Torque teno midi virus 8, Torque teno midi virus 9, Torque teno midi virus 10, Torque teno midi virus 11, Torque teno midi virus 12, Torque teno midi virus 13, Torque teno midi virus 14, Torque teno midi virus 15, Chicken anemia virus, Torque teno sus virus 1a, Torque teno sus virus ab, Torque teno sus virus k2a, Torque teno sus virus k2b, Torque teno seal virus 1, Torque teno seal virus 2, Torque teno seal virus 3, Torque teno seal virus 8, Torque teno seal virus 9, Torque teno zalophus virus 1, Torque teno equus virus 1, Torque teno seal virus 4, Torque teno seal virus 5, Torque teno canis virus, Torque teno douroucouli virus, Avocado sunblotch viroid, Eggplant latent viroid, Apple hammerhead viroid, Chrysanthemum chlorotic mottle viroid, Peach latent mosaic viroid, Adoxophyes honmai nucleopolyhedrovirus, Agrotis ipsilon multiple nucleopolyhedrovirus, Agrotis segetum nucleopolyhedrovirus A, Agrotis segetum nucleopolyhedrovirus B, Antheraea pernyi nucleopolyhedrovirus, Anticarsia gemmatalis multiple nucleopolyhedrovirus, Autographa californica multiple nucleopolyhedrovirus, Bombyx mori nucleopolyhedrovirus, Buzura suppressaria nucleopolyhedrovirus, Catopsilia pomona nucleopolyhedrovirus, Choristoneura fumiferana DEF multiple nucleopolyhedrovirus, Choristoneura fumiferana multiple nucleopolyhedrovirus, Choristoneura murinana nucleopolyhedrovirus, Choristoneura rosaceana nucleopolyhedrovirus, Chrysodeixis chalcites nucleopolyhedrovirus, Chrysodeixis includens nucleopolyhedrovirus, Clanis bilineata nucleopolyhedrovirus, Condylorrhiza vestigialis nucleopolyhedrovirus, Cryptophlebia peltastica nucleopolyhedrovirus, Cyclophragma undans nucleopolyhedrovirus, Ectropis obliqua nucleopolyhedrovirus, Epiphyas postvittana nucleopolyhedrovirus, Euproctis pseudoconspersa nucleopolyhedrovirus, Helicoverpa armigera nucleopolyhedrovirus, Hemileuca species nucleopolyhedrovirus, Hyphantria cunea nucleopolyhedrovirus, Hyposidra talaca nucleopolyhedrovirus, Lambdina fiscellaria nucleopolyhedrovirus, Leucania separata nucleopolyhedrovirus, Lonomia obliqua nucleopolyhedrovirus, Lymantria dispar multiple nucleopolyhedrovirus, Lymantria xylina nucleopolyhedrovirus, Mamestra brassicae multiple nucleopolyhedrovirus, Mamestra configurata nucleopolyhedrovirus A, Mamestra configurata nucleopolyhedrovirus B, Maruca vitrata nucleopolyhedrovirus, Mythimna unipuncta nucleopolyhedrovirus A, Mythimna unipuncta nucleopolyhedrovirus B, Operophtera brumata nucleopolyhedrovirus, Orgyia leucostigma nucleopolyhedrovirus, Orgyia pseudotsugata multiple nucleopolyhedrovirus, Oxyplax ochracea nucleopolyhedrovirus, Peridroma saucia nucleopolyhedrovirus, Perigonia lusca nucleopolyhedrovirus, Spodoptera eridania nucleopolyhedrovirus, Spodoptera exempta nucleopolyhedrovirus, Spodoptera exigua multiple nucleopolyhedrovirus, Spodoptera frugiperda multiple nucleopolyhedrovirus, Spodoptera littoralis nucleopolyhedrovirus, Spodoptera litura nucleopolyhedrovirus, Sucra jujuba nucleopolyhedrovirus, Thysanoplusia orichalcea nucleopolyhedrovirus, Trichoplusia ni single nucleopolyhedrovirus, Urbanus proteus nucleopolyhedrovirus, Wiseana signata nucleopolyhedrovirus, Adoxophyes orana granulovirus, Agrotis segetum granulovirus, Artogeia rapae granulovirus, Choristoneura fumiferana granulovirus, Clostera anachoreta granulovirus, Clostera anastomosis granulovirus A, Clostera anastomosis granulovirus B, Cnaphalocrocis medinalis granulovirus, Cryptophlebia leucotreta granulovirus, Cydia pomonella granulovirus, Diatraea saccharalis granulovirus, Epinotia aporema granulovirus, Erinnyis ello granulovirus, Harrisina brillians granulovirus, Helicoverpa armigera granulovirus, Lacanobia oleracea granulovirus, Mocis latipes granulovirus, Mythimna unipuncta granulovirus A, Mythimna unipuncta granulovirus B, Phthorimaea operculella granulovirus, Plodia interpunctella granulovirus, Plutella xylostella granulovirus, Spodoptera frugiperda granulovirus, Spodoptera litura granulovirus, Trichoplusia ni granulovirus, Xestia c-nigrum granulovirus, Culex nigripalpus nucleopolyhedrovirus, Neodiprion lecontei nucleopolyhedrovirus, Neodiprion sertifer nucleopolyhedrovirus, Acidianus two-tailed virus, Aeropyrum pernix bacilliform virus 1, Flavobacterium virus FLiP, Sulfolobus spindle-shaped virus 1, Sulfolobus spindle-shaped virus 2, Sulfolobus spindle-shaped virus 4, Sulfolobus spindle-shaped virus 5, Sulfolobus spindle-shaped virus 7, Sulfolobus spindle-shaped virus 8, Sulfolobus spindle-shaped virus 9, Acidianus spindle-shaped virus 1, Sulfolobus spindle-shaped virus 6, Pyrobaculum spherical virus, Thermoproteus tenax spherical virus 1, Sulfolobus newzealandicus droplet-shaped virus, Aeropyrum pernix ovoid virus 1, Salterprovirus Hisl, Glossina hytrosavirus, Musca hytrosavirus, White spot syndrome virus, Gryllus bimaculatus nudivirus, Oryctes rhinoceros nudivirus, Heliothis zea nudivirus, Sulfolobus ellipsoid virus 1, Acholeplasma virus L2, Apanteles crassicornis bracovirus, Apanteles fumiferanae bracovirus, Ascogaster argentifrons bracovirus, Ascogaster quadridentata bracovirus, Cardiochiles nigriceps bracovirus, Chelonus altitudinis bracovirus, Chelonus blackburni bracovirus, Chelonus inanitus bracovirus, Chelonus insularis bracovirus, Chelonus near curvimaculatus bracovirus, Chelonus texanus bracovirus, Cotesia congregata bracovirus, Cotesia flavipes bracovirus, Cotesia glomerata bracovirus, Cotesia hyphantriae bracovirus, Cotesia kariyai bracovirus, Cotesia marginiventris bracovirus, Cotesia melanoscela bracovirus, Cotesia rubecula bracovirus, Cotesia schaeferi bracovirus, Diolcogaster facetosa bracovirus, Glyptapanteles flavicoxis bracovirus, Glyptapanteles indiensis bracovirus, Glyptapanteles liparidis bracovirus, Hypomicrogaster canadensis bracovirus, Hypomicrogaster ectdytolophae bracovirus, Microplitis croceipes bracovirus, Microplitis demolitor bracovirus, Phanerotoma flavitestacea bracovirus, Pholetesor ornigis bracovirus, Protapanteles paleacritae bracovirus, Tranosema rostrale bracovirus, Campoletis aprilis ichnovirus, Campoletis flavicincta ichnovirus, Campoletis sonorensis ichnovirus, Casinaria arjuna ichnovirus, Casinaria forcipata ichnovirus, Casinaria infesta ichnovirus, Diadegma acronyctae ichnovirus, Diadegma interruptum ichnovirus, Diadegma terebrans ichnovirus, Enytus montanus ichnovirus, Eriborus terebrans ichnovirus, Glypta fumiferanae ichnovirus, Hyposoter annulipes ichnovirus, Hyposoter exiguae ichnovirus, Hyposoter fugitivus ichnovirus, Hyposoter lymantriae ichnovirus, Hyposoter pilosulus ichnovirus, Hyposoter rivalis ichnovirus, Olesicampe benefactor ichnovirus, Olesicampe geniculatae ichnovirus, Synetaeris tenuifemur ichnovirus, Alphaportoglobovirus SPV2, Sulfolobus alphaportoglobovirus 1, Apple dimple fruit viroid, Apple scar skin viroid, Australian grapevine viroid, Citrus bent leaf viroid, Citrus dwarfing viroid, Citrus viroid V, Citrus viroid VI, Grapevine yellow speckle viroid 1, Grapevine yellow speckle viroid 2, Pear blister canker viroid, Citrus bark cracking viroid, Coconut cadang-cadang viroid, Coconut tinangaja viroid, Hop latent viroid, Coleus blumei viroid 1, Coleus blumei viroid 2, Coleus blumei viroid 3, Dahlia latent viroid, Hop stunt viroid, Chrysanthemum stunt viroid, Citrus exocortis viroid, Columnea latent viroid, Iresine viroid 1, Pepper chat fruit viroid, Potato spindle tuber viroid, Tomato apical stunt viroid, Tomato chlorotic dwarf viroid, Tomato planta macho viroid, Aeropyrum coil-shaped virus, Nitmarvirus NSV1, Ageratum leaf curl Buea betasatellite, Ageratum leaf curl Cameroon betasatellite, Ageratum yellow leaf curl betasatellite, Ageratum yellow vein betasatellite, Ageratum yellow vein India betasatellite, Ageratum yellow vein Sri Lanka betasatellite, Alternanthera yellow vein betasatellite, Andrographis yellow vein leaf curl betasatellite, Bhendi yellow vein mosaic betasatellite, Cardiospermum yellow leaf curl betasatellite, Chili leaf curl betasatellite, Chili leaf curl Jaunpur betasatellite, Chili leaf curl Sri Lanka betasatellite, Cotton leaf curl Gezira betasatellite, Cotton leaf curl Multan betasatellite, Croton yellow vein mosaic betasatellite, Eupatorium yellow vein betasatellite, Eupatorium yellow vein mosaic betasatellite, French bean leaf curl betasatellite, Hedyotis yellow mosaic betasatellite, Honeysuckle yellow vein betasatellite, Honeysuckle yellow vein mosaic betasatellite, Malvastrum leaf curl betasatellite, Malvastrum leaf curl Guangdong betasatellite, Mirabilis leaf curl betasatellite, Momordica yellow mosaic betasatellite, Mungbean yellow mosaic betasatellite, Okra leaf curl Oman betasatellite, Papaya leaf curl betasatellite, Papaya leaf curl China betasatellite, Papaya leaf curl India betasatellite, Rhynchosia yellow mosaic betasatellite, Rose leaf curl betasatellite, Siegesbeckia yellow vein betasatellite, Tobacco curly shoot betasatellite, Tobacco leaf curl betasatellite, Tobacco leaf curl Japan betasatellite, Tobacco leaf curl Patna betasatellite, Tomato leaf curl Bangalore betasatellite, Tomato leaf curl Bangladesh betasatellite, Tomato leaf curl betasatellite, Tomato leaf curl China betasatellite, Tomato leaf curl Gandhinagar betasatellite, Tomato leaf curl Java betasatellite, Tomato leaf curl Joydebpur betasatellite, Tomato leaf curl Laguna betasatellite, Tomato leaf curl Laos betasatellite, Tomato leaf curl Malaysia betasatellite, Tomato leaf curl Nepal betasatellite, Tomato leaf curl Patna betasatellite, Tomato leaf curl Philippine betasatellite, Tomato leaf curl Sri Lanka betasatellite, Tomato leaf curl Yemen betasatellite, Tomato yellow leaf curl China betasatellite, Tomato yellow leaf curl Rajasthan betasatellite, Tomato yellow leaf curl Shandong betasatellite, Tomato yellow leaf curl Thailand betasatellite, Tomato yellow leaf curl Vietnam betasatellite, Tomato yellow leaf curl Yunnan betasatellite, Vernonia yellow vein betasatellite, Vernonia yellow vein Fujian betasatellite, Croton yellow vein deltasatellite, Malvastrum leaf curl deltasatellite, Sida golden yellow vein deltasatellite 1, Sida golden yellow vein deltasatellite 2, Sida golden yellow vein deltasatellite 3, Sweet potato leaf curl deltasatellite 1, Sweet potato leaf curl deltasatellite 2, Sweet potato leaf curl deltasatellite 3, Tomato leaf curl deltasatellite, Tomato yellow leaf distortion deltasatellite 1, Tomato yellow leaf distortion deltasatellite 2, Pyrobaculum filamentous virus 1, Thermoproteus tenax virus 1, Hepatitis delta virus, Heterocapsa circularisquama DNA virus 01, and Rhizidiomyces virus. Other viruses include the ICTV Master species list (https://talk.ictvonline.org/files/master-species-lists/m/msl/9601/), which is incorporated by reference herein.

In further embodiments, the present invention relates to methods of trans-splicing one or more RNA molecule in cells or in vitro. Trans-splicing of independent RNA molecules can be useful for expressing full-length proteins when the nucleic acid sequences encoding said proteins exceed the packaging size for certain plasmids and vectors and for generating multi-domain proteins that are otherwise difficult to express. Further, trans-splicing a single RNA molecule can be useful for deleting sections of RNA that would otherwise be translated into pathological or defective proteins. Additional applications and further discussion of trans-splicing RNA molecules can be found in International Application No. PCT/US2021/016885 (incorporated by reference herein in its entirety).

In one embodiment, the present invention comprises a method of trans-splicing RNA molecules in vitro. In one embodiment, the method comprises contacting one or more fusion editing protein of the present invention with one or more RNA molecule in vitro, in the presence of one or more targeting nucleic acid. In some embodiments, said one or more RNA molecule comprises a first RNA molecule and a second RNA molecule. In one embodiment, said first RNA molecule and said second RNA molecule are cleaved by the fusion editing protein, generating 2′,3′ cyclic phosphate and 5′ hydroxyl RNA termini. In one embodiment, the method further comprises contacting the first RNA molecule and the second RNA molecule with RtcB ligase, as described above. In one embodiment, the 2′,3′ cyclic phosphate termini of the first RNA molecule is ligated to the 5′ hydroxyl termini of the second RNA molecule, thereby generating a trans-spliced RNA molecule.

In one embodiment, the present invention comprises a method of trans-splicing RNA molecules in a cell or tissue. In one embodiment, the method comprises administering to one or more cell or tissue one or more fusion editing protein of the present invention and one or more targeting nucleic acid. In one embodiment, the method comprises administering to one or more cell or tissue one or more nucleic acid encoding a fusion editing protein of the present invention and one or more targeting nucleic acid. In some embodiments, the method further comprises administering one or more RNA molecule to the cell or tissue. In some embodiments, the method further comprises administering RtcB ligase or a nucleic acid encoding RtcB ligase, as described above.

In one embodiment, the present invention comprises a method of treating one or more disease or disorder associated with defective or pathological protein. In one embodiment, the method comprises administering to one or more cell or tissue one or more fusion editing protein of the present invention and one or more targeting nucleic acid. In one embodiment, the method comprises administering to one or more cell or tissue one or more nucleic acid encoding a fusion editing protein of the present invention and one or more targeting nucleic acid. In some embodiments, the method further comprises administering RtcB ligase or a nucleic acid encoding RtcB ligase, as described above.

### Amino Acid and Nucleic Acid Sequences

Table 2 provides a summary of the amino acid and nucleic acid sequences.

TABLE 2
Summary of sequences

| SEQ | | |
|---|---|---|
| ID NO | Type | Description |
| 1 | Amino Acid | PspCas13b |
| 2 | Amino Acid | PspCas13b Truncation |
| 3 | Amino Acid | AspCas13b |
| 4 | Amino Acid | AspCas13c |
| 5 | Amino Acid | BmaCas13a |
| 6 | Amino Acid | BzoCas13b |
| 7 | Amino Acid | CamCas13a |
| 8 | Amino Acid | CcaCas13b |
| 9 | Amino Acid | Cga2Cas13a |
| 10 | Amino Acid | CgaCas13a |
| 11 | Amino Acid | EbaCas13a |
| 12 | Amino Acid | EreCas13a |
| 13 | Amino Acid | EsCas13d |
| 14 | Amino Acid | FbrCas13b |
| 15 | Amino Acid | FnbCas13c |
| 16 | Amino Acid | FndCas13c |
| 17 | Amino Acid | FnfCas13c |
| 18 | Amino Acid | FnsCas13c |
| 19 | Amino Acid | FpeCas13c |
| 20 | Amino Acid | FulCas13c |
| 21 | Amino Acid | HheCas13a |
| 22 | Amino Acid | LbfCas13a |
| 23 | Amino Acid | LbmCas13a |
| 24 | Amino Acid | LbnCas13a |
| 25 | Amino Acid | LbuCas13a |
| 26 | Amino Acid | LseCas13a |
| 27 | Amino Acid | LshCas13a |
| 28 | Amino Acid | LspCas13a |
| 29 | Amino Acid | Lwa2cas13a |
| 30 | Amino Acid | LwaCas13a |
| 31 | Amino Acid | LweCas13a |
| 32 | Amino Acid | PauCas13b |
| 33 | Amino Acid | PbuCas13b |
| 34 | Amino Acid | PgiCas13b |
| 35 | Amino Acid | PguCas13b |
| 36 | Amino Acid | Pin2Cas13b |
| 37 | Amino Acid | Pin3Cas13b |
| 38 | Amino Acid | PinCas13b |
| 39 | Amino Acid | Pprcas13a |
| 40 | Amino Acid | PsaCas13b |
| 41 | Amino Acid | PsmCas13b |
| 42 | Amino Acid | RanCas13b |
| 43 | Amino Acid | RcdCas13a |
| 44 | Amino Acid | RcrCas13a |
| 45 | Amino Acid | RcsCas13a |
| 46 | Amino Acid | UrCas13d |
| 47 | Amino Acid | dPspCas13b |
| 48 | Amino Acid | dPspCas13b truncation |
| 49 | Amino Acid | Rnase 1 (human) |
| 50 | Amino Acid | Rnase 2 (human) |
| 51 | Amino Acid | Rnase 3 (human) |
| 52 | Amino Acid | Rnase 4 (human) |
| 53 | Amino Acid | Rnase 5 (human) |
| 54 | Amino Acid | Rnase 6 (human) |
| 55 | Amino Acid | Rnase 7 (human) |
| 56 | Amino Acid | Rnase 8 (human) |
| 57 | Amino Acid | Synthetic Tandem Dimer RNase 1 (Human, tdRNase 1) |
| 58 | Amino Acid | RNase A (Bovine RNase1) |
| 59 | Amino Acid | RNase 1 (E. coli, ecRNase 1) |
| 60 | Amino Acid | RNase 1B (P. nemaeus) |
| 61 | Amino Acid | txRNase 1 (RNase 1 R39D/N67D/N88A/G89D/R91D) |
| 62 | Amino Acid | txRNase A (RNase A D38R/R39D/N67R/G88R) |
| 63 | Amino Acid | RNase T1 (A. flavus) |
| 64 | Amino Acid | RNase T1, rA cleaving variant No.1 |
| 65 | Amino Acid | RNase T1, rA cleaving variant No.2 |
| 66 | Amino Acid | RNase T1, rA cleaving variant RNase T1-R2 |
| 67 | Amino Acid | Alpha Sarcin (A. giganteus) |
| 68 | Amino Acid | RNase T1, dsRNA cleaving variant RNase Ta |
| 69 | Amino Acid | BARNASE |
| 70 | Amino Acid | RNASE A, 3x S-peptide-P2A-S-protein |
| 71 | Amino Acid | RNase LE |
| 72 | Amino Acid | Rnase T2 (human) |
| 73 | Amino Acid | Onconase (R. pipiens) |
| 74 | Amino Acid | Erns(C171R) (RNase T2, Bovine viral diarrhea virus) |
| 75 | Amino Acid | RNase U2 (U. sphaerogena) |
| 76 | Amino Acid | PIN RNase domain (Human SMG6) |
| 77 | Amino Acid | Synthetic tandem PIN RNase domain (Human SMG6, tdPIN) |
| 78 | Amino Acid | Bovine seminal ribonuclease (Bovind, SRN) |
| 79 | Amino Acid | Synthetic Tandem Dimer Bovine seminal ribonuclease (Bovind, tdSRN) |
| 80 | Amino Acid | RNase V1 |
| 81 | Amino Acid | Mini RNase III (B. subtilis, MiniR3) |
| 82 | Amino Acid | Synthetic Tandem Dimer Mini RNase III (B. subtilis, tdMiniR3) |
| 83 | Amino Acid | RNase III Domain (Mouse, a.a.1648-1846, DICER) |
| 84 | Amino Acid | Synthetic Tandem Dimer RNase III Domain (Mouse, tdDICER) |
| 85 | Amino Acid | Natural tandem RNase III domain (Human DROSHA a.a. 925-1246, DROSHA) |
| 86 | Amino Acid | Natural tandem RNase III domain Dimer (G. intestinalis DICER, a.a. 292-754 |
| | | giDICER) |
| 87 | Amino Acid | Synthetic tandem RNase III domain (E. coli RNC, tdRNC) |
| 88 | Amino Acid | Ribonuclease HI (Sulfurisphaera tokodaii, RNase HI*) |
| 89 | Amino Acid | Ribonuclease HI(D125N)(Sulfurisphaera tokodaii, RNase HI*D125N) |
| 90 | Amino Acid | S-protein (RNase 1, Human) |
| 91 | Amino Acid | S-peptide (RNase 1, Human) |
| 92 | Amino Acid | S-peptide-linker-ERT2 (RNase 1, Human) |
| 93 | Amino Acid | S-protein (RNase 1B, P. nemaeus) |
| 94 | Amino Acid | S-peptide (RNase 1B, P. nemaeus) |
| 95 | Amino Acid | S-peptide-linker-ERT2 (RNase 1B, P. nemaeus) |
| 96 | Amino Acid | S-protein (RNase A, Bovine) |
| 97 | Amino Acid | S-peptide (RNase A, Bovine) |
| 98 | Amino Acid | S-peptide-linker-ERT2 (RNase A, Bovine) |
| 99 | Amino Acid | 3x S-peptide (RNASE A) |
| 100 | Amino Acid | Linker sequence 1 |
| 101 | Amino Acid | Linker sequence 2 |
| 102 | Amino Acid | Linker sequence 3 |
| 103 | Amino Acid | Linker sequence 4 |
| 104 | Amino Acid | Linker sequence 5 |
| 105 | Amino Acid | Linker sequence 6 |
| 106 | Amino Acid | Linker sequence 7 |
| 107 | Amino Acid | Linker sequence 8 |
| 108 | Amino Acid | Linker sequence 9 |
| 109 | Amino Acid | 3xFlag |
| 110 | Amino Acid | Ty1 NLS |
| 111 | Amino Acid | Ty2 NLS |
| 112 | Amino Acid | MAK11 |
| 113 | Amino Acid | 1xSV40 |
| 114 | Amino Acid | 3xSV40 |
| 115 | Amino Acid | NPM |
| 116 | Amino Acid | STH1 |
| 117 | Amino Acid | INO4 |
| 118 | Amino Acid | Ty1-like NLS O28090-0 |
| 119 | Amino Acid | Ty1-like NLS O50087-0 |
| 120 | Amino Acid | Ty1-like NLS O58353-0 |
| 121 | Amino Acid | Ty1-like NLS Q57602-0 |
| 122 | Amino Acid | Ty1-like NLS Q6L1X9-0 |
| 123 | Amino Acid | Ty1-like NLS A0K3M1-0 |
| 124 | Amino Acid | Ty1-like NLS A0LYZ1-0 |
| 125 | Amino Acid | Ty1-like NLS A1B022-0 |
| 126 | Amino Acid | Ty1-like NLS A1V8A7-0 |
| 127 | Amino Acid | Ty1-like NLS A1VIP6-0 |
| 128 | Amino Acid | Ty1-like NLS A2RDW6-0 |
| 129 | Amino Acid | Ty1-like NLS A2S7H2-0 |
| 130 | Amino Acid | Ty1-like NLS A3MRV0-0 |
| 131 | Amino Acid | Ty1-like NLS A3NEI3-0 |
| 132 | Amino Acid | Ty1-like NLS A3P0B7-0 |
| 133 | Amino Acid | Ty1-like NLS A4JAN6-0 |
| 134 | Amino Acid | Ty1-like NLS A4SUV7-0 |
| 135 | Amino Acid | Ty1-like NLS A5FP03-0 |
| 136 | Amino Acid | Ty1-like NLS A5ILZ2-0 |
| 137 | Amino Acid | Ty1-like NLS A6GY20-0 |
| 138 | Amino Acid | Ty1-like NLS A6LLI5-0 |
| 139 | Amino Acid | Ty1-like NLS A6LQX4-0 |
| 140 | Amino Acid | Ty1-like NLS A8F6X2-0 |
| 141 | Amino Acid | Ty1-like NLS A8G6B7-0 |
| 142 | Amino Acid | Ty1-like NLS A9ADI9-0 |
| 143 | Amino Acid | Ty1-like NLS A9IJ08-0 |
| 144 | Amino Acid | Ty1-like NLS A9IXA1-0 |
| 145 | Amino Acid | Ty1-like NLS A9NEN2-0 |
| 146 | Amino Acid | Ty1-like NLS B0S140-0 |
| 147 | Amino Acid | Ty1-like NLS B1JU18-0 |
| 148 | Amino Acid | Ty1-like NLS B1LBA1-0 |
| 149 | Amino Acid | Ty1-like NLS B1W354-0 |
| 150 | Amino Acid | Ty1-like NLS B1XSP7-0 |
| 151 | Amino Acid | Ty1-like NLS B1YRC6-0 |
| 152 | Amino Acid | Ty1-like NLS B2JIH0-0 |
| 153 | Amino Acid | Ty1-like NLS B2T755-0 |
| 154 | Amino Acid | Ty1-like NLS B2UEM3-0 |
| 155 | Amino Acid | Ty1-like NLS B3PLU0-0 |
| 156 | Amino Acid | Ty1-like NLS B3R7T2-0 |
| 157 | Amino Acid | Ty1-like NLS B4E5B6-0 |
| 158 | Amino Acid | Ty1-like NLS B4S3C9-0 |
| 159 | Amino Acid | Ty1-like NLS B7IHT4-0 |
| 160 | Amino Acid | Ty1-like NLS B8E0X6-0 |
| 161 | Amino Acid | Ty1-like NLS B9K7W0-0 |
| 162 | Amino Acid | Ty1-like NLS C1A494-0 |
| 163 | Amino Acid | Ty1-like NLS C5CE41-0 |
| 164 | Amino Acid | Ty1-like NLS O88058-0 |
| 165 | Amino Acid | Ty1-like NLS P0DG92-0 |
| 166 | Amino Acid | Ty1-like NLS P0DG93-0 |
| 167 | Amino Acid | Ty1-like NLS P60554-0 |
| 168 | Amino Acid | Ty1-like NLS P67354-0 |
| 169 | Amino Acid | Ty1-like NLS P75311-0 |
| 170 | Amino Acid | Ty1-like NLS P75471-0 |
| 171 | Amino Acid | Ty1-like NLS P94372-0 |
| 172 | Amino Acid | Ty1-like NLS Q056Y0-0 |
| 173 | Amino Acid | Ty1-like NLS Q057D7-0 |
| 174 | Amino Acid | Ty1-like NLS Q0AYB7-0 |
| 175 | Amino Acid | Ty1-like NLS Q0BJ50-0 |
| 176 | Amino Acid | Ty1-like NLS Q0K610-0 |
| 177 | Amino Acid | Ty1-like NLS Q0STA4-0 |
| 178 | Amino Acid | Ty1-like NLS Q0STL9-0 |
| 179 | Amino Acid | Ty1-like NLS Q0TQV7-0 |
| 180 | Amino Acid | Ty1-like NLS Q0TR88-0 |
| 181 | Amino Acid | Ty1-like NLS Q12GX5-0 |
| 182 | Amino Acid | Ty1-like NLS Q13TG6-0 |
| 183 | Amino Acid | Ty1-like NLS Q1AWG1-0 |
| 184 | Amino Acid | Ty1-like NLS Q1BRU4-0 |
| 185 | Amino Acid | Ty1-like NLS Q1J5X5-0 |
| 186 | Amino Acid | Ty1-like NLS Q1JAY8-0 |
| 187 | Amino Acid | Ty1-like NLS Q1JG57-0 |
| 188 | Amino Acid | Ty1-like NLS Q1JL34-0 |
| 189 | Amino Acid | Ty1-like NLS Q1LI28-0 |
| 190 | Amino Acid | Ty1-like NLS Q2L2H3-0 |
| 191 | Amino Acid | Ty1-like NLS Q2NIH1-0 |
| 192 | Amino Acid | Ty1-like NLS Q2SU23-0 |
| 193 | Amino Acid | Ty1-like NLS Q39KH1-0 |
| 194 | Amino Acid | Ty1-like NLS Q3JMQ8-0 |
| 195 | Amino Acid | Ty1-like NLS Q3YRL8-0 |
| 196 | Amino Acid | Ty1-like NLS Q46WD9-0 |
| 197 | Amino Acid | Ty1-like NLS Q48SQ4-0 |
| 198 | Amino Acid | Ty1-like NLS Q49418-0 |
| 199 | Amino Acid | Ty1-like NLS Q56307-0 |
| 200 | Amino Acid | Ty1-like NLS Q5LEQ4-0 |
| 201 | Amino Acid | Ty1-like NLS Q5WEJ7-0 |
| 202 | Amino Acid | Ty1-like NLS Q5XBA0-0 |
| 203 | Amino Acid | Ty1-like NLS Q62GK1-0 |
| 204 | Amino Acid | Ty1-like NLS Q63Q07-0 |
| 205 | Amino Acid | Ty1-like NLS Q64VP0-0 |
| 206 | Amino Acid | Ty1-like NLS Q6G3V1-0 |
| 207 | Amino Acid | Ty1-like NLS Q6G5M0-0 |
| 208 | Amino Acid | Ty1-like NLS Q6LLQ8-0 |
| 209 | Amino Acid | Ty1-like NLS Q6MDC1-0 |
| 210 | Amino Acid | Ty1-like NLS Q6MDH4-0 |
| 211 | Amino Acid | Ty1-like NLS Q6ME08-0 |
| 212 | Amino Acid | Ty1-like NLS Q73PH4-0 |
| 213 | Amino Acid | Ty1-like NLS Q7MAD1-0 |
| 214 | Amino Acid | Ty1-like NLS Q7UP72-0 |
| 215 | Amino Acid | Ty1-like NLS Q7VTD6-0 |
| 216 | Amino Acid | Ty1-like NLS Q7W2F9-0 |
| 217 | Amino Acid | Ty1-like NLS Q7WRC8-0 |
| 218 | Amino Acid | Ty1-like NLS Q828D0-0 |
| 219 | Amino Acid | Ty1-like NLS Q895M9-0 |
| 220 | Amino Acid | Ty1-like NLS Q8AAP0-0 |
| 221 | Amino Acid | Ty1-like NLS Q8D1X2-0 |
| 222 | Amino Acid | Ty1-like NLS Q8K908-0 |
| 223 | Amino Acid | Ty1-like NLS Q8P0C9-0 |
| 224 | Amino Acid | Ty1-like NLS Q8XKR1-0 |
| 225 | Amino Acid | Ty1-like NLS Q8XL46-0 |
| 226 | Amino Acid | Ty1-like NLS Q8XV09-0 |
| 227 | Amino Acid | Ty1-like NLS Q93Q47-0 |
| 228 | Amino Acid | Ty1-like NLS Q9L0Q6-0 |
| 229 | Amino Acid | Ty1-like NLS Q9L0Q6-1 |
| 230 | Amino Acid | Ty1-like NLS Q9L0Q6-2 |
| 231 | Amino Acid | Ty1-like NLS Q9L0Q6-3 |
| 232 | Amino Acid | Ty1-like NLS Q9L0Q6-4 |
| 233 | Amino Acid | Ty1-like NLS Q9L0Q6-5 |
| 234 | Amino Acid | Ty1-like NLS Q9L0Q6-6 |
| 235 | Amino Acid | Ty1-like NLS Q9X1S8-0 |
| 236 | Amino Acid | Ty1-like NLS A1CNV8-0 |
| 237 | Amino Acid | Ty1-like NLS A1D1R8-0 |
| 238 | Amino Acid | Ty1-like NLS A1D731-0 |
| 239 | Amino Acid | Ty1-like NLS A2QAX7-0 |
| 240 | Amino Acid | Ty1-like NLS A3LQ55-0 |
| 241 | Amino Acid | Ty1-like NLS A5DGY0-0 |
| 242 | Amino Acid | Ty1-like NLS A5DKW3-0 |
| 243 | Amino Acid | Ty1-like NLS A5DLG8-0 |
| 244 | Amino Acid | Ty1-like NLS A5DY34-0 |
| 245 | Amino Acid | Ty1-like NLS A6RBB0-0 |
| 246 | Amino Acid | Ty1-like NLS A6RMZ2-0 |
| 247 | Amino Acid | Ty1-like NLS A6ZL85-0 |
| 248 | Amino Acid | Ty1-like NLS A6ZZJ1-0 |
| 249 | Amino Acid | Ty1-like NLS A7E4K0-0 |
| 250 | Amino Acid | Ty1-like NLS G0S8I1-0 |
| 251 | Amino Acid | Ty1-like NLS O13527-0 |
| 252 | Amino Acid | Ty1-like NLS O13535-0 |
| 253 | Amino Acid | Ty1-like NLS O13658-0 |
| 254 | Amino Acid | Ty1-like NLS O14064-0 |
| 255 | Amino Acid | Ty1-like NLS O14076-0 |
| 256 | Amino Acid | Ty1-like NLS O42668-0 |
| 257 | Amino Acid | Ty1-like NLS O43068-0 |
| 258 | Amino Acid | Ty1-like NLS O74777-0 |
| 259 | Amino Acid | Ty1-like NLS O74862-0 |
| 260 | Amino Acid | Ty1-like NLS O94383-0 |
| 261 | Amino Acid | Ty1-like NLS O94487-0 |
| 262 | Amino Acid | Ty1-like NLS O94585-0 |
| 263 | Amino Acid | Ty1-like NLS O94652-0 |
| 264 | Amino Acid | Ty1-like NLS P0C2I2-0 |
| 265 | Amino Acid | Ty1-like NLS P0C2I3-0 |
| 266 | Amino Acid | Ty1-like NLS P0C2I5-0 |
| 267 | Amino Acid | Ty1-like NLS P0C2I6-0 |
| 268 | Amino Acid | Ty1-like NLS P0C2I7-0 |
| 269 | Amino Acid | Ty1-like NLS P0C2I9-0 |
| 270 | Amino Acid | Ty1-like NLS P0C2J0-0 |
| 271 | Amino Acid | Ty1-like NLS P0C2J1-0 |
| 272 | Amino Acid | Ty1-like NLS P0C2J3-0 |
| 273 | Amino Acid | Ty1-like NLS P0C2J5-0 |
| 274 | Amino Acid | Ty1-like NLS P0CM98-0 |
| 275 | Amino Acid | Ty1-like NLS P0CM99-0 |
| 276 | Amino Acid | Ty1-like NLS P0CX63-0 |
| 277 | Amino Acid | Ty1-like NLS P0CX64-0 |
| 278 | Amino Acid | Ty1-like NLS P13902-0 |
| 279 | Amino Acid | Ty1-like NLS P14746-0 |
| 280 | Amino Acid | Ty1-like NLS P20484-0 |
| 281 | Amino Acid | Ty1-like NLS P22936-0 |
| 282 | Amino Acid | Ty1-like NLS P25384-0 |
| 283 | Amino Acid | Ty1-like NLS P32597-0 |
| 284 | Amino Acid | Ty1-like NLS P36006-0 |
| 285 | Amino Acid | Ty1-like NLS P36080-0 |
| 286 | Amino Acid | Ty1-like NLS P38112-0 |
| 287 | Amino Acid | Ty1-like NLS P47098-0 |
| 288 | Amino Acid | Ty1-like NLS P47100-0 |
| 289 | Amino Acid | Ty1-like NLS P51599-0 |
| 290 | Amino Acid | Ty1-like NLS P53119-0 |
| 291 | Amino Acid | Ty1-like NLS P53123-0 |
| 292 | Amino Acid | Ty1-like NLS P53125-0 |
| 293 | Amino Acid | Ty1-like NLS Q01301-0 |
| 294 | Amino Acid | Ty1-like NLS Q03434-0 |
| 295 | Amino Acid | Ty1-like NLS Q03494-0 |
| 296 | Amino Acid | Ty1-like NLS Q03612-0 |
| 297 | Amino Acid | Ty1-like NLS Q03619-0 |
| 298 | Amino Acid | Ty1-like NLS Q03707-0 |
| 299 | Amino Acid | Ty1-like NLS Q03855-0 |
| 300 | Amino Acid | Ty1-like NLS Q04214-0 |
| 301 | Amino Acid | Ty1-like NLS Q04500-0 |
| 302 | Amino Acid | Ty1-like NLS Q04670-0 |
| 303 | Amino Acid | Ty1-like NLS Q04711-0 |
| 304 | Amino Acid | Ty1-like NLS Q06132-0 |
| 305 | Amino Acid | Ty1-like NLS Q07163-0 |
| 306 | Amino Acid | Ty1-like NLS Q07509-0 |
| 307 | Amino Acid | Ty1-like NLS Q07791-0 |
| 308 | Amino Acid | Ty1-like NLS Q07793-0 |
| 309 | Amino Acid | Ty1-like NLS Q09094-0 |
| 310 | Amino Acid | Ty1-like NLS Q09180-0 |
| 311 | Amino Acid | Ty1-like NLS Q09180-1 |
| 312 | Amino Acid | Ty1-like NLS Q09180-2 |
| 313 | Amino Acid | Ty1-like NLS Q09863-0 |
| 314 | Amino Acid | Ty1-like NLS Q0U8V9-0 |
| 315 | Amino Acid | Ty1-like NLS Q12088-0 |
| 316 | Amino Acid | Ty1-like NLS Q12112-0 |
| 317 | Amino Acid | Ty1-like NLS Q12113-0 |
| 318 | Amino Acid | Ty1-like NLS Q12141-0 |
| 319 | Amino Acid | Ty1-like NLS Q12193-0 |
| 320 | Amino Acid | Ty1-like NLS Q12269-0 |
| 321 | Amino Acid | Ty1-like NLS Q12273-0 |
| 322 | Amino Acid | Ty1-like NLS Q12316-0 |
| 323 | Amino Acid | Ty1-like NLS Q12337-0 |
| 324 | Amino Acid | Ty1-like NLS Q12339-0 |
| 325 | Amino Acid | Ty1-like NLS Q12414-0 |
| 326 | Amino Acid | Ty1-like NLS Q12472-0 |
| 327 | Amino Acid | Ty1-like NLS Q12490-0 |
| 328 | Amino Acid | Ty1-like NLS Q12491-0 |
| 329 | Amino Acid | Ty1-like NLS Q12501-0 |
| 330 | Amino Acid | Ty1-like NLS Q1DNW5-0 |
| 331 | Amino Acid | Ty1-like NLS Q1EA54-0 |
| 332 | Amino Acid | Ty1-like NLS Q2HFA6-0 |
| 333 | Amino Acid | Ty1-like NLS Q2HFA6-1 |
| 334 | Amino Acid | Ty1-like NLS Q2UQI6-0 |
| 335 | Amino Acid | Ty1-like NLS Q4HZ42-0 |
| 336 | Amino Acid | Ty1-like NLS Q4P6I3-0 |
| 337 | Amino Acid | Ty1-like NLS Q4WHF8-0 |
| 338 | Amino Acid | Ty1-like NLS Q4WRV2-0 |
| 339 | Amino Acid | Ty1-like NLS Q4WXQ7-0 |
| 340 | Amino Acid | Ty1-like NLS Q5A2K0-0 |
| 341 | Amino Acid | Ty1-like NLS Q5A310-0 |
| 342 | Amino Acid | Ty1-like NLS Q5ACW8-0 |
| 343 | Amino Acid | Ty1-like NLS Q5B6K3-0 |
| 344 | Amino Acid | Ty1-like NLS Q6BXL7-0 |
| 345 | Amino Acid | Ty1-like NLS Q6C1L3-0 |
| 346 | Amino Acid | Ty1-like NLS Q6C233-0 |
| 347 | Amino Acid | Ty1-like NLS Q6C2J1-0 |
| 348 | Amino Acid | Ty1-like NLS Q6C7C0-0 |
| 349 | Amino Acid | Ty1-like NLS Q6CJY0-0 |
| 350 | Amino Acid | Ty1-like NLS Q6CJY0-1 |
| 351 | Amino Acid | Ty1-like NLS Q6FML5-0 |
| 352 | Amino Acid | Ty1-like NLS Q75F02-0 |
| 353 | Amino Acid | Ty1-like NLS Q7S2A9-0 |
| 354 | Amino Acid | Ty1-like NLS Q7S9J4-0 |
| 355 | Amino Acid | Ty1-like NLS Q7SFJ3-0 |
| 356 | Amino Acid | Ty1-like NLS Q875K1-0 |
| 357 | Amino Acid | Ty1-like NLS Q8SUT1-0 |
| 358 | Amino Acid | Ty1-like NLS Q8SVI7-0 |
| 359 | Amino Acid | Ty1-like NLS Q8SVI7-1 |
| 360 | Amino Acid | Ty1-like NLS Q92393-0 |
| 361 | Amino Acid | Ty1-like NLS Q99109-0 |
| 362 | Amino Acid | Ty1-like NLS Q99231-0 |
| 363 | Amino Acid | Ty1-like NLS Q99337-0 |
| 364 | Amino Acid | Ty1-like NLS Q9USK2-0 |
| 365 | Amino Acid | Ty1-like NLS Q9UTQ5-0 |
| 366 | Amino Acid | Ty1-like NLS A7MD48-0 |
| 367 | Amino Acid | Ty1-like NLS O15446-0 |
| 368 | Amino Acid | Ty1-like NLS O15446-1 |
| 369 | Amino Acid | Ty1-like NLS O15446-2 |
| 370 | Amino Acid | Ty1-like NLS O43148-0 |
| 371 | Amino Acid | Ty1-like NLS O60271-0 |
| 372 | Amino Acid | Ty1-like NLS O75128-0 |
| 373 | Amino Acid | Ty1-like NLS O75400-0 |
| 374 | Amino Acid | Ty1-like NLS O75691-0 |
| 375 | Amino Acid | Ty1-like NLS O75937-0 |
| 376 | Amino Acid | Ty1-like NLS O76021-0 |
| 377 | Amino Acid | Ty1-like NLS O94964-0 |
| 378 | Amino Acid | Ty1-like NLS P23497-0 |
| 379 | Amino Acid | Ty1-like NLS P30414-0 |
| 380 | Amino Acid | Ty1-like NLS P42081-0 |
| 381 | Amino Acid | Ty1-like NLS P46100-0 |
| 382 | Amino Acid | Ty1-like NLS P51608-0 |
| 383 | Amino Acid | Ty1-like NLS P59797-0 |
| 384 | Amino Acid | Ty1-like NLS P82979-0 |
| 385 | Amino Acid | Ty1-like NLS Q12830-0 |
| 386 | Amino Acid | Ty1-like NLS Q13409-0 |
| 387 | Amino Acid | Ty1-like NLS Q13427-0 |
| 388 | Amino Acid | Ty1-like NLS Q15361-0 |
| 389 | Amino Acid | Ty1-like NLS Q15361-1 |
| 390 | Amino Acid | Ty1-like NLS Q53SF7-0 |
| 391 | Amino Acid | Ty1-like NLS Q5M9Q1-0 |
| 392 | Amino Acid | Ty1-like NLS Q5T3I0-0 |
| 393 | Amino Acid | Ty1-like NLS Q5T3I0-1 |
| 394 | Amino Acid | Ty1-like NLS Q68D10-0 |
| 395 | Amino Acid | Ty1-like NLS Q6IPR3-0 |
| 396 | Amino Acid | Ty1-like NLS Q6PD62-0 |
| 397 | Amino Acid | Ty1-like NLS Q6PD62-1 |
| 398 | Amino Acid | Ty1-like NLS Q6PD62-2 |
| 399 | Amino Acid | Ty1-like NLS Q6S8J7-0 |
| 400 | Amino Acid | Ty1-like NLS Q6ZU65-0 |
| 401 | Amino Acid | Ty1-like NLS Q7Z7B0-0 |
| 402 | Amino Acid | Ty1-like NLS Q8N9E0-0 |
| 403 | Amino Acid | Ty1-like NLS Q8NCU4-0 |
| 404 | Amino Acid | Ty1-like NLS Q8NFU7-0 |
| 405 | Amino Acid | Ty1-like NLS Q96DY2-0 |
| 406 | Amino Acid | Ty1-like NLS Q96GD3-0 |
| 407 | Amino Acid | Ty1-like NLS Q96P65-0 |
| 408 | Amino Acid | Ty1-like NLS Q96QC0-0 |
| 409 | Amino Acid | Ty1-like NLS Q9BQG0-0 |
| 410 | Amino Acid | Ty1-like NLS Q9BQG0-1 |
| 411 | Amino Acid | Ty1-like NLS Q9BRU9-0 |
| 412 | Amino Acid | Ty1-like NLS Q9H0S4-0 |
| 413 | Amino Acid | Ty1-like NLS Q9H6F5-0 |
| 414 | Amino Acid | Ty1-like NLS Q9HCK1-0 |
| 415 | Amino Acid | Ty1-like NLS Q9HCK8-0 |
| 416 | Amino Acid | Ty1-like NLS Q9NPI1-0 |
| 417 | Amino Acid | Ty1-like NLS Q9NSV4-0 |
| 418 | Amino Acid | Ty1-like NLS Q9NUL3-0 |
| 419 | Amino Acid | Ty1-like NLS Q9NWT1-0 |
| 420 | Amino Acid | Ty1-like NLS Q9NX58-0 |
| 421 | Amino Acid | Ty1-like NLS Q9UGU5-0 |
| 422 | Amino Acid | Ty1-like NLS Q9UNS1-0 |
| 423 | Amino Acid | Ty1-like NLS Q9Y2X3-0 |
| 424 | Amino Acid | Ty1-like NLS Q9Y6X0-0 |
| 425 | Amino Acid | Ty1-like NLS A0A1I8M2I8-0 |
| 426 | Amino Acid | Ty1-like NLS A1XDC0-0 |
| 427 | Amino Acid | Ty1-like NLS A7S6A5-0 |
| 428 | Amino Acid | Ty1-like NLS A8XI07-0 |
| 429 | Amino Acid | Ty1-like NLS A8XI07-1 |
| 430 | Amino Acid | Ty1-like NLS C0HKU9-0 |
| 431 | Amino Acid | Ty1-like NLS C6KTD2-0 |
| 432 | Amino Acid | Ty1-like NLS O16140-0 |
| 433 | Amino Acid | Ty1-like NLS O17828-0 |
| 434 | Amino Acid | Ty1-like NLS O17966-0 |
| 435 | Amino Acid | Ty1-like NLS O44410-0 |
| 436 | Amino Acid | Ty1-like NLS O44410-1 |
| 437 | Amino Acid | Ty1-like NLS O45244-0 |
| 438 | Amino Acid | Ty1-like NLS P0DP78-0 |
| 439 | Amino Acid | Ty1-like NLS P0DP78-1 |
| 440 | Amino Acid | Ty1-like NLS P0DP79-0 |
| 441 | Amino Acid | Ty1-like NLS P0DP79-1 |
| 442 | Amino Acid | Ty1-like NLS P0DP80-0 |
| 443 | Amino Acid | Ty1-like NLS P0DP80-1 |
| 444 | Amino Acid | Ty1-like NLS P0DP81-0 |
| 445 | Amino Acid | Ty1-like NLS P0DP81-1 |
| 446 | Amino Acid | Ty1-like NLS P14196-0 |
| 447 | Amino Acid | Ty1-like NLS P22058-0 |
| 448 | Amino Acid | Ty1-like NLS P26023-0 |
| 449 | Amino Acid | Ty1-like NLS P26991-0 |
| 450 | Amino Acid | Ty1-like NLS P35978-0 |
| 451 | Amino Acid | Ty1-like NLS P46758-0 |
| 452 | Amino Acid | Ty1-like NLS P46758-1 |
| 453 | Amino Acid | Ty1-like NLS P46867-0 |
| 454 | Amino Acid | Ty1-like NLS P54644-0 |
| 455 | Amino Acid | Ty1-like NLS P54812-0 |
| 456 | Amino Acid | Ty1-like NLS P83212-0 |
| 457 | Amino Acid | Ty1-like NLS Q04621-0 |
| 458 | Amino Acid | Ty1-like NLS Q08696-0 |
| 459 | Amino Acid | Ty1-like NLS Q08696-1 |
| 460 | Amino Acid | Ty1-like NLS Q08696-2 |
| 461 | Amino Acid | Ty1-like NLS Q08696-3 |
| 462 | Amino Acid | Ty1-like NLS Q08696-4 |
| 463 | Amino Acid | Ty1-like NLS Q08696-5 |
| 464 | Amino Acid | Ty1-like NLS Q08696-6 |
| 465 | Amino Acid | Ty1-like NLS Q09223-0 |
| 466 | Amino Acid | Ty1-like NLS Q09595-0 |
| 467 | Amino Acid | Ty1-like NLS Q1ELU8-0 |
| 468 | Amino Acid | Ty1-like NLS Q23120-0 |
| 469 | Amino Acid | Ty1-like NLS Q23272-0 |
| 470 | Amino Acid | Ty1-like NLS Q24537-0 |
| 471 | Amino Acid | Ty1-like NLS Q27450-0 |
| 472 | Amino Acid | Ty1-like NLS Q29DY1-0 |
| 473 | Amino Acid | Ty1-like NLS Q4N4T9-0 |
| 474 | Amino Acid | Ty1-like NLS Q54QQ2-0 |
| 475 | Amino Acid | Ty1-like NLS Q54QQ2-1 |
| 476 | Amino Acid | Ty1-like NLS Q54S20-0 |
| 477 | Amino Acid | Ty1-like NLS Q54US6-0 |
| 478 | Amino Acid | Ty1-like NLS Q54VU4-0 |
| 479 | Amino Acid | Ty1-like NLS Q54XP6-0 |
| 480 | Amino Acid | Ty1-like NLS Q551H0-0 |
| 481 | Amino Acid | Ty1-like NLS Q557G1-0 |
| 482 | Amino Acid | Ty1-like NLS Q55CE0-0 |
| 483 | Amino Acid | Ty1-like NLS Q61R02-0 |
| 484 | Amino Acid | Ty1-like NLS Q75JP5-0 |
| 485 | Amino Acid | Ty1-like NLS Q8I5P7-0 |
| 486 | Amino Acid | Ty1-like NLS Q8I5P7-1 |
| 487 | Amino Acid | Ty1-like NLS Q8IBP1-0 |
| 488 | Amino Acid | Ty1-like NLS Q8ILR9-0 |
| 489 | Amino Acid | Ty1-like NLS Q93591-0 |
| 490 | Amino Acid | Ty1-like NLS Q95Y36-0 |
| 491 | Amino Acid | Ty1-like NLS Q9NBL2-0 |
| 492 | Amino Acid | Ty1-like NLS Q9NDE8-0 |
| 493 | Amino Acid | Ty1-like NLS Q9NDE8-1 |
| 494 | Amino Acid | Ty1-like NLS Q9NDE8-2 |
| 495 | Amino Acid | Ty1-like NLS Q9V5P6-0 |
| 496 | Amino Acid | Ty1-like NLS Q9VDS6-0 |
| 497 | Amino Acid | Ty1-like NLS Q9VGW1-0 |
| 498 | Amino Acid | Ty1-like NLS Q9VH89-0 |
| 499 | Amino Acid | Ty1-like NLS Q9VKM6-0 |
| 500 | Amino Acid | Ty1-like NLS Q9VNH1-0 |
| 501 | Amino Acid | Ty1-like NLS Q9W261-0 |
| 502 | Amino Acid | Ty1-like NLS E1B7L7-0 |
| 503 | Amino Acid | Ty1-like NLS Q08DU1-0 |
| 504 | Amino Acid | Ty1-like NLS Q0III3-0 |
| 505 | Amino Acid | Ty1-like NLS Q17QH9-0 |
| 506 | Amino Acid | Ty1-like NLS Q29S22-0 |
| 507 | Amino Acid | Ty1-like NLS Q2KIQ2-0 |
| 508 | Amino Acid | Ty1-like NLS Q2KJE1-0 |
| 509 | Amino Acid | Ty1-like NLS Q2KJE1-1 |
| 510 | Amino Acid | Ty1-like NLS Q2TBX7-0 |
| 511 | Amino Acid | Ty1-like NLS Q4R7K1-0 |
| 512 | Amino Acid | Ty1-like NLS Q4R8Y5-0 |
| 513 | Amino Acid | Ty1-like NLS Q58DE2-0 |
| 514 | Amino Acid | Ty1-like NLS Q58DU0-0 |
| 515 | Amino Acid | Ty1-like NLS Q5E9U4-0 |
| 516 | Amino Acid | Ty1-like NLS Q5NVM2-0 |
| 517 | Amino Acid | Ty1-like NLS Q5R4V4-0 |
| 518 | Amino Acid | Ty1-like NLS Q5R8B0-0 |
| 519 | Amino Acid | Ty1-like NLS Q5RB69-0 |
| 520 | Amino Acid | Ty1-like NLS Q5RCE6-0 |
| 521 | Amino Acid | Ty1-like NLS Q5TM61-0 |
| 522 | Amino Acid | Ty1-like NLS Q767K9-0 |
| 523 | Amino Acid | Ty1-like NLS Q7YQM3-0 |
| 524 | Amino Acid | Ty1-like NLS Q7YQM4-0 |
| 525 | Amino Acid | Ty1-like NLS Q7YR38-0 |
| 526 | Amino Acid | Ty1-like NLS Q95KD7-0 |
| 527 | Amino Acid | Ty1-like NLS Q95LG8-0 |
| 528 | Amino Acid | Ty1-like NLS Q9N1Q7-0 |
| 529 | Amino Acid | Ty1-like NLS A2WSD3-0 |
| 530 | Amino Acid | Ty1-like NLS A2XVF7-0 |
| 531 | Amino Acid | Ty1-like NLS A2XVF7-1 |
| 532 | Amino Acid | Ty1-like NLS A2XVF7-2 |
| 533 | Amino Acid | Ty1-like NLS A2XVF7-3 |
| 534 | Amino Acid | Ty1-like NLS A3AVH5-0 |
| 535 | Amino Acid | Ty1-like NLS A3AVH5-1 |
| 536 | Amino Acid | Ty1-like NLS A3AVH5-2 |
| 537 | Amino Acid | Ty1-like NLS A3AVH5-3 |
| 538 | Amino Acid | Ty1-like NLS A4QJZ0-0 |
| 539 | Amino Acid | Ty1-like NLS A4QK78-0 |
| 540 | Amino Acid | Ty1-like NLS A4QKG5-0 |
| 541 | Amino Acid | Ty1-like NLS A4QKQ3-0 |
| 542 | Amino Acid | Ty1-like NLS A6MN03-0 |
| 543 | Amino Acid | Ty1-like NLS A8MS85-0 |
| 544 | Amino Acid | Ty1-like NLS A9XMT3-0 |
| 545 | Amino Acid | Ty1-like NLS B8YIE8-0 |
| 546 | Amino Acid | Ty1-like NLS F4HVZ5-0 |
| 547 | Amino Acid | Ty1-like NLS F4IQK5-0 |
| 548 | Amino Acid | Ty1-like NLS F4IQK5-1 |
| 549 | Amino Acid | Ty1-like NLS O22812-0 |
| 550 | Amino Acid | Ty1-like NLS O49323-0 |
| 551 | Amino Acid | Ty1-like NLS O64571-0 |
| 552 | Amino Acid | Ty1-like NLS O64639-0 |
| 553 | Amino Acid | Ty1-like NLS O64639-1 |
| 554 | Amino Acid | Ty1-like NLS O64639-2 |
| 555 | Amino Acid | Ty1-like NLS O65743-0 |
| 556 | Amino Acid | Ty1-like NLS O81072-0 |
| 557 | Amino Acid | Ty1-like NLS P09975-0 |
| 558 | Amino Acid | Ty1-like NLS P0C262-0 |
| 559 | Amino Acid | Ty1-like NLS P29345-0 |
| 560 | Amino Acid | Ty1-like NLS P50888-0 |
| 561 | Amino Acid | Ty1-like NLS P51269-0 |
| 562 | Amino Acid | Ty1-like NLS P51430-0 |
| 563 | Amino Acid | Ty1-like NLS Q06FP6-0 |
| 564 | Amino Acid | Ty1-like NLS Q06FP6-1 |
| 565 | Amino Acid | Ty1-like NLS Q06FP6-2 |
| 566 | Amino Acid | Ty1-like NLS Q06R72-0 |
| 567 | Amino Acid | Ty1-like NLS Q06R98-0 |
| 568 | Amino Acid | Ty1-like NLS Q1KVQ9-0 |
| 569 | Amino Acid | Ty1-like NLS Q1XDL7-0 |
| 570 | Amino Acid | Ty1-like NLS Q38873-0 |
| 571 | Amino Acid | Ty1-like NLS Q3E8X3-0 |
| 572 | Amino Acid | Ty1-like NLS Q3ZJ77-0 |
| 573 | Amino Acid | Ty1-like NLS Q42438-0 |
| 574 | Amino Acid | Ty1-like NLS Q4V3E0-0 |
| 575 | Amino Acid | Ty1-like NLS Q66GN2-0 |
| 576 | Amino Acid | Ty1-like NLS Q6K5K2-0 |
| 577 | Amino Acid | Ty1-like NLS Q6YS30-0 |
| 578 | Amino Acid | Ty1-like NLS Q84WK0-0 |
| 579 | Amino Acid | Ty1-like NLS Q84Y18-0 |
| 580 | Amino Acid | Ty1-like NLS Q8H991-0 |
| 581 | Amino Acid | Ty1-like NLS Q8RWY7-0 |
| 582 | Amino Acid | Ty1-like NLS Q8RWY7-1 |
| 583 | Amino Acid | Ty1-like NLS Q8VZ67-0 |
| 584 | Amino Acid | Ty1-like NLS Q8VZN4-0 |
| 585 | Amino Acid | Ty1-like NLS Q8W0K2-0 |
| 586 | Amino Acid | Ty1-like NLS Q8W490-0 |
| 587 | Amino Acid | Ty1-like NLS Q9CAE4-0 |
| 588 | Amino Acid | Ty1-like NLS Q9FMZ4-0 |
| 589 | Amino Acid | Ty1-like NLS Q9FMZ4-1 |
| 590 | Amino Acid | Ty1-like NLS Q9FRI0-0 |
| 591 | Amino Acid | Ty1-like NLS Q9LKI5-0 |
| 592 | Amino Acid | Ty1-like NLS Q9LUJ5-0 |
| 593 | Amino Acid | Ty1-like NLS Q9LUR0-0 |
| 594 | Amino Acid | Ty1-like NLS Q9LVU8-0 |
| 595 | Amino Acid | Ty1-like NLS Q9LVU8-1 |
| 596 | Amino Acid | Ty1-like NLS Q9LYK7-0 |
| 597 | Amino Acid | Ty1-like NLS Q9M020-0 |
| 598 | Amino Acid | Ty1-like NLS Q9M1L7-0 |
| 599 | Amino Acid | Ty1-like NLS Q9M3V8-0 |
| 600 | Amino Acid | Ty1-like NLS Q9SRQ3-0 |
| 601 | Amino Acid | Ty1-like NLS Q9ZPV5-0 |
| 602 | Amino Acid | Ty1-like NLS B1AQJ2-0 |
| 603 | Amino Acid | Ty1-like NLS D3ZUI5-0 |
| 604 | Amino Acid | Ty1-like NLS D4A666-0 |
| 605 | Amino Acid | Ty1-like NLS E1U8D0-0 |
| 606 | Amino Acid | Ty1-like NLS G3V8T1-0 |
| 607 | Amino Acid | Ty1-like NLS O35821-0 |
| 608 | Amino Acid | Ty1-like NLS O88487-0 |
| 609 | Amino Acid | Ty1-like NLS O88665-0 |
| 610 | Amino Acid | Ty1-like NLS P61364-0 |
| 611 | Amino Acid | Ty1-like NLS P61365-0 |
| 612 | Amino Acid | Ty1-like NLS P83858-0 |
| 613 | Amino Acid | Ty1-like NLS P83861-0 |
| 614 | Amino Acid | Ty1-like NLS Q00566-0 |
| 615 | Amino Acid | Ty1-like NLS Q05CL8-0 |
| 616 | Amino Acid | Ty1-like NLS Q09XV5-0 |
| 617 | Amino Acid | Ty1-like NLS Q3TFK5-0 |
| 618 | Amino Acid | Ty1-like NLS Q3TFK5-1 |
| 619 | Amino Acid | Ty1-like NLS Q3TFK5-2 |
| 620 | Amino Acid | Ty1-like NLS Q3TYA6-0 |
| 621 | Amino Acid | Ty1-like NLS Q3UMF0-0 |
| 622 | Amino Acid | Ty1-like NLS Q498U4-0 |
| 623 | Amino Acid | Ty1-like NLS Q4V7C4-0 |
| 624 | Amino Acid | Ty1-like NLS Q4V8G7-0 |
| 625 | Amino Acid | Ty1-like NLS Q505I5-0 |
| 626 | Amino Acid | Ty1-like NLS Q562C7-0 |
| 627 | Amino Acid | Ty1-like NLS Q566R3-0 |
| 628 | Amino Acid | Ty1-like NLS Q566R3-1 |
| 629 | Amino Acid | Ty1-like NLS Q566R3-2 |
| 630 | Amino Acid | Ty1-like NLS Q58A65-0 |
| 631 | Amino Acid | Ty1-like NLS Q5NBX1-0 |
| 632 | Amino Acid | Ty1-like NLS Q5XG71-0 |
| 633 | Amino Acid | Ty1-like NLS Q5XI01-0 |
| 634 | Amino Acid | Ty1-like NLS Q5XIB5-0 |
| 635 | Amino Acid | Ty1-like NLS Q5XIR6-0 |
| 636 | Amino Acid | Ty1-like NLS Q60848-0 |
| 637 | Amino Acid | Ty1-like NLS Q62018-0 |
| 638 | Amino Acid | Ty1-like NLS Q62018-1 |
| 639 | Amino Acid | Ty1-like NLS Q62187-0 |
| 640 | Amino Acid | Ty1-like NLS Q62871-0 |
| 641 | Amino Acid | Ty1-like NLS Q63520-0 |
| 642 | Amino Acid | Ty1-like NLS Q642C0-0 |
| 643 | Amino Acid | Ty1-like NLS Q68SB1-0 |
| 644 | Amino Acid | Ty1-like NLS Q6AYK5-0 |
| 645 | Amino Acid | Ty1-like NLS Q6NZB0-0 |
| 646 | Amino Acid | Ty1-like NLS Q76KJ5-0 |
| 647 | Amino Acid | Ty1-like NLS Q76KJ5-1 |
| 648 | Amino Acid | Ty1-like NLS Q76KJ5-2 |
| 649 | Amino Acid | Ty1-like NLS Q78WZ7-0 |
| 650 | Amino Acid | Ty1-like NLS Q78WZ7-1 |
| 651 | Amino Acid | Ty1-like NLS Q7TNB4-0 |
| 652 | Amino Acid | Ty1-like NLS Q7TPV4-0 |
| 653 | Amino Acid | Ty1-like NLS Q80WC1-0 |
| 654 | Amino Acid | Ty1-like NLS Q80Z37-0 |
| 655 | Amino Acid | Ty1-like NLS Q811R2-0 |
| 656 | Amino Acid | Ty1-like NLS Q8BKA3-0 |
| 657 | Amino Acid | Ty1-like NLS Q8CJ67-0 |
| 658 | Amino Acid | Ty1-like NLS Q8K214-0 |
| 659 | Amino Acid | Ty1-like NLS Q8K4T4-0 |
| 660 | Amino Acid | Ty1-like NLS Q8R5F3-0 |
| 661 | Amino Acid | Ty1-like NLS Q91X13-0 |
| 662 | Amino Acid | Ty1-like NLS Q9CS72-0 |
| 663 | Amino Acid | Ty1-like NLS Q9CVI2-0 |
| 664 | Amino Acid | Ty1-like NLS Q9CWX9-0 |
| 665 | Amino Acid | Ty1-like NLS Q9CZX5-0 |
| 666 | Amino Acid | Ty1-like NLS Q9D1J3-0 |
| 667 | Amino Acid | Ty1-like NLS Q9D3V1-0 |
| 668 | Amino Acid | Ty1-like NLS Q9DBQ9-0 |
| 669 | Amino Acid | Ty1-like NLS Q9JIX5-0 |
| 670 | Amino Acid | Ty1-like NLS Q9JJ80-0 |
| 671 | Amino Acid | Ty1-like NLS Q9JJ89-0 |
| 672 | Amino Acid | Ty1-like NLS Q9R1C7-0 |
| 673 | Amino Acid | Ty1-like NLS Q9R1X4-0 |
| 674 | Amino Acid | Ty1-like NLS Q9Z180-0 |
| 675 | Amino Acid | Ty1-like NLS Q9Z207-0 |
| 676 | Amino Acid | Ty1-like NLS Q9Z2D6-0 |
| 677 | Amino Acid | Ty1-like NLS A0A1L8GSA2-0 |
| 678 | Amino Acid | Ty1-like NLS A0JP82-0 |
| 679 | Amino Acid | Ty1-like NLS A1A5I1-0 |
| 680 | Amino Acid | Ty1-like NLS A1L2T6-0 |
| 681 | Amino Acid | Ty1-like NLS A2RUV0-0 |
| 682 | Amino Acid | Ty1-like NLS A9JRD8-0 |
| 683 | Amino Acid | Ty1-like NLS E7F568-0 |
| 684 | Amino Acid | Ty1-like NLS F1QFU0-0 |
| 685 | Amino Acid | Ty1-like NLS F1QWK4-0 |
| 686 | Amino Acid | Ty1-like NLS K9JHZ4-0 |
| 687 | Amino Acid | Ty1-like NLS P07193-0 |
| 688 | Amino Acid | Ty1-like NLS P0CB65-0 |
| 689 | Amino Acid | Ty1-like NLS P12957-0 |
| 690 | Amino Acid | Ty1-like NLS P13505-0 |
| 691 | Amino Acid | Ty1-like NLS P21783-0 |
| 692 | Amino Acid | Ty1-like NLS Q28BS0-0 |
| 693 | Amino Acid | Ty1-like NLS Q28BS0-1 |
| 694 | Amino Acid | Ty1-like NLS Q28G05-0 |
| 695 | Amino Acid | Ty1-like NLS Q32N87-0 |
| 696 | Amino Acid | Ty1-like NLS Q3KPW4-0 |
| 697 | Amino Acid | Ty1-like NLS Q4QR29-0 |
| 698 | Amino Acid | Ty1-like NLS Q4QR29-1 |
| 699 | Amino Acid | Ty1-like NLS Q5BL56-0 |
| 700 | Amino Acid | Ty1-like NLS Q5XJK9-0 |
| 701 | Amino Acid | Ty1-like NLS Q5ZIJ0-0 |
| 702 | Amino Acid | Ty1-like NLS Q640I9-0 |
| 703 | Amino Acid | Ty1-like NLS Q6DEU9-0 |
| 704 | Amino Acid | Ty1-like NLS Q6DEU9-1 |
| 705 | Amino Acid | Ty1-like NLS Q6DEU9-2 |
| 706 | Amino Acid | Ty1-like NLS Q6DK85-0 |
| 707 | Amino Acid | Ty1-like NLS Q6DRI7-0 |
| 708 | Amino Acid | Ty1-like NLS Q6DRL5-0 |
| 709 | Amino Acid | Ty1-like NLS Q6NV26-0 |
| 710 | Amino Acid | Ty1-like NLS Q6NWI1-0 |
| 711 | Amino Acid | Ty1-like NLS Q6NYJ3-0 |
| 712 | Amino Acid | Ty1-like NLS Q6P4K1-0 |
| 713 | Amino Acid | Ty1-like NLS Q6WKW9-0 |
| 714 | Amino Acid | Ty1-like NLS Q7ZUF2-0 |
| 715 | Amino Acid | Ty1-like NLS Q7ZW47-0 |
| 716 | Amino Acid | Ty1-like NLS Q7ZXZ0-0 |
| 717 | Amino Acid | Ty1-like NLS Q7ZXZ0-1 |
| 718 | Amino Acid | Ty1-like NLS Q7ZYR8-0 |
| 719 | Amino Acid | Ty1-like NLS Q8AVQ6-0 |
| 720 | Amino Acid | Ty1-like NLS Q9DE07-0 |
| 721 | Amino Acid | Ty1-like NLS P03086-0 |
| 722 | Amino Acid | Ty1-like NLS P09814-0 |
| 723 | Amino Acid | Ty1-like NLS P0CK10-0 |
| 724 | Amino Acid | Ty1-like NLS P15075-0 |
| 725 | Amino Acid | Ty1-like NLS P51724-0 |
| 726 | Amino Acid | Ty1-like NLS P52344-0 |
| 727 | Amino Acid | Ty1-like NLS P52531-0 |
| 728 | Amino Acid | Ty1-like NLS Q5UP41-0 |
| 729 | Amino Acid | Ty1-like NLS Q9DUC0-0 |
| 730 | Amino Acid | Ty1-like NLS Q9XJS3-0 |
| 731 | Amino Acid | eraseR (3xFLAG-Ty1NLS-PspCas13b) |
| 732 | Amino Acid | CRISPRase - 3xFLAG-Ty1NLS-dPspCas13b-linker-RNase |
| 733 | Amino Acid | short CRISPRase - 3xFLAG-Ty1NLS-dPspCas13b-linker-RNase |
| 734 | Nucleic Acid | PspCas13b |
| 735 | Nucleic Acid | PspCas13b Truncation |
| 736 | Nucleic Acid | dPspCas13b |
| 737 | Nucleic Acid | dPspCas13b truncation |
| 738 | Nucleic Acid | Rnase 1 (human) |
| 739 | Nucleic Acid | Rnase 2 (human) |
| 740 | Nucleic Acid | Rnase 3 (human) |
| 741 | Nucleic Acid | Rnase 4 (human) |
| 742 | Nucleic Acid | Rnase 5 (human) |
| 743 | Nucleic Acid | Rnase 6 (human) |
| 744 | Nucleic Acid | Rnase 7 (human) |
| 745 | Nucleic Acid | Rnase 8 (human) |
| 746 | Nucleic Acid | Synthetic Tandem Dimer RNase 1 (Human, tdRNase 1) |
| 747 | Nucleic Acid | RNase A (Bovine RNase1) |
| 748 | Nucleic Acid | RNase 1 (E. coli, ecRNase 1) |
| 749 | Nucleic Acid | RNase 1B (P. nemaeus) |
| 750 | Nucleic Acid | txRNase 1 (RNase 1 R39D/N67D/N88A/G89D/R91D) |
| 751 | Nucleic Acid | txRNase A (RNase A D38R/R39D/N67R/G88R) |
| 752 | Nucleic Acid | RNase T1 (A. flavus) |
| 753 | Nucleic Acid | RNase T1, rA cleaving variant No.1 |
| 754 | Nucleic Acid | RNase T1, rA cleaving variant No.2 |
| 755 | Nucleic Acid | RNase T1, rA cleaving variant RNase T1-R2 |
| 756 | Nucleic Acid | Alpha Sarcin (A. giganteus) |
| 757 | Nucleic Acid | RNase T1, dsRNA cleaving variant RNase Ta |
| 758 | Nucleic Acid | BARNASE |
| 759 | Nucleic Acid | BARNASE + chimeric intron |
| 760 | Nucleic Acid | RNASE A, 3x S-peptide-P2A-S-protein |
| 761 | Nucleic Acid | RNase LE |
| 762 | Nucleic Acid | Rnase T2 (human) |
| 763 | Nucleic Acid | Onconase (R. pipiens) |
| 764 | Nucleic Acid | Erns(C171R) (RNase T2, Bovine viral diarrhea virus) |
| 765 | Nucleic Acid | RNase U2 (U. sphaerogena) |
| 766 | Nucleic Acid | PIN RNase domain (Human SMG6) |
| 767 | Nucleic Acid | Synthetic tandem PIN RNase domain (Human SMG6, tdPIN) |
| 768 | Nucleic Acid | Bovine seminal ribonuclease (Bovind, SRN) |
| 769 | Nucleic Acid | Synthetic Tandem Dimer Bovine seminal ribonuclease (Bovind, SRN) |
| 770 | Nucleic Acid | RNase V1 |
| 771 | Nucleic Acid | Mini RNase III (B. subtilis, MiniR3) |
| 772 | Nucleic Acid | Synthetic Tandem Dimer Mini RNase III (B. subtilis, tdMiniR3) |
| 773 | Nucleic Acid | RNase III Domain (Mouse, a.a.1648-1846, DICER) |
| 774 | Nucleic Acid | Synthetic Tandem Dimer RNase III Domain (Mouse, tdDICER) |
| 775 | Nucleic Acid | Natural tandem RNase III domain (Human DROSHA a.a. 925-1246, DROSHA) |
| 776 | Nucleic Acid | Natural tandem RNase III domain Dimer (G. intestinalis DICER, a.a. 292-754 |
| | | giDICER) |
| 777 | Nucleic Acid | Synthetic tandem RNase III domain (E. coli RNC, tdRNC) |
| 778 | Nucleic Acid | Ribonuclease HI (Sulfurisphaera tokodaii, RNase HI*) |
| 779 | Nucleic Acid | Ribonuclease HI(D125N)(Sulfurisphaera tokodaii, RNase HI*D125N) |
| 780 | Nucleic Acid | S-protein (RNase 1, Human) |
| 781 | Nucleic Acid | S-peptide (RNase 1, Human) |
| 782 | Nucleic Acid | S-peptide-linker-ERT2 (RNase 1, Human) |
| 783 | Nucleic Acid | S-protein (RNase 1B, P. nemaeus) |
| 784 | Nucleic Acid | S-peptide (RNase 1B, P. nemoeus) |
| 785 | Nucleic Acid | S-peptide-linker-ERT2 (RNase 1B, P. nemaeus) |
| 786 | Nucleic Acid | S-protein (RNase A, Bovine) |
| 787 | Nucleic Acid | S-peptide (RNase A, Bovine) |
| 788 | Nucleic Acid | S-peptide-linker-ERT2 (RNase A, Bovine) |
| 789 | Nucleic Acid | 3x S-peptide (RNASE A) |
| 790 | Nucleic Acid | Linker sequence 1 |
| 791 | Nucleic Acid | Linker sequence 2 |
| 792 | Nucleic Acid | Linker sequence 3 |
| 793 | Nucleic Acid | 3xFlag |
| 794 | Nucleic Acid | Ty1 NLS |
| 795 | Nucleic Acid | eraseR (3xFLAG-Ty1NLS-PspCas13b) |
| 796 | Nucleic Acid | CRISPRase - 3xFLAG-Ty1NLS-dPspCas13b-linker-RNase |
| 797 | Nucleic Acid | short CRISPRase - 3xFLAG-Ty1NLS-dPspCas13b-linker-RNase |
| 798 | Nucleic Acid | PspCas13b CAG crRNA target sequences |
| 799 | Nucleic Acid | PspCas13b CAG-f2 crRNA target sequences |
| 800 | Nucleic Acid | PspCas13b CAG-f3 crRNA target sequences |
| 801 | Nucleic Acid | PspCas13b LUC crRNA target sequences |
| 802 | Amino Acid | Nuclear Export Signal (NES) 1 |
| 803 | Nucleic Acid | Nuclear Export Signal (NES) 1 |
| 804 | Amino Acid | NES 2 |
| 805 | Nucleic Acid | NES 2 |
| 806 | Amino Acid | N-terminal 1x Mitochondrial Targeting Sequence |
| 807 | Amino Acid | N-terminal 4x Mitochondrial Targeting Sequence |
| 808 | Amino Acid | N-terminal Secretory Signal Sequence (IL-2) |
| 809 | Amino Acid | N-terminal Myristoylation (Membrane Targeting Sequence) |
| 810 | Amino Acid | ER Localization - N-terminal Calsequestrin leader sequence |
| 811 | Amino Acid | ER Localization - C-terminal KDEL retention signal |
| 812 | Amino Acid | C-terminal Peroxisome Targeting Sequence |
| 813 | Nucleic Acid | N-terminal 1x Mitochondrial Targeting Sequence |
| 814 | Nucleic Acid | N-terminal 4x Mitochondrial Targeting Sequence |
| 815 | Nucleic Acid | N-terminal Secretory Signal Sequence (IL-2) |
| 816 | Nucleic Acid | N-terminal Myristoylation (Membrane Targeting Sequence) |
| 817 | Nucleic Acid | ER Localization - N-terminal Calsequestrin leader sequence |
| 818 | Nucleic Acid | ER Localization - C-terminal KDEL retention signal |
| 819 | Nucleic Acid | C-terminal Peroxisome Targeting Sequence |
| 820 | Nucleic Acid | EEF1A2 (EF1A2) promoter −493 to +1261 |
| 821 | Nucleic Acid | EEF1A2 (EF1A2) promoter −493 to +23 |
| 822 | Nucleic Acid | minimal EEF1A2 ((EF1A2) core promoter −221 to +23 |
| 823 | Nucleic Acid | mini-dCas13X.1 |
| 824 | Nucleic Acid | 3xFLAG-Ty1NLS-mini-dCas13X.1-linker-RNase |
| 825 | Nucleic Acid | RNase-linker-3xFLAG-Ty1NLS-mini-dCas13X.1-linker-RNase |
| 826 | Amino Acid | mini-dCas13X.1 |
| 827 | Amino Acid | 3xFLAG-Ty1NLS-mini-dCas13X.1-linker-RNase |
| 828 | Amino Acid | RNase-linker-3xFLAG-Ty1NLS-mini-dCas13X.1-linker-RNase |

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out certain embodiments of the present invention, and are not to be construed as limiting in any way the remainder of the disclosure.

### Example 1: CRISPRase: Targeted RNA Cleavage with dCas13-RNase Fusion Proteins

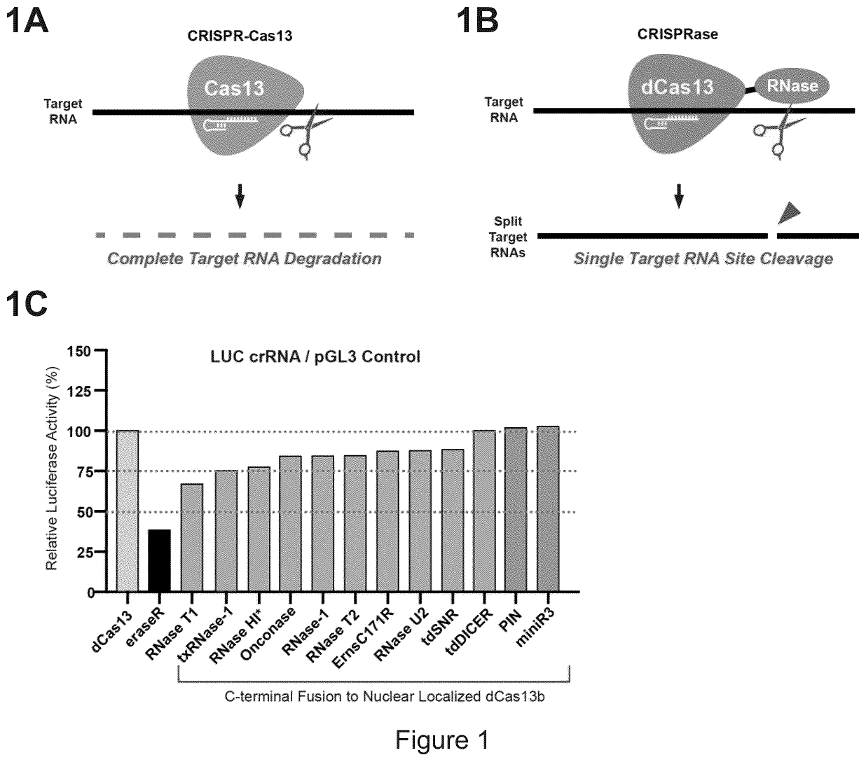

The data presented herein demonstrates the fusion of dCas13 to heterologous RNases for targeted catalytic activity. Remarkably, these data demonstrate that fusion of dCas13 to RNases allows for targeted RNA cleavage. These dCas13-RNase fusion enzymes are termed CRISPRases (FIG. 1B). Since RNase enzymes comprise a large family with diverse substrate and nucleotide specificities, fusions to dCas13 has the potential to allow for new targeted RNA cleavage modalities for both basic research and therapeutic applications.

In general, endoribonucleases are ribonucleases which are capable of cleaving a phosphodiester bond within a polynucleotide chain. RNases are found in all kingdoms of life, as well as infectious viruses, and catalyze diverse biological processes. One remarkable aspect of RNases are their ability to recognize diverse nucleotide substrates, with some specific for single-stranded RNA (ssRNA) (ex. RNase 1b), ssRNA and double-stranded RNA (dsRNA) (ex. RNASE1), dsRNA-specific (ex. RNase III), or specific for RNA in a hybrid RNA:DNA complex (ex. RNase HI). Further, many RNases show sequence specific cleavage, for example, RNase A cleave preferentially at the 3′ end of C and U nucleotides, RNase T1 cleaves at the 3′ end of unpaired G residues, RNase U2 cleaves at the 3′ end of unpaired A residues, etc.

RNases and their catalytic domains are typically small (˜125 amino acids), with some functioning as monomers (RNase T1), and others as homodimers, which allow for binding and cleavage of both strands within dsRNA (RNase1 and RNase III domains). While a few RNase enzymes have been identified which serve intracellular functions (, ex. RNASE2 in the lysosome), most vertebrate RNases encode an N-terminal signal peptide which allows for their extracellular secretion and activity. RNases from the pancreatic RNase family ptRNase, which are among the most well-studied, are secreted and play an important role in RNA digestion.

To determine whether fusion of an RNases to dCas13 would result in targeted RNA cleavage, RNase enzymes with diverse RNA substrate and nucleotide specificities were cloned as fusions to the C-terminal end of dPspCas13b, separated by a long linker sequence. Fusion proteins were co-expressed in mammalian COST cells together with a Luciferase reporter and guide-RNAs targeting either the luciferase coding sequence (Luc crRNA), or with a negative control non-targeting guide-RNA (NC crRNA). Remarkably, many CRISPRases showed specific knockdown of luciferase activity in mammalian cells when targeted with a Luciferase guide-RNA, relative to expression with the non-targeting guide RNA (FIG. 1C). However, some CRISPRases showed little to no activity, which may be due in part to substrate or sequence requirements of the RNases, necessity to homodimerize, requirements for cofactors, such as metal ions or other enzymatic conditions, for example pH, or improper folding. In this regard, among the most potent CRISPRase enzymes at this target site included the fusion to RNase T1, which functions as a monomer and does not require metal ions for activity. Whereas those with little activity were ones predicted to cleave dsRNA substrates and encoded RNase III domains (miniR3 and tdDICER). These data demonstrate that dCas13 fusions to a heterologous RNase enzyme allows for targeted RNA cleavage of an mRNA reporter in mammalian cells. Modifications to both guide RNA and/or CRISPRase fusions proteins may further allow for additional unique RNA cleavage capabilities.

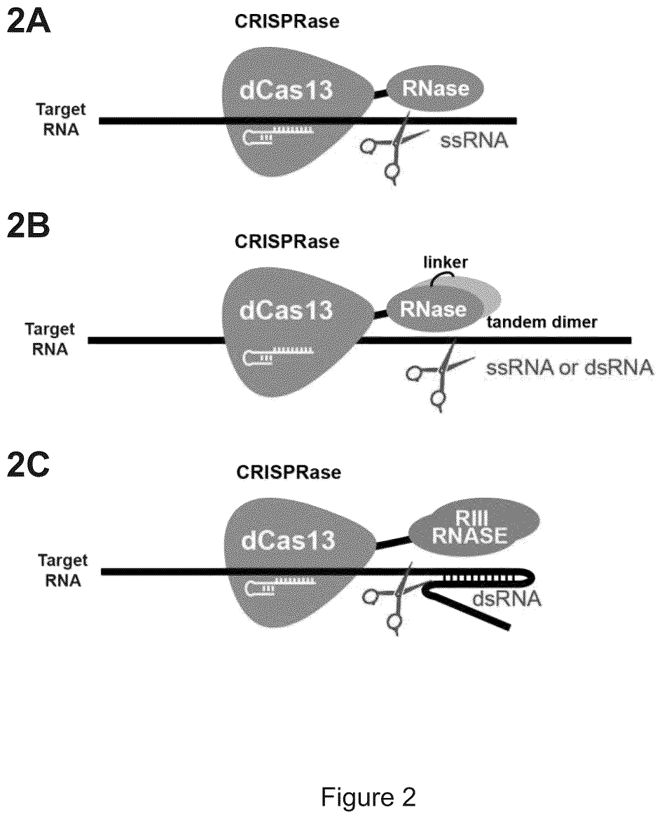

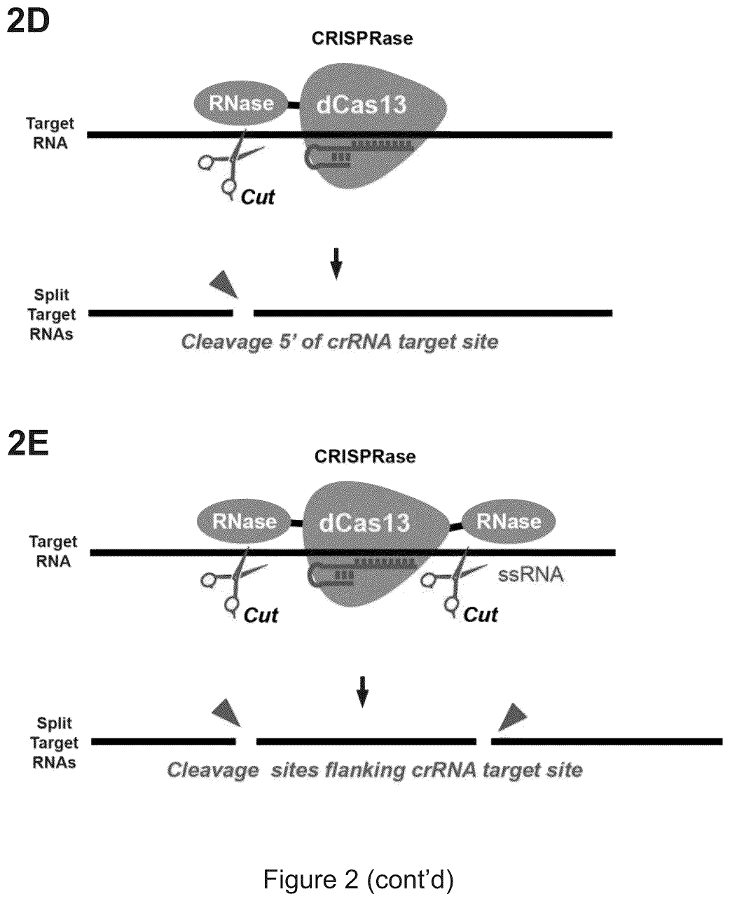

While CRISPR-Cas13 systems show ssRNA specific cleavage, dCas13 fusions to RNases with different substrate specificities may impart their substrate cleavage capabilities. For example, CRISPRases with different substrate cleavage specificities may be generated by fusions to RNases with ssRNA specificity, one with both ssRNA and dsRNA specificity, or dsRNA specificity (FIG. 2A-C). Here, forced tandem dimerization of RNase domains which require dimerization for function may enhance cleavage or promote dsRNA cleavage over ssRNA cleavage. Further, fusion of RNase domains to either N or C-terminal, or both may allow for cleavage at either 5′, 3′ or both 5′ and 3′ locations relative to the guide-RNA target site (FIGS. 2D and E).

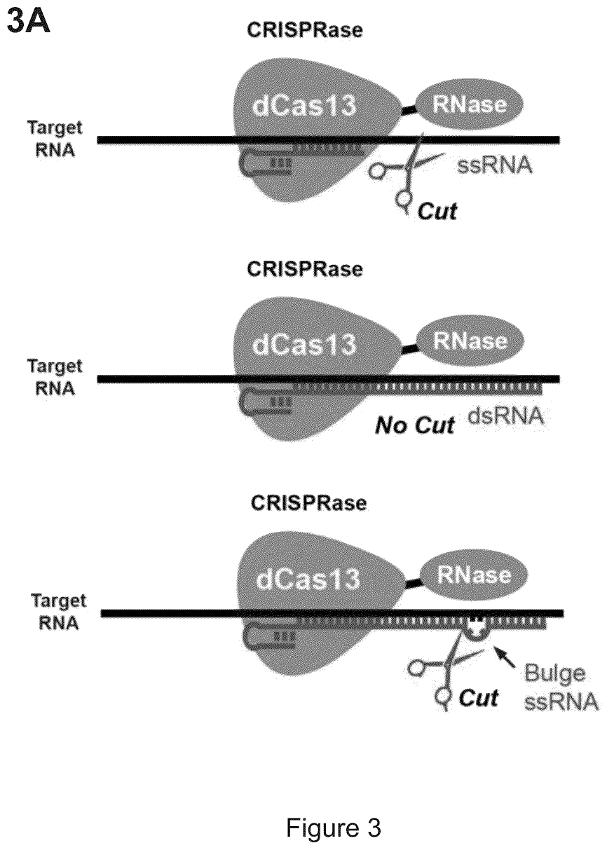

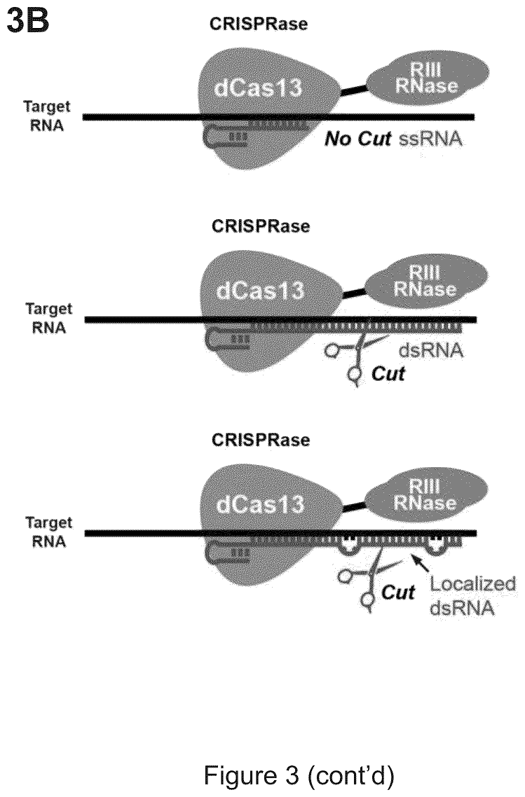

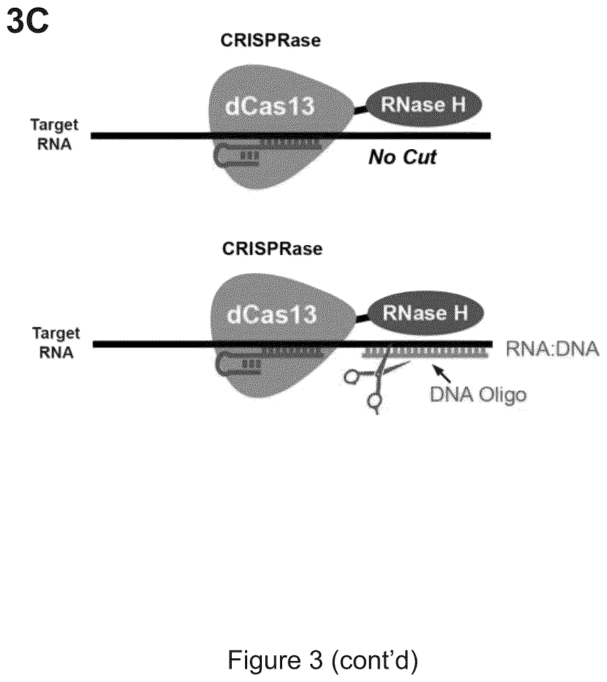

In conjunction with different dCas13-RNase fusion proteins, modifications to the complementary CRISPR guide-RNA may allow for specific RNA substrate cleavage. For example, in FIG. 3A, guide-RNA extensions may block cleavage by ssRNA-specific RNases, or guide-RNAs with an unpaired bulge may allow for precise nucleotide specific substrate cleavage. In contrast, elongated guide-RNAs may enable cleavage by dsRNA-specific RNases, or flanking unpaired bulges could serve to focus dsRNA cleavage (FIG. 3B). Further, fusion of dCas13 with an RNase specific to cleaving RNA within a hybrid DNA:RNA complex may be enabled by delivery of a complementary DNA oligonucleotide (FIG. 3C).

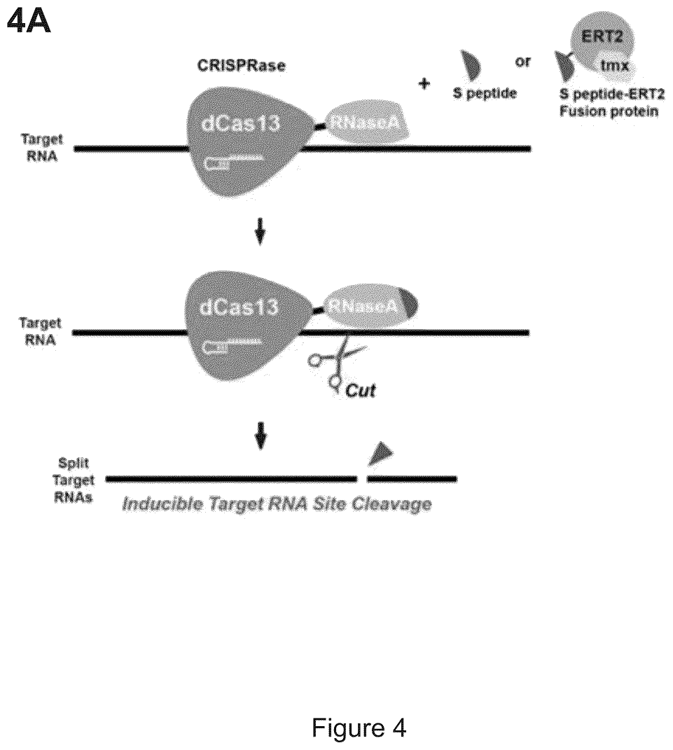

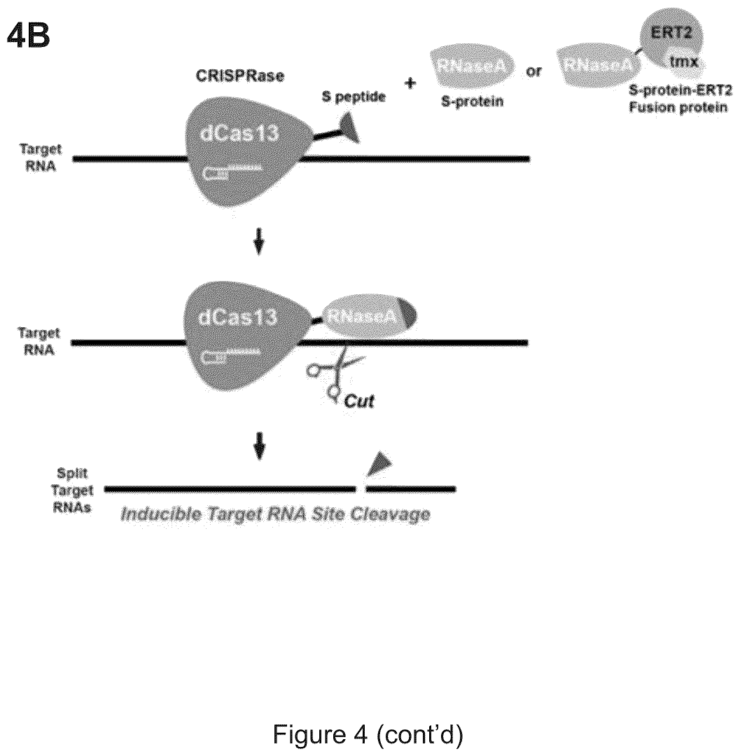

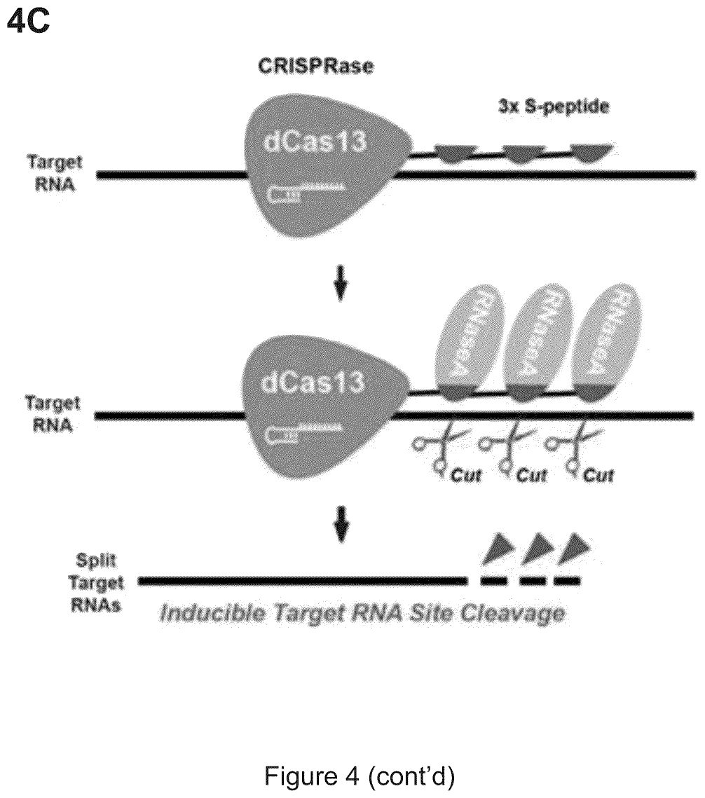

Pancreatic RNases are among the most robust protein structures known, which are well known to survive harsh physiological conditions, including autoclaving. This feature is due in part to the strong interaction between structural residues. Remarkably, RNase1 members of this family are capable of being cleaved into two separate parts (S-peptide and S-protein), which when delivered in trans, regain catalytic activity. Harnessing this strong interaction may allow for inducible RNase catalytic activity, whereby S-protein fused to dCas13, inactive at a target RNA site, can be reactivated by delivery of S-peptide, either alone, or if fused to small-molecule responsive element, such as the tamoxifen (tmx) inducible ERT2 domain (FIG. 4).

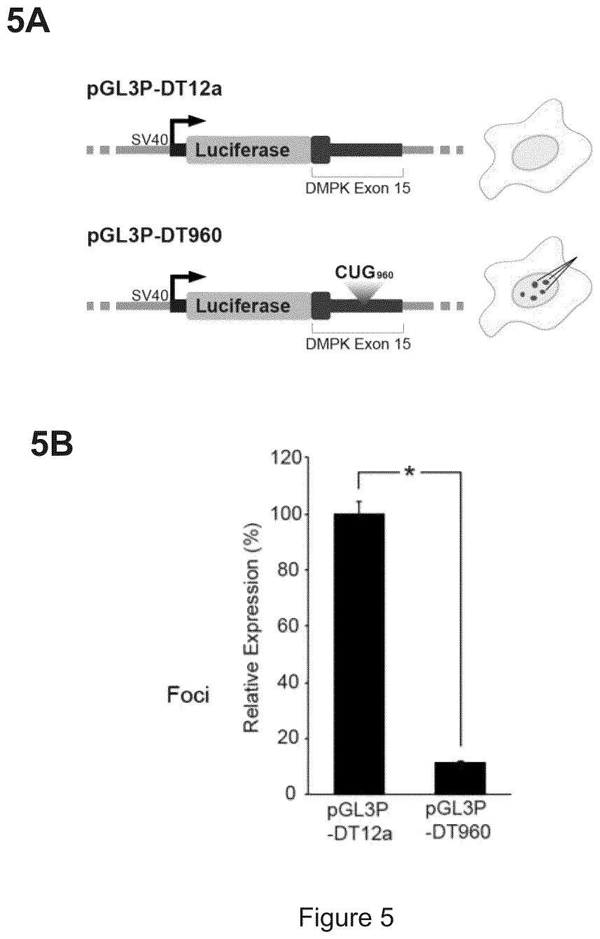

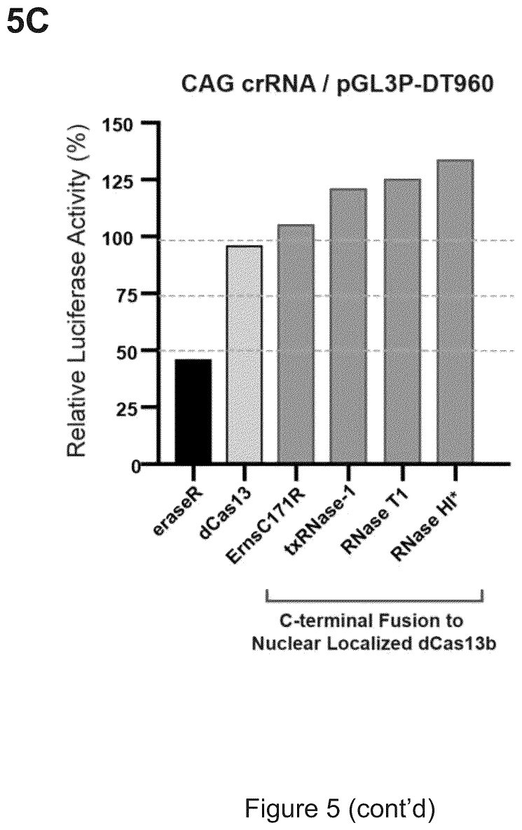

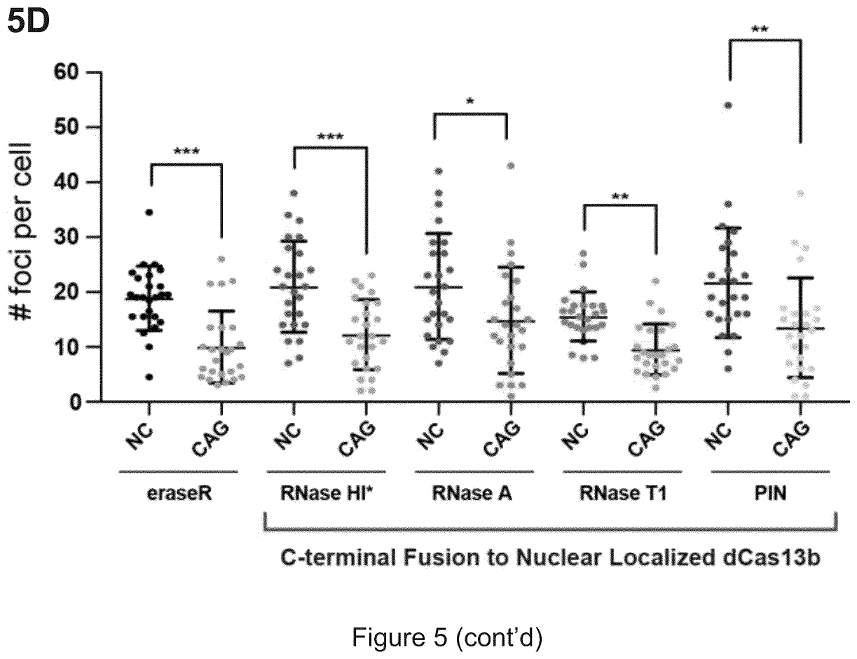

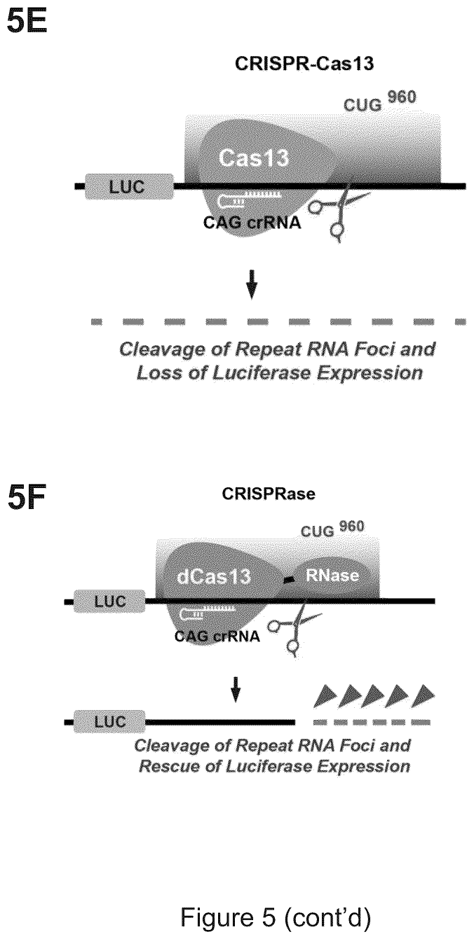

To determine the effects of host gene expression by expression of expanded repeats, a luciferase reporter encoding either 12 or 960 CUG repeats was generated (FIG. 5A). The addition of 960 CUG repeats resulted in significant knockdown of luciferase activity, likely due to sequestration of the reporter RNA within nuclear foci (FIG. 5B). These findings are consistent with the observed decrease in DMPK expression in DM1 patients, despite normal levels of DMPK mRNA. As previously reporter, Cas13 targeted to CUG repeats with a complimentary CAG crRNA resulted in robust decrease in nuclear foci. However, Cas13 also results in decreased luciferase activity, a finding consistent with complete degradation of targeted mRNAs by CRISPR-Cas13 (FIG. 5C-E). Targeting of CRISPRases to repeat foci also resulted in decreased number of foci, however, surprisingly, also resulted in increased luciferase reporter activity (FIGS. 5C and D). These findings are consistent with a model by which CRISPRases induce single site RNA cleavage events, or when targeted to expansion repeats, are capable of cleaving repeat RNA sequences but spare the remainder of the RNA (FIG. 5F). Thus, targeting expansion repeat RNAs with CRISPRases may provide a mechanism for both the elimination of toxic RNA foci and rescue of host gene expression.

### Example 2: Use of CRISPRases for Trans Splicing and Assembly of RNA in Cells and In Vitro

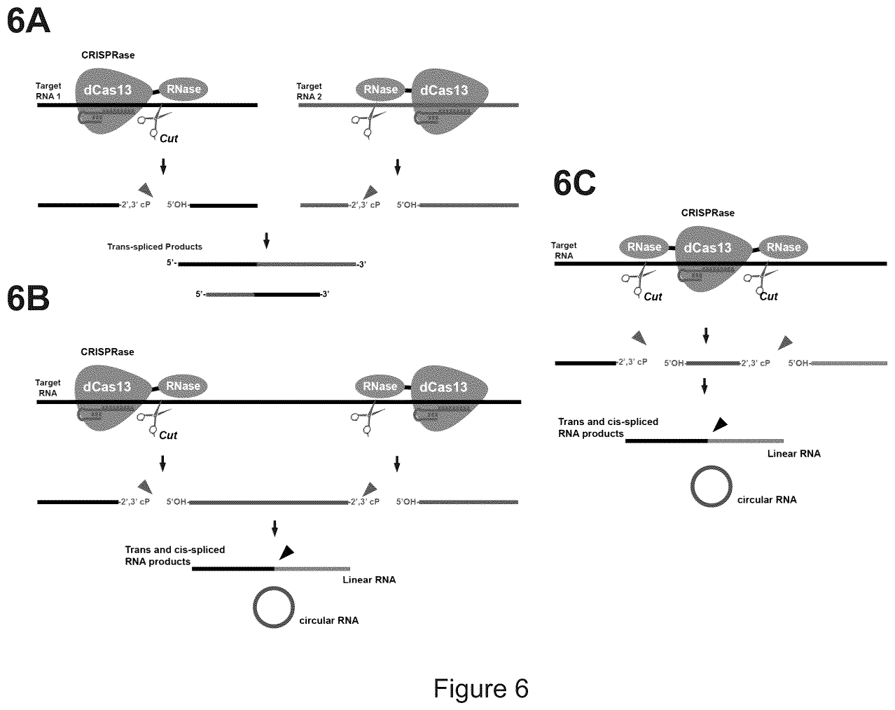

RNAse-mediated RNA cleavage generates 2′,3′ cyclic phosphate and 5′ hydroxyl RNA termini, which mimic those found upon cleavage by autocatalytic RNA sequences, such as ribozymes (Shigematsu M, et al., Front Genet, 2018, 9:562). Based on previous work, it was found that ribozyme-mediated RNA cleavage results in trans-RNA splicing in cells, or in vitro catalyzed by RtcB ligase. Thus, RNAse-cleaved RNAs may also be subject to trans-splicing in cells or in vitro by RtcB. When fused to a sequence specific RNA targeting protein, such as the CRISPRases described herein, targeting of two or more locations may provide a means for trans-splicing of two distinct RNA sequences, or to delete/bypass deleterious sequences within a single RNA sequence (FIG. 6A-C). Targeting of multiple sites could be performed using multiple guide RNAs with an N- or C-terminal RNAse fusion (FIG. 6A-B), or through fusion of multiple RNAses to a single RNA targeting protein (FIG. 6C). Due to the small size of many RNAse sequences, as well the discovery of compact Cas13 orthologs (Xu C, et al., Nat Methods, 2021, 18(5): 499-506), CRISPRases are well within the packaging capacity of many therapeutic viral vectors, notably AAV.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A fusion protein comprising:
a) a CRISPR-associated (Cas) protein; and
b) a RNase protein.

2. The fusion protein of claim 1, wherein the Cas protein is catalytically dead Cas13 (dCas13).

3. The fusion protein of claim 2, wherein dCas13 comprises a sequence selected from SEQ ID NOs: 47-48, or a variant thereof.

4. The fusion protein of claim 1, wherein the RNase comprises a sequence selected from SEQ ID NOs: 49-89, or a variant thereof.

5. The fusion protein of claim 1, wherein the RNase protein is txRNase 1, RNase T1, Ribonuclease HI, PIN RNase, or RNase A.

6. The fusion protein of claim 1, wherein the RNase is a RNase dimer.

7. The fusion protein of claim 6, wherein the RNase dimer comprises a sequence selected from SEQ ID NOs: 57, 77, 79, 82, and 84-87, or a variant thereof.

8. The fusion protein of claim 1, wherein the fusion protein further comprises a nuclear localization signal (NLS).

9. The fusion protein of claim 8, wherein NLS comprises a sequence selected from SEQ ID NOs: 110-730, or a variant thereof.

10. The fusion protein of claim 1, wherein the fusion protein comprises a sequence selected from SEQ ID NOs:732 and 733, and a sequence selected from SEQ ID NOs: 49-89.

11. A nucleic acid molecule comprising a nucleic acid sequence encoding a fusion protein of claim 1.

12. A composition comprising the fusion protein of claim 1 or a nucleic acid molecule encoding the fusion protein

13. The composition of claim 12, wherein the composition further comprises one or more targeting nucleic acid molecules.

14. The composition of claim 13, wherein the targeting nucleic acid molecule is a CRISPR guide RNA (crRNA).

15. The composition of claim 14, wherein the composition further comprises a targeting DNA oligo.

16. A fusion protein comprising:
a) a CRISPR-associated (Cas) protein; and
b) an s-protein.

17. The fusion protein of claim 16, wherein the Cas protein is catalytically dead Cas13 (dCas13).

18. The fusion protein of claim 17, wherein dCas13 comprises a sequence selected from SEQ ID NOs: 47-48, or a variant thereof.

19. The fusion protein of claim 16, wherein the s-protein comprises a sequence selected from SEQ ID NOs: 90, 93, and 96, or a variant thereof.

20. The fusion protein of claim 16, wherein the fusion protein further comprises a nuclear localization signal (NLS)

21. The fusion protein of claim 20, wherein NLS comprises a sequence selected from SEQ ID NOs: 110-730, or a variant thereof.

22. The fusion protein of any of claims 16-21, wherein the fusion protein comprises a sequence selected from SEQ ID NOs:732 and 733, and a sequence selected from SEQ ID NOs: 90, 93, and 96.

23. A nucleic acid molecule comprising a nucleic acid sequence encoding a fusion protein of claim 16.

24. A composition comprising the fusion protein of claim 16 or a nucleic acid molecule encoding the fusion protein.

25. The composition of claim 23, wherein the composition further comprises a s-peptide.

26. The composition of claim 25, wherein the s-peptide comprises a sequence selected from SEQ ID NOs: 83, 84, 86, 87, 89 and 90, or a variant thereof

27. The composition of claim 24, wherein the composition further comprises one or more targeting nucleic acid molecules.

28. The composition of claim 27, wherein the targeting nucleic acid molecule is a CRISPR guide RNA (crRNA).

29. A method of decreasing the number of a RNA transcript or cleaving RNA transcript in a subject, the method comprising administering to the subject:
(a) a composition comprising a fusion protein comprising a CRISPR-associated (Cas) protein; and a RNase protein; or a nucleic acid molecule encoding the fusion protein; or
(b) a composition comprising a fusion protein comprising a CRISPR-associated (Cas) protein; and an s-protein; or a nucleic acid molecule encoding the fusion protein; and
and a guide nucleic acid comprising a sequence complimentary to a target RNA sequence in the RNA transcript.

30. The method of claim 23 being either an in vitro or in vivo method.

31. A method of treating a disease or disorder associated with RNA in a subject, the method comprising administering to the subject:
(a) a composition comprising a fusion protein comprising a CRISPR-associated (Cas) protein; and a RNase protein; or a nucleic acid molecule encoding the fusion protein; or
(b) a composition comprising a fusion protein comprising a CRISPR-associated (Cas) protein; and an s-protein; or a nucleic acid molecule encoding the fusion protein; and a guide nucleic acid comprising a sequence complimentary to a target RNA sequence in the RNA transcript.

32. The method of claim 31, wherein the disease or disorder is Myotonic Dystrophy or an RNA virus infection.
